SEQUENCE LISTING (153 sequences)

SEQ ID NO: 1 - protein, 1400 aa, Homo sapiens
MELLPPLPQSFLLLLLLPAKPAAGEDWQCPRTPYAASRDFDVKYVVPSFSAGGLVQAMVTYEGDRNESAVFVAIRNRLHVLGPDLKSVQSLATGPAGDPGCQTCAACGPGPHGPPGDTDTKVLVLDPALPALVSCGSSLQGRCFLHDLEPQGTAVHLAAPACLFSAHHNRPDDCPDCVASPLGTRVTVVEQGQASYFYVASSLDAAVAASFSPRSVSIRRLKADASGFAPGFVALSVLPKHLVSYSIEYVHSFHTGAFVYFLTVQPASVTDDPSALHTRLARLSATEPELGDYRELVLDCRFAPKRRRRGAPEGGQPYPVLRVAHSAPVGAQLATELSIAEGQEVLFGVFVTGKDGGPGVGPNSVVCAFPIDLLDTLIDEGVERCCESPVHPGLRRGLDFFQSPSFCPNPPGLEALSPNTSCRHFPLLVSSSFSRVDLFNGLLGPVQVTALYVTRLDNVTVAHMGTMDGRILQVELVRSLNYLLYVSNFSLGDSGQPVQRDVSRLGDHLLFASGDQVFQVPIQGPGCRHFLTCGRCLRAWHFMGCGWCGNMCGQQKECPGSWQQDHCPPKLTEFHPHSGPLRGSTRLTLCGSNFYLHPSGLVPEGTHQVTVGQSPCRPLPKDSSKLRPVPRKDFVEEFECELEPLGTQAVGPTNVSLTVTNMPPGKHFRVDGTSVLRGFSFMEPVLIAVQPLFGPRAGGTCLTLEGQSLSVGTSRAVLVNGTECLLARVSEGQLLCATPPGATVASVPLSLQVGGAQVPGSWTFQYREDPVVLSISPNCGYINSHITICGQHLTSAWHLVLSFHDGLRAVESRCERQLPEQQLCRLPEYVVRDPQGWVAGNLSARGDGAAGFTLPGFRFLPPPHPPSANLVPLKPEEHAIKFEYIGLGAVADCVGINVTVGGESCQHEFRGDMVVCPLPPSLQLGQDGAPLQVCVDGECHILGRVVRPGPDGVPQSTLLGILLPLLLLVAALATALVFSYWWRRKQLVLPPNLNDLASLDQTAGATPLPILYSGSDYRSGLALPAIDGLDSTTCVHGASFSDSEDESCVPLLRKESIQLRDLDSALLAEVKDVLIPHERVVTHSDRVIGKGHFGVVYHGEYIDQAQNRIQCAIKSLSRITEMQQVEAFLREGLLMRGLNHPNVLALIGIMLPPEGLPHVLLPYMCHGDLLQFIRSPQRNPTVKDLISFGLQVARGMEYLAEQKFVHRDLAARNCMLDESFTVKVADFGLARDILDREYYSVQQHRHARLPVKWMALESLQTYRFTTKSDVWSFGVLLWELLTRGAPPYRHIDPFDLTHFLAQGRRLPQPEYCPDSLYQVMQQCWEADPAVRPTFRVLVGEVEQIVSALLGDHYVQLPATYMNLGPSTSHEMNVRPEQPQFSPMPGNVRRPRPLSEPPRPT

SEQ ID NO: 2 - protein, 1378 aa, Mus sp.
MGLPLPLLQSSLLLMLLLRLSAASTNLNWQCPRIPYAASRDFSVKYVVPSFSAGGRVQATAAYEDSTNSAVFVATRNHLHVLGPDLQFIENLTTGPIGNPGCQTCASCGPGPHGPPKDTDTLVLVMEPGLPALVSCGSTLQGRCFLHELEPRGKALHLAAPACLFSANNNKPEACTDCVASPLGTRVTVVEQGHASYFYVASSLDPELAASFSPRSVSIRRLKSDTSGFQPGFPSLSVLPKYLASYLIKYVYSFHSGDFVYFLTVQPISVTSPPSALHTRLVRLNAVEPEIGDYRELVLDCHFAPKRRRRGAPEGTQPYPVLQAAHSAPVDAKLAVELSISEGQEVLFGVFVTVKDGGSGMGPNSVVCAFPIYHLNILIEEGVEYCCHSSNSSSLLSRGLDFFQTPSFCPNPPGGEASGPSSRCHYFPLMVHASFTRVDLFNGLLGSVKVTALHVTRLGNVTVAHMGTVDGRVLQVEIARSLNYLLYVSNFSLGSSGQPVHRDVSRLGNDLLFASGDQVFKVPIQGPGCRHFLTCWRCLRAQRFMGCGWCGDRCDRQKECPGSWQQDHCPPEISEFYPHSGPLRGTTRLTLCGSNFYLRPDDVVPEGTHQITVGQSPCRLLPKDSSSPRPGSLKEFIQELECELEPLVTQAVGTTNISLVITNMPAGKHFRVEGISVQEGFSFVEPVLTSIKPDFGPRAGGTYLTLEGQSLSIATSRAALVNGTQCRLEQVNEEQILCVTPPGAGTARVPLHLQIGGAEVPGSWTFHYKEDPIVLDISPKCGYSGSHIMIHGQHLTSAWHFTLSFHDGQSTVESRCAGQFVEQQQRRCRLPEYVVRNPQGWATGNLSVWGDGAAGFTLPGFRFLPPPSPLRAGLVELKPEEHSVKVEYVGLGAVADCVTVNMTVGGEVCQHELRGDVVICPLPPSLQLGKDGVPLQVCVDGGCHILSQVVRSSPGRASQRILLIALLVLILLVAVLAVALIFNSRRRKKQLGAHSLSPTTLSDINDTASGAPNHEESSESRDGTSVPLLRTESIRLQDLDRMLLAEVKDVLIPHEQVVIHTDQVIGKGHFGVVYHGEYTDGAQNQTHCAIKSLSRITEVQEVEAFLREGLLMRGLHHPNILALIGIMLPPEGLPRVLLPYMRHGDLLRFIRSPQRNPTVKDLVSFGLQVACGMEYLAEQKFVHRDLAARNCMLDESFTVKVADFGLARGVLDKEYYSVRQHRHARLPVKWMALESLQTYRFTTKSDVWSFGVLLWELLTRGAPPYPHIDPFDLSHFLAQGRRLPQPEYCPDSLYHVMLRCWEADPAARPTFRALVLEVKQVVASLLGDHYVQLTAAYVNVGPRAVDDGSVPPEQVQPSPQHCRSTSKPRPLSEPPLPT

SEQ ID NO: 3 - DNA, 345 bp, Artificial
  Synthetic VH sequence of M14-H06
gaagttcaattgttagagtctggtggcggtcttgttcagcctggtggttctttacgtctttcttgcgctgcttccggattcactttctctccttacattatgtcttgggttcgccaagctcctggtaaaggtttggagtgggtttcttctatcgtttcttctggtggctggactacttatgctgactccgttaaaggtcgcttcactatctctagagacaactctaagaatactctctacttgcagatgaacagcttaagggctgaggacacagccacatattactgtgcgagaggccgactatttgactactggggccagggaaccctggtcaccgtctcaagc

SEQ ID NO: 4 - protein, 115 aa, Artificial
  Synthetic VH sequence of M14-H06
EVQLLESGGGLVQPGGSLRLSCAASGFTFSPYIMSWVRQAPGKGLEWVSSIVSSGGWTTYADSVKGRFTISRDNSKNTLYLQMNSLRAEDTATYYCARGRLFDYWGQGTLVTVSS

SEQ ID NO: 5 - protein, 5 aa, Artificial
  Synthetic VH CDR1 sequence of M14-H06
PYIMS

SEQ ID NO: 6 - protein, 17 aa, Artificial
  Synthetic VH CDR2 sequence of M14-H06
SIVSSGGWTTYADSVKG

SEQ ID NO: 7 - protein, 6 aa, Artificial
  Synthetic VH CDR3 sequence of M14-H06
GRLFDY

SEQ ID NO: 8 - DNA, 324 bp, Artificial
  Synthetic VL sequence of M14-H06
cagagcgtcttgactcagacaccctcggtgtccgtggcccccggacagacggccaggataacctgtgagggaaacaacattcgaggtcaaagtgtgcattggtaccagcagaagccaggccaggcccctgtagtggtcgtctttgatgacaccgaccggccctcaaggattcctgagcgattctctggctccaagtctgggaacacggccaccctgaccatcatcagggtcgaggccggggatgaggccgactattactgtcaggtgtgggatagttctagtgatcattttgtcttcggaactgggaccacggtcaccgtccta

SEQ ID NO: 9 - protein, 108 aa, Artificial
  Synthetic VL sequence of M14-H06
QSVLTQTPSVSVAPGQTARITCEGNNIRGQSVHWYQQKPGQAPVVVVFDDTDRPSRIPERFSGSKSGNTATLTIIRVEAGDEADYYCQVWDSSSDHFVFGTGTTVTVL

SEQ ID NO: 10 - protein, 11 aa, Artificial
  Synthetic VL CDR1 sequence of M14-H06
EGNNIRGQSVH

SEQ ID NO: 11 - protein, 7 aa, Artificial
  Synthetic VL CDR2 sequence of M14-H06
DDTDRPS

SEQ ID NO: 12 - protein, 11 aa, Artificial
  Synthetic VL CDR3 sequence of M14-H06
QVWDSSSDHFV

SEQ ID NO: 13 - DNA, 363 bp, Artificial
  Synthetic VH sequence of M15-E10
gaagttcaattgttagagtctggtggcggtcttgttcagcctggtggttctttacgtctttcttgcgctgcttccggattcactttctctaagtactttatgatgtgggttcgccaagctcctggtaaaggtttggagtgggtttcttctatctatccttctggtggcgatactacttatgctgactccgttaaaggtcgcttcactatctctagagacaactctaagaatactctctacttgcagatgaacagcttaagggctgaggacacggccgtatattactgtgcccgatggggtatagtgggagttccgaatgcttttgatatctggggccaagggacaatggtcaccgtctcaagc

SEQ ID NO: 14 - protein, 121 aa, Artificial
  Synthetic VH sequence of M15-E10
EVQLLESGGGLVQPGGSLRLSCAASGFTFSKYFMMWVRQAPGKGLEWVSSIYPSGGDTTYADSVKGRFTISRDNSKNTLYLQMNSLRAEDTAVYYCARWGIVGVPNAFDIWGQGTMVTVSS

SEQ ID NO: 15 - protein, 5 aa, Artificial
  Synthetic VH CDR1 sequence of M15-E10
KYFMM

SEQ ID NO: 16 - protein, 17 aa, Artificial
  Synthetic VH CDR2 sequence of M15-E10
SIYPSGGDTTYADSVKG

SEQ ID NO: 17 - protein, 12 aa, Artificial
  Synthetic VH CDR3 sequence of M15-E10
WGIVGVPNAFDI

SEQ ID NO: 18 - DNA, 321 bp, Artificial
  Synthetic VL sequence of M15-E10
gacattcagatgacccagtctccatcttccctgtctgcatctgtaggagacagggtcaccatcacttgccgggcaagtccggacattggcacctatttaaattggtatcaacagaaaccaaggaaagcccctcaactcctgatctatgctgcatccagtttacaaagtggggtcccatcaaggttcagtggcagtggatctgggacagatttcactctcaccatcagcagtctgcaacctgaagattttgcaacttactactgtcaacagagttacactacccctctcaccttcggccaagggacacgactagagcttaaa

SEQ ID NO: 19 - protein, 107 aa, Artificial
  Synthetic VL sequence of M15-E10
DIQMTQSPSSLSASVGDRVTITCRASPDIGTYLNWYQQKPRKAPQLLIYAASSLQSGVPSRFSGSGSGTDFTLTISSLQPEDFATYYCQQSYTTPLTFGQGTRLELK

SEQ ID NO: 20 - protein, 11 aa, Artificial
  Synthetic VL CDR1 sequence of M15-E10
RASPDIGTYLN

SEQ ID NO: 21 - protein, 7 aa, Artificial
  Synthetic VL CDR2 sequence of M15-E10
AASSLQS

SEQ ID NO: 22 - protein, 9 aa, Artificial
  Synthetic VL CDR3 sequence of M15-E10
QQSYTTPLT

SEQ ID NO: 23 - DNA, 363 bp, Artificial
  Synthetic VH sequence of M16-C07
gaagttcaattgttagagtctggtggcggtcttgttcagcctggtggttctttacgtctttcttgcgctgcttccggattcactttctctgattaccagatggagtgggttcgccaagctcctggtaaaggtttggagtgggtttcttatatctattcttctggtggcaagactgtttatgctgactccgttaaaggtcgcttcactatctctagagacaactctaagaatactctctacttgcagatgaacagcttaagggctgaggacacggccgtgtattactgtgcgagacttccgagagtgggagctaccatcataaatgactactggggccagggaaccctggtcaccgtctcaagc

SEQ ID NO: 24 - protein, 121 aa, Artificial
  Synthetic VH sequence of M16-C07
EVQLLESGGGLVQPGGSLRLSCAASGFTFSDYQMEWVRQAPGKGLEWVSYIYSSGGKTVYADSVKGRFTISRDNSKNTLYLQMNSLRAEDTAVYYCARLPRVGATIINDYWGQGTLVTVSS

SEQ ID NO: 25 - protein, 5 aa, Artificial
  Synthetic VH CDR1 sequence of M16-C07
DYQME

SEQ ID NO: 26 - protein, 17 aa, Artificial
  Synthetic VH CDR2 sequence of M16-C07
YIYSSGGKTVYADSVKG

SEQ ID NO: 27 - protein, 12 aa, Artificial
  Synthetic VH CDR3 sequence of M16-C07
LPRVGATIINDY

SEQ ID NO: 28 - DNA, 321 bp, Artificial
  Synthetic VL sequence of M16-C07
gacatccagatgacccagtctccatcttccgtgtctgcatctgtgggagacagagtcaccatcacttgtcgggcgagtcgaggtattagcagctctttagcctggtatcagcagaaaccagggagagcccctaagctcctaatctatgctgcatccagtttccatactggggtcccgtcaaggttcagcggcagtggatctgtgacagaattcactctcaccatcagcagcctgcagcctgaagactttgctacttactattgtcaacagacggacagttttccgctcaccttcggcggagggaccaaggtggaaatcaaa

SEQ ID NO: 29 - protein, 107 aa, Artificial
  Synthetic VL sequence of M16-C07
DIQMTQSPSSVSASVGDRVTITCRASRGISSSLAWYQQKPGRAPKLLIYAASSFHTGVPSRFSGSGSVTEFTLTISSLQPEDFATYYCQQTDSFPLTFGGGTKVEIK

SEQ ID NO: 30 - protein, 11 aa, Artificial
  Synthetic VL CDR1 sequence of M16-C07
RASRGISSSLA

SEQ ID NO: 31 - protein, 7 aa, Artificial
  Synthetic VL CDR2 sequence of M16-C07
AASSFHT

SEQ ID NO: 32 - protein, 9 aa, Artificial
  Synthetic VL CDR3 sequence of M16-C07
QQTDSFPLT

SEQ ID NO: 33 - DNA, 348 bp, Artificial
  Synthetic VH sequence of M23-F10
gaagttcaattgttagagtctggtggcggtcttgttcagcctggtggttctttacgtctttcttgcgctgcttccggattcactttctctaattacatgatgggttgggttcgccaagctcctggtaaaggtttggagtgggtttcttctatctatttttctggtggcaagacttattatgctgactccgttaaaggtcgcttcactatctctagagacaactctaagaatactctctacttgcagatgaacagcttaagggctgaggacacggccgtgtattactgtgcgagagggtacagctggttcgacccctggggccagggcaccctggtcaccgtctcaagc

SEQ ID NO: 34 - protein, 116 aa, Artificial
  Synthetic VH sequence of M23-F10
EVQLLESGGGLVQPGGSLRLSCAASGFTFSNYMMGWVRQAPGKGLEWVSSIYFSGGKTYYADSVKGRFTISRDNSKNTLYLQMNSLRAEDTAVYYCARGYSWFDPWGQGTLVTVSS

SEQ ID NO: 35 - protein, 5 aa, Artificial
  Synthetic VH CDR1 sequence of M23-F10
NYMMG

SEQ ID NO: 36 - protein, 17 aa, Artificial
  Synthetic VH CDR2 sequence of M23-F10
SIYFSGGKTYYADSVKG

SEQ ID NO: 37 - protein, 7 aa, Artificial
  Synthetic VH CDR3 sequence of M23-F10
GYSWFDP

SEQ ID NO: 38 - DNA, 321 bp, Artificial
  Synthetic VL sequence of M23-F10
gacatccagatgacccagtctccatcttccgtgtctgcatctgtaggagacagagtcaccatcacttgtcgggcgagtcagggtattagcagctggttagcctggtatcagcagaaaccagggaaagcccctaagctcctgatctatgctgcatccagtttgcaaagtggggtcccatcaaggttcagcggcagtggatctgggacagatttcactctcaccatcagcagcctgcagcctgaagattttgcaacttactattgtcaacaggctaacagtttcccccggacgttcggccaagggaccaaggtggaaatcaaa

SEQ ID NO: 39 - protein, 107 aa, Artificial
  Synthetic VL sequence of M23-F10
DIQMTQSPSSVSASVGDRVTITCRASQGISSWLAWYQQKPGKAPKLLIYAASSLQSGVPSRFSGSGSGTDFTLTISSLQPEDFATYYCQQANSFPRTFGQGTKVEIK

SEQ ID NO: 40 - protein, 11 aa, Artificial
  Synthetic VL CDR1 sequence of M23-F10
RASQGISSWLA

SEQ ID NO: 41 - protein, 7 aa, Artificial
  Synthetic VL CDR2 sequence of M23-F10
AASSLQS

SEQ ID NO: 42 - protein, 9 aa, Artificial
  Synthetic VL CDR3 sequence of M23-F10
QQANSFPRT

SEQ ID NO: 43 - DNA, 369 bp, Artificial
  Synthetic VH sequence of M80-B03
gaagttcaattgttagagtctggtggcggtcttgttcagcctggtggttctttacgtctttcttgcgctgcttccggattcactttctcttggtaccgtatgacttgggttcgccaagctcctggtaaaggtttggagtgggtttcttatatctcttcttctggtggctggactcattatgctgactccgttaaaggtcgcttcactatctctagagacaactctaagaatactctctacttgcagatgaacagcttaagggctgaggacacggctgtgtattactgtgcgagccctcttggcttctacgagcgagtcgcttatgcttttgatatctggggccaagggacaatggtcaccgtctcaagc

SEQ ID NO: 44 - protein, 123 aa, Artificial
  Synthetic VH sequence of M80-B03
EVQLLESGGGLVQPGGSLRLSCAASGFTFSWYRMTWVRQAPGKGLEWVSYISSSGGWTHYADSVKGRFTISRDNSKNTLYLQMNSLRAEDTAVYYCASPLGFYERVAYAFDIWGQGTMVTVSS

SEQ ID NO: 45 - protein, 5 aa, Artificial
  Synthetic VH CDR1 sequence of M80-B03
WYRMT

SEQ ID NO: 46 - protein, 17 aa, Artificial
  Synthetic VH CDR2 sequence of M80-B03
YISSSGGWTHYADSVKG

SEQ ID NO: 47 - protein, 14 aa, Artificial
  Synthetic VH CDR3 sequence of M80-B03
PLGFYERVAYAFDI

SEQ ID NO: 48 - DNA, 324 bp, Artificial
  Synthetic VL sequence of M80-B03
gacatccagatgacccagtctccatcctccctgtctgcatctgtgggagacagagtcgccatcacttgccgcgcaagtcagagcatcgacacctatttaaattggtatcagcagaaaccagggaaagcccctaaactcctgatctatgctgcatccaagttggaagatggggtcccatcaagattcagtggcagtggaactgggacagatttcactctcaccatcagaagtctgcaacctgaagattttgcaagttatttctgtcaacagagctactctagtccagggatcactttcggccctgggaccaaggtggagatcaaa

SEQ ID NO: 49 - protein, 108 aa, Artificial
  Synthetic VL sequence of M80-B03
DIQMTQSPSSLSASVGDRVAITCRASQSIDTYLNWYQQKPGKAPKLLIYAASKLEDGVPSRFSGSGTGTDFTLTIRSLQPEDFASYFCQQSYSSPGITFGPGTKVEIK

SEQ ID NO: 50 - protein, 11 aa, Artificial
  Synthetic VL CDR1 sequence of M80-B03
RASQSIDTYLN

SEQ ID NO: 51 - protein, 7 aa, Artificial
  Synthetic VL CDR2 sequence of M80-B03
AASKLED

SEQ ID NO: 52 - protein, 10 aa, Artificial
  Synthetic VL CDR3 sequence of M80-B03
QQSYSSPGIT

SEQ ID NO: 53 - DNA, 405 bp, Artificial
  Synthetic VH sequence of 1P3B2
atgggttggatctgtatctttctcttcctcgtgtcagtaactacaggtgtccactctgaggtccagctgcagcagtctggacctgagctggagaagcctggcgcttcagtgaaaatatcctgcaaggcttctggttactcattcactggctacaacatgaactgggtgaagcagagcaatggagagagccttgagtggattggagatattgatccttactatggtggtactaggtacaaccagaagttcaagggcaaggccacattgactgtagacaaatcctccagcacagcctacatgcaactcaagagcctgacatctgaggactctgcagtctattactgtgcaagagaggggaggggttttgcttactggggccaagggactctggtcactgtctctgca

SEQ ID NO: 54 - protein, 116 aa, Artificial
  Synthetic VH sequence of 1P3B2
EVQLQQSGPELEKPGASVKISCKASGYSFTGYNMNWVKQSNGESLEWIGDIDPYYGGTRYNQKFKGKATLTVDKSSSTAYMQLKSLTSEDSAVYYCAREGRGFAYWGQGTLVTVSA

SEQ ID NO: 55 - protein, 5 aa, Artificial
  Synthetic VH CDR1 sequence of 1P3B2
GYNMN

SEQ ID NO: 56 - protein, 17 aa, Artificial
  Synthetic VH CDR2 sequence of 1P3B2
DIDPYYGGTRYNQKFKG

SEQ ID NO: 57 - protein, 7 aa, Artificial
  Synthetic VH CDR3 sequence of 1P3B2
EGRGFAY

SEQ ID NO: 58 - DNA, 387 bp, Artificial
  Synthetic VL sequence of 1P3B2
atgagggcccctgctcagttccttgggttgctgctgctgtggcttacaggtgccagatgtgacatccagatgactcagtctccagcctccctatctgcatctgtgggagaaactgtcaccatcacatgtcgagcaagtgagaatatttacagttatttagcatggtatcagaagaaacagggaaaatctcctcaactcctggtctataatgcaaaaacctcagtagaaggtgtgccatcaaggttcagtggcagtggatcaggcatacagttttctctgaagatcaatagcctgcagcctgaagattttgggagttattactgtcactgtcaacatcattatggtactcttccgacgttcggtggaggcaccaagctggaaatcaaa

SEQ ID NO: 59 - protein, 109 aa, Artificial
  Synthetic VL sequence of 1P3B2
DIQMTQSPASLSASVGETVTITCRASENIYSYLAWYQKKQGKSPQLLVYNAKTSVEGVPSRFSGSGSGIQFSLKINSLQPEDFGSYYCHCQHHYGTLPTFGGGTKLEIK

SEQ ID NO: 60 - protein, 11 aa, Artificial
  Synthetic VL CDR1 sequence of 1P3B2
RASENIYSYLA

SEQ ID NO: 61 - protein, 7 aa, Artificial
  Synthetic VL CDR2 sequence of 1P3B2
NAKTSVE

SEQ ID NO: 62 - protein, 11 aa, Artificial
  Synthetic VL CDR3 sequence of 1P3B2
HCQHHYGTLPT

SEQ ID NO: 63 - DNA, 405 bp, Artificial
  Synthetic VH sequence of 1P4A3
atgggatggagctgggtctttctcttaatcctatcagtaactacaggtgtccactctgaggtccagctgcagcagtctggacctgagctggagaagcctggcgcttcagtgatgatatcctgcaaggcttctggttactcattcactggctacaacatgaactgggtgaagcagagcactggcaagagccttgagtggattggagatattgatccttactatgatggtactaggtacaaccagaagttcaagggcaaggccacattgactgcagacaaatcctccagcacagcctacatgcagctcaagagcctgacatctgaagactctgcagtctattactgtacaagagagggaagagggtttgcttactggggccaagggactctggtcactgtctctgca

SEQ ID NO: 64 - protein, 116 aa, Artificial
  Synthetic VH sequence of 1P4A3
EVQLQQSGPELEKPGASVMISCKASGYSFTGYNMNWVKQSTGKSLEWIGDIDPYYDGTRYNQKFKGKATLTADKSSSTAYMQLKSLTSEDSAVYYCTREGRGFAYWGQGTLVTVSA

SEQ ID NO: 65 - protein, 5 aa, Artificial
  Synthetic VH CDR1 sequence of 1P4A3
GYNMN

SEQ ID NO: 66 - protein, 17 aa, Artificial
  Synthetic VH CDR2 sequence of 1P4A3
DIDPYYDGTRYNQKFKG

SEQ ID NO: 67 - protein, 7 aa, Artificial
  Synthetic VH CDR3 sequence of 1P4A3
EGRGFAY

SEQ ID NO: 68 - DNA, 381 bp, Artificial
  Synthetic VL sequence of 1P4A3
atgaggtccccagctcagttccttgggttgctgctgctgtggcttacaggtgccagatgtgacatccagatgactcagtctccagcctccctatctgcatctgtgggagaaactgtcaccatcacatgtcgagcaagtgagaatatttacagttatttagcatggtatcagcagaaacagggaaaatctcctcagctcctggtctataatgcaaaaaccttagcaggaggtgtgccatcaaggttcagtggcagtggatcaggcacacagttttctctgaagatcaacagcctgcagcctgaagattttgggagttattattgtcaacattattatggtactcctctcacgttcggtgctgggaccaagctggagctgaaa

SEQ ID NO: 69 - protein, 107 aa, Artificial
  Synthetic VL sequence of 1P4A3
DIQMTQSPASLSASVGETVTITCRASENIYSYLAWYQQKQGKSPQLLVYNAKTLAGGVPSRFSGSGSGTQFSLKINSLQPEDFGSYYCQHYYGTPLTFGAGTKLELK

SEQ ID NO: 70 - protein, 11 aa, Artificial
  Synthetic VL CDR1 sequence of 1P4A3
RASENIYSYLA

SEQ ID NO: 71 - protein, 7 aa, Artificial
  Synthetic VL CDR2 sequence of 1P4A3
NAKTLAG

SEQ ID NO: 72 - protein, 9 aa, Artificial
  Synthetic VL CDR3 sequence of 1P4A3
QHYYGTPLT

SEQ ID NO: 73 - DNA, 417 bp, Artificial
  Synthetic VH sequence of 1P5B10
atgggatgcagctgggtaatgctcttcctcctgtcaggaactgcaggtgtccactctgaggtccagctacaacagtctggacctgaactggtgaagcctggagcttcaatgaagatatcctgcagggctgctggtttctcattcactggctacaccatgaactgggtgaagcagagccatggaaagagccttgagtggattggacttattaatcttaacaatggtggtactagccacaaccagaagttcaagggcaaggccacattaactgtagacaagtcatccagcacagcctacatggagctcctcagtctgacatctgaggactctgcagtctattactgtgcaagatggttacgtcgtgggggctatgctatggactactggggtcaaggaatttcagtcaccgtctcctca

SEQ ID NO: 74 - protein, 120 aa, Artificial
  Synthetic VH sequence of 1P5B10
EVQLQQSGPELVKPGASMKISCRAAGFSFTGYTMNWVKQSHGKSLEWIGLINLNNGGTSHNQKFKGKATLTVDKSSSTAYMELLSLTSEDSAVYYCARWLRRGGYAMDYWGQGISVTVSS

SEQ ID NO: 75 - protein, 5 aa, Artificial
  Synthetic VH CDR1 sequence of 1P5B10
GYTMN

SEQ ID NO: 76 - protein, 17 aa, Artificial
  Synthetic VH CDR2 sequence of 1P5B10
LINLNNGGTSHNQKFKG

SEQ ID NO: 77 - protein, 11 aa, Artificial
  Synthetic VH CDR3 sequence of 1P5B10
WLRRGGYAMDY

SEQ ID NO: 78 - DNA, 381 bp, Artificial
  Synthetic VL sequence of 1P5B10
atggtgtcctcagctcagttccttgtatttttgcttttctggattccagcctccagaggtgacatcttgctgactcagtctccagccatcctgtctgtgagtccaggagaaagagtcagtttctcctgcagggccagtcagaacattggcacaagcatacactggtatcagcaaagaacaaatggttctccaaggcttctcataaagtatgcttctgagtctatctctgggatcccttccaggtttagtggcagtggatcagggacagattttactcttagcatcaacagtgtggagtctgaagatattgcagattattactgtcaacaaagtgatagctggccactcacgtttggtgctgggaccaagctggagctgaaa

SEQ ID NO: 79 - protein, 107 aa, Artificial
  Synthetic VL sequence of 1P5B10
DILLTQSPAILSVSPGERVSFSCRASQNIGTSIHWYQQRTNGSPRLLIKYASESISGIPSRFSGSGSGTDFTLSINSVESEDIADYYCQQSDSWPLTFGAGTKLELK

SEQ ID NO: 80 - protein, 11 aa, Artificial
  Synthetic VL CDR1 sequence of 1P5B10
RASQNIGTSIH

SEQ ID NO: 81 - protein, 7 aa, Artificial
  Synthetic VL CDR2 sequence of 1P5B10
YASESIS

SEQ ID NO: 82 - protein, 9 aa, Artificial
  Synthetic VL CDR3 sequence of 1P5B10
QQSDSWPLT

SEQ ID NO: 83 - DNA, 417 bp, Artificial
  Synthetic VH sequence of 1P4A12
atgggatgcagctgtgtaatgctcttcctcctgtcaggaactgcaggtgtccgctctgaggtccagctgcaacagtctggacctgagctggtgaagcctggagcttcaatgaagatatcctgcaaggcttctggttactcattcactggctacaccatgaactgggtgaagcagagccatggaaagaagcttgagtggattggacttattaatccttacaatggtgggactatctacaaccagaagttcaagggcaaggccacattaactgtagacaagtcatccagcacagcctacatggagctcctcagtctgacatctgaggactctgcagtctattactgtgcaagatggttacgacgtgggggctatgctatggactactggggtcaaggagcctcagtcaccgtctcctca

SEQ ID NO: 84 - protein, 120 aa, Artificial
  Synthetic VH sequence of 1P4A12
EVQLQQSGPELVKPGASMKISCKASGYSFTGYTMNWVKQSHGKKLEWIGLINPYNGGTIYNQKFKGKATLTVDKSSSTAYMELLSLTSEDSAVYYCARWLRRGGYAMDYWGQGASVTVSS

SEQ ID NO: 85 - protein, 5 aa, Artificial
  Synthetic VH CDR1 sequence of 1P4A12
GYTMN

SEQ ID NO: 86 - protein, 17 aa, Artificial
  Synthetic VH CDR2 sequence of 1P4A12
LINPYNGGTIYNQKFKG

SEQ ID NO: 87 - protein, 11 aa, Artificial
  Synthetic VH CDR3 sequence of 1P4A12
WLRRGGYAMDY

SEQ ID NO: 88 - DNA, 381 bp, Artificial
  Synthetic VL sequence of 1P4A12
atggtgtcctcagctcagttccttgtatttttgcttttctggattccagcctccagaggtgacatcttgctgactcagtctccagccatcctgtctgtgagtccaggagaaagagtcagtttctcctgcagggccagtcagagcattggcacaagcatacactggtatcagcaaagaacaaatggttctccaaggcttctcataaagtttgcttctgagtctatctctgggatcccttccaggtttagtggcagtggatcagggacagattttactcttagcatcaacagtgtggagtctgaagatattgcagattattactgtcaacaaagtgatagctggccactcacgttcggtgctgggaccaagctggaggtgaaa

SEQ ID NO: 89 - protein, 107 aa, Artificial
  Synthetic VL sequence of 1P4A12
DILLTQSPAILSVSPGERVSFSCRASQSIGTSIHWYQQRTNGSPRLLIKFASESISGIPSRFSGSGSGTDFTLSINSVESEDIADYYCQQSDSWPLTFGAGTKLEVK

SEQ ID NO: 90 - protein, 11 aa, Artificial
  Synthetic VL CDR1 sequence of 1P4A12
RASQSIGTSIH

SEQ ID NO: 91 - protein, 7 aa, Artificial
  Synthetic VL CDR2 sequence of 1P4A12
FASESIS

SEQ ID NO: 92 - protein, 9 aa, Artificial
  Synthetic VL CDR3 sequence of 1P4A12
QQSDSWPLT

SEQ ID NO: 93 - DNA, 423 bp, Artificial
  Synthetic VH sequence of 1P2E7
atgagagtgctgattcttttgtggctgttcacagccttccctggtatcctgtctgatgtgcagcttcaggagtcgggacctggcctggtgaaaccttctcagtctctgtccctcacctgcactgtcactggcgactcaatcaccagtgattatgcctggaactggatccggcagtttccaggaaacaaactggagtggatgggctacataagctacagtggtagcactagctacaacccatctctcaaaagtcgattctctatcactcgagacacatccaagaaccagttcttcctgcagttgaattctgtgactactgaggactcagccacatattactgtgcaagaggggggttctactataggtacgccgggcctgggtttgcttattggggccaagggactctggtcactgtctctgca

SEQ ID NO: 94 - protein, 123 aa, Artificial
  Synthetic VH sequence of 1P2E7
DVQLQESGPGLVKPSQSLSLTCTVTGDSITSDYAWNWIRQFPGNKLEWMGYISYSGSTSYNPSLKSRFSITRDTSKNQFFLQLNSVTTEDSATYYCARGGFYYRYAGPGFAYWGQGTLVTVSA

SEQ ID NO: 95 - protein, 6 aa, Artificial
  Synthetic VH CDR1 sequence of 1P2E7
SDYAWN

SEQ ID NO: 96 - protein, 16 aa, Artificial
  Synthetic VH CDR2 sequence of 1P2E7
YISYSGSTSYNPSLKS

SEQ ID NO: 97 - protein, 14 aa, Artificial
  Synthetic VH CDR3 sequence of 1P2E7
GGFYYRYAGPGFAY

SEQ ID NO: 98 - DNA, 396 bp, Artificial
  Synthetic VL sequence of 1P2E7
atgatgagtcctgcccagttcctgtttctgttagtgctctcgattcaggaaatcaacggtgatgttgtgatgacccagactccactcactttgtcggttaccattggacaaccagcttccatctcttgcaagtcaagtcagagcctcttatatactaatggaaaaacctatttgaattggttgttacagaggccaggccagtctccaaaacgcctaatctatctggtgtctaaattggactctggagtccctgacaggttcagtggcagtggatcagggacagatttcacactgaaaatcagcagagtggaggctgaggatttgggagtttattactgcttgcagagtacacattttccgctcacgttcggtgctgggaccaagctggagctgaaa

SEQ ID NO: 99 - protein, 112 aa, Artificial
  Synthetic VL sequence of 1P2E7
DVVMTQTPLTLSVTIGQPASISCKSSQSLLYTNGKTYLNWLLQRPGQSPKRLIYLVSKLDSGVPDRFSGSGSGTDFTLKISRVEAEDLGVYYCLQSTHFPLTFGAGTKLELK

SEQ ID NO: 100 - protein, 16 aa, Artificial
  Synthetic VL CDR1 sequence of 1P2E7
KSSQSLLYTNGKTYLN

SEQ ID NO: 101 - protein, 7 aa, Artificial
  Synthetic VL CDR2 sequence of 1P2E7
LVSKLDS

SEQ ID NO: 102 - protein, 9 aa, Artificial
  Synthetic VL CDR3 sequence of 1P2E7
LQSTHFPLT

SEQ ID NO: 103 - protein, 19 aa, Artificial
  Synthetic signal sequence of heavy chain variable domain of 1P3B2.2
MGWICIFLFLVSVTTGVHS

SEQ ID NO: 104 - protein, 20 aa, Artificial
  Synthetic signal sequence of light chain variable domain of 1P3B2.2
MRAPAQFLGLLLLWLTGARC

SEQ ID NO: 105 - protein, 19 aa, Artificial
  Synthetic signal sequence of heavy chain variable domain of 1P4A3.3
MGWSWVFLLILSVTTGVHS

SEQ ID NO: 106 - protein, 20 aa, Artificial
  Synthetic signal sequence of light chain variable domain of 1P4A3.3
MRSPAQFLGLLLLWLTGARC

SEQ ID NO: 107 - protein, 19 aa, Artificial
  Synthetic signal sequence of heavy chain variable domain of 1P5B10.3
MGCSWVMLFLLSGTAGVHS

SEQ ID NO: 108 - protein, 20 aa, Artificial
  Synthetic signal sequence of light chain variable domain of 1P5B10.3
MVSSAQFLVFLLFWIPASRG

SEQ ID NO: 109 - protein, 19 aa, Artificial
  Synthetic signal sequence of heavy chain variable domain of 1P4A12.2
MGCSCVMLFLLSGTAGVRS

SEQ ID NO: 110 - protein, 20 aa, Artificial
  Synthetic signal sequence of light chain variable domain of 1P4A12.2
MVSSAQFLVFLLFWIPASRG

SEQ ID NO: 111 - protein, 18 aa, Artificial
  Synthetic signal sequence of heavy chain variable domain of 1P2E7.3
MRVLILLWLFTAFPGILS

SEQ ID NO: 112 - protein, 20 aa, Artificial
  Synthetic signal sequence of light chain variable domain of 1P2E7.3
MMSPAQFLFLLVLSIQEING

SEQ ID NO: 113 - protein, 1291 aa, Homo sapiens
MELLPPLPQSFLLLLLLPAKPAAGEDWQCPRTPYAASRDFDVKYVVPSFSAGGLVQAMVTYEGDRNESAVFVAIRNRLHVLGPDLKSVQSLATGPAGDPGCQTCAACGPGPHGPPGDTDTKVLVLDPALPALVSCGSSLQGRCFLHDLEPQGTAVHLAAPACLFSAHHNRPDDCPDCVASPLGTRVTVVEQGQASYFYVASSLDAAVAASFSPRSVSIRRLKADASGFAPGFVALSVLPKHLVSYSIEYVHSFHTGAFVYFLTVQPASVTDDPSALHTRLARLSATEPELGDYRELVLDCRFAPKRRRRGAPEGGQPYPVLQVAHSAPVGAQLATELSIAEGQEVLFGVFVTGKDGGPGVGPNSVVCAFPIDLLDTLIDEGVERCCESPVHPGLRRGLDFFQSPSFCPNPPGLEALSPNTSCRHFPLLVSSSFSRVDLFNGLLGPVQVTALYVTRLDNVTVAHMGTMDGRILQVELVRSLNYLLYVSNFSLGDSGQPVQRDVSRLGDHLLFASGDQVFQVPIQGPGCRHFLTCGRCLRAWHFMGCGWCGNMCGQQKECPGSWQQDHCPPKLTEEPVLIAVQPLFGPRAGGTCLTLEGQSLSVGTSRAVLVNGTECLLARVSEGQLLCATPPGATVASVPLSLQVGGAQVPGSWTFQYREDPVVLSISPNCGYINSHITICGQHLTSAWHLVLSFHDGLRAVESRCERQLPEQQLCRLPEYVVRDPQGWVAGNLSARGDGAAGFTLPGFRFLPPPHPPSANLVPLKPEEHAIKFEYIGLGAVADCVGINVTVGGESCQHEFRGDMVVCPLPPSLQLGQDGAPLQVCVDGECHILGRVVRPGPDGVPQSTLLGILLPLLLLVAALATALVFSYWWRRKQLVLPPNLNDLASLDQTAGATPLPILYSGSDYRSGLALPAIDGLDSTTCVHGASFSDSEDESCVPLLRKESIQLRDLDSALLAEVKDVLIPHERVVTHSDRVIGKGHFGVVYHGEYIDQAQNRIQCAIKSLSRITEMQQVEAFLREGLLMRGLNHPNVLALIGIMLPPEGLPHVLLPYMCHGDLLQFIRSPQRNPTVKDLISFGLQVARGMEYLAEQKFVHRDLAARNCMLDESFTVKVADFGLARDILDREYYSVQQHRHARLPVKWMALESLQTYRFTTKSDVWSFGVLLWELLTRGAPPYRHIDPFDLTHFLAQGRRLPQPEYCPDSLYQVMQQCWEADPAVRPTFRVLVGEVEQIVSALLGDHYVQLPATYMNLGPSTSHEMNVRPEQPQFSPMPGNVRRPRPLSEPPRPT

SEQ ID NO: 114 - DNA, 360 bp, Artificial
  Synthetic VH sequence of M93-D02
gaagttcaattgttagagtctggtggcggtcttgttcagcctggtggttctttacgtctttcttgcgctgcttccggattcactttctctatttactggatgtcttgggttcgccaagctcctggtaaaggtttggagtgggtttcttctatcggtccttctggtggcatgactgcttatgctgactccgttaaaggtcgcttcactatctctagagacaactctaagaatactctctacttgcagatgaacagcttaagggctgaggacacggccgtatattactgtgcaaggggtagcagctggtacgccgcgtaccattactactggggccagggaaccctggtcaccgtctcaagc

SEQ ID NO: 115 - protein, 120 aa, Artificial
  Synthetic VH sequence of M93-D02
EVQLLESGGGLVQPGGSLRLSCAASGFTFSIYWMSWVRQAPGKGLEWVSSIGPSGGMTAYADSVKGRFTISRDNSKNTLYLQMNSLRAEDTAVYYCARGSSWYAAYHYYWGQGTLVTVSS

SEQ ID NO: 116 - protein, 5 aa, Artificial
  Synthetic VH CDR1 sequence of M93-D02
IYWMS

SEQ ID NO: 117 - protein, 17 aa, Artificial
  Synthetic VH CDR2 sequence of M93-D02
SIGPSGGMTAYADSVKG

SEQ ID NO: 118 - protein, 11 aa, Artificial
  Synthetic VH CDR3 sequence of M93-D02
GSSWYAAYHYY

SEQ ID NO: 119 - DNA, 339 bp, Artificial
  Synthetic VL sequence of M93-D02
gacatccagatgacccagtctccagactccctggctgtgtctctgggcgagcgggccaccatcaactgcaggtccagccagagtgttttatacagttccaacaataagaagtacttagcttggtaccagcagaaagcaggacagccccctaagttgctcatttactgggcatctacccgggcatccggggtccctgaccgattcagtggcagcgggtctgggacagatttcactctcaccatcagcagtctgcaggctgaagatgtggcagtttatttctgtcagcaatattataggactcctccgacgttcggccaagggaccaaggtggaaatcaaa

SEQ ID NO: 120 - protein, 113 aa, Artificial
  Synthetic VL sequence of M93-D02
DIQMTQSPDSLAVSLGERATINCRSSQSVLYSSNNKKYLAWYQQKAGQPPKLLIYWASTRASGVPDRFSGSGSGTDFTLTISSLQAEDVAVYFCQQYYRTPPTFGQGTKVEIK

SEQ ID NO: 121 - protein, 17 aa, Artificial
  Synthetic VL CDR1 sequence of M93-D02
RSSQSVLYSSNNKKYLA

SEQ ID NO: 122 - protein, 7 aa, Artificial
  Synthetic VL CDR2 sequence of M93-D02
WASTRAS

SEQ ID NO: 123 - protein, 9 aa, Artificial
  Synthetic VL CDR3 sequence of M93-D02
QQYYRTPPT

SEQ ID NO: 124 - DNA, 357 bp, Artificial
  Synthetic VH sequence of M96-C05
gaagttcaattgttagagtctggtggcggtcttgttcagcctggtggttctttacgtctttcttgcgctgcttccggattcactttctctcgttactatatgaagtgggttcgccaagctcctggtaaaggtttggagtgggtttctgttatctctccttctggtggcgctactggttatgctgactccgttaaaggtcgcttcactatctctagagacaactctaagaatactctctacttgcagatgaacagcttaagggctgaggacacggccgtgtattactgtgcgggacggtattactatgatagtagtgaggactactggggccagggcaccctggtcaccgtctcaagc

SEQ ID NO: 125 - protein, 119 aa, Artificial
  Synthetic VH sequence of M96-C05
EVQLLESGGGLVQPGGSLRLSCAASGFTFSRYYMKWVRQAPGKGLEWVSVISPSGGATGYADSVKGRFTISRDNSKNTLYLQMNSLRAEDTAVYYCAGRYYYDSSEDYWGQGTLVTVSS

SEQ ID NO: 126 - protein, 5 aa, Artificial
  Synthetic VH CDR1 sequence of M96-C05
RYYMK

SEQ ID NO: 127 - protein, 17 aa, Artificial
  Synthetic VH CDR2 sequence of M96-C05
VISPSGGATGYADSVKG

SEQ ID NO: 128 - protein, 10 aa, Artificial
  Synthetic VH CDR3 sequence of M96-C05
RYYYDSSEDY

SEQ ID NO: 129 - DNA, 321 bp, Artificial
  Synthetic VL sequence of M96-C05
gacatccagatgacccagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcaacttactactgtcaacagagttacactgccccgctcactttcggcggagggaccaaggtggagatcaaa

SEQ ID NO: 130 - protein, 107 aa, Artificial
  Synthetic VL sequence of M96-C05
DIQMTQSPATLSLSPGERATLSCRASQSVSSYLAWYQQKPGQAPRLLIYDASNRATGIPARFSGSGSGTDFTLTISSLEPEDFATYYCQQSYTAPLTFGGGTKVEIK

SEQ ID NO: 131 - protein, 11 aa, Artificial
  Synthetic VL CDR1 sequence of M96-C05
RASQSVSSYLA

SEQ ID NO: 132 - protein, 7 aa, Artificial
  Synthetic VL CDR2 sequence of M96-C05
DASNRAT

SEQ ID NO: 133 - protein, 10 aa, Artificial
  Synthetic VL CDR3 sequence of M96-C05
QQSYTAPLTF

SEQ ID NO: 134 - DNA, 363 bp, Artificial
  Synthetic VH sequence of M97-D03
gaagttcaattgttagagtctggtggcggtcttgttcagcctggtggttctttacgtctttcttgcgctgcttccggattcactttctctcgttaccagatgtggtgggttcgccaagctcctggtaaaggtttggagtgggtttcttctatctctccttctggtggcaagacttggtatgctgactccgttaaaggtcgcttcactatctctagagacaactctaagaatactctctacttgcagatgaacagcttaagggctgaggacacggccgtgtattactgtgcgaaagttttgagtggcatattacggggaaactttgactactggggccagggaaccctggtcaccgtctcaagc

SEQ ID NO: 135 - protein, 121 aa, Artificial
  Synthetic VH sequence of M97-D03
EVQLLESGGGLVQPGGSLRLSCAASGFTFSRYQMWWVRQAPGKGLEWVSSISPSGGKTWYADSVKGRFTISRDNSKNTLYLQMNSLRAEDTAVYYCAKVLSGILRGNFDYWGQGTLVTVSS

SEQ ID NO: 136 - protein, 5 aa, Artificial
  Synthetic VH CDR1 sequence of M97-D03
RYQMW

SEQ ID NO: 137 - protein, 17 aa, Artificial
  Synthetic VH CDR2 sequence of M97-D03
SISPSGGKTWYADSVKG

SEQ ID NO: 138 - protein, 12 aa, Artificial
  Synthetic VH CDR3 sequence of M97-D03
VLSGILRGNFDY

SEQ ID NO: 139 - DNA, 321 bp, Artificial
  Synthetic VL sequence of M97-D03
gacatccagatgacccagtctccagccaccctgtctgtgtctccaggggaaagagccaccctctcctgcagggccagtcagactgttaccagcaacttagcctggtaccagcagaaacctggccaggctcccaggctcctcatctatggggcatccaccagggccactggtatcccagccaggttcagtggcagtgggtctgcgacagagttcactctcatcatcagcagcatgcagtctgaagattttgcagtttattactgtcagcagtataataactggcctctcactttcggcggagggacccaggtggacatcaaa

SEQ ID NO: 140 - protein, 107 aa, Artificial
  Synthetic VL sequence of M97-D03
DIQMTQSPATLSVSPGERATLSCRASQTVTSNLAWYQQKPGQAPRLLIYGASTRATGIPARFSGSGSATEFTLIISSMQSEDFAVYYCQQYNNWPLTFGGGTQVDIK

SEQ ID NO: 141 - protein, 11 aa, Artificial
  Synthetic VL CDR1 sequence of M97-D03
RASQTVTSNLA

SEQ ID NO: 142 - protein, 7 aa, Artificial
  Synthetic VL CDR2 sequence of M97-D03
GASTRAT

SEQ ID NO: 143 - protein, 9 aa, Artificial
  Synthetic VL CDR3 sequence of M97-D03
QQYNNWPLT

SEQ ID NO: 144 - DNA, 357 bp, Artificial
  Synthetic VH sequence of M98-E12
gaagttcaattgttagagtctggtggcggtcttgttcagcctggtggttctttacgtctttcttgcgctgcttccggattcactttctctcgttaccttatgatttgggttcgccaagctcctggtaaaggtttggagtgggtttcttctatcgtttcttctggtggctctacttggtatgctgactccgttaaaggtcgcttcactatctctagagacaactctaagaatactctctacttgcagatgaacagcttaagggctgaggacacggccgtgtattactgtgcgagacaccgtggatatagtggccaactaaactactggggccagggaaccctggtcaccgtctcaagc

SEQ ID NO: 145 - protein, 119 aa, Artificial
  Synthetic VH sequence of M98-E12
EVQLLESGGGLVQPGGSLRLSCAASGFTFSRYLMIWVRQAPGKGLEWVSSIVSSGGSTWYADSVKGRFTISRDNSKNTLYLQMNSLRAEDTAVYYCARHRGYSGQLNYWGQGTLVTVSS

SEQ ID NO: 146 - protein, 5 aa, Artificial
  Synthetic VH CDR1 sequence of M98-E12
RYLMI

SEQ ID NO: 147 - protein, 17 aa, Artificial
  Synthetic VH CDR2 sequence of M98-E12
SIVSSGGSTWYADSVKG

SEQ ID NO: 148 - protein, 10 aa, Artificial
  Synthetic VH CDR3 sequence of M98-E12
HRGYSGQLNY

SEQ ID NO: 149 - DNA, 324 bp, Artificial
  Synthetic VL sequence of M98-E12
gacatccagatgacccagtctccatcctccctgtctgcatctgtgggagacagagtcgccatcacttgccgcgcaagtcagagcatcgacacctatttaaattggtatcagcagaaaccagggaaagcccctaaactcctgatctatgctgcatccaagttggaagacggggtcccatcaagattcagtggcagtggaactgggacagatttcactctcaccatcagaagtctgcaacctgaagattttgcaagttatttctgtcaacagagctactctagtccagggatcactttcggccctgggaccaaggtggagatcaaa

SEQ ID NO: 150 - protein, 108 aa, Artificial; identical to SEQ ID NO: 49
  Synthetic VL sequence of M98-E12

SEQ ID NO: 151 - protein, 11 aa, Artificial
  Synthetic VL CDR1 sequence of M98-E12
RASQSIDTYLN

SEQ ID NO: 152 - protein, 7 aa, Artificial
  Synthetic VL CDR2 sequence of M98-E12
AASKLED

SEQ ID NO: 153 - protein, 10 aa, Artificial
  Synthetic VL CDR3 sequence of M98-E12
QQSYSSPGIT

(19) United States
(12) Patent Application Publication
(10) Pub. No.: US 2009/0226442 A1
(43) Pub. Date: Sep. 10, 2009

(54) RON antibodies and uses thereof

(75) Inventors: Heather Huet, Arlington, MA (US); Veronique Bailly, Lexington, MA (US); Ellen Garber, Cambridge, MA (US); Christilyn Graff, Cambridge, MA (US); Steven Miklasz, Upton, MA (US)

Correspondence Address: STERNE, KESSLER, GOLDSTEIN & FOX, P.L.L.C., 1100 NEW YORK AVE., N.W., WASHINGTON, DC, 20005, US

(73) Assignee: Biogen Idec MA Inc.

(21) Appl. No.: 12/320,296

(22) Filed: Jan. 22, 2009

Related U.S. Application Data
(60) Provisional application No. 61/022,779, filed on Jan. 22, 2008.

(51) Int. Cl. A61K 39/395; C07K 16/00; C07K 17/00; C12N 9/96; C12N 7/00; C07H 21/04; C12N 5/00; C12P 21/04

(52) U.S. Cl. 424/138.1; 530/387.1; 530/387.7; 530/391.3; 530/391.7; 435/188; 435/235.1; 536/23.53; 435/325; 435/69.6

(57) ABSTRACT

The invention relates to antibodies which bind to RON (receptor d'origine nantais, MST1R) and uses thereof, in particular in the diagnosis and treatment of cancer. Specific antibodies which inhibit RON-mediated pro-survival and tumor proliferation pathways, and variants, fragments, and derivatives thereof are provided. Also provided are specific antibodies which block the ability of the ligand, MSP to bind to RON, as well as fragments, variants and derivatives of such antibodies. The invention also includes polynucleotides encoding the above antibodies or fragments, variants or derivatives thereof, as well as vectors and host cells comprising such polynucleotides. The invention further includes methods of diagnosing and treating cancer using antibodies of the invention.

### CROSS-REFERENCE TO RELATED APPLICATIONS

The present application claims the benefit of U.S. Provisional Application No. 61/022,779, filed Jan. 22, 2008, which is incorporated by reference herein in its entirety.

### BACKGROUND OF THE INVENTION

RON (recepteur d'origine nantais, also known as MST1R) is a receptor-type protein tyrosine kinase that is essential to embryonic development and also plays an important role in inflammatory responses (Camp et al. Ann. Surg. Oncol. 12:273-281 (2005)). RON is mostly expressed in epithelial-derived cell types, and it has been suggested that RON, like a number of other receptor-type tyrosine kinases, may play a role in the progression of malignant epithelial cancers (Wang et al. Carcinogensis 23:1291-1297 (2003)).

Receptor-type protein tyrosine kinases generally consist of an extracellular domain which binds to extracellular ligands such as growth factors and hormones, as well an intracellular domain which possesses the kinase functional domain. Receptor-type protein tyrosine kinases have been sub-divided into a number of classes, and RON is a member of the MET family of receptor tyrosine kinases, which also includes Stk, c-Met and c-Sea (Camp et al. Ann. Surg. Oncol. 12:273-281 (2005)). RON and c-Met are the only members of the family found in humans, and they share about 65% homology overall. C-Met is the receptor for hepatocyte growth factor/scatter factor (HGF/SF) and has been fairly well characterized as a protooncogene.

The ligand for RON, macrophage stimulating protein (MSP) has also been identified and shares about 40% homology with the c-Met ligand, HGF/SF. MSP and HGF belong to the plasminogen-prothrombin family, which is characterized by kringle domains. Interestingly, MSP has also been linked with cancer. For example, Welm et al. recently observed an association between MSP and metastasis and poor prognosis in breast cancer (PNAS 104:7507-7575 (2007)).

It has been demonstrated that MSP binds to a particular domain in the extracellular portion of RON called the semaphorin (sema) domain. RON and c-Met are the only receptor tyrosine kinases that have extracellular sema domains, and it has been demonstrated that the sema domain of RON includes its ligand binding site. Binding of MSP to RON causes phosphorylation within the kinase domain of RON, which leads to an increase in RON kinase activity. Alternatively, β₁ integrins can phosphorylate and activate RON through a Src-dependent pathway (Camp et al. Ann. Surg. Oncol. 12:273-281 (2005)). Activation of RON, initiates signaling of a number of pathways, including PI3-K, Ras, src, β-catenin and Fak signaling. Many of the signaling pathways activated by RON are implicated in processes associated with cancer such as proliferation and inhibition of apoptosis.

More recently, RON itself has been implicated in cancer progression for a number of reasons. For example, RON is expressed in a number of human tumors including breast, bladder, colon, ovarian and pancreatic cancers. In addition, RON has been shown in vitro to increase cell proliferation and motility. Furthermore, RON induces tumor growth and metastasis in RON-transgenic mice. (Waltz et al. Cancer Research 66:11967-11974 (2006)). Thus, there is a need for molecules, including anti-RON antibodies, that could inhibit the RON signaling pathways.

### BRIEF SUMMARY OF THE INVENTION

In some embodiments, the invention provides an isolated antibody or antigen-binding fragment thereof which specifically binds to the same RON epitope as a reference monoclonal Fab antibody fragment selected from the group consisting of M14-H06, M15-E10, M16-C07, M23-F10, M80-B03, M93-D02, M96-C05, M97-D03, and M98-E12 or a reference monoclonal antibody selected from the group consisting of 1P2E7, 1P3B2, 1P4A3, 1P4A12 and 1P5B10.

In some embodiments, the invention provides an isolated antibody or antigen-binding fragment thereof which specifically binds to RON, where the antibody or fragment competitively inhibits a reference monoclonal Fab antibody fragment selected from the group consisting of M14-H06, M15-E10, M16-C07, M23-F10, M80-B03, M93-D02, M96-C05, M97-D03, and M98-E12 or a reference monoclonal antibody selected from the group consisting of 1P2E7, 1P3B2, 1P4A3, 1P4A12 and 1P5B10 from binding to RON.

In some embodiments, the invention provides an isolated antibody or antigen-binding fragment thereof which specifically binds to RON, where the antibody or fragment thereof comprises an antigen binding domain identical to that of a monoclonal Fab antibody fragment selected from the group consisting of M14-H06, M15-E10, M16-C07, M23-F10, M80-B03, M93-D02, M96-C05, M97-D03, and M98-E12 or a reference monoclonal antibody selected from the group consisting of 1P2E7, 1P3B2, 1P4A3, 1P4A12 and 1P5B10.

In some embodiments, the invention provides an isolated antibody or fragment thereof which specifically binds to RON, where the heavy chain variable region (VH) of the antibody or fragment thereof comprises an amino acid sequence at least 90% identical to a reference amino acid sequence selected from the group consisting of: SEQ ID NO:4, SEQ ID NO:14, SEQ ID NO:24, SEQ ID NO:34, SEQ ID NO:44, SEQ ID NO:54, SEQ ID NO:64, SEQ ID NO:74, SEQ ID NO:84, SEQ ID NO:94, SEQ ID NO:115, SEQ ID NO:125, SEQ ID NO:135, and SEQ ID NO:145.

In some embodiments, the invention provides an isolated antibody or fragment thereof which specifically binds to RON, where the light chain variable region (VL) of the antibody or fragment thereof comprises an amino acid sequence at least 90% identical to a reference amino acid sequence selected from the group consisting of: SEQ ID NO:9, SEQ ID NO:19, SEQ ID NO:29, SEQ ID NO:39, SEQ ID NO:49, SEQ ID NO:59, SEQ ID NO:69, SEQ ID NO:79, SEQ ID NO:89, SEQ ID NO:99, SEQ ID NO:120, SEQ ID NO: 130, SEQ ID NO:140, and SEQ ID NO:150.

In some embodiments, the invention provides an isolated antibody or fragment thereof which specifically binds to RON, where the VH of the antibody or fragment thereof comprises an amino acid sequence identical, except for 20 or fewer conservative amino acid substitutions, to a reference amino acid sequence selected from the group consisting of: SEQ ID NO:4, SEQ ID NO:14, SEQ ID NO:24, SEQ ID NO:34, SEQ ID NO:44, SEQ ID NO:54, SEQ ID NO:64, SEQ ID NO:74, SEQ ID NO:84, SEQ ID NO:94, SEQ ID NO:115, SEQ ID NO:125, SEQ ID NO:135, and SEQ ID NO:145.

In some embodiments, the invention provides an isolated antibody or fragment thereof which specifically binds to RON, where the VL of the antibody or fragment thereof comprises an amino acid sequence identical, except for 20 or fewer conservative amino acid substitutions, to a reference amino acid sequence selected from the group consisting of: SEQ ID NO:9, SEQ ID NO:19, SEQ ID NO:29, SEQ ID NO:39, SEQ ID NO:49, SEQ ID NO:59, SEQ ID NO:69, SEQ ID NO:79, SEQ ID NO:89, SEQ ID NO:99, SEQ ID NO:120, SEQ ID NO: 130, SEQ ID NO:140, and SEQ ID NO:150.

In some embodiments, the invention provides an isolated antibody or fragment thereof which specifically binds to RON, where the VH of the antibody or fragment thereof comprises an amino acid sequence selected from the group consisting of: SEQ ID NO:4, SEQ ID NO:14, SEQ ID NO:24, SEQ ID NO:34, SEQ ID NO:44, SEQ ID NO:54, SEQ ID NO:64, SEQ ID NO:74, SEQ ID NO:84, SEQ ID NO:94, SEQ ID NO:115, SEQ ID NO:125, SEQ ID NO:135, and SEQ ID NO:145.

In some embodiments, the invention provides an isolated antibody or fragment thereof which specifically binds to RON, where the VL of the antibody or fragment thereof comprises an amino acid sequence selected from the group consisting of: SEQ ID NO:9, SEQ ID NO:19, SEQ ID NO:29, SEQ ID NO:39, SEQ ID NO:49, SEQ ID NO:59, SEQ ID NO:69, SEQ ID NO:79, SEQ ID NO:89, SEQ ID NO:99, SEQ ID NO:120, SEQ ID NO: 130, SEQ ID NO:140, and SEQ ID NO:150.

In some embodiments, the invention provides an isolated antibody or fragment thereof which specifically binds to RON, where the VH and VL of the antibody or fragment thereof comprise, respectively, amino acid sequences at least 90% identical to reference amino acid sequences selected from the group consisting of: SEQ ID NO:4 and SEQ ID NO:9; SEQ ID NO:14 and SEQ ID NO:19; SEQ ID NO:24 and SEQ ID NO:29; SEQ ID NO:34 and SEQ ID NO:39; SEQ ID NO:44 and SEQ ID NO:49; SEQ ID NO:54 and SEQ ID NO:59; SEQ ID NO:64 and SEQ ID NO:69; SEQ ID NO:74 and SEQ ID NO:79; SEQ ID NO:84 and SEQ ID NO:89; SEQ ID NO:94 and SEQ ID NO:99, SEQ ID NO: 115 and SEQ ID NO: 120, SEQ ID NO: 125 and SEQ ID NO:130, SEQ ID NO: 135 and SEQ ID NO: 140, and SEQ ID NO: 145 and SEQ ID NO: 150.

In some embodiments, the invention provides an isolated antibody or fragment thereof which specifically binds to RON, where the VH and VL of the antibody or fragment thereof comprise, respectively, amino acid sequences identical, except for 20 or fewer conservative amino acid substitutions each, to reference amino acid sequences selected from the group consisting of: SEQ ID NO:4 and SEQ ID NO:9; SEQ ID NO:14 and SEQ ID NO:19; SEQ ID NO:24 and SEQ ID NO:29; SEQ ID NO:34 and SEQ ID NO:39; SEQ ID NO:44 and SEQ ID NO:49; SEQ ID NO:54 and SEQ ID NO:59; SEQ ID NO:64 and SEQ ID NO:69; SEQ ID NO:74 and SEQ ID NO:79; SEQ ID NO:84 and SEQ ID NO:89; SEQ ID NO:94 and SEQ ID NO:99, SEQ ID NO: 115 and SEQ ID NO: 120, SEQ ID NO: 125 and SEQ ID NO:130, SEQ ID NO: 135 and SEQ ID NO: 140, and SEQ ID NO: 145 and SEQ ID NO: 150.

In some embodiments, the invention provides an isolated antibody or fragment thereof which specifically binds to RON, where the VH and VL of the antibody or fragment thereof comprise, respectively, amino acid sequences selected from the group consisting of: SEQ ID NO:4 and SEQ ID NO:9; SEQ ID NO:14 and SEQ ID NO:19; SEQ ID NO:24 and SEQ ID NO:29; SEQ ID NO:34 and SEQ ID NO:39; SEQ ID NO:44 and SEQ ID NO:49; SEQ ID NO:54 and SEQ ID NO:59; SEQ ID NO:64 and SEQ ID NO:69; SEQ ID NO:74 and SEQ ID NO:79; SEQ ID NO:84 and SEQ ID NO:89; SEQ ID NO:94 and SEQ ID NO:99, SEQ ID NO: 115 and SEQ ID NO: 120, SEQ ID NO: 125 and SEQ ID NO:130, SEQ ID NO: 135 and SEQ ID NO: 140, and SEQ ID NO: 145 and SEQ ID NO: 150.

In some embodiments, the invention provides an isolated antibody or fragment thereof which specifically binds to RON, where the VH of the antibody or fragment thereof comprises a Kabat heavy chain complementarity determining region-1 (VH-CDR1) amino acid sequence identical, except for two or fewer amino acid substitutions, to a reference VH-CDR1 amino acid sequence selected from the group consisting of: SEQ ID NO: 5, SEQ ID NO: 15, SEQ ID NO: 25, SEQ ID NO: 35, SEQ ID NO: 45, SEQ ID NO: 55, SEQ ID NO: 65, SEQ ID NO: 75, SEQ ID NO: 85, SEQ ID NO: 95, SEQ ID NO: 116, SEQ ID NO:126, SEQ ID NO:136, and SEQ ID NO:146. In further embodiments, the VH-CDR1 amino acid sequence is selected from the group consisting of: SEQ ID NO: 5, SEQ ID NO: 15, SEQ ID NO: 25, SEQ ID NO: 35, SEQ ID NO: 45, SEQ ID NO: 55, SEQ ID NO: 65, SEQ ID NO: 75, SEQ ID NO: 85, SEQ ID NO: 95, SEQ ID NO: 116, SEQ ID NO:126, SEQ ID NO:136, and SEQ ID NO:146.

In some embodiments, the invention provides an isolated antibody or fragment thereof which specifically binds to RON, where the VH of the antibody or fragment thereof comprises a Kabat heavy chain complementarity determining region-2 (VH-CDR2) amino acid sequence identical, except for four or fewer amino acid substitutions, to a reference VH-CDR2 amino acid sequence selected from the group consisting of: SEQ ID NO: 6, SEQ ID NO: 16, SEQ ID NO: 26, SEQ ID NO: 36, SEQ ID NO: 46, SEQ ID NO: 56, SEQ ID NO: 66, SEQ ID NO: 76, SEQ ID NO: 86, SEQ ID NO: 96, SEQ ID NO:117, SEQ ID NO:127, SEQ ID NO:137, and SEQ ID NO:147. In further embodiments, the VH-CDR2 amino acid sequence is selected from the group consisting of: SEQ ID NO: 6, SEQ ID NO: 16, SEQ ID NO: 26, SEQ ID NO: 36, SEQ ID NO: 46, SEQ ID NO: 56, SEQ ID NO: 66, SEQ ID NO: 76, SEQ ID NO: 86, SEQ ID NO: 96, SEQ ID NO:117, SEQ ID NO:127, SEQ ID NO:137, and SEQ ID NO:147.

In some embodiments, the invention provides an isolated antibody or fragment thereof which specifically binds to RON, where the VH of the antibody or fragment thereof comprises a Kabat heavy chain complementarity determining region-3 (VH-CDR3) amino acid sequence identical, except for four or fewer amino acid substitutions, to a reference VH-CDR3 amino acid sequence selected from the group consisting of: SEQ ID NO: 7, SEQ ID NO: 17, SEQ ID NO: 27, SEQ ID NO: 37, SEQ ID NO: 47, SEQ ID NO: 57, SEQ ID NO: 67, SEQ ID NO: 77, SEQ ID NO: 87, SEQ ID NO: 97, SEQ ID NO: 118, SEQ ID NO:128, SEQ ID NO:138, and SEQ ID NO:148. In further embodiments, the VH-CDR3 amino acid sequence is selected from the group consisting of: SEQ ID NO: 7, SEQ ID NO: 17, SEQ ID NO: 27, SEQ ID NO: 37, SEQ ID NO: 47, SEQ ID NO: 57, SEQ ID NO: 67, SEQ ID NO: 77, SEQ ID NO: 87, SEQ ID NO: 97, SEQ ID NO: 118, SEQ ID NO:128, SEQ ID NO:138, and SEQ ID NO:148.

In some embodiments, the invention provides an isolated antibody or fragment thereof which specifically binds to RON, where the VL of the antibody or fragment thereof comprises a Kabat light chain complementarity determining region-1 (VL-CDR1) amino acid sequence identical, except for four or fewer amino acid substitutions, to a reference VL-CDR1 amino acid sequence selected from the group consisting of: SEQ ID NO: 10, SEQ ID NO: 20, SEQ ID NO: 30, SEQ ID NO: 40, SEQ ID NO: 50, SEQ ID NO: 60, SEQ ID NO: 70, SEQ ID NO: 80, SEQ ID NO: 90, SEQ ID NO: 100, SEQ ID NO: 121, SEQ ID NO: 131, SEQ ID NO:141, and SEQ ID NO: 151. In further embodiments, the VL-CDR1 amino acid sequence is selected from the group consisting of: SEQ ID NO: 10, SEQ ID NO: 20, SEQ ID NO: 30, SEQ ID NO: 40, SEQ ID NO: 50, SEQ ID NO: 60, SEQ ID NO: 70, SEQ ID NO: 80, SEQ ID NO: 90, SEQ ID NO: 100, SEQ ID NO: 121, SEQ ID NO: 131, SEQ ID NO:141, and SEQ ID NO: 151.

In some embodiments, the invention provides an isolated antibody or fragment thereof which specifically binds to RON, where the VL of the antibody or fragment thereof comprises a Kabat light chain complementarity determining region-2 (VL-CDR2) amino acid sequence identical, except for two or fewer amino acid substitutions, to a reference VL-CDR2 amino acid sequence selected from the group consisting of: SEQ ID NO: 11, SEQ ID NO: 21, SEQ ID NO: 31, SEQ ID NO: 41, SEQ ID NO: 51, SEQ ID NO: 61, SEQ ID NO: 71, SEQ ID NO: 81, SEQ ID NO: 91, SEQ ID NO: 101, SEQ ID NO:122, SEQ ID NO:132, SEQ ID NO: 142, and SEQ ID NO:152. In further embodiments, the VL-CDR2 amino acid sequence is selected from the group consisting of: SEQ ID NO: 11, SEQ ID NO: 21, SEQ ID NO: 31, SEQ ID NO: 41, SEQ ID NO: 51, SEQ ID NO: 61, SEQ ID NO: 71, SEQ ID NO: 81, SEQ ID NO: 91, SEQ ID NO: 101, SEQ ID NO:122, SEQ ID NO:132, SEQ ID NO: 142, and SEQ ID NO:152.

In some embodiments, the invention provides an isolated antibody or fragment thereof which specifically binds to RON, where the VL of the antibody or fragment thereof comprises a Kabat light chain complementarity determining region-3 (VL-CDR3) amino acid sequence identical, except for four or fewer amino acid substitutions, to a reference VL-CDR3 amino acid sequence selected from the group consisting of: SEQ ID NO: 12, SEQ ID NO: 22, SEQ ID NO: 32, SEQ ID NO: 42, SEQ ID NO: 52, SEQ ID NO: 62, SEQ ID NO: 72, SEQ ID NO: 82, SEQ ID NO: 92, SEQ ID NO: 102, SEQ ID NO:123, SEQ ID NO: 133, SEQ ID NO: 143 and SEQ ID NO: 153. In further embodiments, the VL-CDR3 amino acid sequence is selected from the group consisting of: SEQ ID NO: 12, SEQ ID NO: 22, SEQ ID NO: 32, SEQ ID NO: 42, SEQ ID NO: 52, SEQ ID NO: 62, SEQ ID NO: 72, SEQ ID NO: 82, SEQ ID NO: 92, SEQ ID NO: 102, SEQ ID NO:123, SEQ ID NO: 133, SEQ ID NO: 143 and SEQ ID NO: 153.

In some embodiments, the invention provides an isolated antibody or fragment thereof which specifically binds to RON, where the VH of the antibody or fragment thereof comprises VH-CDR1, VH-CDR2, and VH-CDR3 amino acid sequences selected from the group consisting of: SEQ ID NOs: 5, 6, and 7; SEQ ID NOs: 15, 16, and 17; SEQ ID NOs: 25, 26, and 27; SEQ ID NOs: 35, 36, and 37; SEQ ID NOs: 45, 46, and 47; SEQ ID NOs: 55, 56, and 57; SEQ ID NOs: 65, 66, and 67; SEQ ID NOs: 75, 76, and 77; SEQ ID NOs: 85, 86, and 87; SEQ ID NOs: 95, 96, and 97; SEQ ID NOs: 116, 117, and 118; SEQ ID NOs: 126, 127, and 128; SEQ ID NOs: 136, 137, and 138; and SEQ ID NOs: 146, 147, and 148, except for one, two, three, or four amino acid substitutions in at least one of said VH-CDRs.

In some embodiments, the invention provides an isolated antibody or fragment thereof which specifically binds to RON, where the VH of the antibody or fragment thereof comprises VH-CDR1, VH-CDR2, and VH-CDR3 amino acid sequences selected from the group consisting of: SEQ ID NOs: 5, 6, and 7; SEQ ID NOs: 15, 16, and 17; SEQ ID NOs: 25, 26, and 27; SEQ ID NOs: 35, 36, and 37; SEQ ID NOs: 45, 46, and 47; SEQ ID NOs: 55, 56, and 57; SEQ ID NOs: 65, 66, and 67; SEQ ID NOs: 75, 76, and 77; SEQ ID NOs: 85, 86, and 87; SEQ ID NOs: 95, 96, and 97; SEQ ID NOs: 116, 117, and 118; SEQ ID NOs: 126, 127, and 128; SEQ ID NOs: 136, 137, and 138; and SEQ ID NOs: 146, 147, and 148.

In some embodiments, the invention provides an isolated antibody or fragment thereof which specifically binds to RON, where the VL of the antibody or fragment thereof comprises VL-CDR1, VL-CDR2, and VL-CDR3 amino acid sequences selected from the group consisting of: SEQ ID NOs: 10, 11, and 12; SEQ ID NOs: 20, 21, and 22; SEQ ID NOs: 30, 31, and 32; SEQ ID NOs: 40, 41, and 42; SEQ ID NOs: 50, 51, and 52; SEQ ID NOs: 60, 61, and 62; SEQ ID NOs: 70, 71, and 72; SEQ ID NOs: 80, 81, and 82; SEQ ID NOs: 90, 91, and 92; SEQ ID NOs: 100, 101, and 102; SEQ ID NOs: 121, 122, and 123; SEQ ID NOs: 131, 132, and 133; SEQ ID NOs: 141, 142, and 143; and SEQ ID NOs: 151, 152, and 153, except for one, two, three, or four amino acid substitutions in at least one of said VL-CDRs.

In some embodiments, the invention provides an isolated antibody or fragment thereof which specifically binds to RON, where the VL of the antibody or fragment thereof comprises VL-CDR1, VL-CDR2, and VL-CDR3 amino acid sequences selected from the group consisting of: SEQ ID NOs: 10, 11, and 12; SEQ ID NOs: 20, 21, and 22; SEQ ID NOs: 30, 31, and 32; SEQ ID NOs: 40, 41, and 42; SEQ ID NOs: 50, 51, and 52; SEQ ID NOs: 60, 61, and 62; SEQ ID NOs: 70, 71, and 72; SEQ ID NOs: 80, 81, and 82; SEQ ID NOs: 90, 91, and 92; SEQ ID NOs: 100, 101, and 102; SEQ ID NOs: 121, 122, and 123; SEQ ID NOs: 131, 132, and 133; SEQ ID NOs: 141, 142, and 143; and SEQ ID NOs: 151, 152, and 153.

In various embodiments of the above-described antibodies or fragments thereof, the VH framework regions and/or VL framework regions are human, except for five or fewer amino acid substitutions.

In some embodiments, the above-described antibodies or fragments thereof bind to a linear epitope or a non-linear conformation epitope

In some embodiments, the above-described antibodies or fragments thereof are multivalent, and comprise at least two heavy chains and at least two light chains.

In some embodiments, the above-described antibodies or fragments thereof are multispecific. In further embodiments, the above-described antibodies or fragments thereof are bispecific.

In various embodiments of the above-described antibodies or fragments thereof, the heavy and light chain variable domains are murine. In further embodiments, the heavy and light chain variable domains are from a monoclonal antibody selected from the group consisting of 1P2E7, 1P3B2, 1P4A3, 1P4A12 and 1P5B10.

In various embodiments of the above-described antibodies or fragments thereof, the heavy and light chain variable domains are fully human. In further embodiments, the heavy and light chain variable domains are from a monoclonal Fab antibody fragment selected from the group consisting of M14-H06, M15-E10, M16-C07, M23-F10, M80-B03, M93-D02, M96-C05, M97-D03, and M98-E12.

In various embodiments, the above-described antibodies or fragments thereof are humanized.

In various embodiments, the above-described antibodies or fragments thereof are chimeric.

In various embodiments, the above-described antibodies or fragments thereof are primatized.

In various embodiments, the above-described antibodies or fragments thereof are fully human.

In certain embodiments, the above-described antibodies or fragments thereof are Fab fragments, Fab′ fragments, F(ab)₂ fragments, or Fv fragments.

In certain embodiments, the above-described antibodies are single chain antibodies.

In certain embodiments, the above-described antibodies or fragments thereof comprise light chain constant regions selected from the group consisting of a human kappa constant region and a human lambda constant region.

In certain embodiments, the above-described antibodies or fragments thereof comprise a heavy chain constant region or fragment thereof. In further embodiments, the heavy chain constant region or fragment thereof is selected from the group consisting of human IgG4, IgG4 agly, IgG1, and IgG1agly.

In some embodiments, the above-described antibodies or fragments thereof specifically bind to a RON polypeptide or fragment thereof, or a RON variant polypeptide, with an affinity characterized by a dissociation constant (K_{D}) which is less than the K_{D} for said reference monoclonal antibody. In further embodiments, the dissociation constant (K_{D}) is no greater than 5×10⁻⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

In some embodiments, the above-described antibodies or fragments thereof preferentially bind to a human RON polypeptide or fragment thereof, relative to a murine RON polypeptide or fragment thereof.

In some embodiments, the above described antibodies or fragments thereof bind to RON expressed on the surface of a cell. In further embodiments, the cell is a malignant cell, a neoplastic cell, a tumor cell, a metastatic cell, or a tumor associated macrophage cell.

In some embodiments, the above described antibodies or fragments thereof block MSP from binding to RON.

In some embodiments, the above described antibodies or fragments thereof inhibit MSP-dependent activation of RON.

In some embodiments, the above described antibodies or fragments thereof inhibit MSP-independent activation of RON.

In some embodiments, the above described antibodies or fragments thereof inhibit RON-mediated activation of the Ras/MAPK signaling pathway.

In some embodiments, the above described antibodies or fragments thereof inhibit RON-mediated phosphorylation of ERK or AKT.

In some embodiments, the above described antibodies or fragments thereof inhibit RON-mediated cell proliferation, tumor cell growth, tumor cell migration, tumor cell invasion or tumor cell metastasis.

In some embodiments, the above described antibodies or fragments thereof induce apoptosis.

In further embodiments, the above described antibodies or fragments thereof further comprise a heterologous polypeptide fused thereto.

In some embodiments, the above described antibodies or fragments thereof are conjugated to an agent selected from the group consisting of cytotoxic agent, a therapeutic agent, cytostatic agent, a biological toxin, a prodrug, a peptide, a protein, an enzyme, a virus, a lipid, a biological response modifier, pharmaceutical agent, a lymphokine, a heterologous antibody or fragment thereof, a detectable label, polyethylene glycol (PEG), and a combination of two or more of any said agents. In further embodiments, the cytotoxic agent is selected from the group consisting of a radionuclide, a biotoxin, an enzymatically active toxin, a cytostatic or cytotoxic therapeutic agent, a prodrugs, an immunologically active ligand, a biological response modifier, or a combination of two or more of any said cytotoxic agents. In further embodiments, the detectable label is selected from the group consisting of an enzyme, a fluorescent label, a chemiluminescent label, a bioluminescent label, a radioactive label, or a combination of two or more of any said detectable labels.

In additional embodiments, the invention includes compositions comprising the above-described antibodies or fragments thereof, and a carrier.

Certain embodiments of the invention include an isolated polynucleotide comprising a nucleic acid which encodes an antibody VH polypeptide, where the amino acid sequence of the VH polypeptide is at least 90% identical to a reference amino acid sequence selected from the group consisting of: SEQ ID NO:4, SEQ ID NO:14, SEQ ID NO:24, SEQ ID NO:34, SEQ ID NO:44, SEQ ID NO:54, SEQ ID NO:64, SEQ ID NO:74, SEQ ID NO:84, SEQ ID NO:94, SEQ ID NO:115, SEQ ID NO:125, SEQ ID NO:135, and SEQ ID NO:145; and where an antibody or antigen binding fragment thereof comprising the VH polypeptide specifically binds to RON. In further embodiments, the amino acid sequence of the VH polypeptide is selected from the group consisting of: SEQ ID NO: SEQ ID NO:4, SEQ ID NO:14, SEQ ID NO:24, SEQ ID NO:34, SEQ ID NO:44, SEQ ID NO:54, SEQ ID NO:64, SEQ ID NO:74, SEQ ID NO:84, SEQ ID NO:94, SEQ ID NO:115, SEQ ID NO:125, SEQ ID NO:135, and SEQ ID NO:145.

In certain embodiments, the nucleotide sequence encoding the VH polypeptide is optimized for increased expression without changing the amino acid sequence of the VH polypeptide. In further embodiments, the optimization comprises identification and removal of splice donor and splice acceptor sites and/or optimization of codon usage for the cells expressing the polynucleotide. In further embodiments, the nucleic acid comprises a nucleotide sequence selected from the group consisting of: SEQ ID NO:3, SEQ ID NO:13, SEQ ID NO:23, SEQ ID NO:33, SEQ ID NO:43, SEQ ID NO:53, SEQ ID NO:63, SEQ ID NO:73, SEQ ID NO:83, SEQ ID NO:93, SEQ ID NO:114, SEQ ID NO:124, SEQ ID NO:134, and SEQ ID NO:144.

In some embodiments, the invention provides an isolated polynucleotide comprising a nucleic acid which encodes an antibody VL polypeptide, where the amino acid sequence of the VL polypeptide is at least 90% identical to a reference amino acid sequence selected from the group consisting of: SEQ ID NO:9, SEQ ID NO:19, SEQ ID NO:29, SEQ ID NO:39, SEQ ID NO:49, SEQ ID NO:59, SEQ ID NO:69, SEQ ID NO:79, SEQ ID NO:89, SEQ ID NO: 99, SEQ ID NO:120, SEQ ID NO: 130, SEQ ID NO:140, and SEQ ID NO:150; and where an antibody or antigen binding fragment thereof comprising the VL polypeptide specifically binds to RON. In further embodiments, the amino acid sequence of the VL polypeptide is selected from the group consisting of: SEQ ID NO:9, SEQ ID NO:19, SEQ ID NO:29, SEQ ID NO:39, SEQ ID NO:49, SEQ ID NO:59, SEQ ID NO:69, SEQ ID NO:79, SEQ ID NO:89, SEQ ID NO:99, SEQ ID NO:120, SEQ ID NO: 130, SEQ ID NO:140, and SEQ ID NO:150.

In certain embodiments, the nucleotide sequence encoding the VL polypeptide is optimized for increased expression without changing the amino acid sequence of said VL polypeptide. In further embodiments, the optimization comprises identification and removal of splice donor and splice acceptor sites and/or optimization of codon usage for the cells expressing the polynucleotide. In further embodiments, the nucleic acid comprises a nucleotide sequence selected from the group consisting of: SEQ ID NO:8, SEQ ID NO:18, SEQ ID NO:28, SEQ ID NO:38, SEQ ID NO:48, SEQ ID NO:58, SEQ ID NO:68, SEQ ID NO:78, SEQ ID NO:88, SEQ ID NO:98, SEQ ID NO: 119, SEQ ID NO:129, SEQ ID NO:139, and SEQ ID NO:149.

In certain other embodiments, the invention provides an isolated polynucleotide comprising a nucleic acid which encodes an antibody VH polypeptide, where the amino acid sequence of the VH polypeptide is identical, except for 20 or fewer conservative amino acid substitutions, to a reference amino acid sequence selected from the group consisting of: SEQ ID NO:4, SEQ ID NO:14, SEQ ID NO:24, SEQ ID NO:34, SEQ ID NO:44, SEQ ID NO:54, SEQ ID NO:64, SEQ ID NO:74, SEQ ID NO:84, SEQ ID NO:94, SEQ ID NO:115, SEQ ID NO:125, SEQ ID NO:135, and SEQ ID NO:145; and where an antibody or antigen binding fragment thereof comprising said VH polypeptide specifically binds to RON.

In some embodiments, the invention provides an isolated polynucleotide comprising a nucleic acid which encodes an antibody VL polypeptide, where the amino acid sequence of the VL polypeptide is identical, except for 20 or fewer conservative amino acid substitutions, to a reference amino acid sequence selected from the group consisting of: SEQ ID NO:9, SEQ ID NO:19, SEQ ID NO:29, SEQ ID NO:39, SEQ ID NO:49, SEQ ID NO:59, SEQ ID NO:69, SEQ ID NO:79, SEQ ID NO:89, SEQ ID NO:99, SEQ ID NO:120, SEQ ID NO: 130, SEQ ID NO:140, and SEQ ID NO:150; and wherein an antibody or antigen binding fragment thereof comprising said VL polypeptide specifically binds to RON.

In some embodiments, the invention provides an isolated polynucleotide comprising a nucleic acid which encodes a VH-CDR1 amino acid sequence identical, except for two or fewer amino acid substitutions, to a reference VH-CDR1 amino acid sequence selected from the group consisting of: SEQ ID NO: 5, SEQ ID NO: 15, SEQ ID NO: 25, SEQ ID NO: 35, SEQ ID NO: 45, SEQ ID NO: 55, SEQ ID NO: 65, SEQ ID NO: 75, SEQ ID NO: 85, SEQ ID NO: 95, SEQ ID NO: 116, SEQ ID NO:126, SEQ ID NO:136, and SEQ ID NO:146, and where an antibody or antigen binding fragment thereof comprising the VH-CDR1 specifically binds to RON. In further embodiments, the VH-CDR1 amino acid sequence is selected from the group consisting of: SEQ ID NO: 5, SEQ ID NO: 15, SEQ ID NO: 25, SEQ ID NO: 35, SEQ ID NO: 45, SEQ ID NO: 55, SEQ ID NO: 65, SEQ ID NO: 75, SEQ ID NO: 85, SEQ ID NO: 95, SEQ ID NO: 116, SEQ ID NO:126, SEQ ID NO:136, and SEQ ID NO:146.

In some embodiments, the invention provides an isolated polynucleotide comprising a nucleic acid which encodes a VH-CDR2 amino acid sequence identical, except for four or fewer amino acid substitutions, to a reference VH-CDR2 amino acid sequence selected from the group consisting of: SEQ ID NO: 6, SEQ ID NO: 16, SEQ ID NO: 26, SEQ ID NO: 36, SEQ ID NO: 46, SEQ ID NO: 56, SEQ ID NO: 66, SEQ ID NO: 76, SEQ ID NO: 86, SEQ ID NO: 96, SEQ ID NO:117, SEQ ID NO:127, SEQ ID NO:137, and SEQ ID NO:147, and where an antibody or antigen binding fragment thereof comprising the VH-CDR2 specifically binds to RON. In further embodiments, the VH-CDR2 amino acid sequence is selected from the group consisting of: SEQ ID NO: 6, SEQ ID NO: 16, SEQ ID NO: 26, SEQ ID NO: 36, SEQ ID NO: 46, SEQ ID NO: 56, SEQ ID NO: 66, SEQ ID NO: 76, SEQ ID NO: 86, SEQ ID NO: 96, SEQ ID NO:117, SEQ ID NO:127, SEQ ID NO:137, and SEQ ID NO:147.

In some embodiments, the invention provides an isolated polynucleotide comprising a nucleic acid which encodes a VH-CDR3 amino acid sequence identical, except for four or fewer amino acid substitutions, to a reference VH-CDR3 amino acid sequence selected from the group consisting of: SEQ ID NO: 7, SEQ ID NO: 17, SEQ ID NO: 27, SEQ ID NO: 37, SEQ ID NO: 47, SEQ ID NO: 57, SEQ ID NO: 67, SEQ ID NO: 77, SEQ ID NO: 87, SEQ ID NO: 97, SEQ ID NO: 118, SEQ ID NO:128, SEQ ID NO:138, and SEQ ID NO:148; and where an antibody or antigen binding fragment thereof comprising the VH-CDR3 specifically binds to RON. In further embodiments, the VH-CDR3 amino acid sequence is selected from the group consisting of: SEQ ID NO: 7, SEQ ID NO: 17, SEQ ID NO: 27, SEQ ID NO: 37, SEQ ID NO: 47, SEQ ID NO: 57, SEQ ID NO: 67, SEQ ID NO: 77, SEQ ID NO: 87, SEQ ID NO: 97, SEQ ID NO: 118, SEQ ID NO:128, SEQ ID NO:138, and SEQ ID NO:148.

In some embodiments, the invention provides an isolated polynucleotide comprising a nucleic acid which encodes a VL-CDR1 amino acid sequence identical, except for four or fewer amino acid substitutions, to a reference VL-CDR1 amino acid sequence selected from the group consisting of: SEQ ID NO: 10, SEQ ID NO: 20, SEQ ID NO: 30, SEQ ID NO: 40, SEQ ID NO: 50, SEQ ID NO: 60, SEQ ID NO: 70, SEQ ID NO: 80, SEQ ID NO: 90, SEQ ID NO: 100, SEQ ID NO: 121, SEQ ID NO: 131, SEQ ID NO:141, and SEQ ID NO: 151; and where an antibody or antigen binding fragment thereof comprising the VL-CDR1 specifically binds to RON. In further embodiments, the VL-CDR1 amino acid sequence is selected from the group consisting of: SEQ ID NO: 10, SEQ ID NO: 20, SEQ ID NO: 30, SEQ ID NO: 40, SEQ ID NO: 50, SEQ ID NO: 60, SEQ ID NO: 70, SEQ ID NO: 80, SEQ ID NO: 90, SEQ ID NO: 100, SEQ ID NO: 121, SEQ ID NO: 131, SEQ ID NO:141, and SEQ ID NO: 151.

In some embodiments, the invention provides an isolated polynucleotide comprising a nucleic acid which encodes a VL-CDR2 amino acid sequence identical, except for two or fewer amino acid substitutions, to a reference VL-CDR2 amino acid sequence selected from the group consisting of: SEQ ID NO: 11, SEQ ID NO: 21, SEQ ID NO: 31, SEQ ID NO: 41, SEQ ID NO: 51, SEQ ID NO: 61, SEQ ID NO: 71, SEQ ID NO: 81, SEQ ID NO: 91, SEQ ID NO: 101, SEQ ID NO:122, SEQ ID NO:132, SEQ ID NO: 142, and SEQ ID NO:152; and wherein an antibody or antigen binding fragment thereof comprising said VL-CDR2 specifically binds to RON. In further embodiments, the VL-CDR2 amino acid sequence is selected from the group consisting of: SEQ ID NO: 11, SEQ ID NO: 21, SEQ ID NO: 31, SEQ ID NO: 41, SEQ ID NO: 51, SEQ ID NO: 61, SEQ ID NO: 71, SEQ ID NO: 81, SEQ ID NO: 91, SEQ ID NO: 101, SEQ ID NO:122, SEQ ID NO:132, SEQ ID NO: 142, and SEQ ID NO:152.

In some embodiments, the invention provides an isolated polynucleotide comprising a nucleic acid which encodes a VL-CDR3 amino acid sequence identical, except for four or fewer amino acid substitutions, to a reference VL-CDR3 amino acid sequence selected from the group consisting of: SEQ ID NO: 12, SEQ ID NO: 22, SEQ ID NO: 32, SEQ ID NO: 42, SEQ ID NO: 52, SEQ ID NO: 62, SEQ ID NO: 72, SEQ ID NO: 82, SEQ ID NO: 92, SEQ ID NO: 102, SEQ ID NO:123, SEQ ID NO: 133, SEQ ID NO: 143 and SEQ ID NO: 153; and wherein an antibody or antigen binding fragment thereof comprising said VL-CDR3 specifically binds to RON. In further embodiments, the VL-CDR3 amino acid sequence is selected from the group consisting of: SEQ ID NO: 12, SEQ ID NO: 22, SEQ ID NO: 32, SEQ ID NO: 42, SEQ ID NO: 52, SEQ ID NO: 62, SEQ ID NO: 72, SEQ ID NO: 82, SEQ ID NO: 92, SEQ ID NO: 102, SEQ ID NO:123, SEQ ID NO: 133, SEQ ID NO: 143 and SEQ ID NO: 153.

In some embodiments, the invention provides an isolated polynucleotide comprising a nucleic acid which encodes an antibody VH polypeptide, where the VH polypeptide comprises VH-CDR1, VH-CDR2, and VH-CDR3 amino acid sequences selected from the group consisting of: SEQ ID NOs: 5, 6, and 7; SEQ ID NOs: 15, 16, and 17; SEQ ID NOs: 25, 26, and 27; SEQ ID NOs: 35, 36, and 37; SEQ ID NOs: 45, 46, and 47; SEQ ID NOs: 55, 56, and 57; SEQ ID NOs: 65, 66, and 67; SEQ ID NOs: 75, 76, and 77; SEQ ID NOs: 85, 86, and 87; SEQ ID NOs: 95, 96, and 97; SEQ ID NOs: 116, 117, and 118; SEQ ID NOs: 126, 127, and 128; SEQ ID NOs: 136, 137, and 138; and SEQ ID NOs: 146, 147, and 148; and where an antibody or antigen binding fragment thereof comprising the VH-CDR3 specifically binds to RON.

In some embodiments, the invention provides an isolated polynucleotide comprising a nucleic acid which encodes an antibody VL polypeptide, wherein said VL polypeptide comprises VH-CDR1, VH-CDR2, and VH-CDR3 amino acid sequences selected from the group consisting of: SEQ ID NOs: 10, 11, and 12; SEQ ID NOs: 20, 21, and 22; SEQ ID NOs: 30, 31, and 32; SEQ ID NOs: 40, 41, and 42; SEQ ID NOs: 50, 51, and 52; SEQ ID NOs: 60, 61, and 62; SEQ ID NOs: 70, 71, and 72; SEQ ID NOs: 80, 81, and 82; SEQ ID NOs: 90, 91, and 92; SEQ ID NOs: 100, 101, and 102; SEQ ID NOs: 121, 122, and 123; SEQ ID NOs: 131, 132, and 133; SEQ ID NOs: 141, 142, and 143; and SEQ ID NOs: 151, 152, and 153; and wherein an antibody or antigen binding fragment thereof comprising said VL-CDR3 specifically binds to RON.

In some embodiments, the above-described polynucleotides further comprise a nucleic acid encoding a signal peptide fused to the antibody VH polypeptide or the antibody VL polypeptide.

In certain other embodiments, the above-described polynucleotides further comprise a nucleic acid encoding a heavy chain constant region CH1 domain fused to the VH polypeptide, encoding a heavy chain constant region CH2 domain fused to the VH polypeptide, encoding a heavy chain constant region CH3 domain fused to the VH polypeptide, or encoding a heavy chain hinge region fused to said VH polypeptide. In further embodiments, the heavy chain constant region is selected from the group consisting of human IgG4, IgG4 agly, IgG1 or IgG1 agly.

In some embodiments, the above-described polynucleotides comprise a nucleic acid encoding a light chain constant region domain fused to said VL polypeptide. In further embodiments, the light chain constant region is human kappa.

In various embodiments of the above-described polynucleotides, the antibody or antigen-binding fragment thereof comprising a polypeptide encoded by the nucleic acid specifically binds the same RON epitope as a reference monoclonal Fab antibody fragment selected from the group consisting of M14-H06, M15-E10, M16-C07, M23-F10, and M80-B03 or a reference monoclonal antibody selected from the group consisting of 1P2E7, 1P3B2, 1P4A3, 1P4A12 and 1P5B10.

In various other embodiments of the above-described polynucleotides, the antibody or antigen-binding fragment thereof comprising a polypeptide encoded by the nucleic acid competitively inhibits a reference monoclonal Fab antibody fragment selected from the group consisting of M14-H06, M15-E10, M16-C07, M23-F10, M80-B03, M93-D02, M96-C05, M97-D03, and M98-E12 or a reference monoclonal antibody selected from the group consisting of 1P2E7, 1P3B2, 1P4A3, 1P4A12 and 1P5B10.

In various embodiments of the above-describe polynucleotides, the framework regions of the VH polypeptide or VL polypeptide are human, except for five or fewer amino acid substitutions.

In various embodiments of the above-described polynucleotides, the invention provides an antibody or antigen-binding fragment thereof comprising the polypeptide encoded by the nucleic acid, that binds to a linear epitope or a non-linear conformational epitope.

In various embodiments of the above-described polynucleotides, the antibody or antigen-binding fragment thereof comprising the polypeptide encoded by the nucleic acid is multivalent, and comprises at least two heavy chains and at least two light chains.

In certain embodiments of the above-described polynucleotides, the antibody or antigen-binding fragment thereof comprising the polypeptide encoded by the nucleic acid is multispecific. In further embodiments, the antibody or antigen-binding fragment thereof comprising the polypeptide encoded by the nucleic acid is bispecific.

In various embodiments of the above-described polynucleotides, the antibody or antigen-binding fragment thereof comprising the polypeptide encoded by the nucleic acid comprises heavy and light chain variable domains which are fully human. In further embodiments, the heavy and light chain variable domains are identical to those of a monoclonal Fab antibody fragment selected from the group consisting of M14-H06, M15-E10, M16-C07, M23-F10, M80-B03, M93-D02, M96-C05, M97-D03, and M98-E12.

In various embodiments of the above-described polynucleotides, the antibody or antigen-binding fragment thereof comprising the polypeptide encoded by the nucleic acid comprises heavy and light chain variable domains which are murine. In further embodiments, the heavy and light chain variable domains are identical to those of a monoclonal antibody selected from the group consisting of 1P2E7, 1P3B2, 1P4A3, 1P4A12 and 1P5B10.

In various embodiments of the above-described polynucleotides, the antibody or antigen-binding fragment thereof comprising the polypeptide encoded by the nucleic acid is humanized.

In various embodiments of the above-described polynucleotides, the antibody or antigen-binding fragment thereof comprising the polypeptide encoded by the nucleic acid is primatized.

In various embodiments of the above-described polynucleotides, the antibody or antigen-binding fragment thereof comprising the polypeptide encoded by the nucleic acid is chimeric.

In some embodiments of the above-described polynucleotides, the antibody or antigen-binding fragment thereof comprising the polypeptide encoded by the nucleic acid is fully human.

In various embodiments of the above-described polynucleotides, the antibody or antigen-binding fragment thereof comprising the polypeptide encoded by the nucleic acid is an Fab fragment, an Fab′ fragment, an F(ab)₂ fragment, or an Fv fragment. In certain embodiments of the above-described polynucleotides, the antibody or antigen-binding fragment thereof comprising the polypeptide encoded by the nucleic acid is a single chain antibody.

In some embodiments of the above-described polynucleotides, the antibody or antigen-binding fragment thereof comprising the polypeptide encoded by the nucleic acid specifically binds to an RON polypeptide or fragment thereof, or an RON variant polypeptide, with an affinity characterized by a dissociation constant (K_{D}) no greater than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

In some embodiments of the above-described polynucleotides, the antibody or antigen-binding fragment thereof comprising the polypeptide encoded by the nucleic acid preferentially binds to a human RON polypeptide or fragment thereof, relative to a murine RON polypeptide or fragment thereof.

In some embodiments of the above-described polynucleotides, the antibody or antigen-binding fragment thereof comprising the polypeptide encoded by the nucleic acid binds to RON expressed on the surface of a cell. In further embodiments, the cell is a malignant cell, a neoplastic cell, a tumor cell, or a metastatic cell.

In some embodiments of the above-described polynucleotides, the antibody or antigen-binding fragment thereof comprising the polypeptide encoded by said nucleic acid blocks MSP from binding to RON.

In some embodiments of the above-described polynucleotides, the antibody or antigen-binding fragment thereof comprising the polypeptide encoded by the nucleic acid inhibits MSP-dependent RON activation.

In some embodiments of the above-described polynucleotides, the antibody or antigen-binding fragment thereof comprising the polypeptide encoded by the nucleic acid inhibits MSP-independent RON activation.

In some embodiments of the above-described polynucleotides, the antibody or antigen-binding fragment thereof comprising the polypeptide encoded by the nucleic acid inhibits activation of the Ras/MAPK signaling pathway.

In some embodiments of the above-described polynucleotides, the antibody or antigen-binding fragment thereof comprising the polypeptide encoded by the nucleic acid inhibits RON-mediated phosphorylation of ERK.

In some embodiments of the above-described polynucleotides, the antibody or antigen-binding fragment thereof comprising the polypeptide encoded by the nucleic acid inhibits RON-mediated cell proliferation or tumor cell growth.

In some embodiments of the above-described polynucleotides, the antibody or antigen-binding fragment thereof comprising the polypeptide encoded by the nucleic acid induces apoptosis.

In some embodiments, the above-described polynucleotides further comprise a nucleic acid encoding a heterologous polypeptide.

In some embodiments of the above-described polynucleotides, the antibody or antigen-binding fragment thereof comprising the polypeptide encoded by the nucleic acid is conjugated to an agent selected from the group consisting of cytotoxic agent, a therapeutic agent, cytostatic agent, a biological toxin, a prodrug, a peptide, a protein, an enzyme, a virus, a lipid, a biological response modifier, pharmaceutical agent, a lymphokine, a heterologous antibody or fragment thereof, a detectable label, polyethylene glycol (PEG), and a combination of two or more of any said agents. In further embodiments, the cytotoxic agent is selected from the group consisting of a radionuclide, a biotoxin, an enzymatically active toxin, a cytostatic or cytotoxic therapeutic agent, a prodrugs, an immunologically active ligand, a biological response modifier, or a combination of two or more of any said cytotoxic agents. In certain other embodiments, the detectable label is selected from the group consisting of an enzyme, a fluorescent label, a chemiluminescent label, a bioluminescent label, a radioactive label, or a combination of two or more of any said detectable labels.

In some embodiments, the invention provides compositions comprising the above-described polynucleotides.

In certain other embodiments, the invention provides vectors comprising the above-described polynucleotides. In further embodiments, the polynucleotides are operably associated with a promoter. In additional embodiments, the invention provides host cells comprising such vectors. In further embodiments, the invention provides vectors where the polynucleotide is operably associated with a promoter.

In additional embodiments, the invention provides a method of producing an antibody or fragment thereof which specifically binds RON, comprising culturing a host cell containing a vector comprising the above-described polynucleotides, and recovering said antibody, or fragment thereof. In further embodiments, the invention provides an isolated polypeptide produced by the above-described method.

In some embodiments, the invention provides isolated polypeptides encoded by the above-described polynucleotides.

In further embodiments of the above-described polypeptides, the antibody or fragment thereof comprising the polypeptide specifically binds to RON. Other embodiments include the isolated antibody or fragment thereof comprising the above-described polypeptides.

In some embodiments, the invention provides a composition comprising an isolated VH encoding polynucleotide and an isolated VL encoding polynucleotide, where the VH encoding polynucleotide and the VL encoding polynucleotide, respectively, comprise nucleic acids encoding amino acid sequences at least 90% identical to reference amino acid sequences selected from the group consisting of: SEQ ID NO:4 and SEQ ID NO:9; SEQ ID NO:14 and SEQ ID NO:19; SEQ ID NO:24 and SEQ ID NO:29; SEQ ID NO:34 and SEQ ID NO:39; SEQ ID NO:44 and SEQ ID NO:49; SEQ ID NO:54 and SEQ ID NO:59; SEQ ID NO:64 and SEQ ID NO:69; SEQ ID NO:74 and SEQ ID NO:79; SEQ ID NO:84 and SEQ ID NO:89; SEQ ID NO:94 and SEQ ID NO:99, SEQ ID NO: 115 and SEQ ID NO: 120, SEQ ID NO: 125 and SEQ ID NO:130, SEQ ID NO: 135 and SEQ ID NO: 140, and SEQ ID NO: 145 and SEQ ID NO: 150; and where an antibody or fragment thereof encoded by the VH and VL encoding polynucleotides specifically binds RON. In further embodiments, the VH encoding polynucleotide and said VL encoding polynucleotide, respectively, comprise nucleic acids encoding amino acid sequences selected from the group consisting of: SEQ ID NO:4 and SEQ ID NO:9; SEQ ID NO:14 and SEQ ID NO:19; SEQ ID NO:24 and SEQ ID NO:29; SEQ ID NO:34 and SEQ ID NO:39; SEQ ID NO:44 and SEQ ID NO:49; SEQ ID NO:54 and SEQ ID NO:59; SEQ ID NO:64 and SEQ ID NO:69; SEQ ID NO:74 and SEQ ID NO:79; SEQ ID NO:84 and SEQ ID NO:89; SEQ ID NO:94 and SEQ ID NO:99, SEQ ID NO: 115 and SEQ ID NO: 120, SEQ ID NO: 125 and SEQ ID NO:130, SEQ ID NO: 135 and SEQ ID NO: 140, and SEQ ID NO: 145 and SEQ ID NO: 150.

In certain other embodiments, the invention provides a composition comprising an isolated VH encoding polynucleotide and an isolated VL encoding polynucleotide, where the VH encoding polynucleotide and the VL encoding polynucleotide, respectively, comprise nucleic acids encoding amino acid sequences identical, except for less than 20 conservative amino acid substitutions, to reference amino acid sequences selected from the group consisting of: SEQ ID NO:4 and SEQ ID NO:9; SEQ ID NO:14 and SEQ ID NO:19; SEQ ID NO:24 and SEQ ID NO:29; SEQ ID NO:34 and SEQ ID NO:39; SEQ ID NO:44 and SEQ ID NO:49; SEQ ID NO:54 and SEQ ID NO:59; SEQ ID NO:64 and SEQ ID NO:69; SEQ ID NO:74 and SEQ ID NO:79; SEQ ID NO:84 and SEQ ID NO:89; SEQ ID NO:94 and SEQ ID NO:99, SEQ ID NO: 115 and SEQ ID NO: 120, SEQ ID NO: 125 and SEQ ID NO:130, SEQ ID NO: 135 and SEQ ID NO: 140, and SEQ ID NO: 145 and SEQ ID NO: 150; and where an antibody or fragment thereof encoded by the VH and VL encoding polynucleotides specifically binds RON.

In further embodiments, the VH encoding polynucleotide encodes a VH polypeptide comprising VH-CDR1, VH-CDR2, and VH-CDR3 amino acid sequences selected from the group consisting of: SEQ ID NOs: 5, 6, and 7; SEQ ID NOs: 15, 16, and 17; SEQ ID NOs: 25, 26, and 27; SEQ ID NOs: 35, 36, and 37; SEQ ID NOs: 45, 46, and 47; SEQ ID NOs: 55, 56, and 57; SEQ ID NOs: 65, 66, and 67; SEQ ID NOs: 75, 76, and 77; SEQ ID NOs: 85, 86, and 87; SEQ ID NOs: 95, 96, and 97; SEQ ID NOs: 116, 117, and 118; SEQ ID NOs: 126, 127, and 128; SEQ ID NOs: 136, 137, and 138; and SEQ ID NOs: 146, 147, and 148; where the VL encoding polynucleotide encodes a VL polypeptide comprising VL-CDR1, VL-CDR2, and VL-CDR3 amino acid sequences selected from the group consisting of: SEQ ID NOs: 10, 11, and 12; SEQ ID NOs: 20, 21, and 22; SEQ ID NOs: 30, 31, and 32; SEQ ID NOs: 40, 41, and 42; SEQ ID NOs: 50, 51, and 52; SEQ ID NOs: 60, 61, and 62; SEQ ID NOs: 70, 71, and 72; SEQ ID NOs: 80, 81, and 82; SEQ ID NOs: 90, 91, and 92; SEQ ID NOs: 100, 101, and 102; SEQ ID NOs: 121, 122, and 123; SEQ ID NOs: 131, 132, and 133; SEQ ID NOs: 141, 142, and 143; and SEQ ID NOs: 151, 152, and 153; and where an antibody or fragment thereof encoded by the VH and VL encoding polynucleotides specifically binds RON.

In various embodiments of the above-described compositions, the VH encoding polynucleotide further comprises a nucleic acid encoding a signal peptide fused to the antibody VH polypeptide.

In various embodiments of the above-described compositions, the VL encoding polynucleotide further comprises a nucleic acid encoding a signal peptide fused to the antibody VL polypeptide.

In some embodiments of the above-described compositions, the VH encoding polynucleotide further comprises a nucleic acid encoding a heavy chain constant region CH1 domain fused to the VH polypeptide, further comprises a nucleic acid encoding a heavy chain constant region CH2 domain fused to the VH polypeptide, further comprises a nucleic acid encoding a heavy chain constant region CH3 domain fused to the VH polypeptide, or further comprises a nucleic acid encoding a heavy chain hinge region fused to the VH polypeptide. In further embodiments, the heavy chain constant region is selected from the group consisting of human IgG4, IgG4 agly, IgG1 or IgG1 agly.

In some embodiments of the above-described compositions, the VL encoding polynucleotide further comprises a nucleic acid encoding a light chain constant region domain fused to the VL polypeptide. In further embodiments, the light chain constant region is human kappa.

In some embodiments of the above-described compositions, the antibody or fragment thereof encoded by the VH and VL encoding polynucleotides specifically binds the same RON epitope as a reference monoclonal Fab antibody fragment selected from the group consisting of M14-H06, M15-E10, M16-C07, M23-F10, M80-B03, M93-D02, M96-C05, M97-D03, and M98-E12 or a reference monoclonal antibody selected from the group consisting of 1P2E7, 1P3B2, 1P4A3, 1P4A12 and 1P5B10.

In some embodiments of the above-described compositions, the antibody or fragment thereof encoded by the VH and VL encoding polynucleotides competitively inhibits a reference monoclonal Fab antibody fragment selected from the group consisting of M14-H06, M15-E10, M16-C07, M23-F10, M80-B03, M93-D02, M96-C05, M97-D03, and M98-E12 or a reference monoclonal antibody selected from the group consisting of 1P2E7, 1P3B2, 1P4A3, 1P4A12 and 1P5B10 from binding to RON.

In some embodiments of the above-described compositions, the framework regions of the VH and VL polypeptides are human, except for five or fewer amino acid substitutions.

In some embodiments of the above-described compositions, the antibody or fragment thereof encoded by the VH and VL encoding polynucleotides binds to a linear epitope or a non-linear conformational epitope.

In some embodiments of the above-described compositions, the antibody or fragment thereof encoded by the VH and VL encoding polynucleotides is multivalent, and comprises at least two heavy chains and at least two light chains.

In some embodiments of the above-described compositions, the antibody or fragment thereof encoded by the VH and VL encoding polynucleotides is multispecific. In further embodiments, the antibody or fragment thereof encoded by the VH and VL encoding polynucleotides is bispecific.

In some embodiments of the above-described compositions, the antibody or fragment thereof encoded by the VH and VL encoding polynucleotides comprises heavy and light chain variable domains which are fully human. In further embodiments, the heavy and light chain variable domains are identical to those of a monoclonal Fab antibody fragment selected from the group consisting of M14-H06, M15-E10, M16-C07, M23-F10, M80-B03, M93-D02, M96-C05, M97-D03, and M98-E12.

In some embodiments of the above-described compositions, the antibody or fragment thereof encoded by the VH and VL encoding polynucleotides comprises heavy and light chain variable domains which are murine. In further embodiments, the heavy and light chain variable domains are identical to those of a monoclonal antibody selected from the group consisting of 1P2E7, 1P3B2, 1P4A3, 1P4A12 and 1P5B10.

In various embodiments of the above-described compositions, the antibody or antigen-binding fragment thereof comprising the polypeptide encoded by the nucleic acid is humanized.

In various embodiments of the above-described compositions, the antibody or antigen-binding fragment thereof comprising the polypeptide encoded by the nucleic acid is primatized.

In various embodiments of the above-described compositions, the antibody or antigen-binding fragment thereof comprising the polypeptide encoded by the nucleic acid is chimeric.

In some embodiments of the above-described compositions, the antibody or antigen-binding fragment thereof comprising the polypeptide encoded by the nucleic acid is fully human.

In various embodiments of the above-described compositions, the antibody or antigen-binding fragment thereof comprising the polypeptide encoded by the nucleic acid is an Fab fragment, an Fab′ fragment, an F(ab)₂ fragment, or an Fv fragment. In certain embodiments of the above-described compositions, the antibody or antigen-binding fragment thereof comprising the polypeptide encoded by the nucleic acid is a single chain antibody.

In some embodiments of the above-described compositions, the antibody or antigen-binding fragment thereof comprising the polypeptide encoded by the nucleic acid specifically binds to an RON polypeptide or fragment thereof, or an RON variant polypeptide, with an affinity characterized by a dissociation constant (K_{D}) no greater than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵-M.

In some embodiments of the above-described compositions, the antibody or antigen-binding fragment thereof comprising the polypeptide encoded by the nucleic acid preferentially binds to a human RON polypeptide or fragment thereof, relative to a murine RON polypeptide or fragment thereof.

In some embodiments of the above-described compositions, the antibody or antigen-binding fragment thereof comprising the polypeptide encoded by the nucleic acid binds to RON expressed on the surface of a cell. In further embodiments, the cell is a malignant cell, a neoplastic cell, a tumor cell, a metastatic cell or a tumor associated macrophage.

In some embodiments of the above-described compositions, the antibody or antigen-binding fragment thereof comprising the polypeptide encoded by said nucleic acid blocks MSP from binding to RON.

In some embodiments of the above-described compositions, the antibody or antigen-binding fragment thereof comprising the polypeptide encoded by the nucleic acid inhibits MSP-dependent RON activation.

In some embodiments of the above-described compositions, the antibody or antigen-binding fragment thereof comprising the polypeptide encoded by the nucleic acid inhibits MSP-independent RON activation.

In some embodiments of the above-described compositions, the antibody or antigen-binding fragment thereof comprising the polypeptide encoded by the nucleic acid inhibits activation of the Ras/MAPK signaling pathway.

In some embodiments of the above-described compositions, the antibody or antigen-binding fragment thereof comprising the polypeptide encoded by the nucleic acid inhibits RON-mediated phosphorylation of ERK or AKT.

In some embodiments of the above-described compositions, the antibody or antigen-binding fragment thereof comprising the polypeptide encoded by the nucleic acid inhibits RON-mediated cell proliferation, tumor cell growth, tumor cell migration, tumor cell invasion or tumor cell metastasis.

In some embodiments of the above-described compositions, the antibody or antigen-binding fragment thereof comprising the polypeptide encoded by the nucleic acid induces apoptosis.

In some embodiments, the above-described compositions, the VH encoding polynucleotide, the VL encoding polynucleotide, or both the VH and the VL encoding polynucleotides further comprise a nucleic acid encoding a heterologous polypeptide.

In some embodiments of the above-described compositions, the antibody or antigen-binding fragment thereof comprising the polypeptide encoded by the nucleic acid is conjugated to an agent selected from the group consisting of cytotoxic agent, a therapeutic agent, cytostatic agent, a biological toxin, a prodrug, a peptide, a protein, an enzyme, a virus, a lipid, a biological response modifier, pharmaceutical agent, a lymphokine, a heterologous antibody or fragment thereof, a detectable label, polyethylene glycol (PEG), and a combination of two or more of any said agents. In further embodiments, the cytotoxic agent is selected from the group consisting of a radionuclide, a biotoxin, an enzymatically active toxin, a cytostatic or cytotoxic therapeutic agent, a prodrugs, an immunologically active ligand, a biological response modifier, or a combination of two or more of any said cytotoxic agents. In certain other embodiments, the detectable label is selected from the group consisting of an enzyme, a fluorescent label, a chemiluminescent label, a bioluminescent label, a radioactive label, or a combination of two or more of any said detectable labels.

In some embodiments of the above-described compositions, the VH encoding polynucleotide is contained on a first vector and the VL encoding polynucleotide is contained on a second vector. In further embodiments, the VH encoding polynucleotide is operably associated with a first promoter and the VL encoding polynucleotide is operably associated with a second promoter. In certain other embodiments, the first and second promoters are copies of the same promoter. In further embodiments, the first and second promoters are non-identical.

In various embodiments of the above-described compositions, the first vector and the second vector are contained in a single host cell.

In certain other embodiments of the above-described compositions, the first vector and the second vector are contained in separate host cells.

In some embodiments, the invention provides a method of producing an antibody or fragment thereof which specifically binds RON, comprising culturing the above-described host cells, and recovering the antibody, or fragment thereof.

In other embodiments, the invention provides a method of producing an antibody or fragment thereof which specifically binds RON, comprising co-culturing separate host cells, and recovering the antibody, or fragment thereof. In further embodiments of the above-described method, the invention provides combining the VH and VL encoding polypeptides, and recovering the antibody, or fragment thereof.

In some embodiments, the invention provides an antibody or fragment thereof which specifically binds RON, produced by the above-described methods.

In some embodiments, the invention provides compositions, where the VH encoding polynucleotide and the VL encoding polynucleotide are on the same vector, as well as the vectors therein.

In various embodiments of the above described vectors, the VH encoding polynucleotide and the VL encoding polynucleotide are each operably associated with a promoter.

In various embodiments of the above described vectors, the VH encoding polynucleotide and the VL encoding polynucleotide are fused in frame, are co-transcribed from a single promoter operably associated therewith, and are cotranslated into a single chain antibody or antigen-binding fragment thereof.

In various embodiments of the above described vectors, the VH encoding polynucleotide and said VL encoding polynucleotide are co-transcribed from a single promoter operably associated therewith, but are separately translated. In further embodiments, the vectors further comprise an IRES sequence disposed between the VH encoding polynucleotide and the VL encoding polynucleotide. In certain other embodiments, the polynucleotide encoding a VH and the polynucleotide encoding a VL are separately transcribed, each being operably associated with a separate promoter. In further embodiments, the separate promoters are copies of the same promoter or the separate promoters are non-identical.

In some embodiments, the invention provides host cells comprising the above-described vectors.

In other embodiments, the invention provides a method of producing an antibody or fragment thereof which specifically binds RON, comprising culturing the above-described host cells, and recovering the antibody, or fragment thereof.

In some embodiments, the invention provides an antibody or fragment thereof which specifically binds RON, produced by the above-described methods.

In some embodiments, the invention provides a method for treating a hyperproliferative disorder in an animal, comprising administering to an animal in need of treatment a composition comprising: a) an isolated antibody or fragment as described above; and b) a pharmaceutically acceptable carrier. In further embodiments, the hyperproliferative disease or disorder is selected from the group consisting of cancer, a neoplasm, a tumor, a malignancy, or a metastasis thereof.

In various embodiments of the above-described methods, the antibody or fragment thereof specifically binds to RON expressed on the surface of a malignant cell. In further embodiments, the binding of the antibody or fragment thereof to the malignant cell results in growth inhibition of the malignant cell.

In various embodiments of the above-described methods, the antibody or fragment thereof inhibits RON phosphorylation or inhibits tumor cell proliferation. In further embodiments, the tumor cell proliferation is inhibited through the prevention or retardation of metastatic growth.

In various embodiments of the above-described methods, the antibody or fragment thereof inhibits tumor cell migration. In further embodiments, the tumor cell proliferation is inhibited through the prevention or retardation of tumor spread to adjacent tissues.

In various embodiments of the above-described methods, the hyperproliferative disease or disorder is a neoplasm located in the: prostate, colon, abdomen, bone, breast, digestive system, liver, pancreas, peritoneum, adrenal gland, parathyroid gland, pituitary gland, testicles, ovary, thymus, thyroid, eye, head, neck, central nervous system, peripheral nervous system, lymphatic system, pelvis, skin, soft tissue, spleen, thoracic region, or urogenital tract.

In various embodiments of the above-described methods, the hyperproliferative disease is cancer, said cancer selected from the group consisting of: epithelial squamous cell cancer, melanoma, leukemia, myeloma, stomach cancer, brain cancer, lung cancer, pancreatic cancer, cervical cancer, ovarian cancer, liver cancer, bladder cancer, breast cancer, colon cancer, renal cancer, prostate cancer, testicular cancer, thyroid cancer, and head and neck cancer. In further embodiments, the cancer is selected from the group consisting of stomach cancer, renal cancer, brain cancer, bladder cancer, colon cancer, lung cancer, breast cancer, pancreatic cancer, ovarian cancer, and prostate cancer.

In various embodiments of the above-described methods, the animal is a mammal. In further embodiments, the mammal is a human.

### BRIEF DESCRIPTION OF THE DRAWINGS

FIG. 1 shows a representative FACS binding curve of antibodies bound to SW480 cells.

FIGS. 2A and 2B show the effect of monoclonal murine anti-RON antibodies and human monoclonal anti-RON Fabs on MSP binding to RON.

FIGS. 3A-3C show the effect of anti-RON antibodies on MSP-induced RON phosphorylation in MB-453 breast cancer cells and in BxPC-3 pancreatic cancer cells.

FIGS. 4A-C show the effect of anti-RON antibodies on MSP-independent RON signaling in 293 cells overexpressing wild-type RON, cells expressing a constitutively active kinase domain mutant of RON and in BxPC-3 pancreatic cells.

FIG. 5 shows the effect of anti-RON antibodies on phosphorylation of pAKT in BxPC3 and MDA-MB-453 tumor cells.

FIG. 6 shows a FACS binding curve of antibodies bound to cells expressing human RON splice variant RONdelta160.

FIG. 7 shows the results of an ELISA assay measuring the binding of RON antibodies to soluble RON.

FIG. 8 shows a FACS binding curve of antibodies bound to 293 cells expressing RON.

FIG. 9 is a bar graph depicting the results of a FACS assay measuring antibodies bound to CHO cells expressing RON.

FIG. 10 shows a western blot of ERK and phospho-ERK in tumor cells treated with RON antibodies. The bar graph shows a quantitation of the results in the western blot obtained using antibody 1P3B2.2.

FIG. 11A shows a western blot of AKT and phospho-AKT in tumor cells treated with RON antibodies. FIG. 11B depicts a bar graph showing the quantitation of the results in the western blot obtained using antibody 1P3B2.2.

FIG. 12 shows western blots of phosho-AKT and phospho-ERK in various tumor cells that were untreated, treated with a control antibody, treated with MSP, or treated with MSP and an anti-RON antibody.

FIG. 13A shows the results of an ELISA assay measuring the binding of RON antibodies to soluble human and cyno RON. FIGS. 13B and C show the results of an ELISA measuring the binding of human MSP to soluble human (FIG. 13C) and cyno (FIG. 13B) RON.

FIGS. 14A-C show the results of tumor cell invasion assays using HT1080 and HT1080-RON (FIG. 14A), AGS (FIG. 14B) and MDA-MB-231 (FIG. 14C) cell lines. Higher bars indicate increased cell invasion.

FIG. 15 shows the results of an ELISA assay measuring the binding of RON antibodies to soluble human RON (Sema and PSI domains) and to the PSI domain of RON.

FIG. 16 shows a FACS binding curve of anti-murine RON antibodies binding to 293 cells expressing murine RON.

FIG. 17A shows western blots of AKT and phospho-AKT in CHO cells expressing murine RON and treated with anti-murine RON antibodies. FIG. 17B shows quantitation of the same.

FIGS. 18A-B show FACS binding curves of anti-murine RON antibodies and anti-human RON antibodies binding to 293 cells expressing either murine (FIG. 18A) or human (FIG. 18B) RON proteins.

FIG. 19 is a table showing RON expression levels and increases in phospho-ERK and phospho-AKT in various cancer cell types.

FIG. 20 shows a graph displaying the tumor size in SCID mice administered tumor cells and treated with anti-RON antibody. Arrows indicate time points when antibody was administered.

### DETAILED DESCRIPTION OF THE INVENTION

### I. Definitions

It is to be noted that the term “a” or “an” entity refers to one or more of that entity; for example, “an RON antibody,” is understood to represent one or more RON antibodies. As such, the terms “a” (or “an”), “one or more,” and “at least one” can be used interchangeably herein.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis.

A polypeptide of the invention may be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides may have a defined three-dimensional structure, although they do not necessarily have such structure. Polypeptides with a defined three-dimensional structure are referred to as folded, and polypeptides which do not possess a defined three-dimensional structure, but rather can adopt a large number of different conformations, and are referred to as unfolded. As used herein, the term glycoprotein refers to a protein coupled to at least one carbohydrate moiety that is attached to the protein via an oxygen-containing or a nitrogen-containing side chain of an amino acid residue, e.g., a serine residue or an asparagine residue.

By an “isolated” polypeptide or a fragment, variant, or derivative thereof is intended a polypeptide that is not in its natural milieu. No particular level of purification is required. For example, an isolated polypeptide can be removed from its native or natural environment. Recombinantly produced polypeptides and proteins expressed in host cells are considered isolated for purposed of the invention, as are native or recombinant polypeptides which have been separated, fractionated, or partially or substantially purified by any suitable technique.

Also included as polypeptides of the present invention are fragments, derivatives, analogs, or variants of the foregoing polypeptides, and any combination thereof. The terms “fragment,” “variant,” “derivative” and “analog” when referring to RON antibodies or antibody polypeptides of the present invention include any polypeptides which retain at least some of the antigen-binding properties of the corresponding native antibody or polypeptide. Fragments of polypeptides of the present invention include proteolytic fragments, as well as deletion fragments, in addition to specific antibody fragments discussed elsewhere herein. Variants of RON antibodies and antibody polypeptides of the present invention include fragments as described above, and also polypeptides with altered amino acid sequences due to amino acid substitutions, deletions, or insertions. Variants may occur naturally or be non-naturally occurring Non-naturally occurring variants may be produced using art-known mutagenesis techniques. Variant polypeptides may comprise conservative or non-conservative amino acid substitutions, deletions or additions. Derivatives of RON antibodies and antibody polypeptides of the present invention, are polypeptides which have been altered so as to exhibit additional features not found on the native polypeptide. Examples include fusion proteins. Variant polypeptides may also be referred to herein as “polypeptide analogs.” As used herein a “derivative” of an RON antibody or antibody polypeptide refers to a subject polypeptide having one or more residues chemically derivatized by reaction of a functional side group. Also included as “derivatives” are those peptides which contain one or more naturally occurring amino acid derivatives of the twenty standard amino acids. For example, 4-hydroxyproline may be substituted for proline; 5-hydroxylysine may be substituted for lysine; 3-methylhistidine may be substituted for histidine; homoserine may be substituted for serine; and ornithine may be substituted for lysine.

The term “polynucleotide” is intended to encompass a singular nucleic acid as well as plural nucleic acids, and refers to an isolated nucleic acid molecule or construct, e.g., messenger RNA (mRNA) or plasmid DNA (pDNA). A polynucleotide may comprise a conventional phosphodiester bond or a non-conventional bond (e.g., an amide bond, such as found in peptide nucleic acids (PNA)). The term “nucleic acid” refer to any one or more nucleic acid segments, e.g., DNA or RNA fragments, present in a polynucleotide. By “isolated” nucleic acid or polynucleotide is intended a nucleic acid molecule, DNA or RNA, which has been removed from its native environment. For example, a recombinant polynucleotide encoding an RON antibody contained in a vector is considered isolated for the purposes of the present invention. Further examples of an isolated polynucleotide include recombinant polynucleotides maintained in heterologous host cells or purified (partially or substantially) polynucleotides in solution. Isolated RNA molecules include in vivo or in vitro RNA transcripts of polynucleotides of the present invention. Isolated polynucleotides or nucleic acids according to the present invention further include such molecules produced synthetically. In addition, polynucleotide or a nucleic acid may be or may include a regulatory element such as a promoter, ribosome binding site, or a transcription terminator.

As used herein, a “coding region” is a portion of nucleic acid which consists of codons translated into amino acids. Although a “stop codon” (TAG, TGA, or TAA) is not translated into an amino acid, it may be considered to be part of a coding region, but any flanking sequences, for example promoters, ribosome binding sites, transcriptional terminators, introns, and the like, are not part of a coding region. Two or more coding regions of the present invention can be present in a single polynucleotide construct, e.g., on a single vector, or in separate polynucleotide constructs, e.g., on separate (different) vectors. Furthermore, any vector may contain a single coding region, or may comprise two or more coding regions, e.g., a single vector may separately encode an immunoglobulin heavy chain variable region and an immunoglobulin light chain variable region. In addition, a vector, polynucleotide, or nucleic acid of the invention may encode heterologous coding regions, either fused or unfused to a nucleic acid encoding an RON antibody or fragment, variant, or derivative thereof. Heterologous coding regions include without limitation specialized elements or motifs, such as a secretory signal peptide or a heterologous functional domain.

In certain embodiments, the polynucleotide or nucleic acid is DNA. In the case of DNA, a polynucleotide comprising a nucleic acid which encodes a polypeptide normally may include a promoter and/or other transcription or translation control elements operably associated with one or more coding regions. An operable association is when a coding region for a gene product, e.g., a polypeptide, is associated with one or more regulatory sequences in such a way as to place expression of the gene product under the influence or control of the regulatory sequence(s). Two DNA fragments (such as a polypeptide coding region and a promoter associated therewith) are “operably associated” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter may be a cell-specific promoter that directs substantial transcription of the DNA only in predetermined cells. Other transcription control elements, besides a promoter, for example enhancers, operators, repressors, and transcription termination signals, can be operably associated with the polynucleotide to direct cell-specific transcription. Suitable promoters and other transcription control regions are disclosed herein.

A variety of transcription control regions are known to those skilled in the art. These include, without limitation, transcription control regions which function in vertebrate cells, such as, but not limited to, promoter and enhancer segments from cytomegaloviruses (the immediate early promoter, in conjunction with intron-A), simian virus 40 (the early promoter), and retroviruses (such as Rous sarcoma virus). Other transcription control regions include those derived from vertebrate genes such as actin, heat shock protein, bovine growth hormone and rabbit β-globin, as well as other sequences capable of controlling gene expression in eukaryotic cells. Additional suitable transcription control regions include tissue-specific promoters and enhancers as well as lymphokine-inducible promoters (e.g., promoters inducible by interferons or interleukins).

Similarly, a variety of translation control elements are known to those of ordinary skill in the art. These include, but are not limited to ribosome binding sites, translation initiation and termination codons, and elements derived from picornaviruses (particularly an internal ribosome entry site, or IRES, also referred to as a CITE sequence).

In other embodiments, a polynucleotide of the present invention is RNA, for example, in the form of messenger RNA (mRNA).

Polynucleotide and nucleic acid coding regions of the present invention may be associated with additional coding regions which encode secretory or signal peptides, which direct the secretion of a polypeptide encoded by a polynucleotide of the present invention. According to the signal hypothesis, proteins secreted by mammalian cells have a signal peptide or secretory leader sequence which is cleaved from the mature protein once export of the growing protein chain across the rough endoplasmic reticulum has been initiated. Those of ordinary skill in the art are aware that polypeptides secreted by vertebrate cells generally have a signal peptide fused to the N-terminus of the polypeptide, which is cleaved from the complete or “full length” polypeptide to produce a secreted or “mature” form of the polypeptide. In certain embodiments, the native signal peptide, e.g., an immunoglobulin heavy chain or light chain signal peptide is used, or a functional derivative of that sequence that retains the ability to direct the secretion of the polypeptide that is operably associated with it. Alternatively, a heterologous mammalian signal peptide, or a functional derivative thereof, may be used. For example, the wild-type leader sequence may be substituted with the leader sequence of human tissue plasminogen activator (TPA) or mouse β-glucuronidase.

The present invention is directed to certain RON antibodies, or antigen-binding fragments, variants, or derivatives thereof. Unless specifically referring to full-sized antibodies such as naturally-occurring antibodies, the term “RON antibodies” encompasses full-sized antibodies as well as antigen-binding fragments, variants, analogs, or derivatives of such antibodies, e.g., naturally occurring antibody or immunoglobulin molecules or engineered antibody molecules or fragments that bind antigen in a manner similar to antibody molecules.

The terms “antibody” and “immunoglobulin” are used interchangeably herein. An antibody or immunoglobulin comprises at least the variable domain of a heavy chain, and normally comprises at least the variable domains of a heavy chain and a light chain. Basic immunoglobulin structures in vertebrate systems are relatively well understood. See, e.g., Harlow et al., Antibodies: A Laboratory Manual, (Cold Spring Harbor Laboratory Press, 2nd ed. 1988).

As will be discussed in more detail below, the term “immunoglobulin” comprises various broad classes of polypeptides that can be distinguished biochemically. Those skilled in the art will appreciate that heavy chains are classified as gamma, mu, alpha, delta, or epsilon, (γ, μ, α, δ, ε) with some subclasses among them (e.g., γ1-γ4). It is the nature of this chain that determines the “class” of the antibody as IgG, IgM, IgA IgG, or IgE, respectively. The immunoglobulin subclasses (isotypes) e.g., IgG1, IgG2, IgG3, IgG4, IgA1, etc. are well characterized and are known to confer functional specialization. Modified versions of each of these classes and isotypes are readily discernable to the skilled artisan in view of the instant disclosure and, accordingly, are within the scope of the instant invention. All immunoglobulin classes are clearly within the scope of the present invention, the following discussion will generally be directed to the IgG class of immunoglobulin molecules. With regard to IgG, a standard immunoglobulin molecule comprises two identical light chain polypeptides of molecular weight approximately 23,000 Daltons, and two identical heavy chain polypeptides of molecular weight 53,000-70,000. The four chains are typically joined by disulfide bonds in a “Y” configuration wherein the light chains bracket the heavy chains starting at the mouth of the “Y” and continuing through the variable region.

Light chains are classified as either kappa or lambda (κ, λ). Each heavy chain class may be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B cells or genetically engineered host cells. In the heavy chain, the amino acid sequences run from an N-terminus at the forked ends of the Y configuration to the C-terminus at the bottom of each chain.

Both the light and heavy chains are divided into regions of structural and functional homology. The terms “constant” and “variable” are used functionally. In this regard, it will be appreciated that the variable domains of both the light (VL) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. By convention the numbering of the constant region domains increases as they become more distal from the antigen binding site or amino-terminus of the antibody. The N-terminal portion is a variable region and at the C-terminal portion is a constant region; the CH3 and CL domains actually comprise the carboxy-terminus of the heavy and light chain, respectively.

As indicated above, the variable region allows the antibody to selectively recognize and specifically bind epitopes on antigens. That is, the VL domain and VH domain, or subset of the complementarity determining regions (CDRs), of an antibody combine to form the variable region that defines a three dimensional antigen binding site. This quaternary antibody structure forms the antigen binding site present at the end of each arm of the Y. More specifically, the antigen binding site is defined by three CDRs on each of the VH and VL chains. In some instances, e.g., certain immunoglobulin molecules derived from camelid species or engineered based on camelid immunoglobulins, a complete immunoglobulin molecule may consist of heavy chains only, with no light chains. See, e.g., Hamers-Casterman et al., Nature 363:446-448 (1993).

In naturally occurring antibodies, the six “complementarity determining regions” or “CDRs” present in each antigen binding domain are short, non-contiguous sequences of amino acids that are specifically positioned to form the antigen binding domain as the antibody assumes its three dimensional configuration in an aqueous environment. The remainder of the amino acids in the antigen binding domains, referred to as “framework” regions, show less inter-molecular variability. The framework regions largely adopt a β-sheet conformation and the CDRs form loops which connect, and in some cases form part of, the β-sheet structure. Thus, framework regions act to form a scaffold that provides for positioning the CDRs in correct orientation by inter-chain, non-covalent interactions. The antigen binding domain formed by the positioned CDRs defines a surface complementary to the epitope on the immunoreactive antigen. This complementary surface promotes the non-covalent binding of the antibody to its cognate epitope. The amino acids comprising the CDRs and the framework regions, respectively, can be readily identified for any given heavy or light chain variable region by one of ordinary skill in the art, since they have been precisely defined (see, “Sequences of Proteins of Immunological Interest,” Kabat, E., et al., U.S. Department of Health and Human Services, (1983); and Chothia and Lesk, J. Mol. Biol., 196:901-917 (1987), which are incorporated herein by reference in their entireties).

In the case where there are two or more definitions of a term which is used and/or accepted within the art, the definition of the term as used herein is intended to include all such meanings unless explicitly stated to the contrary. A specific example is the use of the term “complementarity determining region” (“CDR”) to describe the non-contiguous antigen combining sites found within the variable region of both heavy and light chain polypeptides. This particular region has been described by Kabat et al., U.S. Dept. of Health and Human Services, “Sequences of Proteins of Immunological Interest” (1983) and by Chothia et al., J. Mol. Biol. 196:901-917 (1987), which are incorporated herein by reference, where the definitions include overlapping or subsets of amino acid residues when compared against each other. Nevertheless, application of either definition to refer to a CDR of an antibody or variants thereof is intended to be within the scope of the term as defined and used herein. The appropriate amino acid residues which encompass the CDRs as defined by each of the above cited references are set forth below in Table 1 as a comparison. The exact residue numbers which encompass a particular CDR will vary depending on the sequence and size of the CDR. Those skilled in the art can routinely determine which residues comprise a particular CDR given the variable region amino acid sequence of the antibody.

TABLE 1
CDR Definitions¹

| | Kabat | Chothia |
|---|---|---|
| VH CDR1 | 31-35 | 26-32 |
| VH CDR2 | 50-65 | 52-58 |
| VH CDR3 | 95-102 | 95-102 |
| VL CDR1 | 24-34 | 26-32 |
| VL CDR2 | 50-56 | 50-52 |
| VL CDR3 | 89-97 | 91-96 |

¹Numbering of all CDR definitions in Table 1 is according to the numbering conventions set forth by Kabat et al. (see below).

Kabat et al. also defined a numbering system for variable domain sequences that is applicable to any antibody. One of ordinary skill in the art can unambiguously assign this system of “Kabat numbering” to any variable domain sequence, without reliance on any experimental data beyond the sequence itself. As used herein, “Kabat numbering” refers to the numbering system set forth by Kabat et al., U.S. Dept. of Health and Human Services, “Sequence of Proteins of Immunological Interest” (1983). Unless otherwise specified, references to the numbering of specific amino acid residue positions in an RON antibody or antigen-binding fragment, variant, or derivative thereof of the present invention are according to the Kabat numbering system.

In camelid species, the heavy chain variable region, referred to as VHH, forms the entire antigen-binding domain. The main differences between camelid VHH variable regions and those derived from conventional antibodies (VH) include (a) more hydrophobic amino acids in the light chain contact surface of VH as compared to the corresponding region in VHH, (b) a longer CDR3 in VHH, and (c) the frequent occurrence of a disulfide bond between CDR1 and CDR3 in VHH.

Antibodies or antigen-binding fragments, variants, or derivatives thereof of the invention include, but are not limited to, polyclonal, monoclonal, multispecific, human, humanized, primatized, or chimeric antibodies, single chain antibodies, epitope-binding fragments, e.g., Fab, Fab′ and F(ab′)₂, Fd, Fvs, single-chain Fvs (scFv), single-chain antibodies, disulfide-linked Fvs (sdFv), fragments comprising either a VL or VH domain, fragments produced by a Fab expression library, and anti-idiotypic (anti-Id) antibodies (including, e.g., anti-Id antibodies to RON antibodies disclosed herein). ScFv molecules are known in the art and are described, e.g., in U.S. Pat. No. 5,892,019. Immunoglobulin or antibody molecules of the invention can be of any type (e.g., IgG, IgE, IgM, IgD, IgA, and IgY), class (e.g., IgG1, IgG2, IgG3, IgG4, IgA1 and IgA2) or subclass of immunoglobulin molecule.

Antibody fragments, including single-chain antibodies, may comprise the variable region(s) alone or in combination with the entirety or a portion of the following: hinge region, CH1, CH2, and CH3 domains. Also included in the invention are antigen-binding fragments also comprising any combination of variable region(s) with a hinge region, CH1, CH2, and CH3 domains. Antibodies or immunospecific fragments thereof of the present invention may be from any animal origin including birds and mammals. Preferably, the antibodies are human, murine, donkey, rabbit, goat, guinea pig, camel, llama, horse, or chicken antibodies. In another embodiment, the variable region may be condricthoid in origin (e.g., from sharks). As used herein, “human” antibodies include antibodies having the amino acid sequence of a human immunoglobulin and include antibodies isolated from human immunoglobulin libraries or from animals transgenic for one or more human immunoglobulins and that do not express endogenous immunoglobulins, as described infra and, for example in, U.S. Pat. No. 5,939,598 by Kucherlapati et al. A human antibody is still “human” even if amino acid substitutions are made in the antibody.

As used herein, the term “heavy chain portion” includes amino acid sequences derived from an immunoglobulin heavy chain. A polypeptide comprising a heavy chain portion comprises at least one of: a CH1 domain, a hinge (e.g., upper, middle, and/or lower hinge region) domain, a CH2 domain, a CH3 domain, or a variant or fragment thereof. For example, a binding polypeptide for use in the invention may comprise a polypeptide chain comprising a CH1 domain; a polypeptide chain comprising a CH1 domain, at least a portion of a hinge domain, and a CH2 domain; a polypeptide chain comprising a CH1 domain and a CH3 domain; a polypeptide chain comprising a CH1 domain, at least a portion of a hinge domain, and a CH3 domain, or a polypeptide chain comprising a CH1 domain, at least a portion of a hinge domain, a CH2 domain, and a CH3 domain. In another embodiment, a polypeptide of the invention comprises a polypeptide chain comprising a CH3 domain. Further, a binding polypeptide for use in the invention may lack at least a portion of a CH2 domain (e.g., all or part of a CH2 domain). As set forth above, it will be understood by one of ordinary skill in the art that these domains (e.g., the heavy chain portions) may be modified such that they vary in amino acid sequence from the naturally occurring immunoglobulin molecule.

In certain RON antibodies, or antigen-binding fragments, variants, or derivatives thereof disclosed herein, the heavy chain portions of one polypeptide chain of a multimer are identical to those on a second polypeptide chain of the multimer. Alternatively, heavy chain portion-containing monomers of the invention are not identical. For example, each monomer may comprise a different target binding site, forming, for example, a bispecific antibody.

The heavy chain portions of a binding polypeptide for use in the diagnostic and treatment methods disclosed herein may be derived from different immunoglobulin molecules. For example, a heavy chain portion of a polypeptide may comprise a CH1 domain derived from an IgG1 molecule and a hinge region derived from an IgG3 molecule. In another example, a heavy chain portion can comprise a hinge region derived, in part, from an IgG1 molecule and, in part, from an IgG3 molecule. In another example, a heavy chain portion can comprise a chimeric hinge derived, in part, from an IgG1 molecule and, in part, from an IgG4 molecule.

As used herein, the term “light chain portion” includes amino acid sequences derived from an immunoglobulin light chain. Preferably, the light chain portion comprises at least one of a VL or CL domain.

RON antibodies, or antigen-binding fragments, variants, or derivatives thereof disclosed herein may be described or specified in terms of the epitope(s) or portion(s) of an antigen, e.g., a target polypeptide (RON) that they recognize or specifically bind. The portion of a target polypeptide which specifically interacts with the antigen binding domain of an antibody is an “epitope,” or an “antigenic determinant.” A target polypeptide may comprise a single epitope, but typically comprises at least two epitopes, and can include any number of epitopes, depending on the size, conformation, and type of antigen. Furthermore, it should be noted that an “epitope” on a target polypeptide may be or include non-polypeptide elements, e.g., an “epitope may include a carbohydrate side chain.

The minimum size of a peptide or polypeptide epitope for an antibody is thought to be about four to five amino acids. Peptide or polypeptide epitopes preferably contain at least seven, more preferably at least nine and most preferably between at least about 15 to about 30 amino acids. Since a CDR can recognize an antigenic peptide or polypeptide in its tertiary form, the amino acids comprising an epitope need not be contiguous, and in some cases, may not even be on the same peptide chain. In the present invention, peptide or polypeptide epitope recognized by RON antibodies of the present invention contains a sequence of at least 4, at least 5, at least 6, at least 7, more preferably at least 8, at least 9, at least 10, at least 15, at least 20, at least 25, or between about 15 to about 30 contiguous or non-contiguous amino acids of RON.

By “specifically binds,” it is generally meant that an antibody binds to an epitope via its antigen binding domain, and that the binding entails some complementarity between the antigen binding domain and the epitope. According to this definition, an antibody is said to “specifically bind” to an epitope when it binds to that epitope, via its antigen binding domain more readily than it would bind to a random, unrelated epitope. The term “specificity” is used herein to qualify the relative affinity by which a certain antibody binds to a certain epitope. For example, antibody “A” may be deemed to have a higher specificity for a given epitope than antibody “B,” or antibody “A” may be said to bind to epitope “C” with a higher specificity than it has for related epitope “D.”

By “preferentially binds,” it is meant that the antibody specifically binds to an epitope more readily than it would bind to a related, similar, homologous, or analogous epitope. Thus, an antibody which “preferentially binds” to a given epitope would more likely bind to that epitope than to a related epitope, even though such an antibody may cross-react with the related epitope.

By way of non-limiting example, an antibody may be considered to bind a first epitope preferentially if it binds said first epitope with a dissociation constant (K_{D}) that is less than the antibody's K_{D} for the second epitope. In another non-limiting example, an antibody may be considered to bind a first antigen preferentially if it binds the first epitope with an affinity that is at least one order of magnitude less than the antibody's K_{D} for the second epitope. In another non-limiting example, an antibody may be considered to bind a first epitope preferentially if it binds the first epitope with an affinity that is at least two orders of magnitude less than the antibody's K_{D} for the second epitope.

In another non-limiting example, an antibody may be considered to bind a first epitope preferentially if it binds the first epitope with an off rate (k(off)) that is less than the antibody's k(off) for the second epitope. In another non-limiting example, an antibody may be considered to bind a first epitope preferentially if it binds the first epitope with an affinity that is at least one order of magnitude less than the antibody's k(off) for the second epitope. In another non-limiting example, an antibody may be considered to bind a first epitope preferentially if it binds the first epitope with an affinity that is at least two orders of magnitude less than the antibody's k(off) for the second epitope.

An antibody or antigen-binding fragment, variant, or derivative disclosed herein may be said to bind a target polypeptide disclosed herein or a fragment or variant thereof with an off rate (k(off)) of less than or equal to 5×10⁻² sec⁻¹, 10⁻² sec⁻¹, 5×10⁻³ sec⁻¹ or 10⁻³ sec⁻¹. More preferably, an antibody of the invention may be said to bind a target polypeptide disclosed herein or a fragment or variant thereof with an off rate (k(off)) less than or equal to 5×10⁻⁴ sec⁻¹, 10⁻⁴ sec⁻¹, 5×10⁻⁵ sec⁻¹, or 10⁻⁵ sec⁻¹ 5×10⁻⁶ sec⁻¹, 10⁻⁶ sec⁻¹, 5×10⁻⁷ sec⁻¹ or 10⁻⁷ sec⁻¹.

An antibody or antigen-binding fragment, variant, or derivative disclosed herein may be said to bind a target polypeptide disclosed herein or a fragment or variant thereof with an on rate (k(on)) of greater than or equal to 10³ M⁻¹ sec⁻¹, 5×10³ M⁻¹ sec⁻¹, 10⁴ M⁻¹ sec⁻¹ or 5×10⁴ M⁻¹ sec⁻¹. More preferably, an antibody of the invention may be said to bind a target polypeptide disclosed herein or a fragment or variant thereof with an on rate (k(on)) greater than or equal to 10⁵ M⁻¹ sec⁻¹, 5×10⁵ M⁻¹ sec⁻¹, 10⁶ M⁻¹ sec⁻¹, or 5×10⁶ M⁻¹ sec⁻¹ or 10⁷ M⁻¹ sec⁻¹.

An antibody is said to competitively inhibit binding of a reference antibody to a given epitope if it preferentially binds to that epitope to the extent that it blocks, to some degree, binding of the reference antibody to the epitope. Competitive inhibition may be determined by any method known in the art, for example, competition ELISA assays. An antibody may be said to competitively inhibit binding of the reference antibody to a given epitope by at least 90%, at least 80%, at least 70%, at least 60%, or at least 50%.

As used herein, the term “affinity” refers to a measure of the strength of the binding of an individual epitope with the CDR of an immunoglobulin molecule. See, e.g., Harlow et al., Antibodies: A Laboratory Manual, (Cold Spring Harbor Laboratory Press, 2nd ed. 1988) at pages 27-28. As used herein, the term “avidity” refers to the overall stability of the complex between a population of immunoglobulins and an antigen, that is, the functional combining strength of an immunoglobulin mixture with the antigen. See, e.g., Harlow at pages 29-34. Avidity is related to both the affinity of individual immunoglobulin molecules in the population with specific epitopes, and also the valencies of the immunoglobulins and the antigen. For example, the interaction between a bivalent monoclonal antibody and an antigen with a highly repeating epitope structure, such as a polymer, would be one of high avidity.

RON antibodies or antigen-binding fragments, variants or derivatives thereof of the invention may also be described or specified in terms of their cross-reactivity. As used herein, the term “cross-reactivity” refers to the ability of an antibody, specific for one antigen, to react with a second antigen; a measure of relatedness between two different antigenic substances. Thus, an antibody is cross reactive if it binds to an epitope other than the one that induced its formation. The cross reactive epitope generally contains many of the same complementary structural features as the inducing epitope, and in some cases, may actually fit better than the original.

For example, certain antibodies have some degree of cross-reactivity, in that they bind related, but non-identical epitopes, e.g., epitopes with at least 95%, at least 90%, at least 85%, at least 80%, at least 75%, at least 70%, at least 65%, at least 60%, at least 55%, and at least 50% identity (as calculated using methods known in the art and described herein) to a reference epitope. An antibody may be said to have little or no cross-reactivity if it does not bind epitopes with less than 95%, less than 90%, less than 85%, less than 80%, less than 75%, less than 70%, less than 65%, less than 60%, less than 55%, and less than 50% identity (as calculated using methods known in the art and described herein) to a reference epitope. An antibody may be deemed “highly specific” for a certain epitope, if it does not bind any other analog, ortholog, or homolog of that epitope.

RON antibodies or antigen-binding fragments, variants or derivatives thereof of the invention may also be described or specified in terms of their binding affinity to a polypeptide of the invention. Preferred binding affinities include those with a dissociation constant or Kd less than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

RON antibodies or antigen-binding fragments, variants or derivatives thereof of the invention may be “multispecific,” e.g., bispecific, trispecific or of greater multispecificity, meaning that it recognizes and binds to two or more different epitopes present on one or more different antigens (e.g., proteins) at the same time. Thus, whether an RON antibody is “monospecific” or “multispecific,” e.g., “bispecific,” refers to the number of different epitopes with which a binding polypeptide reacts. Multispecific antibodies may be specific for different epitopes of a target polypeptide described herein or may be specific for a target polypeptide as well as for a heterologous epitope, such as a heterologous polypeptide or solid support material.

As used herein the term “valency” refers to the number of potential binding domains, e.g., antigen binding domains, present in an RON antibody, binding polypeptide or antibody. Each binding domain specifically binds one epitope. When an RON antibody, binding polypeptide or antibody comprises more than one binding domain, each binding domain may specifically bind the same epitope, for an antibody with two binding domains, termed “bivalent monospecific,” or to different epitopes, for an antibody with two binding domains, termed “bivalent bispecific.” An antibody may also be bispecific and bivalent for each specificity (termed “bispecific tetravalent antibodies”). In another embodiment, tetravalent minibodies or domain deleted antibodies can be made.

Bispecific bivalent antibodies, and methods of making them, are described, for instance in U.S. Pat. Nos. 5,731,168; 5,807,706; 5,821,333; and U.S. Appl. Publ. Nos. 2003/020734 and 2002/0155537, the disclosures of all of which are incorporated by reference herein. Bispecific tetravalent antibodies, and methods of making them are described, for instance, in WO 02/096948 and WO 00/44788, the disclosures of both of which are incorporated by reference herein. See generally, PCT publications WO 93/17715; WO 92/08802; WO 91/00360; WO 92/05793; Tutt et al., J. Immunol. 147:60-69 (1991); U.S. Pat. Nos. 4,474,893; 4,714,681; 4,925,648; 5,573,920; 5,601,819; Kostelny et al., J. Immunol. 148:1547-1553 (1992).

As previously indicated, the subunit structures and three dimensional configuration of the constant regions of the various immunoglobulin classes are well known. As used herein, the term “VH domain” includes the amino terminal variable domain of an immunoglobulin heavy chain and the term “CH1 domain” includes the first (most amino terminal) constant region domain of an immunoglobulin heavy chain. The CH1 domain is adjacent to the VH domain and is amino terminal to the hinge region of an immunoglobulin heavy chain molecule.

As used herein the term “CH2 domain” includes the portion of a heavy chain molecule that extends, e.g., from about residue 244 to residue 360 of an antibody using conventional numbering schemes (residues 244 to 360, Kabat numbering system; and residues 231-340, EU numbering system; see Kabat E A et al. op. cit. The CH2 domain is unique in that it is not closely paired with another domain. Rather, two N-linked branched carbohydrate chains are interposed between the two CH2 domains of an intact native IgG molecule. It is also well documented that the CH3 domain extends from the CH2 domain to the C-terminal of the IgG molecule and comprises approximately 108 residues.

As used herein, the term “hinge region” includes the portion of a heavy chain molecule that joins the CH1 domain to the CH2 domain. This hinge region comprises approximately 25 residues and is flexible, thus allowing the two N-terminal antigen binding regions to move independently. Hinge regions can be subdivided into three distinct domains: upper, middle, and lower hinge domains (Roux et al., J. Immunol. 161:4083 (1998)).

As used herein the term “disulfide bond” includes the covalent bond formed between two sulfur atoms. The amino acid cysteine comprises a thiol group that can form a disulfide bond or bridge with a second thiol group. In most naturally occurring IgG molecules, the CH1 and CL regions are linked by a disulfide bond and the two heavy chains are linked by two disulfide bonds at positions corresponding to 239 and 242 using the Kabat numbering system (position 226 or 229, EU numbering system).

As used herein, the term “chimeric antibody” will be held to mean any antibody wherein the immunoreactive region or site is obtained or derived from a first species and the constant region (which may be intact, partial or modified in accordance with the instant invention) is obtained from a second species. In preferred embodiments, the target binding region or site will be from a non-human source (e.g. mouse or primate) and the constant region is human.

As used herein, the term “engineered antibody” refers to an antibody in which the variable domain in either the heavy and light chain or both is altered by at least partial replacement of one or more CDRs from an antibody of known specificity and, if necessary, by partial framework region replacement and sequence changing. Although the CDRs may be derived from an antibody of the same class or even subclass as the antibody from which the framework regions are derived, it is envisaged that the CDRs will be derived from an antibody of different class and preferably from an antibody from a different species. An engineered antibody in which one or more “donor” CDRs from a non-human antibody of known specificity is grafted into a human heavy or light chain framework region is referred to herein as a “humanized antibody.” It may not be necessary to replace all of the CDRs with the complete CDRs from the donor variable region to transfer the antigen binding capacity of one variable domain to another. Rather, it may only be necessary to transfer those residues that are necessary to maintain the activity of the target binding site. Given the explanations set forth in, e.g., U.S. Pat. Nos. 5,585,089, 5,693,761, 5,693,762, and 6,180,370, it will be well within the competence of those skilled in the art, either by carrying out routine experimentation or by trial and error testing to obtain a functional engineered or humanized antibody.

As used herein the term “properly folded polypeptide” includes polypeptides (e.g., RON antibodies) in which all of the functional domains comprising the polypeptide are distinctly active. As used herein, the term “improperly folded polypeptide” includes polypeptides in which at least one of the functional domains of the polypeptide is not active. In one embodiment, a properly folded polypeptide comprises polypeptide chains linked by at least one disulfide bond and, conversely, an improperly folded polypeptide comprises polypeptide chains not linked by at least one disulfide bond.

As used herein the term “engineered” includes manipulation of nucleic acid or polypeptide molecules by synthetic means (e.g. by recombinant techniques, in vitro peptide synthesis, by enzymatic or chemical coupling of peptides or some combination of these techniques).

As used herein, the terms “linked,” “fused” or “fusion” are used interchangeably. These terms refer to the joining together of two more elements or components, by whatever means including chemical conjugation or recombinant means. An “in-frame fusion” refers to the joining of two or more polynucleotide open reading frames (ORFs) to form a continuous longer ORF, in a manner that maintains the correct translational reading frame of the original ORFs. Thus, a recombinant fusion protein is a single protein containing two ore more segments that correspond to polypeptides encoded by the original ORFs (which segments are not normally so joined in nature.) Although the reading frame is thus made continuous throughout the fused segments, the segments may be physically or spatially separated by, for example, in-frame linker sequence. For example, polynucleotides encoding the CDRs of an immunoglobulin variable region may be fused, in-frame, but be separated by a polynucleotide encoding at least one immunoglobulin framework region or additional CDR regions, as long as the “fused” CDRs are co-translated as part of a continuous polypeptide.

In the context of polypeptides, a “linear sequence” or a “sequence” is an order of amino acids in a polypeptide in an amino to carboxyl terminal direction in which residues that neighbor each other in the sequence are contiguous in the primary structure of the polypeptide.

The term “expression” as used herein refers to a process by which a gene produces a biochemical, for example, an RNA or polypeptide. The process includes any manifestation of the functional presence of the gene within the cell including, without limitation, gene knockdown as well as both transient expression and stable expression. It includes without limitation transcription of the gene into messenger RNA (mRNA), transfer RNA (tRNA), small hairpin RNA (shRNA), small interfering RNA (siRNA) or any other RNA product, and the translation of such mRNA into polypeptide(s). If the final desired product is a biochemical, expression includes the creation of that biochemical and any precursors. Expression of a gene produces a “gene product.” As used herein, a gene product can be either a nucleic acid, e.g., a messenger RNA produced by transcription of a gene, or a polypeptide which is translated from a transcript. Gene products described herein further include nucleic acids with post transcriptional modifications, e.g., polyadenylation, or polypeptides with post translational modifications, e.g., methylation, glycosylation, the addition of lipids, association with other protein subunits, proteolytic cleavage, and the like.

As used herein, the terms “treat” or “treatment” refer to both therapeutic treatment and prophylactic or preventative measures, wherein the object is to prevent or slow down (lessen) an undesired physiological change or disorder, such as the development or spread of cancer. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sports, or pet animals such as dogs, cats, guinea pigs, rabbits, rats, mice, horses, cattle, cows, and so on.

As used herein, phrases such as “a subject that would benefit from administration of a binding molecule” and “an animal in need of treatment” includes subjects, such as mammalian subjects, that would benefit from administration of a binding molecule used, e.g., for detection of an antigen recognized by a binding molecule (e.g., for a diagnostic procedure) and/or from treatment, i.e., palliation or prevention of a disease such as cancer, with a binding molecule which specifically binds a given target protein. As described in more detail herein, the binding molecule can be used in unconjugated form or can be conjugated, e.g., to a drug, prodrug, or an isotope.

By “hyperproliferative disease or disorder” is meant all neoplastic cell growth and proliferation, whether malignant or benign, including all transformed cells and tissues and all cancerous cells and tissues. Hyperproliferative diseases or disorders include, but are not limited to, precancerous lesions, abnormal cell growths, benign tumors, malignant tumors, and “cancer.” In certain embodiments of the present invention, the hyperproliferative disease or disorder, e.g., the precancerous lesion, abnormal cell growth, benign tumor, malignant tumor, or “cancer” comprises cells which express, over-express, or abnormally express RON.

Additional examples of hyperproliferative diseases, disorders, and/or conditions include, but are not limited to neoplasms, whether benign or malignant, located in the: prostate, colon, abdomen, bone, breast, digestive system, liver, pancreas, peritoneum, endocrine glands (adrenal, parathyroid, pituitary, testicles, ovary, thymus, thyroid), eye, head and neck, nervous (central and peripheral), lymphatic system, pelvic, skin, soft tissue, spleen, thoracic, and urogenital tract. Such neoplasms, in certain embodiments, express, over-express, or abnormally express RON.

Other hyperproliferative disorders include, but are not limited to: hypergammaglobulinemia, lymphoproliferative disorders, paraproteinemias, purpura, sarcoidosis, Sezary Syndrome, Waldenstron's macroglobulinemia, Gaucher's Disease, histiocytosis, and any other hyperproliferative disease, besides neoplasia, located in an organ system listed above. In certain embodiments of the present invention the diseases involve cells which express, over-express, or abnormally express RON.

As used herein, the terms “tumor” or “tumor tissue” refer to an abnormal mass of tissue that results from excessive cell division, in certain cases tissue comprising cells which express, over-express, or abnormally express RON. A tumor or tumor tissue comprises “tumor cells” which are neoplastic cells with abnormal growth properties and no useful bodily function. Tumors, tumor tissue and tumor cells may be benign or malignant. A tumor or tumor tissue may also comprise “tumor-associated non-tumor cells”, e.g., vascular cells which form blood vessels to supply the tumor or tumor tissue. Non-tumor cells may be induced to replicate and develop by tumor cells, for example, the induction of angiogenesis in a tumor or tumor tissue.

As used herein, the term “malignancy” refers to a non-benign tumor or a cancer. As used herein, the term “cancer” connotes a type of hyperproliferative disease which includes a malignancy characterized by deregulated or uncontrolled cell growth. Examples of cancer include, but are not limited to, carcinoma, lymphoma, blastoma, sarcoma, and leukemia or lymphoid malignancies. More particular examples of such cancers are noted below and include: squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial cancer or uterine carcinoma, salivary gland carcinoma, kidney or renal cancer, prostate cancer, vulval cancer, thyroid cancer, hepatic carcinoma, anal carcinoma, penile carcinoma, as well as head and neck cancer. The term “cancer” includes primary malignant cells or tumors (e.g., those whose cells have not migrated to sites in the subject's body other than the site of the original malignancy or tumor) and secondary malignant cells or tumors (e.g., those arising from metastasis, the migration of malignant cells or tumor cells to secondary sites that are different from the site of the original tumor). Cancers conducive to treatment methods of the present invention involves cells which express, over-express, or abnormally express RON.

Other examples of cancers or malignancies include, but are not limited to: Acute Childhood Lymphoblastic Leukemia, Acute Lymphoblastic Leukemia, Acute Lymphocytic Leukemia, Acute Myeloid Leukemia, Adrenocortical Carcinoma, Adult (Primary) Hepatocellular Cancer, Adult (Primary) Liver Cancer, Adult Acute Lymphocytic Leukemia, Adult Acute Myeloid Leukemia, Adult Hodgkin's Disease, Adult Hodgkin's Lymphoma, Adult Lymphocytic Leukemia, Adult Non-Hodgkin's Lymphoma, Adult Primary Liver Cancer, Adult Soft Tissue Sarcoma, AIDS-Related Lymphoma, AIDS-Related Malignancies, Anal Cancer, Astrocytoma, Bile Duct Cancer, Bladder Cancer, Bone Cancer, Brain Stem Glioma, Brain Tumors, Breast Cancer, Cancer of the Renal Pelvis and Ureter, Central Nervous System (Primary) Lymphoma, Central Nervous System Lymphoma, Cerebellar Astrocytoma, Cerebral Astrocytoma, Cervical Cancer, Childhood (Primary) Hepatocellular Cancer, Childhood (Primary) Liver Cancer, Childhood Acute Lymphoblastic Leukemia, Childhood Acute Myeloid Leukemia, Childhood Brain Stem Glioma, Childhood Cerebellar Astrocytoma, Childhood Cerebral Astrocytoma, Childhood Extracranial Germ Cell Tumors, Childhood Hodgkin's Disease, Childhood Hodgkin's Lymphoma, Childhood Hypothalamic and Visual Pathway Glioma, Childhood Lymphoblastic Leukemia, Childhood Medulloblastoma, Childhood Non-Hodgkin's Lymphoma, Childhood Pineal and Supratentorial Primitive Neuroectodermal Tumors, Childhood Primary Liver Cancer, Childhood Rhabdomyosarcoma, Childhood Soft Tissue Sarcoma, Childhood Visual Pathway and Hypothalamic Glioma, Chronic Lymphocytic Leukemia, Chronic Myelogenous Leukemia, Colon Cancer, Cutaneous T-Cell Lymphoma, Endocrine Pancreas Islet Cell Carcinoma, Endometrial Cancer, Ependymoma, Epithelial Cancer, Esophageal Cancer, Ewing's Sarcoma and Related Tumors, Exocrine Pancreatic Cancer, Extracranial Germ Cell Tumor, Extragonadal Germ Cell Tumor, Extrahepatic Bile Duct Cancer, Eye Cancer, Female Breast Cancer, Gaucher's Disease, Gallbladder Cancer, Gastric Cancer, Gastrointestinal Carcinoid Tumor, Gastrointestinal Tumors, Germ Cell Tumors, Gestational Trophoblastic Tumor, Hairy Cell Leukemia, Head and Neck Cancer, Hepatocellular Cancer, Hodgkin's Disease, Hodgkin's Lymphoma, Hypergammaglobulinemia, Hypopharyngeal Cancer, Intestinal Cancers, Intraocular Melanoma, Islet Cell Carcinoma, Islet Cell Pancreatic Cancer, Kaposi's Sarcoma, Kidney Cancer, Laryngeal Cancer, Lip and Oral Cavity Cancer, Liver Cancer, Lung Cancer, Lymphoproliferative Disorders, Macroglobulinemia, Male Breast Cancer, Malignant Mesothelioma, Malignant Thymoma, Medulloblastoma, Melanoma, Mesothelioma, Metastatic Occult Primary Squamous Neck Cancer, Metastatic Primary Squamous Neck Cancer, Metastatic Squamous Neck Cancer, Multiple Myeloma, Multiple Myeloma/Plasma Cell Neoplasm, Myelodysplastic Syndrome, Myelogenous Leukemia, Myeloid Leukemia, Myeloproliferative Disorders, Nasal Cavity and Paranasal Sinus Cancer, Nasopharyngeal Cancer, Neuroblastoma, Non-Hodgkin's Lymphoma During Pregnancy, Nonmelanoma Skin Cancer, Non-Small Cell Lung Cancer, Occult Primary Metastatic Squamous Neck Cancer, Oropharyngeal Cancer, Osteo-/Malignant Fibrous Sarcoma, Osteosarcoma/Malignant Fibrous Histiocytoma, Osteosarcoma/Malignant Fibrous Histiocytoma of Bone, Ovarian Epithelial Cancer, Ovarian Germ Cell Tumor, Ovarian Low Malignant Potential Tumor, Pancreatic Cancer, Paraproteinemias, Purpura, Parathyroid Cancer, Penile Cancer, Pheochromocytoma, Pituitary Tumor, Plasma Cell Neoplasm/Multiple Myeloma, Primary Central Nervous System Lymphoma, Primary Liver Cancer, Prostate Cancer, Rectal Cancer, Renal Cell Cancer, Renal Pelvis and Ureter Cancer, Retinoblastoma, Rhabdomyosarcoma, Salivary Gland Cancer, Sarcoidosis Sarcomas, Sezary Syndrome, Skin Cancer, Small Cell Lung Cancer, Small Intestine Cancer, Soft Tissue Sarcoma, Squamous Neck Cancer, Stomach Cancer, Supratentorial Primitive Neuroectodermal and Pineal Tumors, T-Cell Lymphoma, Testicular Cancer, Thymoma, Thyroid Cancer, Transitional Cell Cancer of the Renal Pelvis and Ureter, Transitional Renal Pelvis and Ureter Cancer, Trophoblastic Tumors, Ureter and Renal Pelvis Cell Cancer, Urethral Cancer, Uterine Cancer, Uterine Sarcoma, Vaginal Cancer, Visual Pathway and Hypothalamic Glioma, Vulvar Cancer, Waldenstrom's Macroglobulinemia, Wilms' Tumor, and any other hyperproliferative disease, besides neoplasia, located in an organ system listed above.

The method of the present invention may be used to treat premalignant conditions and to prevent progression to a neoplastic or malignant state, including but not limited to those disorders described above. Such uses are indicated in conditions known or suspected of preceding progression to neoplasia or cancer, in particular, where non-neoplastic cell growth consisting of hyperplasia, metaplasia, or most particularly, dysplasia has occurred (for review of such abnormal growth conditions, see Robbins and Angell, Basic Pathology, 2d Ed., W. B. Saunders Co., Philadelphia, pp. 68-79 (1976). Such conditions in which cells begin to express, over-express, or abnormally express RON, are particularly treatable by the methods of the present invention.

Hyperplasia is a form of controlled cell proliferation, involving an increase in cell number in a tissue or organ, without significant alteration in structure or function. Hyperplastic disorders which can be treated by the method of the invention include, but are not limited to, angiofollicular mediastinal lymph node hyperplasia, angiolymphoid hyperplasia with eosinophilia, atypical melanocytic hyperplasia, basal cell hyperplasia, benign giant lymph node hyperplasia, cementum hyperplasia, congenital adrenal hyperplasia, congenital sebaceous hyperplasia, cystic hyperplasia, cystic hyperplasia of the breast, denture hyperplasia, ductal hyperplasia, endometrial hyperplasia, fibromuscular hyperplasia, focal epithelial hyperplasia, gingival hyperplasia, inflammatory fibrous hyperplasia, inflammatory papillary hyperplasia, intravascular papillary endothelial hyperplasia, nodular hyperplasia of prostate, nodular regenerative hyperplasia, pseudoepitheliomatous hyperplasia, senile sebaceous hyperplasia, and verrucous hyperplasia.

Metaplasia is a form of controlled cell growth in which one type of adult or fully differentiated cell substitutes for another type of adult cell. Metaplastic disorders which can be treated by the method of the invention include, but are not limited to, agnogenic myeloid metaplasia, apocrine metaplasia, atypical metaplasia, autoparenchymatous metaplasia, connective tissue metaplasia, epithelial metaplasia, intestinal metaplasia, metaplastic anemia, metaplastic ossification, metaplastic polyps, myeloid metaplasia, primary myeloid metaplasia, secondary myeloid metaplasia, squamous metaplasia, squamous metaplasia of amnion, and symptomatic myeloid metaplasia.

Dysplasia is frequently a forerunner of cancer, and is found mainly in the epithelia; it is the most disorderly form of non-neoplastic cell growth, involving a loss in individual cell uniformity and in the architectural orientation of cells. Dysplastic cells often have abnormally large, deeply stained nuclei, and exhibit pleomorphism. Dysplasia characteristically occurs where there exists chronic irritation or inflammation. Dysplastic disorders which can be treated by the method of the invention include, but are not limited to, anhidrotic ectodermal dysplasia, anterofacial dysplasia, asphyxiating thoracic dysplasia, atriodigital dysplasia, bronchopulmonary dysplasia, cerebral dysplasia, cervical dysplasia, chondroectodermal dysplasia, cleidocranial dysplasia, congenital ectodermal dysplasia, craniodiaphysial dysplasia, craniocarpotarsal dysplasia, craniometaphysial dysplasia, dentin dysplasia, diaphysial dysplasia, ectodermal dysplasia, enamel dysplasia, encephalo-ophthalmic dysplasia, dysplasia epiphysialis hemimelia, dysplasia epiphysialis multiplex, dysplasia epiphysialis punctata, epithelial dysplasia, faciodigitogenital dysplasia, familial fibrous dysplasia of jaws, familial white folded dysplasia, fibromuscular dysplasia, fibrous dysplasia of bone, florid osseous dysplasia, hereditary renal-retinal dysplasia, hidrotic ectodermal dysplasia, hypohidrotic ectodermal dysplasia, lymphopenic thymic dysplasia, mammary dysplasia, mandibulofacial dysplasia, metaphysial dysplasia, Mondini dysplasia, monostotic fibrous dysplasia, mucoepithelial dysplasia, multiple epiphysial dysplasia, oculoauriculovertebral dysplasia, oculodentodigital dysplasia, oculovertebral dysplasia, odontogenic dysplasia, opthalmomandibulomelic dysplasia, periapical cemental dysplasia, polyostotic fibrous dysplasia, pseudoachondroplastic spondyloepiphysial dysplasia, retinal dysplasia, septo-optic dysplasia, spondyloepiphysial dysplasia, and ventriculoradial dysplasia.

Additional pre-neoplastic disorders which can be treated by the method of the invention include, but are not limited to, benign dysproliferative disorders (e.g., benign tumors, fibrocystic conditions, tissue hypertrophy, intestinal polyps, colon polyps, and esophageal dysplasia), leukoplakia, keratoses, Bowen's disease, Farmer's Skin, solar cheilitis, and solar keratosis.

In preferred embodiments, the method of the invention is used to inhibit growth, progression, and/or metastasis of cancers, in particular those listed above.

Additional hyperproliferative diseases, disorders, and/or conditions include, but are not limited to, progression, and/or metastases of malignancies and related disorders such as leukemia (including acute leukemias (e.g., acute lymphocytic leukemia, acute myelocytic leukemia (including myeloblastic, promyelocytic, myelomonocytic, monocytic, and erythroleukemia)) and chronic leukemias (e.g., chronic myelocytic (granulocytic) leukemia and chronic lymphocytic leukemia)), polycythemia vera, lymphomas (e.g., Hodgkin's disease and non-Hodgkin's disease), multiple myeloma, Waldenstrom's macroglobulinemia, heavy chain disease, and solid tumors including, but not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, emangioblastoma, acoustic neuroma, oligodendroglioma, menangioma, melanoma, neuroblastoma, and retinoblastoma.

### II. RON

Naturally occurring mature RON (receptor d'origine nantais) is a heterodimer composed of a smaller alpha chain and a larger beta chain that includes a transmembrane domain and a kinase domain. This mature form of RON is created by the cleavage of a single-chain precursor, pro-RON. After cleavage, the alpha and beta chains remain associated through a disulfide linkage.

The following polypeptide sequence was reported as the human RON sequence and has the accession number NP_{—}002438 in Genbank.

Human RON (SEQ ID NO:1):

MELLPPLPQSFLLLLLLPAKPAAGEDWQCPRTPYAASRDFDVKYVVPSFS
AGGLVQAMVTYEGDRNESAVFVAIRNRLHVLGPDLKSVQSLATGPAGDPG
CQTCAACGPGPHGPPGDTDTKVLVLDPALPALVSCGSSLQGRCFLHDLEP
QGTAVHLAAPACLFSAHHNRPDDCPDCVASPLGTRVTVVEQGQASYFYVA
SSLDAAVAASFSPRSVSIRRLKADASGFAPGFVALSVLPKHLVSYSIEYV
HSFHTGAFVYFLTVQPASVTDDPSALHTRLARLSATEPELGDYRELVLDC
RFAPKRRRRGAPEGGQPYPVLRVAHSAPVGAQLATELSIAEGQEVLFGVF
VTGKDGGPGVGPNSVVCAFPIDLLDTLIDEGVERCCESPVHPGLRRGLDF
FQSPSFCPNPPGLEALSPNTSCRHFPLLVSSSFSRVDLFNGLLGPVQVTA
LYVTRLDNVTVAHMGTMDGRILQVELVRSLNYLLYVSNFSLGDSGQPVQR
DVSRLGDHLLFASGDQVFQVPIQGPGCRHFLTCGRCLRAWHFMGCGWCGN
MCGQQKECPGSWQQDHCPPKLTEFHPHSGPLRGSTRLTLCGSNFYLHPSG
LVPEGTHQVTVGQSPCRPLPKDSSKLRPVPRKDFVEEFECELEPLGTQAV
GPTNVSLTVTNMPPGKHFRVDGTSVLRGFSFMEPVLIAVQPLFGPRAGGT
CLTLEGQSLSVGTSRAVLVNGTECLLARVSEGQLLCATPPGATVASVPLS
LQVGGAQVPGSWTFQYREDPVVLSISPNCGYINSHITICGQHLTSAWHLV
LSFHDGLRAVESRCERQLPEQQLCRLPEYVVRDPQGWVAGNLSARGDGAA
GFTLPGFRFLPPPHPPSANLVPLKPEEHAIKFEYIGLGAVADCVGINVTV
GGESCQHEFRGDMVVCPLPPSLQLGQDGAPLQVCVDGECHILGRVVRPGP
DGVPQSTLLGILLPLLLLVAALATALVFSYWWRRKQLVLPPNLNDLASLD
QTAGATPLPILYSGSDYRSGLALPAIDGLDSTTCVHGASFSDSEDESCVP
LLRKESIQLRDLDSALLAEVKDVLIPHERVVTHSDRVIGKGHFGVVYHGE
YIDQAQNRIQCAIKSLSRITEMQQVEAFLREGLLMRGLNHPNVLALIGIM
LPPEGLPHVLLPYMCHGDLLQFIRSPQRNPTVKDLISFGLQVARGMEYLA
EQKFVHRDLAARNCMLDESFTVKVADFGLARDILDREYYSVQQHRHARLP
VKWMALESLQTYRFTTKSDVWSFGVLLWELLTRGAPPYRHIDPFDLTHFL
AQGRRLPQPEYCPDSLYQVMQQCWEADPAVRPTFRVLVGEVEQIVSALLG
DHYVQLPATYMNLGPSTSHEMNVRPEQPQFSPMPGNVRRPRPLSEPPRPT

The mouse RON polypeptide has been reported to have the following sequence and has the accession number NP_{—}033100 in Genbank (SEQ ID NO:2):

MGLPLPLLQSSLLLMLLLRLSAASTNLNWQCPRIPYAASRDFSVKYVVPS
FSAGGRVQATAAYEDSTNSAVFVATRNHLHVLGPDLQFIENLTTGPIGNP
GCQTCASCGPGPHGPPKDTDTLVLVMEPGLPALVSCGSTLQGRCFLHELE
PRGKALHLAAPACLFSANNNKPEACTDCVASPLGTRVTVVEQGHASYFYV
ASSLDPELAASFSPRSVSIRRLKSDTSGFQPGFPSLSVLPKYLASYLIKY
VYSFHSGDFVYFLTVQPISVTSPPSALHTRLVRLNAVEPEIGDYRELVLD
CHFAPKRRRRGAPEGTQPYPVLQAAHSAPVDAKLAVELSISEGQEVLFGV
FVTVKDGGSGMGPNSVVCAFPIYHLNILIEEGVEYCCHSSNSSSLLSRGL
DFFQTPSFCPNPPGGEASGPSSRCHYFPLMVHASFTRVDLFNGLLGSVKV
TALHVTRLGNVTVAHMGTVDGRVLQVEIARSLNYLLYVSNFSLGSSGQPV
HRDVSRLGNDLLFASGDQVFKVPIQGPGCRHFLTCWRCLRAQRFMGCGWC
GDRCDRQKECPGSWQQDHCPPEISEFYPHSGPLRGTTRLTLCGSNFYLRP
DDVVPEGTHQITVGQSPCRLLPKDSSSPRPGSLKEFIQELECELEPLVTQ
AVGTTNISLVITNMPAGKHFRVEGISVQEGFSFVEPVLTSIKPDFGPRAG
GTYLTLEGQSLSIATSRAALVNGTQCRLEQVNEEQILCVTPPGAGTARVP
LHLQIGGAEVPGSWTFHYKEDPIVLDISPKCGYSGSHIMIHGQHLTSAWH
FTLSFHDGQSTVESRCAGQFVEQQQRRCRLPEYVVRNPQGWATGNLSVWG
DGAAGFTLPGFRFLPPPSPLRAGLVELKPEEHSVKVEYVGLGAVADCVTV
NMTVGGEVCQHELRGDVVICPLPPSLQLGKDGVPLQVCVDGGCHILSQVV
RSSPGRASQRILLIALLVLILLVAVLAVALIFNSRRRKKQLGAHSLSPTT
LSDINDTASGAPNHEESSESRDGTSVPLLRTESIRLQDLDRMLLAEVKDV
LIPHEQVVIHTDQVIGKGHFGVVYHGEYTDGAQNQTHCAIKSLSRITEVQ
EVEAFLREGLLMRGLHHPNILALIGIMLPPEGLPRVLLPYMRHGDLLRFI
RSPQRNPTVKDLVSFGLQVACGMEYLAEQKFVHRDLAARNCMLDESFTVK
VADFGLARGVLDKEYYSVRQHRHARLPVKWMALESLQTYRFTTKSDVWSF
GVLLWELLTRGAPPYPHIDPFDLSHFLAQGRRLPQPEYCPDSLYHVMLRC
WEADPAARPTFRALVLEVKQVVASLLGDHYVQLTAAYVNVGPRAVDDGSV
PPEQVQPSPQHCRSTSKPRPLSEPPLPT

The RON polypeptide domain designations used herein are defined as follows:

TABLE 2
Example of RON Polypeptide domains

| Domain | RON (human) | RON (mouse) |
|---|---|---|
| Potential Signal Seq. | 1-24 | 1-23 |
| Extracellular | 25-957 | 25-960 |
| Transmembrane | 958-978 | 961-981 |
| Intracellular | 979-1400 | 982-1378 |
| α-chain | 25-304 | 25-305 |
| β-chain | 310-1400 | 311-1378 |
| Sema domain | 31-522 | 33-524 |
| IPT plexin 1 | 569-671 | 571-673 |
| IPT plexin 2 | 684-767 | 686-769 |
| IPT plexin 3 | 770-860 | 772-864 |
| Kinase domain | 1082-1345 | 1059-1322 |

As one of skill in the art will appreciate, the beginning and ending residues of the domains listed may vary depending upon the computer modeling program used or the method used for determining the domain.

A variant of the human RON sequence, RONdelta160, has also been identified. RONdelta160 (p160) has the following amino acid sequence (SEQ ID NO:113):

MELLPPLPQSFLLLLLLPAKPAAGEDWQCPRTPYAASRDFDVKYVVPSFS
AGGLVQAMVTYEGDRNESAVFVAIRNRLHVLGPDLKSVQSLATGPAGDPG
CQTCAACGPGPHGPPGDTDTKVLVLDPALPALVSCGSSLQGRCFLHDLEP
QGTAVHLAAPACLFSAHHNRPDDCPDCVASPLGTRVTVVEQGQASYFYVA
SSLDAAVAASFSPRSVSIRRLKADASGFAPGFVALSVLPKHLVSYSIEYV
HSFHTGAFVYFLTVQPASVTDDPSALHTRLARLSATEPELGDYRELVLDC
RFAPKRRRRGAPEGGQPYPVLQVAHSAPVGAQLATELSIAEGQEVLFGVF
VTGKDGGPGVGPNSVVCAFPIDLLDTLIDEGVERCCESPVHPGLRRGLDF
FQSPSFCPNPPGLEALSPNTSCRHFPLLVSSSFSRVDLFNGLLGPVQVTA
LYVTRLDNVTVAHMGTMDGRILQVELVRSLNYLLYVSNFSLGDSGQPVQR
DVSRLGDHLLFASGDQVFQVPIQGPGCRHFLTCGRCLRAWHFMGCGWCGN
MCGQQKECPGSWQQDHCPPKLTEEPVLIAVQPLFGPRAGGTCLTLEGQSL
SVGTSRAVLVNGTECLLARVSEGQLLCATPPGATVASVPLSLQVGGAQVP
GSWTFQYREDPVVLSISPNCGYINSHITICGQHLTSAWHLVLSFHDGLRA
VESRCERQLPEQQLCRLPEYVVRDPQGWVAGNLSARGDGAAGFTLPGFRF
LPPPHPPSANLVPLKPEEHAIKFEYIGLGAVADCVGINVTVGGESCQHEF
RGDMVVCPLPPSLQLGQDGAPLQVCVDGECHILGRVVRPGPDGVPQSTLL
GILLPLLLLVAALATALVFSYWWRRKQLVLPPNLNDLASLDQTAGATPLP
ILYSGSDYRSGLALPAIDGLDSTTCVHGASFSDSEDESCVPLLRKESIQL
RDLDSALLAEVKDVLIPHERVVTHSDRVIGKGHFGVVYHGEYIDQAQNRI
QCAIKSLSRITEMQQVEAFLREGLLMRGLNHPNVLALIGIMLPPEGLPHV
LLPYMCHGDLLQFIRSPQRNPTVKDLISFGLQVARGMEYLAEQKFVHRDL
AARNCMLDESFTVKVADFGLARDILDREYYSVQQHRHARLPVKWMALESL
QTYRFTTKSDVWSFGVLLWELLTRGAPPYRHIDPFDLTHFLAQGRRLPQP
EYCPDSLYQVMQQCWEADPAVRPTFRVLVGEVEQIVSALLGDHYVQLPAT
YMNLGPSTSHEMNVRPEQPQFSPMPGNVRRPRPLSEPPRPT

The present invention is also directed to RON antibodies, or antigen-binding fragments, variants, or derivatives thereof which bind specifically, preferentially, or competitively to non-human RON proteins, e.g., RON from rodents or non-human primates.

RON is expressed in a large number of tumor cells, including, but not limited to certain of the following: breast cancer (Camp et al. Ann. Surg. Oncol. 12:273-281 (2005)), colorectal cancer, lung cancer, ovarian cancer, hepatocellular cancer, head and neck squamous cell cancer, thyroid cancer (Wang et al. Journal of Pathology 213:402-411 (2007)), skin cancer, bladder cancer, pancreatic cancer, gastric cancer (O'Toole et al. Cancer Research 66:9162-9710 (2006)), liver cancer and kidney cancer.

### III. RON Antibodies

In one embodiment, the present invention is directed to RON antibodies, or antigen-binding fragments, variants, or derivatives thereof. For example, the present invention includes at least the antigen-binding domains of certain monoclonal antibodies, and fragments, variants, and derivatives thereof shown in Tables 3 and 4. Table 3 lists human anti-RON Fab regions identified from a phage display library. Table 4 lists mouse anti-RON antibodies derived from hybridomas.

TABLE 3
RON-specific human Fabs.

| | Fab |
|---|---|
| 1. | M14-H06 |
| 2. | M15-E10 |
| 3. | M16-C07 |
| 4. | M23-F10 |
| 5. | M80-B03 |
| 6. | M93-D02 |
| 7. | M96-C05 |
| 8. | M97-D03 |
| 9. | M98-E12 |

TABLE 4
RON-specific Murine Monoclonal Antibodies.

| | | | ATCC |
|---|---|---|---|
| | | | Deposit |
| | Antibody | Hybridoma | Designation |
| 1. | 1P2E7 | 1.P2E7.3 | PTA-8816 |
| 2. | 1P3B2 | 1P3B2.2 | PTA-8813 |
| 3. | 1P4A3 | 1.P4A3.3 | PTA-8814 |
| 4. | 1P4A12 | 1.P4A12.2 | PTA-8815 |
| 5. | 1P5B10 | 1.P5B10.3.10 | PTA-8817 |

On Dec. 4, 2007, the following hybridomas were deposited with the American Type Culture Collection (ATCC) in Manassas, Va.: 1.P2E7.3, 1P3B2.2, 1.P4A3.3, 1.P4A12.2 and 1.P5B 10.3.10 and were given the following ATCC Patent Deposit Designations respectively: PTA-8816, PTA-8813, PTA-8814, PTA-8815 and PTA-8817. The deposited hybridoma 1.P2E7.3 produces the monoclonal antibody 1P2E7, described herein. The deposited hybridoma 1P3B2.2 produces the monoclonal antibody 1P4A3, described herein. The deposited hybridoma 1.P4A3.3 produces the monoclonal antibody 1P3B2, described herein. The deposited hybridoma 1.P4A12.2 produces the monoclonal antibody 1P4A12, described herein. The deposited hybridoma 1.P5B10.3.10 produces the monoclonal antibody 1P5B10, described herein.

As used herein, the term “antigen binding domain” includes a site that specifically binds an epitope on an antigen (e.g., an epitope of RON). The antigen binding domain of an antibody typically includes at least a portion of an immunoglobulin heavy chain variable region and at least a portion of an immunoglobulin light chain variable region. The binding site formed by these variable regions determines the specificity of the antibody.

The present invention is more specifically directed to an RON antibody, or antigen-binding fragment, variant or derivatives thereof, where the RON antibody specifically binds to the same RON epitope as a reference monoclonal Fab antibody fragment selected from the group consisting of M14-H06, M15-E10, M16-C07, M23-F10, M80-B03, M93-D02, M96-C05, M97-D03 and M98-E12 or a reference monoclonal antibody selected from the group consisting of 1P2E7, 1P3B2, 1P4A3, 1P4A12 and 1P5B10.

The invention is further drawn to an RON antibody, or antigen-binding fragment, variant or derivatives thereof, where the RON antibody competitively inhibits a reference monoclonal Fab antibody fragment selected from the group consisting of M14-H06, M15-E10, M16-C07, M23-F10, M80-B03, M93-D02, M96-C05, M97-D03 and M98-E12 or a reference monoclonal antibody selected from the group consisting of 1P2E7, 1P3B2, 1P4A3, 1P4A12 and 1P5B10 from binding to RON.

The invention is also drawn to an RON antibody, or antigen-binding fragment, variant or derivatives thereof, where the RON antibody comprises an antigen binding domain identical to that of a monoclonal Fab antibody fragment selected from the group consisting of M14-H06, M15-E10, M16-C07, M23-F10, M80-B03, M93-D02, M96-C05, M97-D03 and M98-E12 or a reference monoclonal antibody selected from the group consisting of 1P2E7, 1P3B2, 1P4A3, 1P4A12 and 1P5B10.

Methods of making antibodies are well known in the art and described herein. Once antibodies to various fragments of, or to the full-length RON without the signal sequence, have been produced, determining which amino acids, or epitope, of RON to which the antibody or antigen binding fragment binds can be determined by epitope mapping protocols as described herein as well as methods known in the art (e.g. double antibody-sandwich ELISA as described in “Chapter 11—Immunology,” Current Protocols in Molecular Biology, Ed. Ausubel et al., v.2, John Wiley & Sons, Inc. (1996)). Additional epitope mapping protocols may be found in Morris, G. Epitope Mapping Protocols, New Jersey: Humana Press (1996), which are both incorporated herein by reference in their entireties. Epitope mapping can also be performed by commercially available means (i.e. ProtoPROBE, Inc. (Milwaukee, Wis.)).

Additionally, antibodies produced which bind to any portion of RON can then be screened for their ability to act as an antagonist of RON for example, to inhibit binding of MSP to RON, to inhibit activation of the PI3-K/Akt pathway, to inhibit activation of the Ras/MAPK signaling pathway, to inhibit activation of the src signaling pathway, to inhibit activation of the β-catenin signaling pathway, to inhibit activation of the Fak pathway, to inhibit phosphorylation of Erk 1/2, to inhibit activation of Erk 1/2, to induce apoptosis, to induce anoikis, to inhibit receptor tyrosine kinase (RTK) activity, to block VEGF secretion, to block activity of other receptor kinases including but not limited to EGFR, TGFβ receptor and Met, to induce cytotoxic nitric oxide (NO) secretion, to induce IL-12 secretion, to block MSP secretion or to inhibit tumor cell growth, proliferation, adhesion, motility, invasion or metastasis. Antibodies can be screened for these and other properties according to methods described in detail in the Examples. Other functions of antibodies of the present invention can be tested using other assays as described in the Examples herein

In other embodiments, the present invention includes an antibody, or antigen-binding fragment, variant, or derivative thereof which specifically or preferentially binds to at least one epitope of RON, where the epitope comprises, consists essentially of, or consists of at least about four to five amino acids of SEQ ID NO:1, SEQ ID NO:2 or SEQ ID NO:113, at least seven, at least nine, or between at least about 15 to about 30 amino acids of SEQ ID NO:1, SEQ ID NO:2 or SEQ ID NO:113. The amino acids of a given epitope of SEQ ID NO:1, SEQ ID NO:2 or SEQ ID NO:113 as described may be, but need not be contiguous or linear. In certain embodiments, at least one epitope of RON comprises, consists essentially of, or consists of a non-linear epitope formed by the extracellular domain of RON as expressed on the surface of a cell or as a soluble fragment, e.g., fused to an IgG Fc region. Thus, in certain embodiments at least one epitope of RON comprises, consists essentially of, or consists of at least 4, at least 5, at least 6, at least 7, at least 8, at least 9, at least 10, at least 15, at least 20, at least 25, between about 15 to about 30, or at least 10, 15, 20, 25, 30, 35, 40, 45, 50, 55, 60, 65, 70, 75, 80, 85, 90, 95, or 100 contiguous or non-contiguous amino acids of SEQ ID NO:1, SEQ ID NO:2 or SEQ ID NO:113, where non-contiguous amino acids form an epitope through protein folding.

In other embodiments, the present invention includes an antibody, or antigen-binding fragment, variant, or derivative thereof which specifically or preferentially binds to at least one epitope of RON, where the epitope comprises, consists essentially of, or consists of, in addition to one, two, three, four, five, six or more contiguous or non-contiguous amino acids of SEQ ID NO:1, SEQ ID NO:2 or SEQ ID NO:113 as described above, and an additional moiety which modifies the protein, e.g., a carbohydrate moiety may be included such that the RON antibody binds with higher affinity to modified target protein than it does to an unmodified version of the protein. Alternatively, the RON antibody does not bind the unmodified version of the target protein at all.

In certain aspects, the present invention is directed to an antibody, or antigen-binding fragment, variant, or derivative thereof which specifically binds to a RON polypeptide or fragment thereof, or an RON variant polypeptide, with an affinity characterized by a dissociation constant (K_{D}) which is less than the K_{D} for a given reference monoclonal antibody.

In certain embodiments, an antibody, or antigen-binding fragment, variant, or derivative thereof of the invention binds specifically to at least one epitope of RON or fragment or variant described above, i.e., binds to such an epitope more readily than it would bind to an unrelated, or random epitope; binds preferentially to at least one epitope of RON or fragment or variant described above, i.e., binds to such an epitope more readily than it would bind to a related, similar, homologous, or analogous epitope; competitively inhibits binding of a reference antibody which itself binds specifically or preferentially to a certain epitope of RON or fragment or variant described above; or binds to at least one epitope of RON or fragment or variant described above with an affinity characterized by a dissociation constant K_{D} of less than about 5×10⁻² M, about 10⁻² M, about 5×10⁻³ M, about 10⁻³ M, about 5×10⁻⁴ M, about 10⁻⁴ M, about 5×10⁻⁵ M, about 10⁻⁵ M, about 5×10⁻⁶ M, about 10⁻⁶ M, about 5×10⁻⁷ M, about 10⁻⁷ M, about 5×10⁻⁸ M, about 10⁻⁸ M, about 5×10⁻⁹ M, about 10⁻⁹ M, about 5×10⁻¹⁰ M, about 10⁻¹⁰ M, about 5×10⁻¹¹ M, about 10⁻¹¹ M, about 5×10⁻¹² M, about 10⁻¹² M, about 5×10⁻¹³ M, about 10⁻¹³ M, about 5×10⁻¹⁴ M, about 10⁻¹⁴ M, about 5×10⁻¹⁵ M, or about 10⁻¹⁵ M. In a particular aspect, the antibody or fragment thereof preferentially binds to a human RON polypeptide or fragment thereof, relative to a murine RON polypeptide or fragment thereof. In another particular aspect, the antibody or fragment thereof preferentially binds to one or more RON polypeptides or fragments thereof, e.g., one or more mammalian RON polypeptides.

As used in the context of antibody binding dissociation constants, the term “about” allows for the degree of variation inherent in the methods utilized for measuring antibody affinity. For example, depending on the level of precision of the instrumentation used, standard error based on the number of samples measured, and rounding error, the term “about 10⁻² M” might include, for example, from 0.05 M to 0.005 M.

In specific embodiments, an antibody, or antigen-binding fragment, immunospecific fragment, variant, or derivative thereof of the invention binds RON polypeptides or fragments or variants thereof with an off rate (k(off)) of less than or equal to 5×10⁻² sec⁻¹, 10⁻² sec⁻¹, 5×10⁻³ sec⁻¹ or 10⁻³ sec⁻¹. Alternatively, an antibody, or antigen-binding fragment, variant, or derivative thereof of the invention binds RON polypeptides or fragments or variants thereof with an off rate (k(off)) of less than or equal to 5×10⁻⁴ sec⁻¹, 10⁻⁴ sec⁻¹, 5×10⁻⁵ sec⁻¹, or 10⁻⁵ sec⁻¹ 5×10⁻⁶ sec⁻¹, 10⁻⁶ sec⁻¹, 5×10⁻⁷ sec⁻¹ or 10⁻⁷ sec⁻¹.

In other embodiments, an antibody, or antigen-binding fragment, immunospecific fragment, variant, or derivative thereof of the invention binds RON polypeptides or fragments or variants thereof with an on rate (k(on)) of greater than or equal to 10³ M⁻¹ sec⁻¹, 5×10³ M⁻¹ sec⁻¹, 10⁴ M⁻¹ sec⁻¹ or 5×10⁴ M⁻¹ sec⁻¹. Alternatively, an antibody, or antigen-binding fragment, variant, or derivative thereof of the invention binds RON polypeptides or fragments or variants thereof with an on rate (k(on)) greater than or equal to 10⁵ M⁻¹ sec⁻¹, 5×10⁵ M⁻¹ sec⁻¹, 10⁶ M⁻¹ sec⁻¹, or 5×106 M⁻¹ sec⁻¹ or 10⁷ M⁻¹ sec⁻¹.

In various embodiments, an RON antibody, or antigen-binding fragment, variant, or derivative thereof as described herein is an antagonist of RON activity. In certain embodiments, for example, binding of an antagonist RON antibody to RON as expressed on a tumor cell or tumor associated macrophage inhibits binding of MSP to RON, inhibits MSP-induced RON signaling, inhibits activation of the PI3-K/Akt pathway, the Ras/MAPK pathway, the src pathway, the Fak pathway or the β-catenin pathway, inhibits phosphorylation or activation of ERK 1/2, blocks VEGF secretion, blocks activity of other receptor kinases including but not limited to EGFR, TGFβ receptor and Met, induces cytotoxic nitric oxide (NO) secretion, induces IL-12 secretion, blocks MSP secretion or inhibits tumor cell proliferation, motility or metastasis or promotes apoptosis or anoikis.

Unless it is specifically noted, as used herein a “fragment thereof” in reference to an antibody refers to an antigen-binding fragment, i.e., a portion of the antibody which specifically binds to the antigen. In one embodiment, an RON antibody, e.g., an antibody of the invention is a bispecific RON antibody, e.g., a bispecific antibody, minibody, domain deleted antibody, or fusion protein having binding specificity for more than one epitope, e.g., more than one antigen or more than one epitope on the same antigen. In one embodiment, a bispecific RON antibody has at least one binding domain specific for at least one epitope on a target polypeptide disclosed herein, e.g., RON. In another embodiment, a bispecific RON antibody has at least one binding domain specific for an epitope on a target polypeptide and at least one target binding domain specific for a drug or toxin. In yet another embodiment, a bispecific RON antibody has at least one binding domain specific for an epitope on a target polypeptide disclosed herein, and at least one binding domain specific for a prodrug. A bispecific RON antibody may be a tetravalent antibody that has two target binding domains specific for an epitope of a target polypeptide disclosed herein and two target binding domains specific for a second target. Thus, a tetravalent bispecific RON antibody may be bivalent for each specificity.

RON antibodies, or antigen-binding fragments, variants, or derivatives thereof of the invention, as known by those of ordinary skill in the art, can comprise a constant region which mediates one or more effector functions. For example, binding of the C1 component of complement to an antibody constant region may activate the complement system. Activation of complement is important in the opsonisation and lysis of cell pathogens. The activation of complement also stimulates the inflammatory response and may also be involved in autoimmune hypersensitivity. Further, antibodies bind to receptors on various cells via the Fc region, with a Fc receptor binding site on the antibody Fc region binding to a Fc receptor (FcR) on a cell. There are a number of Fc receptors which are specific for different classes of antibody, including IgG (gamma receptors), IgE (epsilon receptors), IgA (alpha receptors) and IgM (mu receptors). Binding of antibody to Fc receptors on cell surfaces triggers a number of important and diverse biological responses including engulfment and destruction of antibody-coated particles, clearance of immune complexes, lysis of antibody-coated target cells by killer cells (called antibody-dependent cell-mediated cytotoxicity, or ADCC), release of inflammatory mediators, placental transfer and control of immunoglobulin production.

Accordingly, certain embodiments of the invention include an RON antibody, or antigen-binding fragment, variant, or derivative thereof, in which at least a fraction of one or more of the constant region domains has been deleted or otherwise altered so as to provide desired biochemical characteristics such as reduced effector functions, the ability to non-covalently dimerize, increased ability to localize at the site of a tumor, reduced serum half-life, or increased serum half-life when compared with a whole, unaltered antibody of approximately the same immunogenicity. For example, certain antibodies for use in the diagnostic and treatment methods described herein are domain deleted antibodies which comprise a polypeptide chain similar to an immunoglobulin heavy chain, but which lack at least a portion of one or more heavy chain domains. For instance, in certain antibodies, one entire domain of the constant region of the modified antibody will be deleted, for example, all or part of the CH2 domain will be deleted. For example, RON antibodies that have been altered in the Fc region may deplete macrophages involved in cancer or inflammation. In other embodiments, certain antibodies for use in the diagnostic and treatment methods described herein have a constant region, e.g., an IgG4 heavy chain constant region, which is altered to eliminate glycosylation, referred to elsewhere herein as “agly” antibodies. While not being bound by theory, it is believed that “agly” antibodies may have an improved safety and stability profile in vivo. Methods of producing a glycosylated antibodies, having desired effector function are found for example in WO 2005018572, which is incorporated by reference in its entirety.

In certain RON antibodies, or antigen-binding fragments, variants, or derivatives thereof described herein, the Fc portion may be mutated to decrease effector function using techniques known in the art. For example, the deletion or inactivation (through point mutations or other means) of a constant region domain may reduce Fc receptor binding of the circulating modified antibody thereby increasing tumor localization. In other cases it may be that constant region modifications consistent with the instant invention moderate complement binding and thus reduce the serum half life and nonspecific association of a conjugated cytotoxin. Yet other modifications of the constant region may be used to modify disulfide linkages or oligosaccharide moieties that allow for enhanced localization due to increased antigen specificity or antibody flexibility. The resulting physiological profile, bioavailability and other biochemical effects of the modifications, such as tumor localization, biodistribution and serum half-life, may easily be measured and quantified using well know immunological techniques without undue experimentation.

Modified forms of RON antibodies, or antigen-binding fragments, variants, or derivatives thereof of the invention can be made from whole precursor or parent antibodies using techniques known in the art. Exemplary techniques are discussed in more detail herein.

In certain embodiments both the variable and constant regions of RON antibodies, or antigen-binding fragments, variants, or derivatives thereof are fully human. Fully human antibodies can be made using techniques that are known in the art and as described herein. For example, fully human antibodies against a specific antigen can be prepared by administering the antigen to a transgenic animal which has been modified to produce such antibodies in response to antigenic challenge, but whose endogenous loci have been disabled. Exemplary techniques that can be used to make such antibodies are described in U.S. Pat. Nos. 6,150,584; 6,458,592; 6,420,140. Other techniques are known in the art. Fully human antibodies can likewise be produced by various display technologies, e.g., phage display or other viral display systems, as described in more detail elsewhere herein.

RON antibodies, or antigen-binding fragments, variants, or derivatives thereof of the invention can be made or manufactured using techniques that are known in the art. In certain embodiments, antibody molecules or fragments thereof are “recombinantly produced,” i.e., are produced using recombinant DNA technology. Exemplary techniques for making antibody molecules or fragments thereof are discussed in more detail elsewhere herein.

RON antibodies, or antigen-binding fragments, variants, or derivatives thereof of the invention also include derivatives that are modified, e.g., by the covalent attachment of any type of molecule to the antibody such that covalent attachment does not prevent the antibody from specifically binding to its cognate epitope. For example, but not by way of limitation, the antibody derivatives include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

In certain embodiments, RON antibodies, or antigen-binding fragments, variants, or derivatives thereof of the invention will not elicit a deleterious immune response in the animal to be treated, e.g., in a human. In one embodiment, RON antibodies, or antigen-binding fragments, variants, or derivatives thereof of the invention are modified to reduce their immunogenicity using art-recognized techniques. For example, antibodies can be humanized, primatized, deimmunized, or chimeric antibodies can be made. These types of antibodies are derived from a non-human antibody, typically a murine or primate antibody, that retains or substantially retains the antigen-binding properties of the parent antibody, but which is less immunogenic in humans. This may be achieved by various methods, including (a) grafting the entire non-human variable domains onto human constant regions to generate chimeric antibodies; (b) grafting at least a part of one or more of the non-human complementarity determining regions (CDRs) into a human framework and constant regions with or without retention of critical framework residues; or (c) transplanting the entire non-human variable domains, but “cloaking” them with a human-like section by replacement of surface residues. Such methods are disclosed in Morrison et al., Proc. Natl. Acad. Sci. 81:6851-6855 (1984); Morrison et al., Adv. Immunol. 44:65-92 (1988); Verhoeyen et al., Science 239:1534-1536 (1988); Padlan, Molec. Immun. 28:489-498 (1991); Padlan, Molec. Immun. 31:169-217 (1994), and U.S. Pat. Nos. 5,585,089, 5,693,761, 5,693,762, and 6,190,370, all of which are hereby incorporated by reference in their entirety.

De-immunization can also be used to decrease the immunogenicity of an antibody. As used herein, the term “de-immunization” includes alteration of an antibody to modify T cell epitopes (see, e.g., WO9852976A1, WO0034317A2). For example, VH and VL sequences from the starting antibody are analyzed and a human T cell epitope “map” from each V region showing the location of epitopes in relation to complementarity-determining regions (CDRs) and other key residues within the sequence. Individual T cell epitopes from the T cell epitope map are analyzed in order to identify alternative amino acid substitutions with a low risk of altering activity of the final antibody. A range of alternative VH and VL sequences are designed comprising combinations of amino acid substitutions and these sequences are subsequently incorporated into a range of binding polypeptides, e.g., RON-specific antibodies or immunospecific fragments thereof for use in the diagnostic and treatment methods disclosed herein, which are then tested for function. Typically, between 12 and 24 variant antibodies are generated and tested. Complete heavy and light chain genes comprising modified V and human C regions are then cloned into expression vectors and the subsequent plasmids introduced into cell lines for the production of whole antibody. The antibodies are then compared in appropriate biochemical and biological assays, and the optimal variant is identified.

RON antibodies, or antigen-binding fragments, variants, or derivatives thereof of the invention may be generated by any suitable method known in the art. Polyclonal antibodies to an antigen of interest can be produced by various procedures well known in the art. For example, an RON antibody, e.g., a binding polypeptide, e.g., an RON-specific antibody or immunospecific fragment thereof can be administered to various host animals including, but not limited to, rabbits, mice, rats, chickens, hamsters, goats, donkeys, etc., to induce the production of sera containing polyclonal antibodies specific for the antigen. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum. Such adjuvants are also well known in the art.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, Cold Spring Harbor Laboratory Press, 2nd ed. (1988); Hammerling et al., in: Monoclonal Antibodies and T-Cell Hybridomas Elsevier, N.Y., 563-681 (1981) (said references incorporated by reference in their entireties). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced. Thus, the term “monoclonal antibody” is not limited to antibodies produced through hybridoma technology. Monoclonal antibodies can be prepared using RON knockout mice to increase the regions of epitope recognition. Monoclonal antibodies can be prepared using a wide variety of techniques known in the art including the use of hybridoma and recombinant and phage display technology as described elsewhere herein.

Using art recognized protocols, in one example, antibodies are raised in mammals by multiple subcutaneous or intraperitoneal injections of the relevant antigen (e.g., purified RON or cells or cellular extracts comprising RON) and an adjuvant. This immunization typically elicits an immune response that comprises production of antigen-reactive antibodies from activated splenocytes or lymphocytes. While the resulting antibodies may be harvested from the serum of the animal to provide polyclonal preparations, it is often desirable to isolate individual lymphocytes from the spleen, lymph nodes or peripheral blood to provide homogenous preparations of monoclonal antibodies (MAbs). Preferably, the lymphocytes are obtained from the spleen.

In this well known process (Kohler et al., Nature 256:495 (1975)) the relatively short-lived, or mortal, lymphocytes from a mammal which has been injected with antigen are fused with an immortal tumor cell line (e.g., a myeloma cell line), thus, producing hybrid cells or “hybridomas” which are both immortal and capable of producing the genetically coded antibody of the B cell. The resulting hybrids are segregated into single genetic strains by selection, dilution, and regrowth with each individual strain comprising specific genes for the formation of a single antibody. They produce antibodies, which are homogeneous against a desired antigen and, in reference to their pure genetic parentage, are termed “monoclonal.”

Hybridoma cells thus prepared are seeded and grown in a suitable culture medium that preferably contains one or more substances that inhibit the growth or survival of the unfused, parental myeloma cells. Those skilled in the art will appreciate that reagents, cell lines and media for the formation, selection and growth of hybridomas are commercially available from a number of sources and standardized protocols are well established. Generally, culture medium in which the hybridoma cells are growing is assayed for production of monoclonal antibodies against the desired antigen. Preferably, the binding specificity of the monoclonal antibodies produced by hybridoma cells is determined by in vitro assays such as immunoprecipitation, radioimmunoassay (RIA) or enzyme-linked immunoabsorbent assay (ELISA). After hybridoma cells are identified that produce antibodies of the desired specificity, affinity and/or activity, the clones may be subcloned by limiting dilution procedures and grown by standard methods (Goding, Monoclonal Antibodies: Principles and Practice, Academic Press, pp 59-103 (1986)). It will further be appreciated that the monoclonal antibodies secreted by the subclones may be separated from culture medium, ascites fluid or serum by conventional purification procedures such as, for example, protein-A, hydroxylapatite chromatography, gel electrophoresis, dialysis or affinity chromatography.

Antibody fragments that recognize specific epitopes may be generated by known techniques. For example, Fab and F(ab′)₂ fragments may be produced recombinantly or by proteolytic cleavage of immunoglobulin molecules, using enzymes such as papain (to produce Fab fragments) or pepsin (to produce F(ab′)₂ fragments). F(ab′)₂ fragments contain the variable region, the light chain constant region and the CH1 domain of the heavy chain.

Those skilled in the art will also appreciate that DNA encoding antibodies or antibody fragments (e.g., antigen binding sites) may also be derived from antibody libraries, such as phage display libraries. In a particular, such phage can be utilized to display antigen-binding domains expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. Phage used in these methods are typically filamentous phage including fd and M13 binding domains expressed from phage with Fab, Fv OE DAB (individual Fv region from light or heavy chains) or disulfide stabilized Fv antibody domains recombinantly fused to either the phage gene III or gene VIII protein. Exemplary methods are set forth, for example, in EP 368 684 B1; U.S. Pat. No. 5,969,108, Hoogenboom, H. R. and Chames, Immunol. Today 21:371 (2000); Nagy et al. Nat. Med. 8:801 (2002); Huie et al., Proc. Natl. Acad. Sci. USA 98:2682 (2001); Lui et al., J. Mol. Biol. 315:1063 (2002), each of which is incorporated herein by reference. Several publications (e.g., Marks et al., Bio/Technology 10:779-783 (1992)) have described the production of high affinity human antibodies by chain shuffling, as well as combinatorial infection and in vivo recombination as a strategy for constructing large phage libraries. In another embodiment, Ribosomal display can be used to replace bacteriophage as the display platform (see, e.g., Hanes et al., Nat. Biotechnol. 18:1287 (2000); Wilson et al., Proc. Natl. Acad. Sci. USA 98:3750 (2001); or Irving et al., J. Immunol. Methods 248:31 (2001)). In yet another embodiment, cell surface libraries can be screened for antibodies (Boder et al., Proc. Natl. Acad. Sci. USA 97:10701 (2000); Daugherty et al., J. Immunol. Methods 243:211 (2000)). Such procedures provide alternatives to traditional hybridoma techniques for the isolation and subsequent cloning of monoclonal antibodies.

In phage display methods, functional antibody domains are displayed on the surface of phage particles, which carry the polynucleotide sequences encoding them. For example, DNA sequences encoding VH and VL regions are amplified or otherwise isolated from animal cDNA libraries (e.g., human or murine cDNA libraries of lymphoid tissues) or synthetic cDNA libraries. In certain embodiments, the DNA encoding the VH and VL regions are joined together by an scFv linker by PCR and cloned into a phagemid vector (e.g., p CANTAB 6 or pComb 3 HSS). The vector is electroporated in E. coli and the E. coli is infected with helper phage. Phage used in these methods are typically filamentous phage including fd and M13 and the VH or VL regions are usually recombinantly fused to either the phage gene III or gene VIII. Phage expressing an antigen binding domain that binds to an antigen of interest (i.e., an RON polypeptide or a fragment thereof) can be selected or identified with antigen, e.g., using labeled antigen or antigen bound or captured to a solid surface or bead.

Additional examples of phage display methods that can be used to make the antibodies include those disclosed in Brinkman et al., J. Immunol. Methods 182:41-50 (1995); Ames et al., J. Immunol. Methods 184:177-186 (1995); Kettleborough et al., Eur. J. Immunol. 24:952-958 (1994); Persic et al., Gene 187:9-18 (1997); Burton et al., Advances in Immunology 57:191-280 (1994); PCT Application No. PCT/GB91/01134; PCT publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108; each of which is incorporated herein by reference in its entirety.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria. For example, techniques to recombinantly produce Fab, Fab′ and F(ab′)₂ fragments can also be employed using methods known in the art such as those disclosed in PCT publication WO 92/22324; Mullinax et al., BioTechniques 12(6):864-869 (1992); and Sawai et al., AJRI 34:26-34 (1995); and Better et al., Science 240:1041-1043 (1988) (said references incorporated by reference in their entireties).

Examples of techniques which can be used to produce single-chain Fvs and antibodies include those described in U.S. Pat. Nos. 4,946,778 and 5,258,498; Huston et al., Methods in Enzymology 203:46-88 (1991); Shu et al., PNAS 90:7995-7999 (1993); and Skerra et al., Science 240:1038-1040 (1988). For some uses, including in vivo use of antibodies in humans and in vitro detection assays, it may be preferable to use chimeric, humanized, or human antibodies. A chimeric antibody is a molecule in which different portions of the antibody are derived from different animal species, such as antibodies having a variable region derived from a murine monoclonal antibody and a human immunoglobulin constant region. Methods for producing chimeric antibodies are known in the art. See, e.g., Morrison, Science 229:1202 (1985); Oi et al., BioTechniques 4:214 (1986); Gillies et al., J. Immunol. Methods 125:191-202 (1989); U.S. Pat. Nos. 5,807,715; 4,816,567; and 4,816397, which are incorporated herein by reference in their entireties. In addition, techniques developed for the production of “chimeric antibodies” (Morrison et al., Proc. Natl. Acad. Sci. 81:851-855 (1984); Neuberger et al., Nature 312:604-608 (1984); Takeda et al., Nature 314:452-454 (1985)) by splicing genes from a mouse antibody molecule of appropriate antigen specificity together with genes from a human antibody molecule of appropriate biological activity can be used. As used herein, a chimeric antibody is a molecule in which different portions are derived from different animal species, such as those having a variable region derived from a murine monoclonal antibody and a human immunoglobulin constant region, e.g., humanized antibodies.

Humanized antibodies are antibody molecules from non-human species antibody that binds the desired antigen having one or more complementarity determining regions (CDRs) from the non-human species and framework regions from a human immunoglobulin molecule. Often, framework residues in the human framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; Riechmann et al., Nature 332:323 (1988), which are incorporated herein by reference in their entireties.) Antibodies can be humanized using a variety of techniques known in the art including, for example, CDR-grafting (EP 239,400; PCT publication WO 91/09967; U.S. Pat. Nos. 5,225,539; 5,530,101; and 5,585,089), veneering or resurfacing (EP 592,106; EP 519,596; Padlan, Molecular Immunology 28(4/5):489-498 (1991); Studnicka et al., Protein Engineering 7(6):805-814 (1994); Roguska. et al., PNAS 91:969-973 (1994)), and chain shuffling (U.S. Pat. No. 5,565,332).

Completely human antibodies are particularly desirable for therapeutic treatment of human patients. Human antibodies can be made by a variety of methods known in the art including phage display methods described above using antibody libraries derived from human immunoglobulin sequences. See also, U.S. Pat. Nos. 4,444,887 and 4,716,111; and PCT publications WO 98/46645, WO 98/50433, WO 98/24893, WO 98/16654, WO 96/34096, WO 96/33735, and WO 91/10741; each of which is incorporated herein by reference in its entirety.

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring that express human antibodies. The transgenic mice are immunized in the normal fashion with a selected antigen, e.g., all or a portion of a desired target polypeptide. Monoclonal antibodies directed against the antigen can be obtained from the immunized, transgenic mice using conventional hybridoma technology. The human immunoglobulin transgenes harbored by the transgenic mice rearrange during B-cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar Int. Rev. Immunol. 13:65-93 (1995). For a detailed discussion of this technology for producing human antibodies and human monoclonal antibodies and protocols for producing such antibodies, see, e.g., PCT publications WO 98/24893; WO 96/34096; WO 96/33735; U.S. Pat. Nos. 5,413,923; 5,625,126; 5,633,425; 5,569,825; 5,661,016; 5,545,806; 5,814,318; and 5,939,598, which are incorporated by reference herein in their entirety. In addition, companies such as Abgenix, Inc. (Freemont, Calif.) and GenPharm (San Jose, Calif.) can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope. (Jespers et al., Bio/Technology 12:899-903 (1988). See also, U.S. Pat. No. 5,565,332.)

Further, antibodies to target polypeptides of the invention can, in turn, be utilized to generate anti-idiotype antibodies that “mimic” target polypeptides using techniques well known to those skilled in the art. (See, e.g., Greenspan & Bona, FASEB J. 7(5):437-444 (1989) and Nissinoff, J. Immunol. 147(8):2429-2438 (1991)). For example, antibodies which bind to and competitively inhibit polypeptide multimerization and/or binding of a polypeptide of the invention to a ligand can be used to generate anti-idiotypes that “mimic” the polypeptide multimerization and/or binding domain and, as a consequence, bind to and neutralize polypeptide and/or its ligand. Such neutralizing anti-idiotypes or Fab fragments of such anti-idiotypes can be used in therapeutic regimens to neutralize polypeptide ligand. For example, such anti-idiotypic antibodies can be used to bind a desired target polypeptide and/or to bind its ligands/receptors, and thereby block its biological activity.

In another embodiment, DNA encoding desired monoclonal antibodies may be readily isolated and sequenced using conventional procedures (e.g., by using oligonucleotide probes that are capable of binding specifically to genes encoding the heavy and light chains of murine antibodies). The isolated and subcloned hybridoma cells serve as a preferred source of such DNA. Once isolated, the DNA may be placed into expression vectors, which are then transfected into prokaryotic or eukaryotic host cells such as, but not limited to, E. coli cells, simian COS cells, Chinese Hamster Ovary (CHO) cells or myeloma cells that do not otherwise produce immunoglobulins. More particularly, the isolated DNA (which may be synthetic as described herein) may be used to clone constant and variable region sequences for the manufacture antibodies as described in Newman et al., U.S. Pat. No. 5,658,570, filed Jan. 25, 1995, which is incorporated by reference herein. Essentially, this entails extraction of RNA from the selected cells, conversion to cDNA, and amplification by PCR using Ig specific primers. Suitable primers for this purpose are also described in U.S. Pat. No. 5,658,570. As will be discussed in more detail below, transformed cells expressing the desired antibody may be grown up in relatively large quantities to provide clinical and commercial supplies of the immunoglobulin.

In one embodiment, an RON antibody of the invention comprises at least one heavy or light chain CDR of an antibody molecule. In another embodiment, an RON antibody of the invention comprises at least two CDRs from one or more antibody molecules. In another embodiment, an RON antibody of the invention comprises at least three CDRs from one or more antibody molecules. In another embodiment, an RON antibody of the invention comprises at least four CDRs from one or more antibody molecules. In another embodiment, an RON antibody of the invention comprises at least five CDRs from one or more antibody molecules. In another embodiment, an RON antibody of the invention comprises at least six CDRs from one or more antibody molecules. Exemplary antibody molecules comprising at least one CDR that can be included in the subject RON antibodies are described herein.

In a specific embodiment, the amino acid sequence of the heavy and/or light chain variable domains may be inspected to identify the sequences of the complementarity determining regions (CDRs) by methods that are well know in the art, e.g., by comparison to known amino acid sequences of other heavy and light chain variable regions to determine the regions of sequence hypervariability. Using routine recombinant DNA techniques, one or more of the CDRs may be inserted within framework regions, e.g., into human framework regions to humanize a non-human antibody. The framework regions may be naturally occurring or consensus framework regions, and preferably human framework regions (see, e.g., Chothia et al., J. Mol. Biol. 278:457-479 (1998) for a listing of human framework regions). Preferably, the polynucleotide generated by the combination of the framework regions and CDRs encodes an antibody that specifically binds to at least one epitope of a desired polypeptide, e.g., RON. Preferably, one or more amino acid substitutions may be made within the framework regions, and, preferably, the amino acid substitutions improve binding of the antibody to its antigen. Additionally, such methods may be used to make amino acid substitutions or deletions of one or more variable region cysteine residues participating in an intrachain disulfide bond to generate antibody molecules lacking one or more intrachain disulfide bonds. Other alterations to the polynucleotide are encompassed by the present invention and within the skill of the art.

Alternatively, techniques described for the production of single chain antibodies (U.S. Pat. No. 4,694,778; Bird, Science 242:423-442 (1988); Huston et al., Proc. Natl. Acad. Sci. USA 85:5879-5883 (1988); and Ward et al., Nature 334:544-554 (1989)) can be adapted to produce single chain antibodies. Single chain antibodies are formed by linking the heavy and light chain fragments of the Fv region via an amino acid bridge, resulting in a single chain antibody. Techniques for the assembly of functional Fv fragments in E coli may also be used (Skerra et al., Science 242:1038-1041 (1988)).

Yet other embodiments of the present invention comprise the generation of human or substantially human antibodies in transgenic animals (e.g., mice) that are incapable of endogenous immunoglobulin production (see e.g., U.S. Pat. Nos. 6,075,181, 5,939,598, 5,591,669 and 5,589,369 each of which is incorporated herein by reference). For example, it has been described that the homozygous deletion of the antibody heavy-chain joining region in chimeric and germ-line mutant mice results in complete inhibition of endogenous antibody production. Transfer of a human immunoglobulin gene array to such germ line mutant mice will result in the production of human antibodies upon antigen challenge. Another preferred means of generating human antibodies using SCID mice is disclosed in U.S. Pat. No. 5,811,524 which is incorporated herein by reference. It will be appreciated that the genetic material associated with these human antibodies may also be isolated and manipulated as described herein.

Yet another highly efficient means for generating recombinant antibodies is disclosed by Newman, Biotechnology 10: 1455-1460 (1992). Specifically, this technique results in the generation of primatized antibodies that contain monkey variable domains and human constant sequences. This reference is incorporated by reference in its entirety herein. Moreover, this technique is also described in commonly assigned U.S. Pat. Nos. 5,658,570, 5,693,780 and 5,756,096 each of which is incorporated herein by reference.

In another embodiment, lymphocytes can be selected by micromanipulation and the variable genes isolated. For example, peripheral blood mononuclear cells can be isolated from an immunized mammal and cultured for about 7 days in vitro. The cultures can be screened for specific IgGs that meet the screening criteria. Cells from positive wells can be isolated. Individual Ig-producing B cells can be isolated by FACS or by identifying them in a complement-mediated hemolytic plaque assay. Ig-producing B cells can be micromanipulated into a tube and the VH and VL genes can be amplified using, e.g., RT-PCR. The VH and VL genes can be cloned into an antibody expression vector and transfected into cells (e.g., eukaryotic or prokaryotic cells) for expression.

Alternatively, antibody-producing cell lines may be selected and cultured using techniques well known to the skilled artisan. Such techniques are described in a variety of laboratory manuals and primary publications. In this respect, techniques suitable for use in the invention as described below are described in Current Protocols in Immunology, Coligan et al., Eds., Green Publishing Associates and Wiley-Interscience, John Wiley and Sons, New York (1991) which is herein incorporated by reference in its entirety, including supplements.

Antibodies of the present invention can be produced by any method known in the art for the synthesis of antibodies, in particular, by chemical synthesis or preferably, by recombinant expression techniques as described herein.

In one embodiment, an RON antibody, or antigen-binding fragment, variant, or derivative thereof of the invention comprises a synthetic constant region wherein one or more domains are partially or entirely deleted (“domain-deleted antibodies”). In certain embodiments compatible modified antibodies will comprise domain deleted constructs or variants wherein the entire CH2 domain has been removed (ΔCH2 constructs). For other embodiments a short connecting peptide may be substituted for the deleted domain to provide flexibility and freedom of movement for the variable region. Those skilled in the art will appreciate that such constructs are particularly preferred due to the regulatory properties of the CH2 domain on the catabolic rate of the antibody. Domain deleted constructs can be derived using a vector encoding an IgG₁ human constant domain (see, e.g., WO 02/060955A2 and WO02/096948A2). This vector is engineered to delete the CH2 domain and provide a synthetic vector expressing a domain deleted IgG₁ constant region.

In certain embodiments, RON antibodies, or antigen-binding fragments, variants, or derivatives thereof of the invention are minibodies. Minibodies can be made using methods described in the art (see, e.g., U.S. Pat. No. 5,837,821 or WO 94/09817A1).

In one embodiment, an RON antibody, or antigen-binding fragment, variant, or derivative thereof of the invention comprises an immunoglobulin heavy chain having deletion or substitution of a few or even a single amino acid as long as it permits association between the monomeric subunits. For example, the mutation of a single amino acid in selected areas of the CH2 domain may be enough to substantially reduce Fc binding and thereby increase tumor localization. Similarly, it may be desirable to simply delete that part of one or more constant region domains that control the effector function (e.g. complement binding) to be modulated. Such partial deletions of the constant regions may improve selected characteristics of the antibody (serum half-life) while leaving other desirable functions associated with the subject constant region domain intact. Moreover, as alluded to above, the constant regions of the disclosed antibodies may be synthetic through the mutation or substitution of one or more amino acids that enhances the profile of the resulting construct. In this respect it may be possible to disrupt the activity provided by a conserved binding site (e.g. Fc binding) while substantially maintaining the configuration and immunogenic profile of the modified antibody. Yet other embodiments comprise the addition of one or more amino acids to the constant region to enhance desirable characteristics such as effector function or provide for more cytotoxin or carbohydrate attachment. In such embodiments it may be desirable to insert or replicate specific sequences derived from selected constant region domains.

The present invention also provides antibodies that comprise, consist essentially of, or consist of, variants (including derivatives) of antibody molecules (e.g., the VH regions and/or VL regions) described herein, which antibodies or fragments thereof immunospecifically bind to an RON polypeptide or fragment or variant thereof. Standard techniques known to those of skill in the art can be used to introduce mutations in the nucleotide sequence encoding an RON antibody, including, but not limited to, site-directed mutagenesis and PCR-mediated mutagenesis which result in amino acid substitutions. Preferably, the variants (including derivatives) encode less than 50 amino acid substitutions, less than 40 amino acid substitutions, less than 30 amino acid substitutions, less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the reference VH region, VH-CDR1, VH-CDR2, VH-CDR3, VL region, VL-CDR1, VL-CDR2, or VL-CDR3. A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity (e.g., the ability to bind an RON polypeptide).

For example, it is possible to introduce mutations only in framework regions or only in CDR regions of an antibody molecule. Introduced mutations may be silent or neutral missense mutations, i.e., have no, or little, effect on an antibody's ability to bind antigen, indeed some such mutations do not alter the amino acid sequence whatsoever. These types of mutations may be useful to optimize codon usage, or improve a hybridoma's antibody production. Codon-optimized coding regions encoding RON antibodies of the present invention are disclosed elsewhere herein. Alternatively, non-neutral missense mutations may alter an antibody's ability to bind antigen. The location of most silent and neutral missense mutations is likely to be in the framework regions, while the location of most non-neutral missense mutations is likely to be in CDR, though this is not an absolute requirement. One of skill in the art would be able to design and test mutant molecules with desired properties such as no alteration in antigen binding activity or alteration in binding activity (e.g., improvements in antigen binding activity or change in antibody specificity). Following mutagenesis, the encoded protein may routinely be expressed and the functional and/or biological activity of the encoded protein, (e.g., ability to immunospecifically bind at least one epitope of an RON polypeptide) can be determined using techniques described herein or by routinely modifying techniques known in the art.

### IV. Polynucleotides Encoding RON Antibodies

The present invention also provides for nucleic acid molecules encoding RON antibodies, or antigen-binding fragments, variants, or derivatives thereof of the invention.

In one embodiment, the present invention provides an isolated polynucleotide comprising, consisting essentially of, or consisting of a nucleic acid encoding an immunoglobulin heavy chain variable region (VH), where at least one of the CDRs of the heavy chain variable region or at least two of the VH-CDRs of the heavy chain variable region are at least 80%, 85%, 90% or 95% identical to reference heavy chain VH-CDR1, VH-CDR2, or VH-CDR3 amino acid sequences from monoclonal RON antibodies disclosed herein. Alternatively, the VH-CDR1, VH-CDR2, and VH-CDR3 regions of the VH are at least 80%, 85%, 90% or 95% identical to reference heavy chain VH-CDR1, VH-CDR2, and VH-CDR3 amino acid sequences from monoclonal RON antibodies disclosed herein. Thus, according to this embodiment a heavy chain variable region of the invention has VH-CDR1, VH-CDR2, or VH-CDR3 polypeptide sequences related to the polypeptide sequences of SEQ ID NO: 5, SEQ ID NO: 15, SEQ ID NO: 25, SEQ ID NO: 35, SEQ ID NO: 45, SEQ ID NO: 55, SEQ ID NO: 65, SEQ ID NO: 75, SEQ ID NO: 85, SEQ ID NO: 95, SEQ ID NO:116, SEQ ID NO:126, SEQ ID NO:136, and SEQ ID NO:146; SEQ ID NO: 6, SEQ ID NO: 16, SEQ ID NO: 26, SEQ ID NO: 36, SEQ ID NO: 46, SEQ ID NO: 56, SEQ ID NO: 66, SEQ ID NO: 76, SEQ ID NO: 86, SEQ ID NO: 96, SEQ ID NO:117, SEQ ID NO:127, SEQ ID NO:137, and SEQ ID NO:147; and SEQ ID NO: 7, SEQ ID NO: 17, SEQ ID NO: 27, SEQ ID NO: 37, SEQ ID NO: 47, SEQ ID NO: 57, SEQ ID NO: 67, SEQ ID NO: 77, SEQ ID NO: 87, SEQ ID NO: 97, SEQ ID NO: 118, SEQ ID NO:128, SEQ ID NO:138, and SEQ ID NO:148.

In another embodiment, the present invention provides an isolated polynucleotide comprising, consisting essentially of, or consisting of a nucleic acid encoding an immunoglobulin light chain variable region (VL), where at least one of the VL-CDRs of the light chain variable region or at least two of the VL-CDRs of the light chain variable region are at least 80%, 85%, 90% or 95% identical to reference light chain VL-CDR1, VL-CDR2, or VL-CDR3 amino acid sequences from monoclonal RON antibodies disclosed herein. Alternatively, the VL-CDR1, VL-CDR2, and VL-CDR3 regions of the VL are at least 80%, 85%, 90% or 95% identical to reference light chain VL-CDR1, VL-CDR2, and VL-CDR3 amino acid sequences from monoclonal RON antibodies disclosed herein. Thus, according to this embodiment a light chain variable region of the invention has VL-CDR1, VL-CDR2, or VL-CDR3 polypeptide sequences related to the polypeptide sequences of SEQ ID NO: 10, SEQ ID NO: 20, SEQ ID NO: 30, SEQ ID NO: 40, SEQ ID NO: 50, SEQ ID NO: 60, SEQ ID NO: 70, SEQ ID NO: 80, SEQ ID NO: 90, SEQ ID NO: 100, SEQ ID NO:121, SEQ ID NO:131, SEQ ID NO:141, and SEQ ID NO:151; SEQ ID NO: 11, SEQ ID NO: 21, SEQ ID NO: 31, SEQ ID NO: 41, SEQ ID NO: 51, SEQ ID NO: 61, SEQ ID NO: 71, SEQ ID NO: 81, SEQ ID NO: 91, SEQ ID NO: 101, SEQ ID NO:122, SEQ ID NO:132, SEQ ID NO: 142, and SEQ ID NO:152; and. SEQ ID NO: 12, SEQ ID NO: 22, SEQ ID NO: 32, SEQ ID NO: 42, SEQ ID NO: 52, SEQ ID NO: 62, SEQ ID NO: 72, SEQ ID NO: 82, SEQ ID NO: 92, SEQ ID NO: 102, SEQ ID NO:123, SEQ ID NO: 133, SEQ ID NO: 143 and SEQ ID NO: 153.

As known in the art, “sequence identity” between two polypeptides or two polynucleotides is determined by comparing the amino acid or nucleic acid sequence of one polypeptide or polynucleotide to the sequence of a second polypeptide or polynucleotide. When discussed herein, whether any particular polypeptide is at least about 40%, 45%, 50%, 55%, 60%, 65%, 70%, 75%, 80%, 85%, 90% or 95% identical to another polypeptide can be determined using methods and computer programs/software known in the art such as, but not limited to, the BESTFIT program (Wisconsin Sequence Analysis Package, Version 8 for Unix, Genetics Computer Group, University Research Park, 575 Science Drive, Madison, Wis. 53711). BESTFIT uses the local homology algorithm of Smith and Waterman, Advances in Applied Mathematics 2:482-489 (1981), to find the best segment of homology between two sequences. When using BESTFIT or any other sequence alignment program to determine whether a particular sequence is, for example, 95% identical to a reference sequence according to the present invention, the parameters are set, of course, such that the percentage of identity is calculated over the full length of the reference polypeptide sequence and that gaps in homology of up to 5% of the total number of amino acids in the reference sequence are allowed.

In certain embodiments, an antibody or antigen-binding fragment comprising the VH encoded by the polynucleotide specifically or preferentially binds to RON. In certain embodiments the nucleotide sequence encoding the VH polypeptide is altered without altering the amino acid sequence encoded thereby. For instance, the sequence may be altered for improved codon usage in a given species, to remove splice sites, or the remove restriction enzyme sites. Sequence optimizations such as these are described in the examples and are well known and routinely carried out by those of ordinary skill in the art.

In certain embodiments, an antibody or antigen-binding fragment comprising the VH encoded by the polynucleotide specifically or preferentially binds to RON.

In some embodiments, the invention provides an isolated polynucleotide comprising a nucleic acid which encodes an antibody VH polypeptide, where the VH polypeptide comprises VH-CDR1, VH-CDR2, and VH-CDR3 amino acid sequences selected from the group consisting of: SEQ ID NOs: 5, 6, and 7; SEQ ID NOs: 15, 16, and 17; SEQ ID NOs: 25, 26, and 27; SEQ ID NOs: 35, 36, and 37; SEQ ID NOs: 45, 46, and 47; SEQ ID NOs: 55, 56, and 57; SEQ ID NOs: 65, 66, and 67; SEQ ID NOs: 75, 76, and 77; SEQ ID NOs: 85, 86, and 87; SEQ ID NOs: 95, 96, and 97; SEQ ID NOs: 116, 117, and 118; SEQ ID NOs: 126, 127, and 128; SEQ ID NOs: 136, 137, and 138; and SEQ ID NOs: 146, 147, and 148; and where an antibody or antigen binding fragment thereof comprising the VH-CDR3 specifically binds to RON.

In certain embodiments, an antibody or antigen-binding fragment thereof comprising, consisting essentially of, or consisting of a VH encoded by one or more of the polynucleotides described above specifically or preferentially binds to the same RON epitope as a reference monoclonal Fab antibody fragment selected from the group consisting of M14-H06, M15-E10, M16-C07, M23-F10, M80-B03, M93-D02, M96-C05, M97-D03 and M98-E12 or a reference monoclonal antibody selected from the group consisting of 1P2E7, 1P3B2, 1P4A3, 1P4A12 and 1P5B10, or will competitively inhibit such a monoclonal antibody or fragment from binding to RON.

In certain embodiments, an antibody or antigen-binding fragment thereof comprising, consisting essentially of, or consisting of a VH encoded by one or more of the polynucleotides described above specifically or preferentially binds to an RON polypeptide or fragment thereof, or a RON variant polypeptide, with an affinity characterized by a dissociation constant (KD) no greater than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

In certain embodiments, an antibody or antigen-binding fragment comprising the VL encoded by the polynucleotide specifically or preferentially binds to RON.

In some embodiments, the invention provides an isolated polynucleotide comprising a nucleic acid which encodes an antibody VL polypeptide, wherein said VL polypeptide comprises VL-CDR1, VL-CDR2, and VL-CDR3 amino acid sequences selected from the group consisting of: SEQ ID NOs: 10, 11, and 12; SEQ ID NOs: 20, 21, and 22; SEQ ID NOs: 30, 31, and 32; SEQ ID NOs: 40, 41, and 42; SEQ ID NOs: 50, 51, and 52; SEQ ID NOs: 60, 61, and 62; SEQ ID NOs: 70, 71, and 72; SEQ ID NOs: 80, 81, and 82; SEQ ID NOs: 90, 91, and 92; SEQ ID NOs: 100, 101, and 102; SEQ ID NOs: 121, 122, and 123; SEQ ID NOs: 131, 132, and 133; SEQ ID NOs: 141, 142, and 143; and SEQ ID NOs: 151, 152, and 153; and wherein an antibody or antigen binding fragment thereof comprising said VL-CDR3 specifically binds to RON.

In certain embodiments, an antibody or antigen-binding fragment thereof comprising, consisting essentially of, or consisting of a VL encoded by one or more of the polynucleotides described above specifically or preferentially binds to the same RON epitope as a reference monoclonal Fab antibody fragment selected from the group consisting of M14-H06, M15-E10, M16-C07, M23-F10, M80-B03, M93-D02, M96-C05, M97-D03 and M98-E12 or a reference monoclonal antibody selected from the group consisting of 1P2E7, 1P3B2, 1P4A3, 1P4A12 and 1P5B10 or will competitively inhibit such a monoclonal antibody or fragment from binding to RON.

In certain embodiments, an antibody or antigen-binding fragment thereof comprising, consisting essentially of, or consisting of a VL encoded by one or more of the polynucleotides described above specifically or preferentially binds to an RON polypeptide or fragment thereof, or a RON variant polypeptide, with an affinity characterized by a dissociation constant (K_{D}) no greater than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

In a further embodiment, the present invention includes an isolated polynucleotide comprising, consisting essentially of, or consisting of a nucleic acid encoding a VH at least 80%, 85%, 90% 95% or 100% identical to a reference VH polypeptide sequence selected from the group consisting of SEQ ID NOs: 4, 14, 24, 34, 44, 54, 64, 74, 84, 94, 115, 125, 135 and 145. In certain embodiments, an antibody or antigen-binding fragment comprising the VH encoded by the polynucleotide specifically or preferentially binds to RON.

In another aspect, the present invention includes an isolated polynucleotide comprising, consisting essentially of, or consisting of a nucleic acid sequence encoding a VH having a polypeptide sequence selected from the group consisting of SEQ ID NOs: 4, 14, 24, 34, 44, 54, 64, 74, 84, 94, 115, 125, 135 and 145. In certain embodiments, an antibody or antigen-binding fragment comprising the VH encoded by the polynucleotide specifically or preferentially binds to RON.

In a further embodiment, the present invention includes an isolated polynucleotide comprising, consisting essentially of, or consisting of a VH-encoding nucleic acid at least 80%, 85%, 90% 95% or 100% identical to a reference nucleic acid sequence selected from the group consisting of SEQ ID NOs: 3, 13, 23, 33, 43, 53, 63, 73, 83, 93, 114, 124, 134 and 144. In certain embodiments, an antibody or antigen-binding fragment comprising the VH encoded by such polynucleotides specifically or preferentially binds to RON.

In another aspect, the present invention includes an isolated polynucleotide comprising, consisting essentially of, or consisting of a nucleic acid sequence encoding a VH of the invention, where the amino acid sequence of the VH is selected from the group consisting of SEQ ID NOs: 4, 14, 24, 34, 44, 54, 64, 74, 84, 94, 115, 125, 135 and 145. The present invention further includes an isolated polynucleotide comprising, consisting essentially of, or consisting of a nucleic acid sequence encoding a VH of the invention, where the sequence of the nucleic acid is selected from the group consisting of SEQ ID NOs: 3, 13, 23, 33, 43, 53, 63, 73, 83, 93, 114, 124, 134 and 144. In certain embodiments, an antibody or antigen-binding fragment comprising the VH encoded by such polynucleotides specifically or preferentially binds to RON.

In certain embodiments, an antibody or antigen-binding fragment thereof comprising, consisting essentially of, or consisting of a VH encoded by one or more of the polynucleotides described above specifically or preferentially binds to the same RON epitope as a reference monoclonal Fab antibody fragment selected from the group consisting of M14-H06, M15-E10, M16-C07, M23-F10, M80-B03, M93-D02, M96-C05, M97-D03 and M98-E12 or a reference monoclonal antibody selected from the group consisting of 1P2E7, 1P3B2, 1P4A3, 1P4A12 and 1P5B10, or will competitively inhibit such a monoclonal antibody or fragment from binding to RON, or will competitively inhibit such a monoclonal antibody from binding to RON.

In certain embodiments, an antibody or antigen-binding fragment thereof comprising, consisting essentially of, or consisting of a VH encoded by one or more of the polynucleotides described above specifically or preferentially binds to an RON polypeptide or fragment thereof, or a RON variant polypeptide, with an affinity characterized by a dissociation constant (K_{D}) no greater than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

In a further embodiment, the present invention includes an isolated polynucleotide comprising, consisting essentially of, or consisting of a nucleic acid encoding a VL at least 80%, 85%, 90% 95% or 100% identical to a reference VL polypeptide sequence having an amino acid sequence selected from the group consisting of SEQ ID NOs: 9, 19, 29, 39, 49, 59, 69, 79, 89, 99, 120, 130, 140, and 150. In a further embodiment, the present invention includes an isolated polynucleotide comprising, consisting essentially of, or consisting of a VL-encoding nucleic acid at least 80%, 85%, 90% 95% or 100% identical to a reference nucleic acid sequence selected from the group consisting of SEQ ID NOs: 8, 18, 28, 38, 48, 58, 68, 78, 88, 98, 119, 129, 139 and 149. In certain embodiments, an antibody or antigen-binding fragment comprising the VL encoded by such polynucleotides specifically or preferentially binds to RON.

In another aspect, the present invention includes an isolated polynucleotide comprising, consisting essentially of, or consisting of a nucleic acid sequence encoding a VL having a polypeptide sequence selected from the group consisting of SEQ ID NOs: 9, 19, 29, 39, 49, 59, 69, 79, 89, 99, 120, 130, 140, and 150. The present invention further includes an isolated polynucleotide comprising, consisting essentially of, or consisting of a nucleic acid sequence encoding a VL of the invention, where the sequence of the nucleic acid is selected from the group consisting of SEQ ID NOs: 8, 18, 28, 38, 48, 58, 68, 78, 88, 98, 119, 129, 139 and 149. In certain embodiments, an antibody or antigen-binding fragment comprising the VL encoded by such polynucleotides specifically or preferentially binds to RON.

In certain embodiments, an antibody or antigen-binding fragment thereof comprising, consisting essentially of, or consisting of a VL encoded by one or more of the polynucleotides described above specifically or preferentially binds to the same RON epitope as a reference monoclonal Fab antibody fragment selected from the group consisting of M14-H06, M15-E10, M16-C07, M23-F10, M80-B03, M93-D02, M96-C05, M97-D03 and M98-E12 or a reference monoclonal antibody selected from the group consisting of 1P2E7, 1P3B2, 1P4A3, 1P4A12 and 1P5B10, or will competitively inhibit such a monoclonal antibody or fragment from binding to RON.

In certain embodiments, an antibody or antigen-binding fragment thereof comprising, consisting essentially of, or consisting of a VL encoded by one or more of the polynucleotides described above specifically or preferentially binds to an RON polypeptide or fragment thereof, or a RON variant polypeptide, with an affinity characterized by a dissociation constant (K_{D}) no greater than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

Any of the polynucleotides described above may further include additional nucleic acids, encoding, e.g., a signal peptide to direct secretion of the encoded polypeptide, antibody constant regions as described herein, or other heterologous polypeptides as described herein.

Also, as described in more detail elsewhere herein, the present invention includes compositions comprising the polynucleotides comprising one or more of the polynucleotides described above. In one embodiment, the invention includes compositions comprising a first polynucleotide and second polynucleotide wherein said first polynucleotide encodes a VH polypeptide as described herein and wherein said second polynucleotide encodes a VL polypeptide as described herein. Specifically a composition which comprises, consists essentially of, or consists of a VH polynucleotide, and a VL polynucleotide, wherein the VH polynucleotide and the VL polynucleotide encode polypeptides, respectively at least 80%, 85%, 90% 95% or 100% identical to reference VH and VL polypeptide amino acid sequences selected from the group consisting of SEQ ID NOs: 4 and 9, 14 and 19, 24 and 29, 34 and 39, 44 and 49, 54 and 59, 64 and 69, 74 and 79, 84 and 89, 94 and 99, 115 and 120, 125 and 130, 135 and 140, and 145 and 150. Or alternatively, a composition which comprises, consists essentially of, or consists of a VH polynucleotide, and a VL polynucleotide at least 80%, 85%, 90% 95% or 100% identical, respectively, to reference VL and VL nucleic acid sequences selected from the group consisting of SEQ ID NOs: 3 and 8, 13 and 18, 23 and 28, 33 and 38, 43 and 48, 53 and 58, 63 and 68, 73 and 78, 83 and 88, 93 and 98, 114 and 119, 124 and 129, 134 and 139, and 144 and 149. In certain embodiments, an antibody or antigen-binding fragment comprising the VH and VL encoded by the polynucleotides in such compositions specifically or preferentially binds to RON.

The present invention also includes fragments of the polynucleotides of the invention, as described elsewhere. Additionally polynucleotides which encode fusion polynucleotides, Fab fragments, and other derivatives, as described herein, are also contemplated by the invention.

The polynucleotides may be produced or manufactured by any method known in the art. For example, if the nucleotide sequence of the antibody is known, a polynucleotide encoding the antibody may be assembled from chemically synthesized oligonucleotides (e.g., as described in Kutmeier et al., BioTechniques 17:242 (1994)), which, briefly, involves the synthesis of overlapping oligonucleotides containing portions of the sequence encoding the antibody, annealing and ligating of those oligonucleotides, and then amplification of the ligated oligonucleotides by PCR.

Alternatively, a polynucleotide encoding an RON antibody, or antigen-binding fragment, variant, or derivative thereof may be generated from nucleic acid from a suitable source. If a clone containing a nucleic acid encoding a particular antibody is not available, but the sequence of the antibody molecule is known, a nucleic acid encoding the antibody may be chemically synthesized or obtained from a suitable source (e.g., an antibody cDNA library, or a cDNA library generated from, or nucleic acid, preferably poly A+RNA, isolated from, any tissue or cells expressing the antibody or other RON antibody, such as hybridoma cells selected to express an antibody) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the antibody or other RON antibody. Amplified nucleic acids generated by PCR may then be cloned into replicable cloning vectors using any method well known in the art.

Once the nucleotide sequence and corresponding amino acid sequence of the RON antibody, or antigen-binding fragment, variant, or derivative thereof is determined, its nucleotide sequence may be manipulated using methods well known in the art for the manipulation of nucleotide sequences, e.g., recombinant DNA techniques, site directed mutagenesis, PCR, etc. (see, for example, the techniques described in Sambrook et al., Molecular Cloning, A Laboratory Manual, 2d Ed., Cold Spring Harbor Laboratory, Cold Spring Harbor, N.Y. (1990) and Ausubel et al., eds., Current Protocols in Molecular Biology, John Wiley & Sons, NY (1998), which are both incorporated by reference herein in their entireties), to generate antibodies having a different amino acid sequence, for example to create amino acid substitutions, deletions, and/or insertions.

A polynucleotide encoding an RON antibody, or antigen-binding fragment, variant, or derivative thereof can be composed of any polyribonucleotide or polydeoxyribonucleotide, which may be unmodified RNA or DNA or modified RNA or DNA. For example, a polynucleotide encoding RON antibody, or antigen-binding fragment, variant, or derivative thereof can be composed of single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, a polynucleotide encoding an RON antibody, or antigen-binding fragment, variant, or derivative thereof can be composed of triple-stranded regions comprising RNA or DNA or both RNA and DNA. A polynucleotide encoding an RON antibody, or antigen-binding fragment, variant, or derivative thereof may also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

An isolated polynucleotide encoding a non-natural variant of a polypeptide derived from an immunoglobulin (e.g., an immunoglobulin heavy chain portion or light chain portion) can be created by introducing one or more nucleotide substitutions, additions or deletions into the nucleotide sequence of the immunoglobulin such that one or more amino acid substitutions, additions or deletions are introduced into the encoded protein. Mutations may be introduced by standard techniques, such as site-directed mutagenesis and PCR-mediated mutagenesis. Preferably, conservative amino acid substitutions are made at one or more non-essential amino acid residues.

### V. RON Antibody Polypeptides

The present invention is further directed to isolated polypeptides which make up RON antibodies, and polynucleotides encoding such polypeptides. RON antibodies of the present invention comprise polypeptides, e.g., amino acid sequences encoding RON-specific antigen binding regions derived from immunoglobulin molecules. A polypeptide or amino acid sequence “derived from” a designated protein refers to the origin of the polypeptide having a certain amino acid sequence. In certain cases, the polypeptide or amino acid sequence which is derived from a particular starting polypeptide or amino acid sequence has an amino acid sequence that is essentially identical to that of the starting sequence, or a portion thereof, wherein the portion consists of at least 10-20 amino acids, at least 20-30 amino acids, at least 30-50 amino acids, or which is otherwise identifiable to one of ordinary skill in the art as having its origin in the starting sequence.

In one embodiment, the present invention provides an isolated polypeptide comprising, consisting essentially of, or consisting of an immunoglobulin heavy chain variable region (VH), where at least one of VH-CDRs of the heavy chain variable region or at least two of the VH-CDRs of the heavy chain variable region are at least 80%, 85%, 90% or 95% identical to reference heavy chain VH-CDR1, VH-CDR2 or VH-CDR3 amino acid sequences from monoclonal RON antibodies disclosed herein. Alternatively, the VH-CDR1, VH-CDR2 and VH-CDR3 regions of the VH are at least 80%, 85%, 90% or 95% identical to reference heavy chain VH-CDR1, VH-CDR2 and VH-CDR3 amino acid sequences from monoclonal RON antibodies disclosed herein. While the VH-CDRs can be defined by the Kabat system, other CDR definitions, e.g., VH-CDRs defined by the Chothia system, are also included in the present invention. In certain embodiments, an antibody or antigen-binding fragment comprising the VH specifically or preferentially binds to RON.

In some embodiments, the invention provides an isolated polypeptide comprising, consisting essentially of, or consisting of an immunoglobulin heavy chain variable region (VH) in which the VH-CDR1, VH-CDR2 and VH-CDR3 regions have polypeptide sequences selected from the group consisting of: SEQ ID NOs: 5, 6, and 7; SEQ ID NOs: 15, 16, and 17; SEQ ID NOs: 25, 26, and 27; SEQ ID NOs: 35, 36, and 37; SEQ ID NOs: 45, 46, and 47; SEQ ID NOs: 55, 56, and 57; SEQ ID NOs: 65, 66, and 67; SEQ ID NOs: 75, 76, and 77; SEQ ID NOs: 85, 86, and 87; SEQ ID NOs: 95, 96, and 97; SEQ ID NOs: 116, 117, and 118; SEQ ID NOs: 126, 127, and 128; SEQ ID NOs: 136, 137, and 138; and SEQ ID NOs: 146, 147, and 148, except for one, two, three, four, five or six amino acid substitutions in at least one of said VH-CDRs.

In some embodiments, the invention provides an isolated polypeptide comprising, consisting essentially of, or consisting of an immunoglobulin heavy chain variable region (VH) in which the VH-CDR1, VH-CDR2 and VH-CDR3 regions have polypeptide sequences selected from the group consisting of: SEQ ID NOs: 5, 6, and 7; SEQ ID NOs: 15, 16, and 17; SEQ ID NOs: 25, 26, and 27; SEQ ID NOs: 35, 36, and 37; SEQ ID NOs: 45, 46, and 47; SEQ ID NOs: 55, 56, and 57; SEQ ID NOs: 65, 66, and 67; SEQ ID NOs: 75, 76, and 77; SEQ ID NOs: 85, 86, and 87; SEQ ID NOs: 95, 96, and 97; SEQ ID NOs: 116, 117, and 118; SEQ ID NOs: 126, 127, and 128; SEQ ID NOs: 136, 137, and 138; and SEQ ID NOs: 146, 147, and 148.

In a further embodiment, the present invention includes an isolated polypeptide comprising, consisting essentially of, or consisting of a VH polypeptide at least 80%, 85%, 90% 95% or 100% identical to a reference VH polypeptide amino acid sequence selected from the group consisting of SEQ ID NOs: 4, 14, 24, 34, 44, 54, 64, 74, 84, 94, 115, 125, 135 and 145. In certain embodiments, an antibody or antigen-binding fragment comprising the VH polypeptide specifically or preferentially binds to RON.

In another aspect, the present invention includes an isolated polypeptide comprising, consisting essentially of, or consisting of a VH polypeptide selected from the group consisting of SEQ ID NOs: 4, 14, 24, 34, 44, 54, 64, 74, 84, 94, 115, 125, 135 and 145. In certain embodiments, an antibody or antigen-binding fragment comprising the VH polypeptide specifically or preferentially binds to RON.

In certain embodiments, an antibody or antigen-binding fragment thereof comprising, consisting essentially of, or consisting of a one or more of the VH polypeptides described above specifically or preferentially binds to the same RON epitope as a reference monoclonal Fab antibody fragment selected from the group consisting of M14-H06, M15-E10, M16-C07, M23-F10, M80-B03, M93-D02, M96-C05, M97-D03 and M98-E12 or a reference monoclonal antibody selected from the group consisting of 1P2E7, 1P3B2, 1P4A3, 1P4A12 and 1P5B10, or will competitively inhibit such a monoclonal antibody or fragment from binding to RON.

In certain embodiments, an antibody or antigen-binding fragment thereof comprising, consisting essentially of, or consisting of one or more of the VH polypeptides described above specifically or preferentially binds to an RON polypeptide or fragment thereof, or a RON variant polypeptide, with an affinity characterized by a dissociation constant (K_{D}) no greater than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

In another embodiment, the present invention provides an isolated polypeptide comprising, consisting essentially of, or consisting of an immunoglobulin light chain variable region (VL), where at least one of the VL-CDRs of the light chain variable region or at least two of the VL-CDRs of the light chain variable region are at least 80%, 85%, 90% or 95% identical to reference light chain VL-CDR1, VL-CDR2 or VL-CDR3 amino acid sequences from monoclonal RON antibodies disclosed herein. Alternatively, the VL-CDR1, VL-CDR2 and VL-CDR3 regions of the VL are at least 80%, 85%, 90% or 95% identical to reference light chain VL-CDR1, VL-CDR2 and VL-CDR3 amino acid sequences from monoclonal RON antibodies disclosed herein. While the VL-CDRs can be defined by the Kabat system, other CDR definitions, e.g., VL-CDRs defined by the Chothia system, are also included in the present invention. In certain embodiments, an antibody or antigen-binding fragment comprising the VL polypeptide specifically or preferentially binds to RON.

In some embodiments, the invention provides an isolated polypeptide comprising, consisting essentially of, or consisting of an immunoglobulin heavy chain variable region (VL) in which the VL-CDR1, VL-CDR2 and VL-CDR3 regions have polypeptide sequences selected from the group consisting of: SEQ ID NOs: 10, 11, and 12; SEQ ID NOs: 20, 21, and 22; SEQ ID NOs: 30, 31, and 32; SEQ ID NOs: 40, 41, and 42; SEQ ID NOs: 50, 51, and 52; SEQ ID NOs: 60, 61, and 62; SEQ ID NOs: 70, 71, and 72; SEQ ID NOs: 80, 81, and 82; SEQ ID NOs: 90, 91, and 92; SEQ ID NOs: 100, 101, and 102; SEQ ID NOs: 121, 122, and 123; SEQ ID NOs: 131, 132, and 133; SEQ ID NOs: 141, 142, and 143; and SEQ ID NOs: 151, 152, and 153, except for one, two, three, four, five or six amino acid substitutions in at least one of said VL-CDRs.

In some embodiments, the invention provides an isolated polypeptide comprising, consisting essentially of, or consisting of an immunoglobulin heavy chain variable region (VL) in which the VL-CDR1, VL-CDR2 and VL-CDR3 regions have polypeptide sequences selected from the group consisting of: SEQ ID NOs: SEQ ID NOs: 10, 11, and 12; SEQ ID NOs: 20, 21, and 22; SEQ ID NOs: 30, 31, and 32; SEQ ID NOs: 40, 41, and 42; SEQ ID NOs: 50, 51, and 52; SEQ ID NOs: 60, 61, and 62; SEQ ID NOs: 70, 71, and 72; SEQ ID NOs: 80, 81, and 82; SEQ ID NOs: 90, 91, and 92; SEQ ID NOs: 100, 101, and 102; SEQ ID NOs: 121, 122, and 123; SEQ ID NOs: 131, 132, and 133; SEQ ID NOs: 141, 142, and 143; and SEQ ID NOs: 151, 152, and 153.

In a further embodiment, the present invention includes an isolated polypeptide comprising, consisting essentially of, or consisting of a VL polypeptide at least 80%, 85%, 90% 95% or 100% identical to a reference VL polypeptide sequence selected from the group consisting of SEQ ID NOs: 9, 19, 29, 39, 49, 59, 69, 79, 89, 99, 120, 130, 140, and 150. In certain embodiments, an antibody or antigen-binding fragment comprising the VL polypeptide specifically or preferentially binds to RON.

In another aspect, the present invention includes an isolated polypeptide comprising, consisting essentially of, or consisting of a VL polypeptide selected from the group consisting of SEQ ID NOs: 9, 19, 29, 39, 49, 59, 69, 79, 89, 99, 120, 130, 140, and 150. In certain embodiments, an antibody or antigen-binding fragment comprising the VL polypeptide specifically or preferentially binds to RON.

In certain embodiments, an antibody or antigen-binding fragment thereof comprising, consisting essentially of, one or more of the VL polypeptides described above specifically or preferentially binds to the same RON epitope as a reference monoclonal Fab antibody fragment selected from the group consisting of M14-H06, M15-E10, M16-C07, M23-F10, M80-B03, M93-D02, M96-C05, M97-D03 and M98-E12 or a reference monoclonal antibody selected from the group consisting of 1P2E7, 1P3B2, 1P4A3, 1P4A12 and 1P5B10, or will competitively inhibit such a monoclonal antibody or fragment from binding to RON.

In certain embodiments, an antibody or antigen-binding fragment thereof comprising, consisting essentially of, or consisting of a one or more of the VL polypeptides described above specifically or preferentially binds to an RON polypeptide or fragment thereof, or a RON variant polypeptide, with an affinity characterized by a dissociation constant (K_{D}) no greater than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

In other embodiments, an antibody or antigen-binding fragment thereof comprises, consists essentially of or consists of a VH polypeptide, and a VL polypeptide, where the VH polypeptide and the VL polypeptide, respectively are at least 80%, 85%, 90% 95% or 100% identical to reference VH and VL polypeptide amino acid sequences selected from the group consisting of SEQ ID NOs: 4 and 9, 14 and 19, 24 and 29, 34 and 39, 44 and 49, 54 and 59, 64 and 69, 74 and 79, 84 and 89, 94 and 99, 115 and 120, 125 and 130, 135 and 140, and 145 and 150. In certain embodiments, an antibody or antigen-binding fragment comprising these VH and VL polypeptides specifically or preferentially binds to RON.

Any of the polypeptides described above may further include additional polypeptides, e.g., a signal peptide to direct secretion of the encoded polypeptide, antibody constant regions as described herein, or other heterologous polypeptides as described herein. Additionally, polypeptides of the invention include polypeptide fragments as described elsewhere. Additionally polypeptides of the invention include fusion polypeptide, Fab fragments, and other derivatives, as described herein.

Also, as described in more detail elsewhere herein, the present invention includes compositions comprising the polypeptides described above.

It will also be understood by one of ordinary skill in the art that RON antibody polypeptides as disclosed herein may be modified such that they vary in amino acid sequence from the naturally occurring binding polypeptide from which they were derived. For example, a polypeptide or amino acid sequence derived from a designated protein may be similar, e.g., have a certain percent identity to the starting sequence, e.g., it may be 60%, 70%, 75%, 80%, 85%, 90%, or 95% identical to the starting sequence.

Furthermore, nucleotide or amino acid substitutions, deletions, or insertions leading to conservative substitutions or changes at “non-essential” amino acid regions may be made. For example, a polypeptide or amino acid sequence derived from a designated protein may be identical to the starting sequence except for one or more individual amino acid substitutions, insertions, or deletions, e.g., one, two, three, four, five, six, seven, eight, nine, ten, fifteen, twenty or more individual amino acid substitutions, insertions, or deletions. a polypeptide or amino acid sequence derived from a designated protein may be identical to the starting sequence except for one or more individual amino acid substitutions, insertions, or deletions, e.g., one, two, three, four, five, six, seven, eight, nine, ten, fifteen, twenty or more individual amino acid substitutions, insertions, or deletions. In other embodiments, a polypeptide or amino acid sequence derived from a designated protein may be identical to the starting sequence except for two or fewer, three or fewer, four or fewer, five or fewer, six or fewer, seven or fewer, eight or fewer, nine or fewer, ten or fewer, fifteen or fewer, or twenty or fewer individual amino acid substitutions, insertions, or deletions. In certain embodiments, a polypeptide or amino acid sequence derived from a designated protein has one to five, one to ten, one to fifteen, or one to twenty individual amino acid substitutions, insertions, or deletions relative to the starting sequence.

Certain RON antibody polypeptides of the present invention comprise, consist essentially of, or consist of an amino acid sequence derived from a human amino acid sequence. However, certain RON antibody polypeptides comprise one or more contiguous amino acids derived from another mammalian species. For example, an RON antibody of the present invention may include a primate heavy chain portion, hinge portion, or antigen binding region. In another example, one or more murine-derived amino acids may be present in a non-murine antibody polypeptide, e.g., in an antigen binding site of an RON antibody. In another example, the antigen binding site of an RON antibody is fully murine. In certain therapeutic applications, RON-specific antibodies, or antigen-binding fragments, variants, or analogs thereof are designed so as to not be immunogenic in the animal to which the antibody is administered.

In certain embodiments, an RON antibody polypeptide comprises an amino acid sequence or one or more moieties not normally associated with an antibody. Exemplary modifications are described in more detail below. For example, a single-chain fv antibody fragment of the invention may comprise a flexible linker sequence, or may be modified to add a functional moiety (e.g., PEG, a drug, a toxin, or a label).

An RON antibody polypeptide of the invention may comprise, consist essentially of, or consist of a fusion protein. Fusion proteins are chimeric molecules which comprise, for example, an immunoglobulin antigen-binding domain with at least one target binding site, and at least one heterologous portion, i.e., a portion with which it is not naturally linked in nature. The amino acid sequences may normally exist in separate proteins that are brought together in the fusion polypeptide or they may normally exist in the same protein but are placed in a new arrangement in the fusion polypeptide. Fusion proteins may be created, for example, by chemical synthesis, or by creating and translating a polynucleotide in which the peptide regions are encoded in the desired relationship.

The term “heterologous” as applied to a polynucleotide or a polypeptide, means that the polynucleotide or polypeptide is derived from a distinct entity from that of the rest of the entity to which it is being compared. For instance, as used herein, a “heterologous polypeptide” to be fused to an RON antibody, or an antigen-binding fragment, variant, or analog thereof is derived from a non-immunoglobulin polypeptide of the same species, or an immunoglobulin or non-immunoglobulin polypeptide of a different species.

A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential amino acid residue in an immunoglobulin polypeptide is preferably replaced with another amino acid residue from the same side chain family. In another embodiment, a string of amino acids can be replaced with a structurally similar string that differs in order and/or composition of side chain family members.

Alternatively, in another embodiment, mutations may be introduced randomly along all or part of the immunoglobulin coding sequence, such as by saturation mutagenesis, and the resultant mutants can be incorporated into RON antibodies for use in the diagnostic and treatment methods disclosed herein and screened for their ability to bind to the desired antigen, e.g., RON.

### VI. Fusion Proteins and Antibody Conjugates

As discussed in more detail elsewhere herein, RON antibodies, or antigen-binding fragments, variants, or derivatives thereof of the invention may further be recombinantly fused to a heterologous polypeptide at the N- or C-terminus or chemically conjugated (including covalent and non-covalent conjugations) to polypeptides or other compositions. For example, RON-specific RON antibodies may be recombinantly fused or conjugated to molecules useful as labels in detection assays and effector molecules such as heterologous polypeptides, drugs, radionuclides, or toxins. See, e.g., PCT publications WO 92/08495; WO 91/14438; WO 89/12624; U.S. Pat. No. 5,314,995; and EP 396,387.

RON antibodies, or antigen-binding fragments, variants, or derivatives thereof of the invention include derivatives that are modified, i.e., by the covalent attachment of any type of molecule to the antibody such that covalent attachment does not prevent the antibody binding RON. For example, but not by way of limitation, the antibody derivatives include antibodies that have been modified, e.g., by glycosylation, acetylation, pegylation, phosphylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

RON antibodies, or antigen-binding fragments, variants, or derivatives thereof of the invention can be composed of amino acids joined to each other by peptide bonds or modified peptide bonds, i.e., peptide isosteres, and may contain amino acids other than the 20 gene-encoded amino acids. RON-specific antibodies may be modified by natural processes, such as posttranslational processing, or by chemical modification techniques which are well known in the art. Such modifications are well described in basic texts and in more detailed monographs, as well as in a voluminous research literature. Modifications can occur anywhere in the RON-specific antibody, including the peptide backbone, the amino acid side-chains and the amino or carboxyl termini, or on moieties such as carbohydrates. It will be appreciated that the same type of modification may be present in the same or varying degrees at several sites in a given RON-specific antibody. Also, a given RON-specific antibody may contain many types of modifications. RON-specific antibodies may be branched, for example, as a result of ubiquitination, and they may be cyclic, with or without branching. Cyclic, branched, and branched cyclic RON-specific antibodies may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. (See, e.g., Proteins—Structure And Molecular Properties, T. E. Creighton, W. H. Freeman and Company, New York 2nd Ed., (1993); Posttranslational Covalent Modification Of Proteins, B. C. Johnson, Ed., Academic Press, New York, pgs. 1-12 (1983); Seifter et al., Meth Enzymol 182:626-646 (1990); Rattan et al., Ann NY Acad Sci 663:48-62 (1992)).

The present invention also provides for fusion proteins comprising an RON antibody, or antigen-binding fragment, variant, or derivative thereof, and a heterologous polypeptide. The heterologous polypeptide to which the antibody is fused may be useful for function or is useful to target the RON polypeptide expressing cells. In one embodiment, a fusion protein of the invention comprises, consists essentially of, or consists of, a polypeptide having the amino acid sequence of any one or more of the VH regions of an antibody of the invention or the amino acid sequence of any one or more of the VL regions of an antibody of the invention or fragments or variants thereof, and a heterologous polypeptide sequence. In another embodiment, a fusion protein for use in the diagnostic and treatment methods disclosed herein comprises, consists essentially of, or consists of a polypeptide having the amino acid sequence of any one, two, three of the VH-CDRs of an RON-specific antibody, or fragments, variants, or derivatives thereof, or the amino acid sequence of any one, two, three of the VL-CDRs of an RON-specific antibody, or fragments, variants, or derivatives thereof, and a heterologous polypeptide sequence. In one embodiment, the fusion protein comprises a polypeptide having the amino acid sequence of a VH-CDR3 of an RON-specific antibody of the present invention, or fragment, derivative, or variant thereof, and a heterologous polypeptide sequence, which fusion protein specifically binds to at least one epitope of RON. In another embodiment, a fusion protein comprises a polypeptide having the amino acid sequence of at least one VH region of an RON-specific antibody of the invention and the amino acid sequence of at least one VL region of an RON-specific antibody of the invention or fragments, derivatives or variants thereof, and a heterologous polypeptide sequence. Preferably, the VH and VL regions of the fusion protein correspond to a single source antibody (or scFv or Fab fragment) which specifically binds at least one epitope of RON. In yet another embodiment, a fusion protein for use in the diagnostic and treatment methods disclosed herein comprises a polypeptide having the amino acid sequence of any one, two, three or more of the VH CDRs of an RON-specific antibody and the amino acid sequence of any one, two, three or more of the VL CDRs of an RON-specific antibody, or fragments or variants thereof, and a heterologous polypeptide sequence. Preferably, two, three, four, five, six, or more of the VH-CDR(s) or VL-CDR(s) correspond to single source antibody (or scFv or Fab fragment) of the invention. Nucleic acid molecules encoding these fusion proteins are also encompassed by the invention.

Exemplary fusion proteins reported in the literature include fusions of the T cell receptor (Gascoigne et al., Proc. Natl. Acad. Sci. USA 84:2936-2940 (1987)); CD4 (Capon et al., Nature 337:525-531 (1989); Traunecker et al., Nature 339:68-70 (1989); Zettmeissl et al., DNA Cell Biol. USA 9:347-353 (1990); and Byrn et al., Nature 344:667-670 (1990)); L-selectin (homing receptor) (Watson et al., J. Cell. Biol. 110:2221-2229 (1990); and Watson et al., Nature 349:164-167 (1991)); CD44 (Aruffo et al., Cell 61:1303-1313 (1990)); CD28 and B7 (Linsley et al., J. Exp. Med. 173:721-730 (1991)); CTLA-4 (Lisley et al., J. Exp. Med. 174:561-569 (1991)); CD22 (Stamenkovic et al., Cell 66:1133-1144 (1991)); TNF receptor (Ashkenazi et al., Proc. Natl. Acad. Sci. USA 88:10535-10539 (1991); Lesslauer et al., Eur. J. Immunol. 27:2883-2886 (1991); and Peppel et al., J. Exp. Med. 174:1483-1489 (1991)); and IgE receptor a (Ridgway and Gorman, J. Cell. Biol. Vol. 115, Abstract No. 1448 (1991)).

As discussed elsewhere herein, RON antibodies, or antigen-binding fragments, variants, or derivatives thereof of the invention may be fused to heterologous polypeptides to increase the in vivo half life of the polypeptides or for use in immunoassays using methods known in the art. For example, in one embodiment, PEG can be conjugated to the RON antibodies of the invention to increase their half-life in vivo. Leong, S. R., et al., Cytokine 16:106 (2001); Adv. in Drug Deliv. Rev. 54:531 (2002); or Weir et al., Biochem. Soc. Transactions 30:512 (2002).

Moreover, RON antibodies, or antigen-binding fragments, variants, or derivatives thereof of the invention can be fused to marker sequences, such as a peptide to facilitate their purification or detection. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., Proc. Natl. Acad. Sci. USA 86:821-824 (1989), for instance, hexa-histidine provides for convenient purification of the fusion protein. Other peptide tags useful for purification include, but are not limited to, the “HA” tag, which corresponds to an epitope derived from the influenza hemagglutinin protein (Wilson et al., Cell 37:767 (1984)) and the “flag” tag.

Fusion proteins can be prepared using methods that are well known in the art (see for example U.S. Pat. Nos. 5,116,964 and 5,225,538). The precise site at which the fusion is made may be selected empirically to optimize the secretion or binding characteristics of the fusion protein. DNA encoding the fusion protein is then transfected into a host cell for expression.

RON antibodies of the present invention may be used in non-conjugated form or may be conjugated to at least one of a variety of molecules, e.g., to improve the therapeutic properties of the molecule, to facilitate target detection, or for imaging or therapy of the patient. RON antibodies, or antigen-binding fragments, variants, or derivatives thereof of the invention can be labeled or conjugated either before or after purification, when purification is performed.

In particular, RON antibodies, or antigen-binding fragments, variants, or derivatives thereof of the invention may be conjugated to therapeutic agents, prodrugs, peptides, proteins, enzymes, viruses, lipids, biological response modifiers, pharmaceutical agents, or PEG.

Those skilled in the art will appreciate that conjugates may also be assembled using a variety of techniques depending on the selected agent to be conjugated. For example, conjugates with biotin are prepared e.g. by reacting a binding polypeptide with an activated ester of biotin such as the biotin N-hydroxysuccinimide ester. Similarly, conjugates with a fluorescent marker may be prepared in the presence of a coupling agent, e.g. those listed herein, or by reaction with an isothiocyanate, preferably fluorescein-isothiocyanate. Conjugates of the RON antibodies, or antigen-binding fragments, variants, or derivatives thereof of the invention are prepared in an analogous manner.

The present invention further encompasses RON antibodies, or antigen-binding fragments, variants, or derivatives thereof of the invention conjugated to a diagnostic or therapeutic agent. The RON antibodies can be used diagnostically to, for example, monitor the development or progression of a neurological disease as part of a clinical testing procedure to, e.g., determine the efficacy of a given treatment and/or prevention regimen. Detection can be facilitated by coupling the RON antibody, or antigen-binding fragment, variant, or derivative thereof to a detectable substance. Examples of detectable substances include various enzymes, prosthetic groups, fluorescent materials, luminescent materials, bioluminescent materials, radioactive materials, positron emitting metals using various positron emission tomographies, and nonradioactive paramagnetic metal ions. See, e.g., U.S. Pat. No. 4,741,900 for metal ions which can be conjugated to antibodies for use as diagnostics according to the present invention. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include ¹²⁵I, ¹³¹I, ¹¹¹In or ⁹⁹Tc.

An RON antibody, or antigen-binding fragment, variant, or derivative thereof also can be detectably labeled by coupling it to a chemiluminescent compound. The presence of the chemiluminescent-tagged RON antibody is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

One of the ways in which an RON antibody, or antigen-binding fragment, variant, or derivative thereof can be detectably labeled is by linking the same to an enzyme and using the linked product in an enzyme immunoassay (EIA) (Voller, A., “The Enzyme Linked Immunosorbent Assay (ELISA)” Microbiological Associates Quarterly Publication, Walkersville, Md., Diagnostic Horizons 2:1-7 (1978)); Voller et al., J. Clin. Pathol. 31:507-520 (1978); Butler, J. E., Meth. Enzymol. 73:482-523 (1981); Maggio, E. (ed.), Enzyme Immunoassay, CRC Press, Boca Raton, Fla., (1980); Ishikawa, E. et al., (eds.), Enzyme Immunoassay, Kgaku Shoin, Tokyo (1981). The enzyme, which is bound to the RON antibody will react with an appropriate substrate, preferably a chromogenic substrate, in such a manner as to produce a chemical moiety which can be detected, for example, by spectrophotometric, fluorimetric or by visual means. Enzymes which can be used to detectably label the antibody include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. Additionally, the detection can be accomplished by colorimetric methods which employ a chromogenic substrate for the enzyme. Detection may also be accomplished by visual comparison of the extent of enzymatic reaction of a substrate in comparison with similarly prepared standards.

Detection may also be accomplished using any of a variety of other immunoassays. For example, by radioactively labeling the RON antibody, or antigen-binding fragment, variant, or derivative thereof, it is possible to detect the antibody through the use of a radioimmunoassay (RIA) (see, for example, Weintraub, B., Principles of Radioimmunoassays, Seventh Training Course on Radioligand Assay Techniques, The Endocrine Society, (March, 1986)), which is incorporated by reference herein). The radioactive isotope can be detected by means including, but not limited to, a gamma counter, a scintillation counter, or autoradiography.

An RON antibody, or antigen-binding fragment, variant, or derivative thereof can also be detectably labeled using fluorescence emitting metals such as 152Eu, or others of the lanthanide series. These metals can be attached to the antibody using such metal chelating groups as diethylenetriaminepentacetic acid (DTPA) or ethylenediaminetetraacetic acid (EDTA).

Techniques for conjugating various moieties to an RON antibody, or antigen-binding fragment, variant, or derivative thereof are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. (1985); Hellstrom et al., “Antibodies For Drug Delivery”, in Controlled Drug Delivery (2nd Ed.), Robinson et al. (eds.), Marcel Dekker, Inc., pp. 623-53 (1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), Academic Press pp. 303-16 (1985), and Thorpe et al., “The Preparation And Cytotoxic Properties Of Antibody-Toxin Conjugates”, Immunol. Rev. 62:119-58 (1982).

In particular, binding molecules, e.g., binding polypeptides, e.g., RON-specific antibodies or immunospecific fragments thereof for use in the diagnostic and treatment methods disclosed herein may be conjugated to cytotoxins (such as radioisotopes, cytotoxic drugs, or toxins) therapeutic agents, cytostatic agents, biological toxins, prodrugs, peptides, proteins, enzymes, viruses, lipids, biological response modifiers, pharmaceutical agents, immunologically active ligands (e.g., lymphokines or other antibodies wherein the resulting molecule binds to both the neoplastic cell and an effector cell such as a T cell), or PEG. In another embodiment, a binding molecule, e.g., a binding polypeptide, e.g., an RON-specific antibody or immunospecific fragment thereof for use in the diagnostic and treatment methods disclosed herein can be conjugated to a molecule that decreases vascularization of tumors. In other embodiments, the disclosed compositions may comprise binding molecules, e.g., binding polypeptides, e.g., RON-specific antibodies or immunospecific fragments thereof coupled to drugs or prodrugs. Still other embodiments of the present invention comprise the use of binding molecules, e.g., binding polypeptides, e.g., RON-specific antibodies or immunospecific fragments thereof conjugated to specific biotoxins or their cytotoxic fragments such as ricin, gelonin, pseudomonas exotoxin or diphtheria toxin. The selection of which conjugated or unconjugated binding molecule to use will depend on the type and stage of cancer, use of adjunct treatment (e.g., chemotherapy or external radiation) and patient condition. It will be appreciated that one skilled in the art could readily make such a selection in view of the teachings herein.

It will be appreciated that, in previous studies, anti-tumor antibodies labeled with isotopes have been used successfully to destroy cells in solid tumors as well as lymphomas/leukemias in animal models, and in some cases in humans. Exemplary radioisotopes include: ⁹⁰Y, ¹²⁵I, ¹³¹I, ¹²³I, ¹¹¹In, ¹⁰⁵Rh, ¹⁵³Sm, ⁶⁷Cu, ⁶⁷Ga, ¹⁶⁶Ho, ¹⁷⁷Lu, ¹⁸⁶Re and ¹⁸⁸Re. The radionuclides act by producing ionizing radiation which causes multiple strand breaks in nuclear DNA, leading to cell death. The isotopes used to produce therapeutic conjugates typically produce high energy α- or β-particles which have a short path length. Such radionuclides kill cells to which they are in close proximity, for example neoplastic cells to which the conjugate has attached or has entered. They have little or no effect on non-localized cells. Radionuclides are essentially non-immunogenic.

With respect to the use of radiolabeled conjugates in conjunction with the present invention, binding molecules, e.g., binding polypeptides, e.g., RON-specific antibodies or immunospecific fragments thereof may be directly labeled (such as through iodination) or may be labeled indirectly through the use of a chelating agent. As used herein, the phrases “indirect labeling” and “indirect labeling approach” both mean that a chelating agent is covalently attached to a binding molecule and at least one radionuclide is associated with the chelating agent. Such chelating agents are typically referred to as bifunctional chelating agents as they bind both the polypeptide and the radioisotope. Particularly preferred chelating agents comprise 1-isothiocycmatobenzyl-3-methyldiothelene triaminepentaacetic acid (“MX-DTPA”) and cyclohexyl diethylenetriamine pentaacetic acid (“CHX-DTPA”) derivatives. Other chelating agents comprise P-DOTA and EDTA derivatives. Particularly preferred radionuclides for indirect labeling include ¹¹¹In and ⁹⁰Y.

As used herein, the phrases “direct labeling” and “direct labeling approach” both mean that a radionuclide is covalently attached directly to a polypeptide (typically via an amino acid residue). More specifically, these linking technologies include random labeling and site-directed labeling. In the latter case, the labeling is directed at specific sites on the polypeptide, such as the N-linked sugar residues present only on the Fc portion of the conjugates. Further, various direct labeling techniques and protocols are compatible with the instant invention. For example, Technetium-99 labeled polypeptides may be prepared by ligand exchange processes, by reducing pertechnate (TcO₄⁻) with stannous ion solution, chelating the reduced technetium onto a Sephadex column and applying the binding polypeptides to this column, or by batch labeling techniques, e.g. by incubating pertechnate, a reducing agent such as SnCl₂, a buffer solution such as a sodium-potassium phthalate-solution, and the antibodies. In any event, preferred radionuclides for directly labeling antibodies are well known in the art and a particularly preferred radionuclide for direct labeling is ¹³¹I covalently attached via tyrosine residues. Binding molecules, e.g., binding polypeptides, e.g., RON-specific antibodies or immunospecific fragments thereof for use in the diagnostic and treatment methods disclosed herein may be derived, for example, with radioactive sodium or potassium iodide and a chemical oxidizing agent, such as sodium hypochlorite, chloramine T or the like, or an enzymatic oxidizing agent, such as lactoperoxidase, glucose oxidase and glucose.

Patents relating to chelators and chelator conjugates are known in the art. For instance, U.S. Pat. No. 4,831,175 of Gansow is directed to polysubstituted diethylenetriaminepentaacetic acid chelates and protein conjugates containing the same, and methods for their preparation. U.S. Pat. Nos. 5,099,069, 5,246,692, 5,286,850, 5,434,287 and 5,124,471 of Gansow also relate to polysubstituted DTPA chelates. These patents are incorporated herein by reference in their entireties. Other examples of compatible metal chelators are ethylenediaminetetraacetic acid (EDTA), diethylenetriaminepentaacetic acid (DPTA), 1,4,8,11-tetraazatetradecane, 1,4,8,11-tetraazatetradecane-1,4,8,11-tetraacetic acid, 1-oxa-4,7,12,15-tetraazaheptadecane-4,7,12,15-tetraacetic acid, or the like. Cyclohexyl-DTPA or CHX-DTPA is particularly preferred and is exemplified extensively below. Still other compatible chelators, including those yet to be discovered, may easily be discerned by a skilled artisan and are clearly within the scope of the present invention.

Compatible chelators, including the specific bifunctional chelator used to facilitate chelation U.S. Pat. Nos. 6,682,134, 6,399,061, and 5,843,439, incorporated herein by reference in their entireties, are preferably selected to provide high affinity for trivalent metals, exhibit increased tumor-to-non-tumor ratios and decreased bone uptake as well as greater in vivo retention of radionuclide at target sites, i.e., B-cell lymphoma tumor sites. However, other bifunctional chelators that may or may not possess all of these characteristics are known in the art and may also be beneficial in tumor therapy.

It will also be appreciated that, in accordance with the teachings herein, binding molecules may be conjugated to different radiolabels for diagnostic and therapeutic purposes. To this end the aforementioned U.S. Pat. Nos. 6,682,134, 6,399,061, and 5,843,439 disclose radiolabeled therapeutic conjugates for diagnostic “imaging” of tumors before administration of therapeutic antibody. “In2B8” conjugate comprises a murine monoclonal antibody, 2B8, specific to human CD20 antigen, that is attached to ¹¹¹In via a bifunctional chelator, i.e., MX-DTPA (diethylenetriaminepentaacetic acid), which comprises a 1:1 mixture of 1-isothiocyanatobenzyl-3-methyl-DTPA and 1-methyl-3-isothiocyanatobenzyl-DTPA. ¹¹¹In is particularly preferred as a diagnostic radionuclide because between about 1 to about 10 mCi can be safely administered without detectable toxicity; and the imaging data is generally predictive of subsequent ⁹⁰Y-labeled antibody distribution. Most imaging studies utilize 5 mCi ¹¹¹In-labeled antibody, because this dose is both safe and has increased imaging efficiency compared with lower doses, with optimal imaging occurring at three to six days after antibody administration. See, for example, Murray, J. Nuc. Med. 26: 3328 (1985) and Carraguillo et al., J. Nuc. Med. 26: 67 (1985).

As indicated above, a variety of radionuclides are applicable to the present invention and those skilled in the can readily determine which radionuclide is most appropriate under various circumstances. For example, ¹³¹I is a well known radionuclide used for targeted immunotherapy. However, the clinical usefulness of ¹³¹I can be limited by several factors including: eight-day physical half-life; dehalogenation of iodinated antibody both in the blood and at tumor sites; and emission characteristics (e.g., large gamma component) which can be suboptimal for localized dose deposition in tumor. With the advent of superior chelating agents, the opportunity for attaching metal chelating groups to proteins has increased the opportunities to utilize other radionuclides such as ¹¹¹In and ⁹⁰Y. ⁹⁰Y provides several benefits for utilization in radioimmunotherapeutic applications: the 64 hour half-life of ⁹⁰Y is long enough to allow antibody accumulation by tumor and, unlike e.g., ¹³¹I, ⁹⁰Y is a pure beta emitter of high energy with no accompanying gamma irradiation in its decay, with a range in tissue of 100 to 1,000 cell diameters. Furthermore, the minimal amount of penetrating radiation allows for outpatient administration of ⁹⁰Y-labeled antibodies. Additionally, internalization of labeled antibody is not required for cell killing, and the local emission of ionizing radiation should be lethal for adjacent tumor cells lacking the target molecule.

Additional preferred agents for conjugation to binding molecules, e.g., binding polypeptides, e.g., RON-specific antibodies or immunospecific fragments thereof are cytotoxic drugs, particularly those which are used for cancer therapy. As used herein, “a cytotoxin or cytotoxic agent” means any agent that is detrimental to the growth and proliferation of cells and may act to reduce, inhibit or destroy a cell or malignancy. Exemplary cytotoxins include, but are not limited to, radionuclides, biotoxins, enzymatically active toxins, cytostatic or cytotoxic therapeutic agents, prodrugs, immunologically active ligands and biological response modifiers such as cytokines. Any cytotoxin that acts to retard or slow the growth of immunoreactive cells or malignant cells is within the scope of the present invention.

Exemplary cytotoxins include, in general, cytostatic agents, alkylating agents, anti-metabolites, anti-proliferative agents, tubulin binding agents, hormones and hormone antagonists, and the like. Exemplary cytostatics that are compatible with the present invention include alkylating substances, such as mechlorethamine, triethylenephosphoramide, cyclophosphamide, ifosfamide, chlorambucil, busulfan, melphalan or triaziquone, also nitrosourea compounds, such as carmustine, lomustine, or semustine. Other preferred classes of cytotoxic agents include, for example, the maytansinoid family of drugs. Other preferred classes of cytotoxic agents include, for example, the anthracycline family of drugs, the vinca drugs, the mitomycins, the bleomycins, the cytotoxic nucleosides, the pteridine family of drugs, diynenes, and the podophyllotoxins. Particularly useful members of those classes include, for example, adriamycin, caminomycin, daunorubicin (daunomycin), doxorubicin, aminopterin, methotrexate, methopterin, mithramycin, streptonigrin, dichloromethotrexate, mitomycin C, actinomycin-D, porfiromycin, 5-fluorouracil, floxuridine, ftorafur, 6-mercaptopurine, cytarabine, cytosine arabinoside, podophyllotoxin, or podophyllotoxin derivatives such as etoposide or etoposide phosphate, melphalan, vinblastine, vincristine, leurosidine, vindesine, leurosine and the like. Still other cytotoxins that are compatible with the teachings herein include taxol, taxane, cytochalasin B, gramicidin D, ethidium bromide, emetine, tenoposide, colchicin, dihydroxy anthracin dione, mitoxantrone, procaine, tetracaine, lidocaine, propranolol, and puromycin and analogs or homologs thereof. Hormones and hormone antagonists, such as corticosteroids, e.g. prednisone, progestins, e.g. hydroxyprogesterone or medroprogesterone, estrogens, e.g. diethylstilbestrol, antiestrogens, e.g. tamoxifen, androgens, e.g. testosterone, and aromatase inhibitors, e.g. aminogluthetimide are also compatible with the teachings herein. One skilled in the art may make chemical modifications to the desired compound in order to make reactions of that compound more convenient for purposes of preparing conjugates of the invention.

One example of particularly preferred cytotoxins comprise members or derivatives of the enediyne family of anti-tumor antibiotics, including calicheamicin, esperamicins or dynemicins. These toxins are extremely potent and act by cleaving nuclear DNA, leading to cell death. Unlike protein toxins which can be cleaved in vivo to give many inactive but immunogenic polypeptide fragments, toxins such as calicheamicin, esperamicins and other enediynes are small molecules which are essentially non-immunogenic. These non-peptide toxins are chemically-linked to the dimers or tetramers by techniques which have been previously used to label monoclonal antibodies and other molecules. These linking technologies include site-specific linkage via the N-linked sugar residues present only on the Fc portion of the constructs. Such site-directed linking methods have the advantage of reducing the possible effects of linkage on the binding properties of the constructs.

As previously alluded to, compatible cytotoxins for preparation of conjugates may comprise a prodrug. As used herein, the term “prodrug” refers to a precursor or derivative form of a pharmaceutically active substance that is less cytotoxic to tumor cells compared to the parent drug and is capable of being enzymatically activated or converted into the more active parent form. Prodrugs compatible with the invention include, but are not limited to, phosphate-containing prodrugs, thiophosphate-containing prodrugs, sulfate containing prodrugs, peptide containing prodrugs, β-lactam-containing prodrugs, optionally substituted phenoxyacetamide-containing prodrugs or optionally substituted phenylacetamide-containing prodrugs, 5-fluorocytosine and other 5-fluorouridine prodrugs that can be converted to the more active cytotoxic free drug. Further examples of cytotoxic drugs that can be derivatized into a prodrug form for use in the present invention comprise those chemotherapeutic agents described above.

Among other cytotoxins, it will be appreciated that binding molecules, e.g., binding polypeptides, e.g., RON-specific antibodies or immunospecific fragments thereof disclosed herein can also be associated with or conjugated to a biotoxin such as ricin subunit A, abrin, diptheria toxin, botulinum, cyanginosins, saxitoxin, shigatoxin, tetanus, tetrodotoxin, trichothecene, verrucologen or a toxic enzyme. Preferably, such constructs will be made using genetic engineering techniques that allow for direct expression of the antibody-toxin construct. Other biological response modifiers that may be associated with the binding molecules, e.g., binding polypeptides, e.g., RON-specific antibodies or immunospecific fragments thereof disclosed herein comprise cytokines such as lymphokines and interferons. In view of the instant disclosure it is submitted that one skilled in the art could readily form such constructs using conventional techniques.

Another class of compatible cytotoxins that may be used in association with or conjugated to the disclosed binding molecules, e.g., binding polypeptides, e.g., RON-specific antibodies or immunospecific fragments thereof, are radiosensitizing drugs that may be effectively directed to tumor or immunoreactive cells. Such drugs enhance the sensitivity to ionizing radiation, thereby increasing the efficacy of radiotherapy. An antibody conjugate internalized by the tumor cell would deliver the radiosensitizer nearer the nucleus where radiosensitization would be maximal. The unbound radiosensitizer linked binding molecules of the invention would be cleared quickly from the blood, localizing the remaining radiosensitization agent in the target tumor and providing minimal uptake in normal tissues. After rapid clearance from the blood, adjunct radiotherapy would be administered in one of three ways: 1.) external beam radiation directed specifically to the tumor, 2.) radioactivity directly implanted in the tumor or 3.) systemic radioimmunotherapy with the same targeting antibody. A potentially attractive variation of this approach would be the attachment of a therapeutic radioisotope to the radiosensitized immunoconjugate, thereby providing the convenience of administering to the patient a single drug.

In certain embodiments, a moiety that enhances the stability or efficacy of a binding molecule, e.g., a binding polypeptide, e.g., an RON-specific antibody or immunospecific fragment thereof can be conjugated. For example, in one embodiment, PEG can be conjugated to the binding molecules of the invention to increase their half-life in vivo. Leong, S. R., et al., Cytokine 16:106 (2001); Adv. in Drug Deliv. Rev. 54:531 (2002); or Weir et al., Biochem. Soc. Transactions 30:512 (2002).

The present invention further encompasses the use of binding molecules, e.g., binding polypeptides, e.g., RON-specific antibodies or immunospecific fragments conjugated to a diagnostic or therapeutic agent. The binding molecules can be used diagnostically to, for example, monitor the development or progression of a tumor as part of a clinical testing procedure to, e.g., determine the efficacy of a given treatment and/or prevention regimen. Detection can be facilitated by coupling the binding molecule, e.g., binding polypeptide, e.g., RON-specific antibody or immunospecific fragment thereof to a detectable substance. Examples of detectable substances include various enzymes, prosthetic groups, fluorescent materials, luminescent materials, bioluminescent materials, radioactive materials, positron emitting metals using various positron emission tomographies, and nonradioactive paramagnetic metal ions. See, for example, U.S. Pat. No. 4,741,900 for metal ions which can be conjugated to antibodies for use as diagnostics according to the present invention. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include ¹²⁵I, ¹³¹I, ¹¹¹In or ⁹⁹Tc.

A binding molecule, e.g., a binding polypeptide, e.g., an RON-specific antibody or immunospecific fragment thereof also can be detectably labeled by coupling it to a chemiluminescent compound. The presence of the chemiluminescent-tagged binding molecule is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

One of the ways in which a binding molecule, e.g., a binding polypeptide, e.g., an RON-specific antibody or immunospecific fragment thereof can be detectably labeled is by linking the same to an enzyme and using the linked product in an enzyme immunoassay (EIA) (Voller, A., “The Enzyme Linked Immunosorbent Assay (ELISA)” Microbiological Associates Quarterly Publication, Walkersville, Md., Diagnostic Horizons 2:1-7 (1978)); Voller et al., J. Clin. Pathol. 31:507-520 (1978); Butler, J. E., Meth. Enrymol. 73:482-523 (1981); Maggio, E. (ed.), Enzyme Immunoassay, CRC Press, Boca Raton, Fla., (1980); Ishikawa, E. et al., (eds.), Enzyme Immunoassay, Kgaku Shoin, Tokyo (1981). The enzyme, which is bound to the binding molecule will react with an appropriate substrate, preferably a chromogenic substrate, in such a manner as to produce a chemical moiety which can be detected, for example, by spectrophotometric, fluorimetric or by visual means. Enzymes which can be used to detectably label the antibody include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. Additionally, the detection can be accomplished by colorimetric methods which employ a chromogenic substrate for the enzyme. Detection may also be accomplished by visual comparison of the extent of enzymatic reaction of a substrate in comparison with similarly prepared standards.

Detection may also be accomplished using any of a variety of other immunoassays. For example, by radioactively labeling the binding molecule, e.g., binding polypeptide, e.g., RON-specific antibody or immunospecific fragment thereof, it is possible to detect cancer antigens through the use of a radioimmunoassay (RIA) (see, for example, Weintraub, B., Principles of Radioimmunoassays, Seventh Training Course on Radioligand Assay Techniques, The Endocrine Society, (March, 1986)), which is incorporated by reference herein). The radioactive isotope can be detected by means including, but not limited to, a gamma counter, a scintillation counter, or autoradiography.

A binding molecule, e.g., a binding polypeptide, e.g., an RON-specific antibody or immunospecific fragment thereof can also be detectably labeled using fluorescence emitting metals such as 152Eu, or others of the lanthanide series. These metals can be attached to the antibody using such metal chelating groups as diethylenetriaminepentacetic acid (DTPA) or ethylenediaminetetraacetic acid (EDTA).

Techniques for conjugating various moieties to a binding molecule, e.g., a binding polypeptide, e.g., an RON-specific antibody or immunospecific fragment thereof are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. (1985); Hellstrom et al., “Antibodies For Drug Delivery”, in Controlled Drug Delivery (2nd Ed.), Robinson et al. (eds.), Marcel Dekker, Inc., pp. 623-53 (1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), Academic Press pp. 303-16 (1985), and Thorpe et al., “The Preparation And Cytotoxic Properties Of Antibody-Toxin Conjugates”, Immunol. Rev. 62:119-58 (1982).

### VII. Expression of Antibody Polypeptides

As is well known, RNA may be isolated from the original hybridoma cells or from other transformed cells by standard techniques, such as guanidinium isothiocyanate extraction and precipitation followed by centrifugation or chromatography. Where desirable, mRNA may be isolated from total RNA by standard techniques such as chromatography on oligo dT cellulose. Suitable techniques are familiar in the art.

In one embodiment, cDNAs that encode the light and the heavy chains of the antibody may be made, either simultaneously or separately, using reverse transcriptase and DNA polymerase in accordance with well known methods. PCR may be initiated by consensus constant region primers or by more specific primers based on the published heavy and light chain DNA and amino acid sequences. As discussed above, PCR also may be used to isolate DNA clones encoding the antibody light and heavy chains. In this case the libraries may be screened by consensus primers or larger homologous probes, such as mouse constant region probes.

DNA, typically plasmid DNA, may be isolated from the cells using techniques known in the art, restriction mapped and sequenced in accordance with standard, well known techniques set forth in detail, e.g., in the foregoing references relating to recombinant DNA techniques. Of course, the DNA may be synthetic according to the present invention at any point during the isolation process or subsequent analysis.

Following manipulation of the isolated genetic material to provide RON antibodies, or antigen-binding fragments, variants, or derivatives thereof of the invention, the polynucleotides encoding the RON antibodies are typically inserted in an expression vector for introduction into host cells that may be used to produce the desired quantity of RON antibody.

Recombinant expression of an antibody, or fragment, derivative or analog thereof, e.g., a heavy or light chain of an antibody which binds to a target molecule described herein, e.g., RON, requires construction of an expression vector containing a polynucleotide that encodes the antibody. Once a polynucleotide encoding an antibody molecule or a heavy or light chain of an antibody, or portion thereof (preferably containing the heavy or light chain variable domain), of the invention has been obtained, the vector for the production of the antibody molecule may be produced by recombinant DNA technology using techniques well known in the art. Thus, methods for preparing a protein by expressing a polynucleotide containing an antibody encoding nucleotide sequence are described herein. Methods which are well known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding an antibody molecule of the invention, or a heavy or light chain thereof, or a heavy or light chain variable domain, operably linked to a promoter. Such vectors may include the nucleotide sequence encoding the constant region of the antibody molecule (see, e.g., PCT Publication WO 86/05807; PCT Publication WO 89/01036; and U.S. Pat. No. 5,122,464) and the variable domain of the antibody may be cloned into such a vector for expression of the entire heavy or light chain.

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes both heavy and light chain polypeptides. In such situations, the light chain is advantageously placed before the heavy chain to avoid an excess of toxic free heavy chain (Proudfoot, Nature 322:52 (1986); Kohler, Proc. Natl. Acad. Sci. USA 77:2197 (1980)). The coding sequences for the heavy and light chains may comprise cDNA or genomic DNA.

The term “vector” or “expression vector” is used herein to mean vectors used in accordance with the present invention as a vehicle for introducing into and expressing a desired gene in a host cell. As known to those skilled in the art, such vectors may easily be selected from the group consisting of plasmids, phages, viruses and retroviruses. In general, vectors compatible with the instant invention will comprise a selection marker, appropriate restriction sites to facilitate cloning of the desired gene and the ability to enter and/or replicate in eukaryotic or prokaryotic cells.

For the purposes of this invention, numerous expression vector systems may be employed. For example, one class of vector utilizes DNA elements which are derived from animal viruses such as bovine papilloma virus, polyoma virus, adenovirus, vaccinia virus, baculovirus, retroviruses (RSV, MMTV or MOMLV) or SV40 virus. Others involve the use of polycistronic systems with internal ribosome binding sites. Additionally, cells which have integrated the DNA into their chromosomes may be selected by introducing one or more markers which allow selection of transfected host cells. The marker may provide for prototrophy to an auxotrophic host, biocide resistance (e.g., antibiotics) or resistance to heavy metals such as copper. The selectable marker gene can either be directly linked to the DNA sequences to be expressed, or introduced into the same cell by cotransformation. Additional elements may also be needed for optimal synthesis of mRNA. These elements may include signal sequences, splice signals, as well as transcriptional promoters, enhancers, and termination signals.

In particularly preferred embodiments the cloned variable region genes are inserted into an expression vector along with the heavy and light chain constant region genes (preferably human) synthetic as discussed above. In one embodiment, this is effected using a proprietary expression vector of Biogen IDEC, Inc., referred to as NEOSPLA (disclosed in U.S. Pat. No. 6,159,730). This vector contains the cytomegalovirus promoter/enhancer, the mouse beta globin major promoter, the SV40 origin of replication, the bovine growth hormone polyadenylation sequence, neomycin phosphotransferase exon 1 and exon 2, the dihydrofolate reductase gene and leader sequence. This vector has been found to result in very high level expression of antibodies upon incorporation of variable and constant region genes, transfection in CHO cells, followed by selection in G418 containing medium and methotrexate amplification. Of course, any expression vector which is capable of eliciting expression in eukaryotic cells may be used in the present invention. Examples of suitable vectors include, but are not limited to plasmids pcDNA3, pHCMV/Zeo, pCR3.1, pEF1/His, pIND/GS, pRc/HCMV2, pSV40/Zeo2, pTRACER-HCMV, pUB6NV5-His, pVAX1, and pZeoSV2 (available from Invitrogen, San Diego, Calif.), and plasmid pCI (available from Promega, Madison, Wis.). In general, screening large numbers of transformed cells for those which express suitably high levels if immunoglobulin heavy and light chains is routine experimentation which can be carried out, for example, by robotic systems. Vector systems are also taught in U.S. Pat. Nos. 5,736,137 and 5,658,570, each of which is incorporated by reference in its entirety herein. This system provides for high expression levels, e.g., >30 pg/cell/day. Other exemplary vector systems are disclosed e.g., in U.S. Pat. No. 6,413,777.

In other preferred embodiments the RON antibodies, or antigen-binding fragments, variants, or derivatives thereof of the invention may be expressed using polycistronic constructs such as those disclosed in United States Patent Application Publication No. 2003-0157641 A1, filed Nov. 18, 2002 and incorporated herein in its entirety. In these novel expression systems, multiple gene products of interest such as heavy and light chains of antibodies may be produced from a single polycistronic construct. These systems advantageously use an internal ribosome entry site (IRES) to provide relatively high levels of RON antibodies, e.g., binding polypeptides, e.g., RON-specific antibodies or immunospecific fragments thereof in eukaryotic host cells. Compatible IRES sequences are disclosed in U.S. Pat. No. 6,193,980 which is also incorporated herein. Those skilled in the art will appreciate that such expression systems may be used to effectively produce the full range of RON antibodies disclosed in the instant application.

More generally, once the vector or DNA sequence encoding a monomeric subunit of the RON antibody has been prepared, the expression vector may be introduced into an appropriate host cell. Introduction of the plasmid into the host cell can be accomplished by various techniques well known to those of skill in the art. These include, but are not limited to, transfection (including electrophoresis and electroporation), protoplast fusion, calcium phosphate precipitation, cell fusion with enveloped DNA, microinjection, and infection with intact virus. See, Ridgway, A. A. G. “Mammalian Expression Vectors” Vectors, Rodriguez and Denhardt, Eds., Butterworths, Boston, Mass., Chapter 24.2, pp. 470-472 (1988). Typically, plasmid introduction into the host is via electroporation. The host cells harboring the expression construct are grown under conditions appropriate to the production of the light chains and heavy chains, and assayed for heavy and/or light chain protein synthesis. Exemplary assay techniques include enzyme-linked immunosorbent assay (ELISA), radioimmunoassay (RIA), or fluorescence-activated cell sorter analysis (FACS), immunohistochemistry and the like.

The expression vector is transferred to a host cell by conventional techniques and the transfected cells are then cultured by conventional techniques to produce an antibody for use in the methods described herein. Thus, the invention includes host cells containing a polynucleotide encoding an antibody of the invention, or a heavy or light chain thereof, operably linked to a heterologous promoter. In preferred embodiments for the expression of double-chained antibodies, vectors encoding both the heavy and light chains may be co-expressed in the host cell for expression of the entire immunoglobulin molecule, as detailed below.

As used herein, “host cells” refers to cells which harbor vectors constructed using recombinant DNA techniques and encoding at least one heterologous gene. In descriptions of processes for isolation of antibodies from recombinant hosts, the terms “cell” and “cell culture” are used interchangeably to denote the source of antibody unless it is clearly specified otherwise. In other words, recovery of polypeptide from the “cells” may mean either from spun down whole cells, or from the cell culture containing both the medium and the suspended cells.

A variety of host-expression vector systems may be utilized to express antibody molecules for use in the methods described herein. Such host-expression systems represent vehicles by which the coding sequences of interest may be produced and subsequently purified, but also represent cells which may, when transformed or transfected with the appropriate nucleotide coding sequences, express an antibody molecule of the invention in situ. These include but are not limited to microorganisms such as bacteria (e.g., E. coli, B. subtilis) transformed with recombinant bacteriophage DNA, plasmid DNA or cosmid DNA expression vectors containing antibody coding sequences; yeast (e.g., Saccharomyces, Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BLK, 293, 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Preferably, bacterial cells such as Escherichia coli, and more preferably, eukaryotic cells, especially for the expression of whole recombinant antibody molecule, are used for the expression of a recombinant antibody molecule. For example, mammalian cells such as Chinese hamster ovary cells (CHO), in conjunction with a vector such as the major intermediate early gene promoter element from human cytomegalovirus is an effective expression system for antibodies (Foecking et al., Gene 45:101 (1986); Cockett et al., Bio/Technology 8:2 (1990)).

The host cell line used for protein expression is often of mammalian origin; those skilled in the art are credited with ability to preferentially determine particular host cell lines which are best suited for the desired gene product to be expressed therein. Exemplary host cell lines include, but are not limited to, CHO (Chinese Hamster Ovary), DG44 and DUXB11 (Chinese Hamster Ovary lines, DHFR minus), HELA (human cervical carcinoma), CVI (monkey kidney line), COS (a derivative of CVI with SV40 T antigen), VERY, BHK (baby hamster kidney), MDCK, 293, WI38, R1610 (Chinese hamster fibroblast) BALBC/3T3 (mouse fibroblast), HAK (hamster kidney line), SP2/O (mouse myeloma), P3x63-Ag3.653 (mouse myeloma), BFA-1c1BPT (bovine endothelial cells), RAJI (human lymphocyte) and 293 (human kidney). CHO cells are particularly preferred. Host cell lines are typically available from commercial services, the American Tissue Culture Collection or from published literature.

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used.

For long-term, high-yield production of recombinant proteins, stable expression is preferred. For example, cell lines which stably express the antibody molecule may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which stably express the antibody molecule.

A number of selection systems may be used, including but not limited to the herpes simplex virus thymidine kinase (Wigler et al., Cell 11:223 (1977)), hypoxanthine-guanine phosphoribosyltransferase (Szybalska & Szybalski, Proc. Natl. Acad. Sci. USA 48:202 (1992)), and adenine phosphoribosyltransferase (Lowy et al., Cell 22:817 1980) genes can be employed in tk-, hgprt- or aprt-cells, respectively. Also, anti-metabolite resistance can be used as the basis of selection for the following genes: dhfr, which confers resistance to methotrexate (Wigler et al., Natl. Acad. Sci. USA 77:357 (1980); O'Hare et al., Proc. Natl. Acad. Sci. USA 78:1527 (1981)); gpt, which confers resistance to mycophenolic acid (Mulligan & Berg, Proc. Natl. Acad. Sci. USA 78:2072 (1981)); neo, which confers resistance to the aminoglycoside G-418 Clinical Pharmacy 12:488-505; Wu and Wu, Biotherapy 3:87-95 (1991); Tolstoshev, Ann. Rev. Pharmacol. Toxicol. 32:573-596 (1993); Mulligan, Science 260:926-932 (1993); and Morgan and Anderson, Ann. Rev. Biochem. 62:191-217 (1993); TIB TECH 11(5):155-215 (May, 1993); and hygro, which confers resistance to hygromycin (Santerre et al., Gene 30:147 (1984). Methods commonly known in the art of recombinant DNA technology which can be used are described in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds), Current Protocols in Human Genetics, John Wiley & Sons, NY (1994); Colberre-Garapin et al., J. Mol. Biol. 150:1 (1981), which are incorporated by reference herein in their entireties.

The expression levels of an antibody molecule can be increased by vector amplification (for a review, see Bebbington and Hentschel, The use of vectors based on gene amplification for the expression of cloned genes in mammalian cells in DNA cloning, Academic Press, New York, Vol. 3. (1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., Mol. Cell. Biol. 3:257 (1983)).

In vitro production allows scale-up to give large amounts of the desired polypeptides. Techniques for mammalian cell cultivation under tissue culture conditions are known in the art and include homogeneous suspension culture, e.g. in an airlift reactor or in a continuous stirrer reactor, or immobilized or entrapped cell culture, e.g. in hollow fibers, microcapsules, on agarose microbeads or ceramic cartridges. If necessary and/or desired, the solutions of polypeptides can be purified by the customary chromatography methods, for example gel filtration, ion-exchange chromatography, chromatography over DEAE-cellulose or (immuno-)affinity chromatography, e.g., after preferential biosynthesis of a synthetic hinge region polypeptide or prior to or subsequent to the HIC chromatography step described herein.

Genes encoding RON antibodies, or antigen-binding fragments, variants, or derivatives thereof of the invention can also be expressed non-mammalian cells such as bacteria or insect or yeast or plant cells. Bacteria which readily take up nucleic acids include members of the enterobacteriaceae, such as strains of Escherichia coli or Salmonella; Bacillaceae, such as Bacillus subtilis; Pneumococcus; Streptococcus, and Haemophilus influenzae. It will further be appreciated that, when expressed in bacteria, the heterologous polypeptides typically become part of inclusion bodies. The heterologous polypeptides must be isolated, purified and then assembled into functional molecules. Where tetravalent forms of antibodies are desired, the subunits will then self-assemble into tetravalent antibodies (WO02/096948A2).

In bacterial systems, a number of expression vectors may be advantageously selected depending upon the use intended for the antibody molecule being expressed. For example, when a large quantity of such a protein is to be produced, for the generation of pharmaceutical compositions of an antibody molecule, vectors which direct the expression of high levels of fusion protein products that are readily purified may be desirable. Such vectors include, but are not limited, to the E. coli expression vector pUR278 (Ruther et al., EMBO J. 2:1791 (1983)), in which the antibody coding sequence may be ligated individually into the vector in frame with the lacZ coding region so that a fusion protein is produced; pIN vectors (Inouye & Inouye, Nucleic Acids Res. 13:3101-3109 (1985); Van Heeke & Schuster, J. Biol. Chem. 24:5503-5509 (1989)); and the like. pGEX vectors may also be used to express foreign polypeptides as fusion proteins with glutathione S-transferase (GST). In general, such fusion proteins are soluble and can easily be purified from lysed cells by adsorption and binding to a matrix glutathione-agarose beads followed by elution in the presence of free glutathione. The pGEX vectors are designed to include thrombin or factor Xa protease cleavage sites so that the cloned target gene product can be released from the GST moiety.

In addition to prokaryotes, eukaryotic microbes may also be used. Saccharomyces cerevisiae, or common baker's yeast, is the most commonly used among eukaryotic microorganisms although a number of other strains are commonly available, e.g., Pichia pastoris.

For expression in Saccharomyces, the plasmid YRp7, for example, (Stinchcomb et al., Nature 282:39 (1979); Kingsman et al., Gene 7:141 (1979); Tschemper et al., Gene 10:157 (1980)) is commonly used. This plasmid already contains the TRP1 gene which provides a selection marker for a mutant strain of yeast lacking the ability to grow in tryptophan, for example ATCC No. 44076 or PEP4-1 (Jones, Genetics 85:12 (1977)). The presence of the trpl lesion as a characteristic of the yeast host cell genome then provides an effective environment for detecting transformation by growth in the absence of tryptophan.

In an insect system, Autographa californica nuclear polyhedrosis virus (AcNPV) is typically used as a vector to express foreign genes. The virus grows in Spodoptera frugiperda cells. The antibody coding sequence may be cloned individually into non-essential regions (for example the polyhedrin gene) of the virus and placed under control of an AcNPV promoter (for example the polyhedrin promoter).

Once an antibody molecule of the invention has been recombinantly expressed, it may be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. Alternatively, a preferred method for increasing the affinity of antibodies of the invention is disclosed in US 2002 0123057 A1.

### VIII. Treatment Methods Using Therapeutic RON-Specific Antibodies, or Immunospecific Fragments Thereof

One embodiment of the present invention provides methods for treating a hyperproliferative disease or disorder, e.g., cancer, a malignancy, a tumor, or a metastasis thereof, in an animal suffering from such disease or predisposed to contract such disease, the method comprising, consisting essentially of, or consisting of administering to the animal an effective amount of an antibody or immunospecific fragment thereof, that binds to RON or a variant of RON. Suitable antibodies include all antibodies and antigen-specific fragments thereof described herein. Examples include, but are not limited to, an isolated antibody or antigen-binding fragment thereof which specifically binds to the same RON epitope as a reference monoclonal Fab antibody fragment selected from the group consisting of M14-H06, M15-E10, M16-C07, M23-F10, M80-B03, M93-D02, M96-C05, M97-D03 and M98-E12 or a reference monoclonal antibody selected from the group consisting of 1P2E7, 1P3B2, 1P4A3, 1P4A12 and 1P5B10, an isolated antibody or antigen-binding fragment thereof which specifically binds to RON, where the antibody or fragment thereof competitively inhibits a reference monoclonal Fab antibody fragment selected from the group consisting of M14-H06, M15-E10, M16-C07, M23-F10, M80-B03, M93-D02, M96-C05, M97-D03 and M98-E12 or a reference monoclonal antibody selected from the group consisting of 1P2E7, 1P3B2, 1P4A3, 1P4A12 and 1P5B10, from binding to RON, or an isolated antibody or antigen-binding fragment thereof which specifically binds to RON, where the antibody or fragment thereof comprises an antigen binding domain identical to that of a monoclonal Fab antibody fragment selected from the group consisting of M14-H06, M15-E10, M16-C07, M23-F10, M80-B03, M93-D02, M96-C05, M97-D03 and M98-E12 or a reference monoclonal antibody selected from the group consisting of 1P2E7, 1P3B2, 1P4A3, 1P4A12 and 1P5B10.

In certain embodiments an antibody of the present invention which specifically binds to RON or a variant thereof inhibits MSP from binding to RON. In a further embodiment, an antibody of the present invention which specifically binds to RON or a variant thereof expressed on a cell, in particular a tumor cell or tumor associated macrophage, inhibits activation of downstream signal transduction molecules involved in cell proliferation, motility and/or metastasis. Such molecules include, but are not limited to PI3-K, Akt, mTOR and Rac. In a further embodiment, an antibody of the present invention which specifically binds to RON or a variant thereof expressed on a cell, in particular a tumor cell or tumor associated macrophage, inhibits activation of the Ras/MAPK signaling pathway. In a further embodiment, an antibody of the present invention which specifically binds to RON or a variant thereof expressed on a cell, in particular a tumor cell or tumor associated macrophage, inhibits activation of the src signaling pathway. In a further embodiment, an antibody of the present invention which specifically binds to RON or a variant thereof expressed on a cell, in particular a tumor cell or tumor associated macrophage, inhibits activation of the β-catenin signaling pathway. In still a further embodiment, an antibody of the present invention which specifically binds to RON or a variant thereof expressed on a cell, in particular a tumor cell or tumor associated macrophage, inhibits the interaction of RON with MSP. In another embodiment, an antibody of the present invention which specifically binds to RON or a variant thereof expressed on a cell, in particular a tumor cell or tumor associated macrophage, inhibits the activation of a RON-activated pathway including the PI3-K/Akt pathway, the Ras/MAPK pathway, the src pathway, the Fak pathway and the β-catenin signaling pathway. In yet another embodiment, an antibody of the present invention which specifically binds to RON or a variant thereof expressed on a cell, in particular a tumor cell or tumor associated macrophage, inhibits phosphorylation or activation of Erk 1/2. In yet a further embodiment, an antibody of the present invention which specifically binds to RON or a variant thereof expressed on a cell, in particular a tumor cell or tumor associated macrophage, inhibits cell proliferation, motility, and/or metastasis. In yet a further embodiment, an antibody of the present invention which specifically binds to RON or a variant thereof expressed on a cell, in particular a tumor cell or tumor associated macrophage, promotes apoptosis or anoikis. In yet a further embodiment, an antibody of the present invention which specifically binds to RON or a variant thereof expressed on a cell, in particular a tumor cell or tumor associated macrophage, blocks VEGF secretion. In yet a further embodiment, an antibody of the present invention which specifically binds to RON or a variant thereof expressed on a cell, in particular a tumor cell or tumor associated macrophage, blocks activity of other receptor kinases including but not limited to EGFR, TGFβ receptor and Met. In yet a further embodiment, an antibody of the present invention which specifically binds to RON or a variant thereof expressed on a cell, in particular a tumor cell or tumor associated macrophage, induces cytotoxic nitric oxide (NO) secretion. In yet a further embodiment, an antibody of the present invention which specifically binds to RON or a variant thereof expressed on a cell, in particular a tumor cell or tumor associated macrophage, induces IL-12 secretion. In yet a further embodiment, an antibody of the present invention which specifically binds to RON or a variant thereof expressed on a cell, in particular a tumor cell or tumor associated macrophage, blocks MSP secretion.

An antibody of the present invention which specifically binds to RON or a variant thereof, to be used in treatment methods disclosed herein can be prepared and used as a therapeutic agent that stops, reduces, prevents, or inhibits cellular activities involved in cellular hyperproliferation, e.g., cellular activities that induce the altered or abnormal pattern of vascularization that is often associated with hyperproliferative diseases or disorders.

Antibodies or immunospecific fragments thereof of the present invention include, but are not limited to monoclonal, chimeric or humanized antibodies, and fragments of antibodies that bind specifically to tumor-associated proteins such as RON. The antibodies may be monovalent, bivalent, polyvalent, or bifunctional antibodies, and the antibody fragments include Fab F(ab′)₂, and Fv.

Therapeutic antibodies according to the invention can be used in unlabeled or unconjugated form, or can be coupled or linked to cytotoxic moieties such as radiolabels and biochemical cytotoxins to produce agents that exert therapeutic effects.

In certain embodiments, an antibody, or immunospecific fragment thereof of the invention includes an antigen binding domain. An antigen binding domain is formed by antibody variable regions that vary from one antibody to another. Naturally occurring antibodies comprise at least two antigen binding domains, i.e., they are at least bivalent. As used herein, the term “antigen binding domain” includes a site that specifically binds an epitope on an antigen (e.g., a cell surface or soluble antigen). The antigen binding domain of an antibody typically includes at least a portion of an immunoglobulin heavy chain variable region and at least a portion of an immunoglobulin light chain variable region. The binding site formed by these variable regions determines the specificity of the antibody.

The present invention provides methods for treating various hyperproliferative disorders, e.g., by inhibiting tumor growth, in a mammal, comprising, consisting essentially of, or consisting of administering to the mammal an effective amount of a antibody or antigen-binding fragment thereof which specifically or preferentially binds to RON, e.g., human RON.

The present invention is more specifically directed to a method of treating a hyperproliferative disease, e.g., inhibiting or preventing tumor formation, tumor growth, tumor invasiveness, and/or metastasis formation, in an animal, e.g., a mammal, e.g., a human, comprising, consisting essentially of, or consisting of administering to an animal in need thereof an effective amount of a an antibody or immunospecific fragment thereof, which specifically or preferentially binds to one or more epitopes of RON.

In other embodiments, the present invention includes a method for treating a hyperproliferative disease, e.g., inhibiting tumor formation, tumor growth, tumor invasiveness, and/or metastasis formation in an animal, e.g., a human patient, where the method comprises administering to an animal in need of such treatment an effective amount of a composition comprising, consisting essentially of, or consisting of, in addition to a pharmaceutically acceptable carrier, an antibody, or immunospecific fragment thereof, which specifically binds to at least one epitope of RON, where the epitope comprises, consists essentially of, or consists of at least about four to five amino acids amino acids of SEQ ID NO:1, SEQ ID NO:2 or SEQ ID NO:113, at least seven, at least nine, or between at least about 15 to about 30 amino acids of SEQ ID NO:1, SEQ ID NO:2 or SEQ ID NO:113. The amino acids of a given epitope of SEQ ID NO:1, SEQ ID NO:2 or SEQ ID NO:113 as described may be, but need not be contiguous.

In certain embodiments, the at least one epitope of RON comprises, consists essentially of, or consists of a non-linear epitope formed by the extracellular domain of RON as expressed on the surface of a cell. Thus, in certain embodiments the at least one epitope of RON comprises, consists essentially of, or consists of at least 4, at least 5, at least 6, at least 7, at least 8, at least 9, at least 10, at least 15, at least 20, at least 25, between about 15 to about 30, or at least 10, 15, 20, 25, 30, 35, 40, 45, 50, 55, 60, 65, 70, 75, 80, 85, 90, 95, or 100 contiguous or non-contiguous amino acids of SEQ ID NO:1, SEQ ID NO:2 or SEQ ID NO:113, where non-contiguous amino acids form an epitope through protein folding.

In other embodiments, the present invention includes a method for treating a hyperproliferative disease, e.g., inhibiting tumor formation, tumor growth, tumor invasiveness, and/or metastasis formation in an animal, e.g., a human patient, where the method comprises administering to an animal in need of such treatment an effective amount of a composition comprising, consisting essentially of, or consisting of, in addition to a pharmaceutically acceptable carrier, an antibody, or immunospecific fragment thereof, which specifically binds to at least one epitope of RON, where the epitope comprises, consists essentially of, or consists of, in addition to one, two, three, four, five, six or more contiguous or non-contiguous amino acids of SEQ ID NO:1, SEQ ID NO:2 or SEQ ID NO:113 as described above, and an additional moiety which modifies the protein, e.g., a carbohydrate moiety may be included such that the binding molecule binds with higher affinity to modified target protein than it does to an unmodified version of the protein. Alternatively, the binding molecule does not bind the unmodified version of the target protein at all.

More specifically, the present invention provides a method of treating cancer in a human, comprising administering to a human in need of treatment a composition comprising an effective amount of an RON-specific antibody or immunospecific fragment thereof, and a pharmaceutically acceptable carrier. Types of cancer to be treated include, but are not limited to, stomach cancer, renal cancer, brain cancer, bladder cancer, colon cancer, lung cancer, breast cancer, pancreatic cancer, ovarian cancer, and prostate cancer.

In certain embodiments, an antibody or fragment thereof binds specifically to at least one epitope of RON or fragment or variant described above, i.e., binds to such an epitope more readily than it would bind to an unrelated, or random epitope; binds preferentially to at least one epitope of RON or fragment or variant described above, i.e., binds to such an epitope more readily than it would bind to a related, similar, homologous, or analogous epitope; competitively inhibits binding of a reference antibody which itself binds specifically or preferentially to a certain epitope of RON or fragment or variant described above; or binds to at least one epitope of RON or fragment or variant described above with an affinity characterized by a dissociation constant K_{D} of less than about 5×10⁻² M, about 10⁻² M, about 5×10⁻³ M, about 10⁻³ M, about 5×10⁻⁴ M, about 10⁻⁴ M, about 5×10⁻⁵ M, about 10⁻⁵ M, about 5×10⁻⁶ M, about 10⁻⁶ M, about 5×10⁻⁷ M, about 10⁻⁷ M, about 5×10⁻⁸ M, about 10⁻⁸ M, about 5×10⁻⁹ M, about 10⁻⁹ M, about 5×10⁻¹⁰ M, about 10⁻¹⁰ M, about 5×10⁻¹¹ M, about 10⁻¹¹ M, about 5×10⁻¹² M, about 10⁻¹² M, about 5×10⁻¹³ M, about 10⁻¹³ M, about 5×10⁻¹⁴ M, about 10⁻¹⁴ M, about 5×10⁻¹⁵ M, or about 10⁻¹⁵ M. As used in the context of antibody binding dissociation constants, the term “about” allows for the degree of variation inherent in the methods utilized for measuring antibody affinity. For example, depending on the level of precision of the instrumentation used, standard error based on the number of samples measured, and rounding error, the term “about 10⁻² M” might include, for example, from 0.05 M to 0.005 M. In certain embodiments, antibodies and fragments thereof of the present invention cross-react with RON proteins of other species from which they were raised, e.g., an antibody or fragment thereof which specifically binds to human RON also binds to murine RON. Other suitable antibodies or fragments thereof of the present invention include those that are highly species specific.

In specific embodiments, antibodies or immunospecific fragments thereof disclosed herein bind RON polypeptides or fragments or variants thereof with an off rate (k(off)) of less than or equal to 5×10⁻² sec⁻¹, 10⁻² sec⁻¹, 5×10⁻³ sec⁻¹ or 10⁻³ sec⁻¹. Other antibodies or immunospecific fragments thereof disclosed herein bind RON polypeptides or fragments or variants thereof with an off rate (k(off)) of less than or equal to 5×10⁻⁴ sec⁻¹, 10⁻⁴ sec⁻¹, 5×10⁵ sec⁻¹, or 10⁻⁵ sec⁻¹ 5×10⁻⁶ sec⁻¹, 10⁻⁶ sec⁻¹, 5×10⁻⁷ sec⁻¹ or 10⁻⁷ sec⁻¹.

In other embodiments, antibodies or immunospecific fragments thereof disclosed herein bind RON polypeptides or fragments or variants thereof with an on rate (k(on)) of greater than or equal to 10³ M⁻¹ sec⁻¹, 5×10³ M⁻¹ sec⁻¹, 10⁴ M⁻¹ sec⁻¹ or 5×10⁴ M⁻¹ sec⁻¹. Other antibodies or immunospecific fragments thereof for use in the diagnostic and treatment methods disclosed herein bind RON polypeptides or fragments or variants thereof with an on rate (k(on)) greater than or equal to 10⁵ M⁻¹ sec⁻¹, 5×10⁵ M⁻¹ sec⁻¹, 10⁶ M⁻¹ sec⁻¹, or 5×10⁶ M⁻¹ sec⁻¹ or 10⁷ M⁻¹ sec⁻¹.

In various embodiments, one or more binding molecules as described above is an antagonist of RON activity, for example, binding of an antagonist RON antibody to RON as expressed on a tumor cell inhibits binding of MSP, inhibits activation of molecules downstream in the signal transduction pathway, e.g., PI3-K, Akt, Ras, MAPK, src, Fak, β-catenin, and ERK 1/2, or inhibits tumor cell proliferation, motility or metastasis.

### IX. Diagnostic or Prognostic Methods Using RON-Specific Binding Molecules and Nucleic Acid Amplification Assays

RON-specific antibodies, or fragments, derivatives, or analogs thereof, can be used for diagnostic purposes to detect, diagnose, or monitor diseases, disorders, and/or conditions associated with the aberrant expression and/or activity of RON. RON expression is increased in tumor tissue and other neoplastic conditions.

RON-specific antibodies or fragments thereof, are useful for diagnosis, treatment, prevention and/or prognosis of hyperproliferative disorders in mammals, preferably humans. Such disorders include, but are not limited to, cancer, neoplasms, tumors and/or as described under elsewhere herein, especially RON-associated cancers such as stomach cancer, brain cancer, bladder cancer, colon cancer, lung cancer, breast cancer, pancreatic cancer, ovarian cancer, and prostate cancer.

For example, as disclosed herein, RON expression is associated with at least stomach, brain, bladder, colon, lung, breast, pancreatic, ovarian, and prostate tumor tissues. Accordingly, antibodies (and antibody fragments) directed against RON may be used to detect particular tissues expressing increased levels of RON. These diagnostic assays may be performed in vivo or in vitro, such as, for example, on blood samples, biopsy tissue or autopsy tissue.

Thus, the invention provides a diagnostic method useful during diagnosis of a cancers and other hyperproliferative disorders, which involves measuring the expression level of RON protein or transcript in tissue or other cells or body fluid from an individual and comparing the measured expression level with a standard RON expression levels in normal tissue or body fluid, whereby an increase in the expression level compared to the standard is indicative of a disorder.

One embodiment provides a method of detecting the presence of abnormal hyperproliferative cells, e.g., precancerous or cancerous cells, in a fluid or tissue sample, comprising assaying for the expression of RON in tissue or body fluid samples of an individual and comparing the presence or level of RON expression in the sample with the presence or level of RON expression in a panel of standard tissue or body fluid samples, where detection of RON expression or an increase in RON expression over the standards is indicative of aberrant hyperproliferative cell growth.

More specifically, the present invention provides a method of detecting the presence of abnormal hyperproliferative cells in a body fluid or tissue sample, comprising (a) assaying for the expression of RON in tissue or body fluid samples of an individual using RON-specific antibodies or immunospecific fragments thereof of the present invention, and (b) comparing the presence or level of RON expression in the sample with a the presence or level of RON expression in a panel of standard tissue or body fluid samples, whereby detection of RON expression or an increase in RON expression over the standards is indicative of aberrant hyperproliferative cell growth.

With respect to cancer, the presence of a relatively high amount of RON protein in biopsied tissue from an individual may indicate the presence of a tumor or other malignant growth, may indicate a predisposition for the development of such malignancies or tumors, or may provide a means for detecting the disease prior to the appearance of actual clinical symptoms. A more definitive diagnosis of this type may allow health professionals to employ preventative measures or aggressive treatment earlier thereby preventing the development or further progression of the cancer.

RON-specific antibodies of the present invention can be used to assay protein levels in a biological sample using classical immunohistological methods known to those of skill in the art (e.g., see Jalkanen, et al., J. Cell. Biol. 101:976-985 (1985); Jalkanen, et al., J. Cell Biol. 105:3087-3096 (1987)). Other antibody-based methods useful for detecting protein expression include immunoassays, such as the enzyme linked immunosorbent assay (ELISA) and the radioimmunoassay (RIA). Suitable antibody assay labels are known in the art and include enzyme labels, such as, glucose oxidase; radioisotopes, such as iodine (¹²⁵I, ¹²¹I), carbon (¹⁴C), sulfur (³⁵S), tritium (³H), indium (¹¹²In), and technetium (⁹⁹Tc); luminescent labels, such as luminol; and fluorescent labels, such as fluorescein and rhodamine, and biotin. Suitable assays are described in more detail elsewhere herein.

One aspect of the invention is a method for the in vivo detection or diagnosis of a hyperproliferative disease or disorder associated with aberrant expression of RON in an animal, preferably a mammal and most preferably a human. In one embodiment, diagnosis comprises: a) administering (for example, parenterally, subcutaneously, or intraperitoneally) to a subject an effective amount of a labeled antibody or fragment thereof of the present invention, which specifically binds to RON; b) waiting for a time interval following the administering for permitting the labeled binding molecule to preferentially concentrate at sites in the subject where RON is expressed (and for unbound labeled molecule to be cleared to background level); c) determining background level; and d) detecting the labeled molecule in the subject, such that detection of labeled molecule above the background level indicates that the subject has a particular disease or disorder associated with aberrant expression of RON. Background level can be determined by various methods including comparing the amount of labeled molecule detected to a standard value previously determined for a particular system.

It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of, e.g., ⁹⁹Tc. The labeled binding molecule, e.g., antibody or antibody fragment, will then preferentially accumulate at the location of cells which contain the specific protein. In vivo tumor imaging is described in S. W. Burchiel et al., “Immunopharmacokinetics of Radiolabeled Antibodies and Their Fragments.” (Chapter 13 in Tumor Imaging: The Radiochemical Detection of Cancer, S. W. Burchiel and B. A. Rhodes, eds., Masson Publishing Inc. (1982).

Depending on several variables, including the type of label used and the mode of administration, the time interval following the administration for permitting the labeled molecule to preferentially concentrate at sites in the subject and for unbound labeled molecule to be cleared to background level is 6 to 48 hours or 6 to 24 hours or 6 to 12 hours. In another embodiment the time interval following administration is 5 to 20 days or 7 to 10 days.

Presence of the labeled molecule can be detected in the patient using methods known in the art for in vivo scanning. These methods depend upon the type of label used. Skilled artisans will be able to determine the appropriate method for detecting a particular label. Methods and devices that may be used in the diagnostic methods of the invention include, but are not limited to, computed tomography (CT), whole body scan such as position emission tomography (PET), magnetic resonance imaging (MRI), and sonography.

In a specific embodiment, the binding molecule is labeled with a radioisotope and is detected in the patient using a radiation responsive surgical instrument (Thurston et al., U.S. Pat. No. 5,441,050). In another embodiment, the binding molecule is labeled with a fluorescent compound and is detected in the patient using a fluorescence responsive scanning instrument. In another embodiment, the binding molecule is labeled with a positron emitting metal and is detected in the patent using positron emission-tomography. In yet another embodiment, the binding molecule is labeled with a paramagnetic label and is detected in a patient using magnetic resonance imaging (MRI).

Antibody labels or markers for in vivo imaging of RON expression include those detectable by X-radiography, nuclear magnetic resonance imaging (NMR), MRI, CAT-scans or electron spin resonance imaging (ESR). For X-radiography, suitable labels include radioisotopes such as barium or cesium, which emit detectable radiation but are not overtly harmful to the subject. Suitable markers for NMR and ESR. include those with a detectable characteristic spin, such as deuterium, which may be incorporated into the antibody by labeling of nutrients for the relevant hybridoma. Where in vivo imaging is used to detect enhanced levels of RON expression for diagnosis in humans, it may be preferable to use human antibodies or “humanized” chimeric monoclonal antibodies as described elsewhere herein.

In a related embodiment to those described above, monitoring of an already diagnosed disease or disorder is carried out by repeating any one of the methods for diagnosing the disease or disorder, for example, one month after initial diagnosis, six months after initial diagnosis, one year after initial diagnosis, etc.

Where a diagnosis of a disorder, including diagnosis of a tumor, has already been made according to conventional methods, detection methods as disclosed herein are useful as a prognostic indicator, whereby patients continuing to exhibiting enhanced RON expression will experience a worse clinical outcome relative to patients whose expression level decreases nearer the standard level.

By “assaying the expression level of the tumor associated RON polypeptide” is intended qualitatively or quantitatively measuring or estimating the level of RON polypeptide in a first biological sample either directly (e.g., by determining or estimating absolute protein level) or relatively (e.g., by comparing to the cancer associated polypeptide level in a second biological sample). Preferably, RON polypeptide expression level in the first biological sample is measured or estimated and compared to a standard RON polypeptide level, the standard being taken from a second biological sample obtained from an individual not having the disorder or being determined by averaging levels from a population of individuals not having the disorder. As will be appreciated in the art, once the “standard” RON polypeptide level is known, it can be used repeatedly as a standard for comparison.

By “biological sample” is intended any biological sample obtained from an individual, cell line, tissue culture, or other source of cells potentially expressing RON. As indicated, biological samples include body fluids (such as sera, plasma, urine, synovial fluid and spinal fluid), and other tissue sources which contain cells potentially expressing RON. Methods for obtaining tissue biopsies and body fluids from mammals are well known in the art.

In an additional embodiment, antibodies, or immunospecific fragments of antibodies directed to a conformational epitope of RON may be used to quantitatively or qualitatively detect the presence of RON gene products or conserved variants or peptide fragments thereof. This can be accomplished, for example, by immunofluorescence techniques employing a fluorescently labeled antibody coupled with light microscopic, flow cytometric, or fluorimetric detection.

Cancers that may be diagnosed, and/or prognosed using the methods described above include but are not limited to, stomach cancer, renal cancer, brain cancer, bladder cancer, colon cancer, lung cancer, breast cancer, pancreatic cancer, ovarian cancer, and prostate cancer.

### X. Immunoassays

RON-specific antibodies or immunospecific fragments thereof disclosed herein may be assayed for immunospecific binding by any method known in the art. The immunoassays which can be used include but are not limited to competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g., Ausubel et al., eds, Current Protocols in Molecular Biology, John Wiley & Sons, Inc., New York, Vol. 1 (1994), which is incorporated by reference herein in its entirety). Exemplary immunoassays are described briefly below (but are not intended by way of limitation).

Immunoprecipitation protocols generally comprise lysing a population of cells in a lysis buffer such as RIPA buffer (1% NP-40 or Triton X-100, 1% sodium deoxycholate, 0.1% SDS, 0.15 M NaCl, 0.01 M sodium phosphate at pH 7.2, 1% Trasylol) supplemented with protein phosphatase and/or protease inhibitors (e.g., EDTA, PMSF, aprotinin, sodium vanadate), adding the antibody of interest to the cell lysate, incubating for a period of time (e.g., 1-4 hours) at 4.degree. C., adding protein A and/or protein G sepharose beads to the cell lysate, incubating for about an hour or more at 4.degree. C., washing the beads in lysis buffer and resuspending the beads in SDS/sample buffer. The ability of the antibody of interest to immunoprecipitate a particular antigen can be assessed by, e.g., western blot analysis. One of skill in the art would be knowledgeable as to the parameters that can be modified to increase the binding of the antibody to an antigen and decrease the background (e.g., pre-clearing the cell lysate with sepharose beads). For further discussion regarding immunoprecipitation protocols see, e.g., Ausubel et al., eds, Current Protocols in Molecular Biology, John Wiley & Sons, Inc., New York, Vol. 1 (1994) at 10.16.1.

Western blot analysis generally comprises preparing protein samples, electrophoresis of the protein samples in a polyacrylamide gel (e.g., 8%-20% SDS-PAGE depending on the molecular weight of the antigen), transferring the protein sample from the polyacrylamide gel to a membrane such as nitrocellulose, PVDF or nylon, blocking the membrane in blocking solution (e.g., PBS with 3% BSA or non-fat milk), washing the membrane in washing buffer (e.g., PBS-Tween 20), blocking the membrane with primary antibody (the antibody of interest) diluted in blocking buffer, washing the membrane in washing buffer, blocking the membrane with a secondary antibody (which recognizes the primary antibody, e.g., an anti-human antibody) conjugated to an enzymatic substrate (e.g., horseradish peroxidase or alkaline phosphatase) or radioactive molecule (e.g., 32p or 1251) diluted in blocking buffer, washing the membrane in wash buffer, and detecting the presence of the antigen. One of skill in the art would be knowledgeable as to the parameters that can be modified to increase the signal detected and to reduce the background noise. For further discussion regarding western blot protocols see, e.g., Ausubel et al., eds, Current Protocols in Molecular Biology, John Wiley & Sons, Inc., New York Vol. 1 (1994) at 10.8.1.

ELISAs comprise preparing antigen, coating the well of a 96 well microtiter plate with the antigen, adding the antibody of interest conjugated to a detectable compound such as an enzymatic substrate (e.g., horseradish peroxidase or alkaline phosphatase) to the well and incubating for a period of time, and detecting the presence of the antigen. In ELISAs the antibody of interest does not have to be conjugated to a detectable compound; instead, a second antibody (which recognizes the antibody of interest) conjugated to a detectable compound may be added to the well. Further, instead of coating the well with the antigen, the antibody may be coated to the well. In this case, a second antibody conjugated to a detectable compound may be added following the addition of the antigen of interest to the coated well. One of skill in the art would be knowledgeable as to the parameters that can be modified to increase the signal detected as well as other variations of ELISAs known in the art. For further discussion regarding ELISAs see, e.g., Ausubel et al., eds, Current Protocols in Molecular Biology, John Wiley & Sons, Inc., New York, Vol. 1 (1994) at 11.2.1.

The binding affinity of an antibody to an antigen and the off-rate of an antibody-antigen interaction can be determined by competitive binding assays. One example of a competitive binding assay is a radioimmunoassay comprising the incubation of labeled antigen (e.g., ³H or ¹²⁵I) with the antibody of interest in the presence of increasing amounts of unlabeled antigen, and the detection of the antibody bound to the labeled antigen. The affinity of the antibody of interest for a particular antigen and the binding off-rates can be determined from the data by Scatchard plot analysis. Competition with a second antibody can also be determined using radioimmunoassays. In this case, the antigen is incubated with antibody of interest is conjugated to a labeled compound (e.g., ³H or ¹²⁵I) in the presence of increasing amounts of an unlabeled second antibody.

RON-specific antibodies may, additionally, be employed histologically, as in immunofluorescence, immunoelectron microscopy or non-immunological assays, for in situ detection of cancer antigen gene products or conserved variants or peptide fragments thereof. In situ detection may be accomplished by removing a histological specimen from a patient, and applying thereto a labeled RON-specific antibody or fragment thereof, preferably applied by overlaying the labeled antibody (or fragment) onto a biological sample. Through the use of such a procedure, it is possible to determine not only the presence of RON protein, or conserved variants or peptide fragments, but also its distribution in the examined tissue. Using the present invention, those of ordinary skill will readily perceive that any of a wide variety of histological methods (such as staining procedures) can be modified in order to achieve such in situ detection.

Immunoassays and non-immunoassays for RON gene products or conserved variants or peptide fragments thereof will typically comprise incubating a sample, such as a biological fluid, a tissue extract, freshly harvested cells, or lysates of cells which have been incubated in cell culture, in the presence of a detectably labeled antibody capable of binding to RON or conserved variants or peptide fragments thereof, and detecting the bound antibody by any of a number of techniques well-known in the art.

The biological sample may be brought in contact with and immobilized onto a solid phase support or carrier such as nitrocellulose, or other solid support which is capable of immobilizing cells, cell particles or soluble proteins. The support may then be washed with suitable buffers followed by treatment with the detectably labeled RON-specific antibody. The solid phase support may then be washed with the buffer a second time to remove unbound antibody. Optionally the antibody is subsequently labeled. The amount of bound label on solid support may then be detected by conventional means.

By “solid phase support or carrier” is intended any support capable of binding an antigen or an antibody. Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present invention. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat such as a sheet, test strip, etc. Preferred supports include polystyrene beads. Those skilled in the art will know many other suitable carriers for binding antibody or antigen, or will be able to ascertain the same by use of routine experimentation.

The binding activity of a given lot of RON-specific antibody may be determined according to well known methods. Those skilled in the art will be able to determine operative and optimal assay conditions for each determination by employing routine experimentation.

There are a variety of methods available for measuring the affinity of an antibody-antigen interaction, but relatively few for determining rate constants. Most of the methods rely on either labeling antibody or antigen, which inevitably complicates routine measurements and introduces uncertainties in the measured quantities.

Surface plasmon resonance (SPR) as performed on BIAcore offers a number of advantages over conventional methods of measuring the affinity of antibody-antigen interactions: (i) no requirement to label either antibody or antigen; (ii) antibodies do not need to be purified in advance, cell culture supernatant can be used directly; (iii) real-time measurements, allowing rapid semi-quantitative comparison of different monoclonal antibody interactions, are enabled and are sufficient for many evaluation purposes; (iv) biospecific surface can be regenerated so that a series of different monoclonal antibodies can easily be compared under identical conditions; (v) analytical procedures are fully automated, and extensive series of measurements can be performed without user intervention. BIAapplications Handbook, version AB (reprinted 1998), BIACORE code No. BR-1001-86; BIAtechnology Handbook, version AB (reprinted 1998), BIACORE code No. BR-1001-84.

SPR based binding studies require that one member of a binding pair be immobilized on a sensor surface. The binding partner immobilized is referred to as the ligand. The binding partner in solution is referred to as the analyte. In some cases, the ligand is attached indirectly to the surface through binding to another immobilized molecule, which is referred as the capturing molecule. SPR response reflects a change in mass concentration at the detector surface as analytes bind or dissociate.

Based on SPR, real-time BIAcore measurements monitor interactions directly as they happen. The technique is well suited to determination of kinetic parameters. Comparative affinity ranking is extremely simple to perform, and both kinetic and affinity constants can be derived from the sensorgram data.

When analyte is injected in a discrete pulse across a ligand surface, the resulting sensorgram can be divided into three essential phases: (i) Association of analyte with ligand during sample injection; (ii) Equilibrium or steady state during sample injection, where the rate of analyte binding is balanced by dissociation from the complex; (iii) Dissociation of analyte from the surface during buffer flow.

The association and dissociation phases provide information on the kinetics of analyte-ligand interaction (kₐ and k_{d}, the rates of complex formation and dissociation, k_{d}/kₐ=K_{D}). The equilibrium phase provides information on the affinity of the analyte-ligand interaction (K_{D}).

BIAevaluation software provides comprehensive facilities for curve fitting using both numerical integration and global fitting algorithms. With suitable analysis of the data, separate rate and affinity constants for interaction can be obtained from simple BIAcore investigations. The range of affinities measurable by this technique is very broad ranging from mM to pM.

Epitope specificity is an important characteristic of a monoclonal antibody. Epitope mapping with BIAcore, in contrast to conventional techniques using radioimmunoassay, ELISA or other surface adsorption methods, does not require labeling or purified antibodies, and allows multi-site specificity tests using a sequence of several monoclonal antibodies. Additionally, large numbers of analyses can be processed automatically.

Pair-wise binding experiments test the ability of two MAbs to bind simultaneously to the same antigen. MAbs directed against separate epitopes will bind independently, whereas MAbs directed against identical or closely related epitopes will interfere with each other's binding. These binding experiments with BIAcore are straightforward to carry out.

For example, one can use a capture molecule to bind the first Mab, followed by addition of antigen and second MAb sequentially. The sensorgrams will reveal: 1. how much of the antigen binds to first Mab, 2. to what extent the second MAb binds to the surface-attached antigen, 3. if the second MAb does not bind, whether reversing the order of the pair-wise test alters the results.

Peptide inhibition is another technique used for epitope mapping. This method can complement pair-wise antibody binding studies, and can relate functional epitopes to structural features when the primary sequence of the antigen is known. Peptides or antigen fragments are tested for inhibition of binding of different MAbs to immobilized antigen. Peptides which interfere with binding of a given MAb are assumed to be structurally related to the epitope defined by that MAb.

### XI. Pharmaceutical Compositions and Administration Methods

Methods of preparing and administering RON-specific antibodies or immunospecific fragments thereof to a subject in need thereof are well known to or are readily determined by those skilled in the art. The route of administration of the binding molecule, e.g., binding polypeptide, e.g., RON-specific antibody or immunospecific fragment thereof may be, for example, oral, parenteral, by inhalation or topical. The term parenteral as used herein includes, e.g., intravenous, intraarterial, intraperitoneal, intramuscular, subcutaneous, rectal or vaginal administration. While all these forms of administration are clearly contemplated as being within the scope of the invention, a form for administration would be a solution for injection, in particular for intravenous or intraarterial injection or drip. Usually, a suitable pharmaceutical composition for injection may comprise a buffer (e.g. acetate, phosphate or citrate buffer), a surfactant (e.g. polysorbate), optionally a stabilizer agent (e.g. human albumin), etc. However, in other methods compatible with the teachings herein, binding molecules, e.g., binding polypeptides, e.g., RON-specific antibodies or immunospecific fragments thereof can be delivered directly to the site of the adverse cellular population thereby increasing the exposure of the diseased tissue to the therapeutic agent.

Preparations for parenteral administration includes sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. In the subject invention, pharmaceutically acceptable carriers include, but are not limited to, 0.01-0.1M and preferably 0.05M phosphate buffer or 0.8% saline. Other common parenteral vehicles include sodium phosphate solutions, Ringer's dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives may also be present such as for example, antimicrobials, antioxidants, chelating agents, and inert gases and the like.

More particularly, pharmaceutical compositions suitable for injectable use include sterile aqueous solutions (where water soluble) or dispersions and sterile powders for the extemporaneous preparation of sterile injectable solutions or dispersions. In such cases, the composition must be sterile and should be fluid to the extent that easy syringability exists. It should be stable under the conditions of manufacture and storage and will preferably be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Suitable formulations for use in the therapeutic methods disclosed herein are described in Remington's Pharmaceutical Sciences, Mack Publishing Co., 16th ed. (1980).

Prevention of the action of microorganisms can be achieved by various antibacterial and antifungal agents, for example, parabens, chlorobutanol, phenol, ascorbic acid, thimerosal and the like. In many cases, it will be preferable to include isotonic agents, for example, sugars, polyalcohols, such as mannitol, sorbitol, or sodium chloride in the composition. Prolonged absorption of the injectable compositions can be brought about by including in the composition an agent which delays absorption, for example, aluminum monostearate and gelatin.

In any case, sterile injectable solutions can be prepared by incorporating an active compound (e.g., a binding molecule, e.g., a binding polypeptide, e.g., RON-specific antibody or immunospecific fragment thereof, by itself or in combination with other active agents) in the required amount in an appropriate solvent with one or a combination of ingredients enumerated herein, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yields a powder of an active ingredient plus any additional desired ingredient from a previously sterile-filtered solution thereof. The preparations for injections are processed, filled into containers such as ampoules, bags, bottles, syringes or vials, and sealed under aseptic conditions according to methods known in the art. Further, the preparations may be packaged and sold in the form of a kit such as those described in co-pending U.S. Ser. No. 09/259,337 (US-2002-0102208 A1), which is incorporated herein by reference in its entirety. Such articles of manufacture will preferably have labels or package inserts indicating that the associated compositions are useful for treating a subject suffering from, or predisposed to autoimmune or neoplastic disorders.

Effective doses of the compositions of the present invention, for treatment of hyperproliferative disorders as described herein vary depending upon many different factors, including means of administration, target site, physiological state of the patient, whether the patient is human or an animal, other medications administered, and whether treatment is prophylactic or therapeutic. Usually, the patient is a human but non-human mammals including transgenic mammals can also be treated. Treatment dosages may be titrated using routine methods known to those of skill in the art to optimize safety and efficacy.

For treatment of hyperproliferative disorders with an antibody or fragment thereof, the dosage can range, e.g., from about 0.0001 to 100 mg/kg, and more usually 0.01 to 5 mg/kg (e.g., 0.02 mg/kg, 0.25 mg/kg, 0.5 mg/kg, 0.75 mg/kg, 1 mg/kg, 2 mg/kg, etc.), of the host body weight. For example dosages can be 1 mg/kg body weight or 10 mg/kg body weight or within the range of 1-10 mg/kg, preferably at least 1 mg/kg. Doses intermediate in the above ranges are also intended to be within the scope of the invention. Subjects can be administered such doses daily, on alternative days, weekly or according to any other schedule determined by empirical analysis. An exemplary treatment entails administration in multiple dosages over a prolonged period, for example, of at least six months. Additional exemplary treatment regimes entail administration once per every two weeks or once a month or once every 3 to 6 months. Exemplary dosage schedules include 1-10 mg/kg or 15 mg/kg on consecutive days, 30 mg/kg on alternate days or 60 mg/kg weekly. In some methods, two or more monoclonal antibodies with different binding specificities are administered simultaneously, in which case the dosage of each antibody administered falls within the ranges indicated.

RON-specific antibodies or immunospecific fragments thereof disclosed herein can be administered on multiple occasions. Intervals between single dosages can be weekly, monthly or yearly. Intervals can also be irregular as indicated by measuring blood levels of target polypeptide or target molecule in the patient. In some methods, dosage is adjusted to achieve a plasma polypeptide concentration of 1-1000 μg/ml and in some methods 25-300 μg/ml. Alternatively, binding molecules can be administered as a sustained release formulation, in which case less frequent administration is required. Dosage and frequency vary depending on the half-life of the antibody in the patient. The half-life of a binding molecule can also be prolonged via fusion to a stable polypeptide or moiety, e.g., albumin or PEG. In general, humanized antibodies show the longest half-life, followed by chimeric antibodies and nonhuman antibodies. In one embodiment, the binding molecules of the invention can be administered in unconjugated form, In another embodiment, the binding molecules, e.g., binding polypeptides, e.g., RON-specific antibodies or immunospecific fragments thereof for use in the methods disclosed herein can be administered multiple times in conjugated form. In still another embodiment, the binding molecules of the invention can be administered in unconjugated form, then in conjugated form, or vise versa.

The dosage and frequency of administration can vary depending on whether the treatment is prophylactic or therapeutic. In prophylactic applications, compositions comprising antibodies or a cocktail thereof are administered to a patient not already in the disease state or in a pre-disease state to enhance the patient's resistance. Such an amount is defined to be a “prophylactic effective dose.” In this use, the precise amounts again depend upon the patient's state of health and general immunity, but generally range from 0.1 to 25 mg per dose, especially 0.5 to 2.5 mg per dose. A relatively low dosage is administered at relatively infrequent intervals over a long period of time. Some patients continue to receive treatment for the rest of their lives.

In therapeutic applications, a relatively high dosage (e.g., from about 1 to 400 mg/kg of binding molecule, e.g., antibody per dose, with dosages of from 5 to 25 mg being more commonly used for radioimmunoconjugates and higher doses for cytotoxin-drug conjugated molecules) at relatively short intervals is sometimes required until progression of the disease is reduced or terminated, and preferably until the patient shows partial or complete amelioration of symptoms of disease. Thereafter, the patent can be administered a prophylactic regime.

In one embodiment, a subject can be treated with a nucleic acid molecule encoding an RON-specific antibody or immunospecific fragment thereof (e.g., in a vector). Doses for nucleic acids encoding polypeptides range from about 10 ng to 1 g, 100 ng to 100 mg, 1 μg to 10 mg, or 30-300 μg DNA per patient. Doses for infectious viral vectors vary from 10-100, or more, virions per dose.

Therapeutic agents can be administered by parenteral, topical, intravenous, oral, subcutaneous, intraarterial, intracranial, intraperitoneal, intranasal or intramuscular means for prophylactic and/or therapeutic treatment. In some methods, agents are injected directly into a particular tissue where RON-expressing cells have accumulated, for example intracranial injection. Intramuscular injection or intravenous infusion are preferred for administration of antibody. In some methods, particular therapeutic antibodies are injected directly into the cranium. In some methods, antibodies are administered as a sustained release composition or device, such as a Medipad™ device.

RON antibodies or fragments thereof of the invention can optionally be administered in combination with other agents that are effective in treating the disorder or condition in need of treatment (e.g., prophylactic or therapeutic).

Effective single treatment dosages (i.e., therapeutically effective amounts) of ⁹⁰Y-labeled binding polypeptides range from between about 5 and about 75 mCi, more preferably between about 10 and about 40 mCi. Effective single treatment non-marrow ablative dosages of ¹³¹I-labeled antibodies range from between about 5 and about 70 mCi, more preferably between about 5 and about 40 mCi. Effective single treatment ablative dosages (i.e., may require autologous bone marrow transplantation) of ¹³¹I-labeled antibodies range from between about 30 and about 600 mCi, more preferably between about 50 and less than about 500 mCi. In conjunction with a chimeric antibody, owing to the longer circulating half life vis-à-vis murine antibodies, an effective single treatment non-marrow ablative dosages of iodine-131 labeled chimeric antibodies range from between about 5 and about 40 mCi, more preferably less than about 30 mCi. Imaging criteria for, e.g., the ¹¹¹In label, are typically less than about 5 mCi.

While a great deal of clinical experience has been gained with ¹³¹I and ⁹⁰Y, other radiolabels are known in the art and have been used for similar purposes. Still other radioisotopes are used for imaging. For example, additional radioisotopes which are compatible with the scope of the instant invention include, but are not limited to, ¹²³I, ¹²⁵I, ³²P, ⁵⁷Co, ⁶⁴Cu, ⁶⁷Cu, ⁷⁷Br, ⁸¹Rb, ⁸¹Kr, ⁸⁷Sr, ¹¹³In, ¹²⁷Cs, ¹²⁹Cs, ¹³²I, ¹⁹⁷Hg, ²⁰³Pb, ²⁰⁶Bi, ¹⁷⁷Lu, ¹⁸⁶Re, ²¹²Pb, ²¹²Bi, 47Sc, ¹⁰⁵Rh, ¹⁰⁹Pd, ¹⁵³Sm, ¹⁸⁸Re, ¹⁹⁹Au, ²²⁵Ac, ²¹¹At, and ²¹³Bi. In this respect alpha, gamma and beta emitters are all compatible with in the instant invention. Further, in view of the instant disclosure it is submitted that one skilled in the art could readily determine which radionuclides are compatible with a selected course of treatment without undue experimentation. To this end, additional radionuclides which have already been used in clinical diagnosis include ¹²⁵I, ¹²³I, ⁹⁹Tc, ⁴³K, ⁵²Fe, ⁶⁷Ga, ⁶⁸Ga, as well as ¹¹¹In. Antibodies have also been labeled with a variety of radionuclides for potential use in targeted immunotherapy (Peirersz et al. Immunol. Cell Biol. 65: 111-125 (1987)). These radionuclides include ¹⁸⁸Re and ¹⁸⁶Re as well as ¹⁹⁹Au and ⁶⁷Cu to a lesser extent. U.S. Pat. No. 5,460,785 provides additional data regarding such radioisotopes and is incorporated herein by reference.

Whether or not RON-specific antibodies or immunospecific fragments thereof disclosed herein are used in a conjugated or unconjugated form, it will be appreciated that a major advantage of the present invention is the ability to use these molecules in myelosuppressed patients, especially those who are undergoing, or have undergone, adjunct therapies such as radiotherapy or chemotherapy. That is, the beneficial delivery profile (i.e. relatively short serum dwell time, high binding affinity and enhanced localization) of the molecules makes them particularly useful for treating patients that have reduced red marrow reserves and are sensitive to myelotoxicity. In this regard, the unique delivery profile of the molecules make them very effective for the administration of radiolabeled conjugates to myelosuppressed cancer patients. As such, the RON-specific antibodies or immunospecific fragments thereof disclosed herein are useful in a conjugated or unconjugated form in patients that have previously undergone adjunct therapies such as external beam radiation or chemotherapy. In other preferred embodiments, binding molecules, e.g., binding polypeptides, e.g., RON-specific antibodies or immunospecific fragments thereof (again in a conjugated or unconjugated form) may be used in a combined therapeutic regimen with chemotherapeutic agents. Those skilled in the art will appreciate that such therapeutic regimens may comprise the sequential, simultaneous, concurrent or coextensive administration of the disclosed antibodies or other binding molecules and one or more chemotherapeutic agents. Particularly preferred embodiments of this aspect of the invention will comprise the administration of a radiolabeled binding polypeptide.

While RON-specific antibodies or immunospecific fragments thereof may be administered as described immediately above, it must be emphasized that in other embodiments conjugated and unconjugated binding molecules may be administered to otherwise healthy patients as a first line therapeutic agent. In such embodiments binding molecules may be administered to patients having normal or average red marrow reserves and/or to patients that have not, and are not, undergoing adjunct therapies such as external beam radiation or chemotherapy.

However, as discussed above, selected embodiments of the invention comprise the administration of RON-specific antibodies or immunospecific fragments thereof to myelosuppressed patients or in combination or conjunction with one or more adjunct therapies such as radiotherapy or chemotherapy (i.e. a combined therapeutic regimen). As used herein, the administration of RON-specific antibodies or immunospecific fragments thereof in conjunction or combination with an adjunct therapy means the sequential, simultaneous, coextensive, concurrent, concomitant or contemporaneous administration or application of the therapy and the disclosed binding molecules. Those skilled in the art will appreciate that the administration or application of the various components of the combined therapeutic regimen may be timed to enhance the overall effectiveness of the treatment. For example, chemotherapeutic agents could be administered in standard, well known courses of treatment followed within a few weeks by radioimmunoconjugates described herein. Conversely, cytotoxin-conjugated binding molecules could be administered intravenously followed by tumor localized external beam radiation. In yet other embodiments, binding molecules may be administered concurrently with one or more selected chemotherapeutic agents in a single office visit. A skilled artisan (e.g. an experienced oncologist) would be readily be able to discern effective combined therapeutic regimens without undue experimentation based on the selected adjunct therapy and the teachings of the instant specification.

In this regard it will be appreciated that the combination of a binding molecule (with or without cytotoxin) and the chemotherapeutic agent may be administered in any order and within any time frame that provides a therapeutic benefit to the patient. That is, the chemotherapeutic agent and RON-specific antibody or immunospecific fragment thereof, may be administered in any order or concurrently. In selected embodiments RON-specific antibodies or immunospecific fragments thereof of the present invention will be administered to patients that have previously undergone chemotherapy. In yet other embodiments, RON-specific antibodies or immunospecific fragments thereof of the present invention will be administered substantially simultaneously or concurrently with the chemotherapeutic treatment. For example, the patient may be given the binding molecule while undergoing a course of chemotherapy. In preferred embodiments the binding molecule will be administered within 1 year of any chemotherapeutic agent or treatment. In other preferred embodiments the polypeptide will be administered within 10, 8, 6, 4, or 2 months of any chemotherapeutic agent or treatment. In still other preferred embodiments the binding molecule will be administered within 4, 3, 2 or 1 week of any chemotherapeutic agent or treatment. In yet other embodiments the binding molecule will be administered within 5, 4, 3, 2 or 1 days of the selected chemotherapeutic agent or treatment. It will further be appreciated that the two agents or treatments may be administered to the patient within a matter of hours or minutes (i.e. substantially simultaneously).

Moreover, in accordance with the present invention a myelosuppressed patient shall be held to mean any patient exhibiting lowered blood counts. Those skilled in the art will appreciate that there are several blood count parameters conventionally used as clinical indicators of myelosuppression and one can easily measure the extent to which myelosuppression is occurring in a patient. Examples of art accepted myelosuppression measurements are the Absolute Neutrophil Count (ANC) or platelet count. Such myelosuppression or partial myeloablation may be a result of various biochemical disorders or diseases or, more likely, as the result of prior chemotherapy or radiotherapy. In this respect, those skilled in the art will appreciate that patients who have undergone traditional chemotherapy typically exhibit reduced red marrow reserves. As discussed above, such subjects often cannot be treated using optimal levels of cytotoxin (i.e. radionuclides) due to unacceptable side effects such as anemia or immunosuppression that result in increased mortality or morbidity.

More specifically conjugated or unconjugated RON-specific antibodies or immunospecific fragments thereof of the present invention may be used to effectively treat patients having ANCs lower than about 2000/mm³ or platelet counts lower than about 150,000/mm³. More preferably RON-specific antibodies or immunospecific fragments thereof of the present invention may be used to treat patients having ANCs of less than about 1500/mm³, less than about 1000/mm³ or even more preferably less than about 500/mm³. Similarly, RON-specific antibodies or immunospecific fragments thereof of the present invention may be used to treat patients having a platelet count of less than about 75,000/mm³, less than about 50,000/mm³ or even less than about 10,000/mm³. In a more general sense, those skilled in the art will easily be able to determine when a patient is myelosuppressed using government implemented guidelines and procedures.

As indicated above, many myelosuppressed patients have undergone courses of treatment including chemotherapy, implant radiotherapy or external beam radiotherapy. In the case of the latter, an external radiation source is for local irradiation of a malignancy. For radiotherapy implantation methods, radioactive reagents are surgically located within the malignancy, thereby selectively irradiating the site of the disease. In any event, RON-specific antibodies or immunospecific fragments thereof of the present invention may be used to treat disorders in patients exhibiting myelosuppression regardless of the cause.

In this regard it will further be appreciated that RON-specific antibodies or immunospecific fragments thereof of the present invention may be used in conjunction or combination with any chemotherapeutic agent or agents (e.g. to provide a combined therapeutic regimen) that eliminates, reduces, inhibits or controls the growth of neoplastic cells in vivo. As discussed, such agents often result in the reduction of red marrow reserves. This reduction may be offset, in whole or in part, by the diminished myelotoxicity of the compounds of the present invention that advantageously allow for the aggressive treatment of neoplasias in such patients. In other embodiments, radiolabeled immunoconjugates disclosed herein may be effectively used with radiosensitizers that increase the susceptibility of the neoplastic cells to radionuclides. For example, radiosensitizing compounds may be administered after the radiolabeled binding molecule has been largely cleared from the bloodstream but still remains at therapeutically effective levels at the site of the tumor or tumors.

With respect to these aspects of the invention, exemplary chemotherapeutic agents that are compatible with the instant invention include alkylating agents, vinca alkaloids (e.g., vincristine and vinblastine), procarbazine, methotrexate and prednisone. The four-drug combination MOPP (mechlethamine (nitrogen mustard), vincristine (Oncovin), procarbazine and prednisone) is very effective in treating various types of lymphoma and comprises a preferred embodiment of the present invention. In MOPP-resistant patients, ABVD (e.g., adriamycin, bleomycin, vinblastine and dacarbazine), ChlVPP (chlorambucil, vinblastine, procarbazine and prednisone), CABS (lomustine, doxorubicin, bleomycin and streptozotocin), MOPP plus ABVD, MOPP plus ABV (doxorubicin, bleomycin and vinblastine) or BCVPP (carmustine, cyclophosphamide, vinblastine, procarbazine and prednisone) combinations can be used. Arnold S. Freedman and Lee M. Nadler, Malignant Lymphomas, in Harrison's Principles of Internal Medicine 1774-1788 (Kurt J. Isselbacher et al., eds., 13^{th} ed. 1994) and V. T. DeVita et al., (1997) and the references cited therein for standard dosing and scheduling. These therapies can be used unchanged, or altered as needed for a particular patient, in combination with one or more RON-specific antibodies or immunospecific fragments thereof of the present invention.

Additional regimens that are useful in the context of the present invention include use of single alkylating agents such as cyclophosphamide or chlorambucil, or combinations such as CVP (cyclophosphamide, vincristine and prednisone), CHOP (CVP and doxorubicin), C-MOPP (cyclophosphamide, vincristine, prednisone and procarbazine), CAP-BOP (CHOP plus procarbazine and bleomycin), m-BACOD (CHOP plus methotrexate, bleomycin and leucovorin), ProMACE-MOPP (prednisone, methotrexate, doxorubicin, cyclophosphamide, etoposide and leucovorin plus standard MOPP), ProMACE-CytaBOM (prednisone, doxorubicin, cyclophosphamide, etoposide, cytarabine, bleomycin, vincristine, methotrexate and leucovorin) and MACOP-B (methotrexate, doxorubicin, cyclophosphamide, vincristine, fixed dose prednisone, bleomycin and leucovorin). Those skilled in the art will readily be able to determine standard dosages and scheduling for each of these regimens. CHOP has also been combined with bleomycin, methotrexate, procarbazine, nitrogen mustard, cytosine arabinoside and etoposide. Other compatible chemotherapeutic agents include, but are not limited to, 2-chlorodeoxyadenosine (2-CDA), 2′-deoxycoformycin and fludarabine.

For patients with intermediate- and high-grade malignancies, who fail to achieve remission or relapse, salvage therapy is used. Salvage therapies employ drugs such as cytosine arabinoside, cisplatin, carboplatin, etoposide and ifosfamide given alone or in combination. In relapsed or aggressive forms of certain neoplastic disorders the following protocols are often used: IMVP-16 (ifosfamide, methotrexate and etoposide), MIME (methyl-gag, ifosfamide, methotrexate and etoposide), DHAP (dexamethasone, high dose cytarabine and cisplatin), ESHAP (etoposide, methylpredisolone, HD cytarabine, cisplatin), CEPP(B) (cyclophosphamide, etoposide, procarbazine, prednisone and bleomycin) and CAMP (lomustine, mitoxantrone, cytarabine and prednisone) each with well known dosing rates and schedules.

The amount of chemotherapeutic agent to be used in combination with the RON-specific antibodies or immunospecific fragments thereof of the present invention may vary by subject or may be administered according to what is known in the art. See, for example, Bruce A Chabner et al., Antineoplastic Agents, in Goodman & Gilman's The Pharmacological Basis of Therapeutics 1233-1287 (Joel G. Hardman et al., eds., 9^{th} ed. (1996)).

In another embodiment, an RON-specific antibody or immunospecific fragment thereof of the present invention is administered in conjunction with a biologic. Biologics useful in the treatment of cancers are known in the art and a binding molecule of the invention may be administered, for example, in conjunction with such known biologics.

For example, the FDA has approved the following biologics for the treatment of breast cancer: Herceptin® (trastuzumab, Genentech Inc., South San Francisco, Calif.; a humanized monoclonal antibody that has anti-tumor activity in HER2-positive breast cancer); Faslodex® (fulvestrant, AstraZeneca Pharmaceuticals, LP, Wilmington, Del.; an estrogen-receptor antagonist used to treat breast cancer); Arimidex® (anastrozole, AstraZeneca Pharmaceuticals, LP; a nonsteroidal aromatase inhibitor which blocks aromatase, an enzyme needed to make estrogen); Aromasin® (exemestane, Pfizer Inc., New York, N.Y.; an irreversible, steroidal aromatase inactivator used in the treatment of breast cancer); Femara® (letrozole, Novartis Pharmaceuticals, East Hanover, N.J.; a nonsteroidal aromatase inhibitor approved by the FDA to treat breast cancer); and Nolvadex® (tamoxifen, AstraZeneca Pharmaceuticals, LP; a nonsteroidal antiestrogen approved by the FDA to treat breast cancer). Other biologics with which the binding molecules of the invention may be combined include: Avastin™ (bevacizumab, Genentech Inc.; the first FDA-approved therapy designed to inhibit angiogenesis); and Zevalin® (ibritumomab tiuxetan, Biogen Idec, Cambridge, Mass.; a radiolabeled monoclonal antibody currently approved for the treatment of B-cell lymphomas).

In addition, the FDA has approved the following biologics for the treatment of colorectal cancer: Avastin™; Erbitux™ (cetuximab, ImClone Systems Inc., New York, N.Y., and Bristol-Myers Squibb, New York, N.Y.; is a monoclonal antibody directed against the epidermal growth factor receptor (EGFR)); Gleevec® (imatinib mesylate; a protein kinase inhibitor); and Ergamisol® (levamisole hydrochloride, Janssen Pharmaceutica Products, LP, Titusville, N.J.; an immunomodulator approved by the FDA in 1990 as an adjuvant treatment in combination with 5-fluorouracil after surgical resection in patients with Dukes' Stage C colon cancer).

For use in treatment of Non-Hodgkin's Lymphomas currently approved therapies include: Bexxar® (tositumomab and iodine I-131 tositumomab, GlaxoSmithKline, Research Triangle Park, NC; a multi-step treatment involving a mouse monoclonal antibody (tositumomab) linked to a radioactive molecule (iodine I-131)); Intron® (interferon alfa-2b, Schering Corporation, Kenilworth, N.J.; a type of interferon approved for the treatment of follicular non-Hodgkin's lymphoma in conjunction with anthracycline-containing combination chemotherapy (e.g., cyclophosphamide, doxorubicin, vincristine, and prednisone [CHOP])); Rituxan® (rituximab, Genentech Inc., South San Francisco, Calif., and Biogen Idec, Cambridge, Mass.; a monoclonal antibody approved for the treatment of non-Hodgkin's lymphoma; Ontak® (denileukin diftitox, Ligand Pharmaceuticals Inc., San Diego, Calif.; a fusion protein consisting of a fragment of diphtheria toxin genetically fused to interleukin-2); and Zevalin® (ibritumomab tiuxetan, Biogen Idec; a radiolabeled monoclonal antibody approved by the FDA for the treatment of B-cell non-Hodgkin's lymphomas).

For treatment of Leukemia, exemplary biologics which may be used in combination with the binding molecules of the invention include Gleevec®; Campath®-1H (alemtuzumab, Berlex Laboratories, Richmond, Calif.; a type of monoclonal antibody used in the treatment of chronic Lymphocytic leukemia). In addition, Genasense (oblimersen, Genta Corporation, Berkley Heights, N.J.; a BCL-2 antisense therapy under development to treat leukemia may be used (e.g., alone or in combination with one or more chemotherapy drugs, such as fludarabine and cyclophosphamide) may be administered with the claimed binding molecules.

For the treatment of lung cancer, exemplary biologics include Tarceva™ (erlotinib HCL, OSI Pharmaceuticals Inc., Melville, N.Y.; a small molecule designed to target the human epidermal growth factor receptor 1 (HER1) pathway).

For the treatment of multiple myeloma, exemplary biologics include Velcade® Velcade (bortezomib, Millennium Pharmaceuticals, Cambridge Mass.; a proteasome inhibitor). Additional biologics include Thalidomid® (thalidomide, Clegene Corporation, Warren, N.J.; an immunomodulatory agent and appears to have multiple actions, including the ability to inhibit the growth and survival of myeloma cells and anti-angiogenesis).

Other exemplary biologics include the MOAB IMC-C225, developed by ImClone Systems, Inc., New York, N.Y.

As previously discussed, RON-specific antibodies or immunospecific fragments thereof of the present invention, or recombinants thereof may be administered in a pharmaceutically effective amount for the in vivo treatment of mammalian hyperproliferative disorders. In this regard, it will be appreciated that the disclosed antibodies will be formulated so as to facilitate administration and promote stability of the active agent. Preferably, pharmaceutical compositions in accordance with the present invention comprise a pharmaceutically acceptable, non-toxic, sterile carrier such as physiological saline, non-toxic buffers, preservatives and the like. For the purposes of the instant application, a pharmaceutically effective amount of RON-specific antibodies or immunospecific fragments thereof of the present invention, or recombinant thereof, conjugated or unconjugated to a therapeutic agent, shall be held to mean an amount sufficient to achieve effective binding to a target and to achieve a benefit, e.g., to ameliorate symptoms of a disease or disorder or to detect a substance or a cell. In the case of tumor cells, the binding molecule will be preferably be capable of interacting with selected immunoreactive antigens on neoplastic or immunoreactive cells, or on non neoplastic cells, e.g., vascular cells associated with neoplastic cells. and provide for an increase in the death of those cells. Of course, the pharmaceutical compositions of the present invention may be administered in single or multiple doses to provide for a pharmaceutically effective amount of the binding molecule.

In keeping with the scope of the present disclosure, RON-specific antibodies or immunospecific fragments thereof of the present invention may be administered to a human or other animal in accordance with the aforementioned methods of treatment in an amount sufficient to produce a therapeutic or prophylactic effect. The RON-specific antibodies or immunospecific fragments thereof of the present invention can be administered to such human or other animal in a conventional dosage form prepared by combining the antibody of the invention with a conventional pharmaceutically acceptable carrier or diluent according to known techniques. It will be recognized by one of skill in the art that the form and character of the pharmaceutically acceptable carrier or diluent is dictated by the amount of active ingredient with which it is to be combined, the route of administration and other well-known variables. Those skilled in the art will further appreciate that a cocktail comprising one or more species of binding molecules according to the present invention may prove to be particularly effective.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2nd Ed., Sambrook et al., ed., Cold Spring Harbor Laboratory Press: (1989); Molecular Cloning: A Laboratory Manual, Sambrook et al., ed., Cold Springs Harbor Laboratory, New York (1992), DNA Cloning, D. N. Glover ed., Volumes I and II (1985); Oligonucleotide Synthesis, M. J. Gait ed., (1984); Mullis et al. U.S. Pat. No. 4,683,195; Nucleic Acid Hybridization, B. D. Hames & S. J. Higgins eds. (1984); Transcription And Translation, B. D. Hames & S. J. Higgins eds. (1984); Culture Of Animal Cells, R. I. Freshney, Alan R. Liss, Inc., (1987); Immobilized Cells And Enzymes, IRL Press, (1986); B. Perbal, A Practical Guide To Molecular Cloning (1984); the treatise, Methods In Enzymology, Academic Press, Inc., N.Y.; Gene Transfer Vectors For Mammalian Cells, J. H. Miller and M. P. Calos eds., Cold Spring Harbor Laboratory (1987); Methods In Enzymology, Vols. 154 and 155 (Wu et al. eds.); Immunochemical Methods In Cell And Molecular Biology, Mayer and Walker, eds., Academic Press, London (1987); Handbook Of Experimental Immunology, Volumes I-IV, D. M. Weir and C. C. Blackwell, eds., (1986); Manipulating the Mouse Embryo, Cold Spring Harbor Laboratory Press, Cold Spring Harbor, N.Y., (1986); and in Ausubel et al., Current Protocols in Molecular Biology, John Wiley and Sons, Baltimore, Md. (1989).

General principles of antibody engineering are set forth in Antibody Engineering, 2nd edition, C. A. K. Borrebaeck, Ed., Oxford Univ. Press (1995). General principles of protein engineering are set forth in Protein Engineering, A Practical Approach, Rickwood, D., et al., Eds., IRL Press at Oxford Univ. Press, Oxford, Eng. (1995). General principles of antibodies and antibody-hapten binding are set forth in: Nisonoff, A., Molecular Immunology, 2nd ed., Sinauer Associates, Sunderland, Mass. (1984); and Steward, M. W., Antibodies, Their Structure and Function, Chapman and Hall, New York, N.Y. (1984). Additionally, standard methods in immunology known in the art and not specifically described are generally followed as in Current Protocols in Immunology, John Wiley & Sons, New York; Stites et al. (eds), Basic and Clinical-Immunology (8th ed.), Appleton & Lange, Norwalk, Conn. (1994) and Mishell and Shiigi (eds), Selected Methods in Cellular Immunology, W.H. Freeman and Co., New York (1980).

Standard reference works setting forth general principles of immunology include Current Protocols in Immunology, John Wiley & Sons, New York; Klein, J., Immunology: The Science of Self-Nonself Discrimination, John Wiley & Sons, New York (1982); Kennett, R., et al., eds., Monoclonal Antibodies, Hybridoma: A New Dimension in Biological Analyses, Plenum Press, New York (1980); Campbell, A., “Monoclonal Antibody Technology” in Burden, R., et al., eds., Laboratory Techniques in Biochemistry and Molecular Biology, Vol. 13, Elsevere, Amsterdam (1984), Kuby Immunology 4^{th} ed. Ed. Richard A. Goldsby, Thomas J. Kindt and Barbara A. Osborne, H. Freemand & Co. (2000); Roitt, I., Brostoff, J. and Male D., Immunology 6^{th} ed. London: Mosby (2001); Abbas A., Abul, A. and Lichtman, A., Cellular and Molecular Immunology Ed. 5, Elsevier Health Sciences Division (2005); Kontermann and Dubel, Antibody Engineering, Springer Verlan (2001); Sambrook and Russell, Molecular Cloning: A Laboratory Manual. Cold Spring Harbor Press (2001); Lewin, Genes VIII, Prentice Hall (2003); Harlow and Lane, Antibodies: A Laboratory Manual, Cold Spring Harbor Press (1988); Dieffenbach and Dveksler, PCR Primer Cold Spring Harbor Press (2003).

All of the references cited above, as well as all references cited herein, are incorporated herein by reference in their entireties.

### EXAMPLES

### Example 1

### Generation and Conversion of Phage-Display-Derived Fab Antibodies

Recombinant human and murine RON ectodomain proteins were used to screen a human naïve phagemid Fab library containing 3.5×10¹⁰ unique clones (Nat. Biotechnol. 23(3):344-8 (2005)). Biotinylated RON proteins were captured on streptavidin-coated magnetic beads prior to incubation with the phage library. Selections were performed as described previously (Nat Biotechnol. 23(3):344-8 (2005)). Three distinct panning arms were followed using the human RON sema ectodomain and the full-length murine ectodomain fused to human IgG1 Fc (muRON-Fc) (R&D Systems, Inc.) Arm 1 panned on the human RON sema domain protein for 3 rounds. Arm 2 panned on the muRON-Fc protein for three rounds. Arm 3 panned on the human protein for two rounds, and the murine protein for the third round. After 3 rounds of panning, the 479 bp gene III stump was removed by MluI digestion, and the vector was religated for soluble Fab expression in TG1 cells. ELISA analysis of 960 clones from the human RON sema domain panning (arm 1) yielded 314 positive clones, containing 52 unique sequences. ELISA analysis of 1920 clones from arms 2 and 3 yielded 282 positive clones, containing 65 unique sequences. Unique clones were purified and binding was reconfirmed to recombinant human RON sema ectodomain and muRON-Fc by ELISA as well as CHO cells stably transfected with full-length human and murine RON. Based on the binding data, four clones isolated from arm 1 and five clones isolated from arms 2 and 3 were selected for further analysis: M93-D02, M96-C05, M97-D03, M98-E12, M14-H06, M15-E10, M16-C07, M23-F10 and M80-B03.

### Example 2

### Generation of Murine Monoclonal Antibodies

Immunization and Blood Sampling

Nine week old female Rbf mice (Jackson Labs, Bar Harbor, Me.) were immunized intraperitoneally (IP) with 40.5 μg of hRON-10 His (R&D Systems, Inc.) immobilized onto 25 μl Talon metal chelating resin (Clonetech) with a final bead density of 1.6 mg/ml resin. Subsequently, two additional injections were administered at 25 μg IP, also with a final bead density of 1.6 mg/ml resin. The two 25 μg injections were followed by a pre-fusion administration of 50 μg IP in RIBI adjuvant three days prior to harvesting lymphatic tissue.

Serum samples from the immunized mice were collected before the first immunization, seven days after the first boost, after each of the following two immunizations and just prior to lymphocyte collection via the retro orbital plexus collection method. Serum titers were measured using an ELISA and a FACS assay.

Solid Phase Assay (ELISA)

Maxisorp, 96-well microtiter plates (Nunc) were coated overnight with 50 μl/well of a 1 μg/ml sample of hRON-10 His in Dulbecco's Phosphate Buffer, pH 7.2. Plates were then emptied and washed three times with a solution of 0.05% Tween-20 in PBS, pH 7.2, using an Embla automated plate washer (Skatron). Following the wash procedure, wells were filled with a filtered 1% BSA in PBS, pH 7.2, blocking solution and allowed to incubate for 1 hour at room temperature. Following the plate block, the plates were flicked clear and 50 μl serial dilutions of serum, pre-serum, control samples or diluted mAb supernatants in blocking buffer (from monoclonal producing hybridomas) was immediately added; ELISA plates were allowed to incubate for 1 hour at room temperature. Plates were washed four times as described above, after which 50 μl 1:5000 dilution of goat anti-mouse IgG-HRP (Jackson Labs) in blocking buffer was added to each well and again allowed to incubate for 1 hour at room temperature. Plates were washed five times as described above, followed by the addition of 50 μl Ultra TMB (Pierce) substrate and then allowed to catalyze for approximately 10 minutes. The enzyme reaction was killed by adding equal volume 2.0 NH₂SO₄, and plates were read at 450 nm on a Spectramax 384 Plus (Molecular Devices) automated plate scanner. ELISA analysis was completed using Softmax Pro (Molecular Devices) analysis software.

Flow Cytometric Assay

Flow cytometry assays were carried out on SW480 and HT1080 cell lines. Cell isolation was performed by lifting cell monolayers from T162 flasks (Nunclon) by first removing the growth media and washing the cell monolayer gently with 20 ml of sterile PBS twice to remove cell debris and metabolized growth media. After each wash, the PBS washes were gently removed by aspiration. Then, 10 ml of the non-enzymatic Cell Dissociation Buffer (Sigma, cat C5914) was added to each flask and allowed to incubate for 2-5 minutes with periodic tapping and shaking to release the cell monolayer from the tissue culture plastic. Fifteen to 20 ml growth media was added to each flask and the cells were gently mixed prior to transfer into a 50 ml sterile tube. Cells were centrifuged at 1200 RPM for 4 minutes to sediment the cell pellet. Cells were washed in 20 ml sterile PBS twice prior to counting and suspending into FACS buffer (1% BSA in PBS).

In order to visualize fluorescence, cell staining was performed. All cell staining steps were performed on ice or at 4° C. First, the washed cells were counted using trypan blue exclusion on a hemocytometer and suspended to 1-2×10⁶/ml in FACS buffer and plated 50 ul per well into either 96-well V or U bottom plates. Next, 50 μl of the primary antibody (diluted purified control antibody, serum antibody or mAb supernatant) was added to each well and the plate was left on ice for 30 minutes. The cells were washed three times. Each wash cycle was conducted by adding 150 μl FAC buffer to each well, centrifuging the plate at 1300 rpm for 4 minutes, discarding the supernatant and tapping the cell pellets loose, adding 200 μl FACS buffer to each well, and then centrifuging the plate at 1300 rpm for 4 minutes. After the third wash, the supernatant was discarded and the cell pellets were tapped loose. Next, 50 μl of a working solution (1:200 as per manufacturer recommendation) of the secondary antibody was added to each well. Species-specific fluorescein (FITC) or phycoerythrin (PE)-conjugated second antibody from Jackson Lab were used. After addition of the secondary antibody, the plates were covered with foil, shaken and then stored on ice or at 4° C. for 30 minutes. The cells were then washed three times (as described above) before fixing. To fix the cells, 200 μl of fixative buffer (1% paraformaldehyde in PBS) was added to each well. Then, the plates were then shaken and read by flow cytometry or stored at 4° C. Plates were read in 48 or 72 hours. The following antibody controls were used in these assays: mouse immune serum, mouse pre serum, murine mAb 691 (R&D).

Hybridoma Development

FL653, an APRT-derivative of a Ig-/HGPRT-Balb/c mouse myeloma cell line, and SP2/0-Ag14, an Ig-/HGPRT-Balb/c mouse myeloma cell line, were cultured in 10% fetal bovine serum in Dulbecco's modified Eagle's medium (DMEM, Sigma Chemical Co., St. Louis, Mo.) containing 4500 mg/L glucose, L-glutamine, and 20 μg/ml 8-azaguanine (Sigma Chemical Co.) for at least 10 days prior to lymphocyte cell fusion experiments. Myeloma cells were cultured in a Series II™ model water jacketed incubator (Form a Scientific, Marietta, Ohio) which had been programmed to maintain a 37° C., 98% humid environment with a 7% CO₂ in air atmosphere.

Mice that screened positive in ELISA and FACS for antibodies specific to the hRON 10 His antigen were sacrificed and splenic B-lymphocytes aseptically harvested. Splenic B-lymphocytes were washed and prepared for use in PEG mediated lymphocyte somatic cell fusions, being fused to either the FL653 or SP2/0-Ag14 myeloma, as per Kennet, et al. (Monoclonal Antibody: A New Dimension in Biological Analyses, Plenum Press, New York (1992)). Fused cells were plated into 24-well sterile tissue culture plates (Corning Glass Works, Corning, N.Y.), and fed with Adenine, Aminopterin and Thymidine (AAT) or Hypoxanthine, Aminopterin and Thymidine containing culture media, for FL653 or SP2/0-Ag14 myeloma based fusions respectively. The cell culture environment was maintained at 37° C., 98% humid environment with a 7% CO₂ in air atmosphere.

After 10 days, AAT or HAT resistant cultures were isolated and screened by ELISA for immunoreactivity specific to both ELISA (R&D form) and FACS (SW480 hRON+/HT1080 hRON−) forms of hRON, as previously described. Positive cultures were subsequently cloned, expanded and frozen. Cloning was performed by limiting dilution (˜1 cell/well) and microscopically scored upon growth to assure clonal origin of the selected clones. Clones that screened positive on both ELISA and FACS format assays were expanded for freezing, subclass characterized using IsoStrip Assay (Roche), assayed for monoclonal production level and transferred to 1 Liter Cell Bags (Lampire Biologicals Laboratories, Inc.) for milligram level quantities of the selected monoclonal antibodies.

### Example 3

### Cloning of Murine Anti-Human RON Function-Blocking mAbs

### Cloning of Murine Hybridoma Immunoglobulin Variable Regions

Total cellular RNA from murine hybridoma cells was prepared using a Qiagen RNeasy mini kit following the manufacturer's recommended protocol. cDNAs encoding the variable regions of the heavy and light chains were cloned by RT-PCR from total cellular RNA, using random hexamers for priming of first strand cDNA. For PCR amplification of the murine immunoglobulin variable domains with intact signal sequences, a cocktail of degenerate forward primers hybridizing to multiple murine immunoglobulin gene family signal sequences and a single back primer specific for the 5′ end of the murine constant domain. The PCR products were gel-purified and subcloned into Invitrogen's pCR2.1TOPO vector using their TOPO cloning kit following the manufacturer's recommended protocol. Inserts from multiple independent subclones were sequenced to establish a consensus sequence. Deduced mature immunoglobulin N-termini were consistent with those determined by Edman degradation from the hybridoma. Assignment to specific subgroups is based upon BLAST analysis using consensus immunoglobulin variable domain sequences from the Kabat database. CDRs are designated using the Kabat definitions.

### 1P3B2.2

Shown below as SEQ ID NO:54 is the 1P3B2.2 mature heavy chain variable domain protein sequence, with CDRs underlined:

| | |
|---|---|
| 1 | EVQLQQSGPE LEKPGASVKI SCKASGYSFT GYNMNWVKQS NGESLEWIGD |
| 51 | IDPYYGGTRY NQKFKGKATL TVDKSSSTAY MQLKSLTSED SAVYYCAREG |
| 101 | RGFAYWGQGT LVTVSA |

This is a murine subgroup II(A) heavy chain. Shown below as SEQ ID NO:53 is the DNA sequence of the 1P3B2.2 heavy chain variable domain (from pYL363), with its signal sequence underlined (heavy chain encoded signal is MGWICIFLFLVSVTTGVHS (SEQ ID NO:103)):

| | |
|---|---|
| 1 | ATGGGTTGGATCTGTATCTTTCTCTTCCTCGTGTCAGTAACTACAGGTGT |
| 51 | CCACTCTGAGGTCCAGCTGCAGCAGTCTGGACCTGAGCTGGAGAAGCCTG |
| 101 | GCGCTTCAGTGAAAATATCCTGCAAGGCTTCTGGTTACTCATTCACTGGC |
| 151 | TACAACATGAACTGGGTGAAGCAGAGCAATGGAGAGAGCCTTGAGTGGAT |
| 201 | TGGAGATATTGATCCTTACTATGGTGGTACTAGGTACAACCAGAAGTTCA |
| 251 | AGGGCAAGGCCACATTGACTGTAGACAAATCCTCCAGCACAGCCTACATG |
| 301 | CAACTCAAGAGCCTGACATCTGAGGACTCTGCAGTCTATTACTGTGCAAG |
| 351 | AGAGGGGAGGGGTTTTGCTTACTGGGGCCAAGGGACTCTGGTCACTGTCT |
| 401 | CTGCA |

Shown below as SEQ ID NO:59 is the 1P3B2.2 mature light chain variable domain protein sequence, with CDRs underlined:

| | |
|---|---|
| 1 | DIQMTQSPASLSASVGETVTITCRASENIYSYLAWYQKKQGKSPQLLVYN |
| 51 | AKTSVEGVPSRFSGSGSGIQFSLKINSLQPEDFGSYYCHCQHHYGTLPTF |
| 101 | GGGTKLEIK |

This is a murine subgroup V kappa light chain. (Note the unusual unpaired cysteine in CDR3.) Shown below as SEQ ID NO:58 is the DNA sequence of the light chain variable domain (from pYL359), with its signal sequence underlined (light chain encoded signal is MRAPAQFLGL LLLWLTGARC (SEQ ID NO:104)):

| | |
|---|---|
| 1 | ATGAGGGCCCCTGCTCAGTTCCTTGGGTTGCTGCTGCTGTGGCTTACAGG |
| 51 | TGCCAGATGTGACATCCAGATGACTCAGTCTCCAGCCTCCCTATCTGCAT |
| 101 | CTGTGGGAGAAACTGTCACCATCACATGTCGAGCAAGTGAGAATATTTAC |
| 151 | AGTTATTTAGCATGGTATCAGAAGAAACAGGGAAAATCTCCTCAACTCCT |
| 201 | GGTCTATAATGCAAAAACCTCAGTAGAAGGTGTGCCATCAAGGTTCAGTG |
| 251 | GCAGTGGATCAGGCATACAGTTTTCTCTGAAGATCAATAGCCTGCAGCCT |
| 301 | GAAGATTTTGGGAGTTATTACTGTCACTGTCAACATCATTATGGTACTCT |
| 351 | TCCGACGTTCGGTGGAGGCACCAAGCTGGAAATCAAA |

### 1P4A3.3

Shown below as SEQ ID NO:64 is the 1P4A3.3 mature heavy chain variable domain protein sequence, with CDRs underlined:

| | |
|---|---|
| 1 | EVQLQQSGPE LEKPGASVMI SCKASGYSFT GYNMNWVKQS TGKSLEWIGD |
| 51 | IDPYYDGTRY NQKFKGKATL TADKSSSTAY MQLKSLTSED SAVYYCTREG |
| 101 | RGFAYWGQGT LVTVSA |

This is a murine subgroup II(A) heavy chain. Shown below as SEQ ID NO:63 is the DNA sequence of the 1P4A3.3 heavy chain variable domain (from pYL367), with its signal sequence underlined (heavy chain encoded signal is MGWSWVFLLI LSVTTGVHS (SEQ ID NO:105)):

| | |
|---|---|
| 1 | ATGGGATGGA GCTGGGTCTT TCTCTTAATC CTATCAGTAA CTACAGGTGT |
| 51 | CCACTCTGAG GTCCAGCTGC AGCAGTCTGG ACCTGAGCTG GAGAAGCCTG |
| 101 | GCGCTTCAGT GATGATATCC TGCAAGGCTT CTGGTTACTC ATTCACTGGC |
| 151 | TACAACATGA ACTGGGTGAA GCAGAGCACT GGCAAGAGCC TTGAGTGGAT |
| 201 | TGGAGATATT GATCCTTACT ATGATGGTAC TAGGTACAAC CAGAAGTTCA |
| 251 | AGGGCAAGGC CACATTGACT GCAGACAAAT CCTCCAGCAC AGCCTACATG |
| 301 | CAGCTCAAGA GCCTGACATC TGAAGACTCT GCAGTCTATT ACTGTACAAG |
| 351 | AGAGGGAAGA GGGTTTGCTT ACTGGGGCCA AGGGACTCTG GTCACTGTCT |
| 401 | CTGCA |

Shown below as SEQ ID NO:69 is the 1P4A3.3 mature light chain variable domain protein sequence, with CDRs underlined:

| | |
|---|---|
| 1 | DIQMTQSPAS LSASVGETVT ITCRASENIY SYLAWYQQKQ GKSPQLLVYN |
| 51 | AKTLAGGVPS RFSGSGSGTQ FSLKINSLQP EDFGSYYCQH YYGTPLTFGA |
| 101 | GTKLELK |

This is a murine subgroup V kappa light chain. Shown below as SEQ ID NO:68 is the DNA sequence of the light chain variable domain (from pYL360), with its signal sequence underlined (light chain encoded signal is MRSPAQFLGL LLLWLTGARC (SEQ ID NO:106)):

| | |
|---|---|
| 1 | ATGAGGTCCC CAGCTCAGTT CCTTGGGTTG CTGCTGCTGT GGCTTACAGG |
| 51 | TGCCAGATGT GACATCCAGA TGACTCAGTC TCCAGCCTCC CTATCTGCAT |
| 101 | CTGTGGGAGA AACTGTCACC ATCACATGTC GAGCAAGTGA GAATATTTAC |
| 151 | AGTTATTTAG CATGGTATCA GCAGAAACAG GGAAAATCTC CTCAGCTCCT |
| 201 | GGTCTATAAT GCAAAAACCT TAGCAGGAGG TGTGCCATCA AGGTTCAGTG |
| 251 | GCAGTGGATC AGGCACACAG TTTTCTCTGA AGATCAACAG CCTGCAGCCT |
| 301 | GAAGATTTTG GGAGTTATTA TTGTCAACAT TATTATGGTA CTCCTCTCAC |
| 351 | GTTCGGTGCT GGGACCAAGC TGGAGCTGAA A |

The 1P3B2.2 and 1P4A3.3 mAbs are highly homologous. Shown below is the alignment of the heavy chains of 1P3B2.2 (upper) and 1P4A3.3 (lower), which share 94.8% identity:

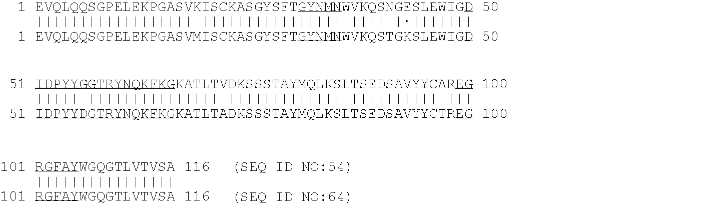

Shown below is the alignment of the light chains of 1P3B2.2 (upper) and 1P4A3.3 (lower), which share 90.7% identity:

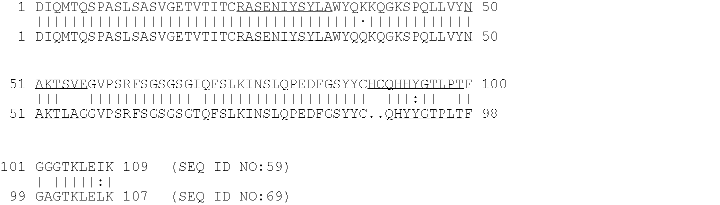

### 1P5B10.3

Shown below as SEQ ID NO:74 is the 1P5B10.3 mature heavy chain variable domain protein sequence with CDRs underlined:

| | |
|---|---|
| 1 | EVQLQQSGPE LVKPGASMKI SCRAAGFSFT GYTMNWVKQS HGKSLEWIGL |
| 51 | INLNNGGTSH NQKFKGKATL TVDKSSSTAY MELLSLTSED SAVYYCARWL |
| 101 | RRGGYAMDYW GQGISVTVSS |

This is a murine subgroup II(A) heavy chain. Shown below as SEQ ID NO:73 is the DNA sequence of the 1P5B10.3 heavy chain variable domain (from pYL358), with its signal sequence underlined (heavy chain encoded signal is MGCSWVMLFL LSGTAGVHS (SEQ ID NO:107)):

| | |
|---|---|
| 1 | ATGGGATGCA GCTGGGTAAT GCTCTTCCTC CTGTCAGGAA CTGCAGGTGT |
| 51 | CCACTCTGAG GTCCAGCTAC AACAGTCTGG ACCTGAACTG GTGAAGCCTG |
| 101 | GAGCTTCAAT GAAGATATCC TGCAGGGCTG CTGGTTTCTC ATTCACTGGC |
| 151 | TACACCATGA ACTGGGTGAA GCAGAGCCAT GGAAAGAGCC TTGAGTGGAT |
| 201 | TGGACTTATT AATCTTAACA ATGGTGGTAC TAGCCACAAC CAGAAGTTCA |
| 251 | AGGGCAAGGC CACATTAACT GTAGACAAGT CATCCAGCAC AGCCTACATG |
| 301 | GAGCTCCTCA GTCTGACATC TGAGGACTCT GCAGTCTATT ACTGTGCAAG |
| 351 | ATGGTTACGT CGTGGGGGCT ATGCTATGGA CTACTGGGGT CAAGGAATTT |
| 401 | CAGTCACCGT CTCCTCA |

Shown below as SEQ ID NO:79 is the 1P5B10.3 mature light chain variable domain protein sequence, with CDRs underlined:

| | |
|---|---|
| 1 | DILLTQSPAI LSVSPGERVS FSCRASQNIG TSIHWYQQRT NGSPRLLIKY |
| 51 | ASESISGIPS RFSGSGSGTD FTLSINSVES EDIADYYCQQ SDSWPLTFGA |
| 101 | GTKLELK |

This is a murine subgroup I kappa light chain. Shown below as SEQ ID NO:78 is the DNA sequence of the light chain variable domain (from pYL368), with its signal sequence underlined (light chain encoded signal is MVSSAQFLVF LLFWIPASRG (SEQ ID NO:108)):

| | |
|---|---|
| 1 | ATGGTGTCCT CAGCTCAGTT CCTTGTATTT TTGCTTTTCT GGATTCCAGC |
| 51 | CTCCAGAGGT GACATCTTGC TGACTCAGTC TCCAGCCATC CTGTCTGTGA |
| 101 | GTCCAGGAGA AAGAGTCAGT TTCTCCTGCA GGGCCAGTCA GAACATTGGC |
| 151 | ACAAGCATAC ACTGGTATCA GCAAAGAACA AATGGTTCTC CAAGGCTTCT |
| 201 | CATAAAGTAT GCTTCTGAGT CTATCTCTGG GATCCCTTCC AGGTTTAGTG |
| 251 | GCAGTGGATC AGGGACAGAT TTTACTCTTA GCATCAACAG TGTGGAGTCT |
| 301 | GAAGATATTG CAGATTATTA CTGTCAACAA AGTGATAGCT GGCCACTCAC |
| 351 | GTTTGGTGCT GGGACCAAGC TGGAGCTGAA A |

### 1P4A12.2

Shown below as SEQ ID NO:84 is the 1P4A12.2 mature heavy chain variable domain protein sequence, with CDRs underlined:

| | |
|---|---|
| 1 | EVQLQQSGPE LVKPGASMKI SCKASGYSFT GYTMNWVKQS HGKKLEWIGL |
| 51 | INPYNGGTIY NQKFKGKATL TVDKSSSTAY MELLSLTSED SAVYYCARWL |
| 101 | RRGGYAMDYW GQGASVTVSS |

This is a murine subgroup II(A) heavy chain. Shown below as SEQ ID NO:83 is the DNA sequence of the 1P5B10.3 heavy chain variable domain (from pCN558), with its signal sequence underlined (heavy chain encoded signal is MGCSCVMLFL LSGTAGVRS (SEQ ID NO:109)):

| | |
|---|---|
| 1 | ATGGGATGCAGCTGTGTAATGCTCTTCCTCCTGTCAGGAACTGCAGGTGT |
| 51 | CCGCTCTGAGGTCCAGCTGCAACAGTCTGGACCTGAGCTGGTGAAGCCTG |
| 101 | GAGCTTCAATGAAGATATCCTGCAAGGCTTCTGGTTACTCATTCACTGGC |
| 151 | TACACCATGAACTGGGTGAAGCAGAGCCATGGAAAGAAGCTTGAGTGGAT |
| 201 | TGGACTTATTAATCCTTACAATGGTGGGACTATCTACAACCAGAAGTTCA |
| 251 | AGGGCAAGGCCACATTAACTGTAGACAAGTCATCCAGCACAGCCTACATG |
| 301 | GAGCTCCTCAGTCTGACATCTGAGGACTCTGCAGTCTATTACTGTGCAAG |
| 351 | ATGGTTACGACGTGGGGGCTATGCTATGGACTACTGGGGTCAAGGAGCCT |
| 401 | CAGTCACCGTCTCCTCA |

Shown below as SEQ ID NO:89 is the 1P5B10.3 mature light chain variable domain protein sequence, with CDRs underlined:

| | |
|---|---|
| 1 | DILLTQSPAI LSVSPGERVS FSCRASQSIG TSIHWYQQRT NGSPRLLIKF |
| 51 | ASESISGIPS RFSGSGSGTD FTLSINSVES EDIADYYCQQ SDSWPLTFGA |
| 101 | GTKLEVK |

This is a murine subgroup kappa I light chain. Shown below as SEQ ID NO:88 is the DNA sequence of the light chain variable domain (from pCN559), with its signal sequence underlined (light chain encoded signal is MVSSAQFLVF LLFWIPASRG (SEQ ID NO:110)):

| | |
|---|---|
| 1 | ATGGTGTCCTCAGCTCAGTTCCTTGTATTTTTGCTTTTCTGGATTCCAGC |
| 51 | CTCCAGAGGTGACATCTTGCTGACTCAGTCTCCAGCCATCCTGTCTGTGA |
| 101 | GTCCAGGAGAAAGAGTCAGTTTCTCCTGCAGGGCCAGTCAGAGCATTGGC |
| 151 | ACAAGCATACACTGGTATCAGCAAAGAACAAATGGTTCTCCAAGGCTTCT |
| 201 | CATAAAGTTTGCTTCTGAGTCTATCTCTGGGATCCCTTCCAGGTTTAGTG |
| 251 | GCAGTGGATCAGGGACAGATTTTACTCTTAGCATCAACAGTGTGGAGTCT |
| 301 | GAAGATATTGCAGATTATTACTGTCAACAAAGTGATAGCTGGCCACTCAC |
| 351 | GTTCGGTGCTGGGACCAAGCTGGAGGTGAAA |

The 1P5B10.3 and 1P4A12.2 mAbs are highly homologous. Shown below is the alignment of the heavy chains of 1P5B10.3 (upper) and 1P4A12.2 (lower), which share 92.5% identity:

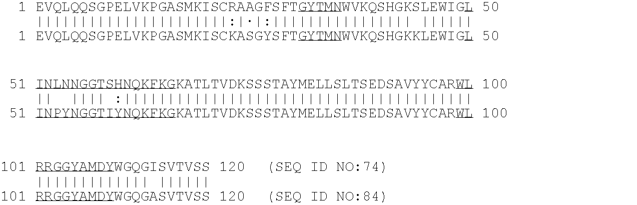

Shown below is the alignment of the light chains of 1P5B10.3 (upper) and 1P4A12.2 (lower), which share 97.2% identity:

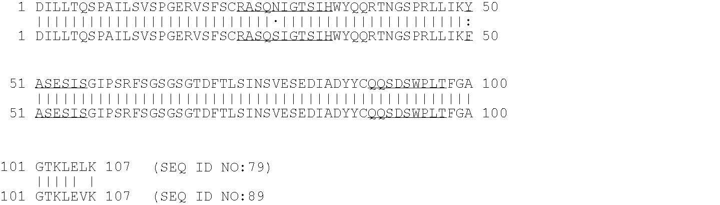

### 1P2E7.3

Shown below as SEQ ID NO:94 is the 1P2E7.3 mature heavy chain variable domain protein sequence, with CDRs underlined:

| | |
|---|---|
| 1 | DVQLQESGPGLVKPSQSLSLTCTVTGDSITSDYAWNWIRQFPGNKLEWMG |
| 51 | YISYSGSTSYNPSLKSRFSITRDTSKNQFFLQLNSVTTEDSATYYCARGG |
| 101 | FYYRYAGPGFAYWGQGTLVTVSA |

This is a murine subgroup I(A) heavy chain. Shown below as SEQ ID NO:93 is the DNA sequence of the 1P2E7.3 heavy chain variable domain (from pCN556), with its signal sequence underlined (heavy chain encoded signal is MRVLILLWLF TAFPGILS (SEQ ID NO:111)):

| | |
|---|---|
| 1 | ATGAGAGTGC TGATTCTTTT GTGGCTGTTC ACAGCCTTCC CTGGTATCCT |
| 51 | GTCTGATGTG CAGCTTCAGG AGTCGGGACC TGGCCTGGTG AAACCTTCTC |
| 101 | AGTCTCTGTC CCTCACCTGC ACTGTCACTG GCGACTCAAT CACCAGTGAT |
| 151 | TATGCCTGGA ACTGGATCCG GCAGTTTCCA GGAAACAAAC TGGAGTGGAT |
| 201 | GGGCTACATA AGCTACAGTG GTAGCACTAG CTACAACCCA TCTCTCAAAA |
| 251 | GTCGATTCTC TATCACTCGA GACACATCCA AGAACCAGTT CTTCCTGCAG |
| 301 | TTGAATTCTG TGACTACTGA GGACTCAGCC ACATATTACT GTGCAAGAGG |
| 351 | GGGGTTCTAC TATAGGTACG CCGGGCCTGG GTTTGCTTAT TGGGGCCAAG |
| 401 | GGACTCTGGT CACTGTCTCT GCA |

Shown below as SEQ ID NO:99 is the 1P2E7.3 mature light chain variable domain protein sequence, with CDRs underlined:

| | |
|---|---|
| 1 | DVVMTQTPLT LSVTIGQPAS ISCKSSQSLL YTNGKTYLNW LLQRPGQSPK |
| 51 | RLIYLVSKLD SGVPDRFSGS GSGTDFTLKI SRVEAEDLGV YYCLQSTHFP |
| 101 | LTFGAGTKLE LK |

This is a murine subgroup II kappa light chain. Shown below as SEQ ID NO:99 is the DNA sequence of the 1P2E7.3 light chain variable domain (from pCN557), with its signal sequence underlined (heavy chain encoded signal is MMSPAQFLFL LVLSIQEING (SEQ ID NO:112)):

| | |
|---|---|
| 1 | ATGATGAGTCCTGCCCAGTTCCTGTTTCTGTTAGTGCTCTCGATTCAGGA |
| 51 | AATCAACGGTGATGTTGTGATGACCCAGACTCCACTCACTTTGTCGGTTA |
| 101 | CCATTGGACAACCAGCTTCCATCTCTTGCAAGTCAAGTCAGAGCCTCTTA |
| 151 | TATACTAATGGAAAAACCTATTTGAATTGGTTGTTACAGAGGCCAGGCCA |
| 201 | GTCTCCAAAACGCCTAATCTATCTGGTGTCTAAATTGGACTCTGGAGTCC |
| 251 | CTGACAGGTTCAGTGGCAGTGGATCAGGGACAGATTTCACACTGAAAATC |
| 301 | AGCAGAGTGGAGGCTGAGGATTTGGGAGTTTATTACTGCTTGCAGAGTAC |
| 351 | ACATTTTCCGCTCACGTTCG GTGCTGGGAC CAAGCTGGAG CTGAAA |

### Example 4

### Binding of Murine Monoclonal Antibodies to RON Expressing Tumor Cells by Flow Cytometry

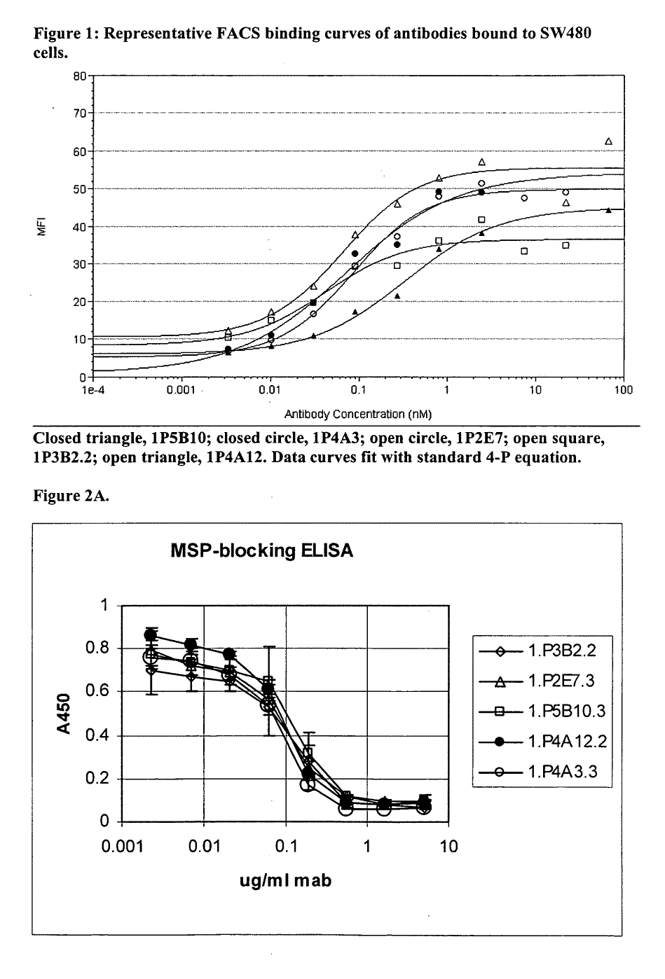

The binding of RON antibodies to RON expressing tumor cells was analyzed by flow cytometry, and an apparent affinity was calculated. Confluent SW480 cells were detached with 5 mM EDTA, washed 1× with FACS buffer (1% FCS, 0.05% sodium azide, 1×PBS) and resuspended at 0.5-1.0×10⁷/ml. The cells were added to a 96-well V bottom polypropylene plate at 100 μl/well. Antibody dilutions were made up in FACS buffer at 2× final concentration and added to the plate at 100 μl/well. The plate was incubated for 1 hour at 4° C., followed by washing 3× with FACS buffer. The cell pellets were then resuspended in 150 μl of PE labeled goat anti-murine IgG H & L secondary antibody, diluted 1/200, and incubated for 1 hour at 4° C. The cells were then washed 1× with FACS buffer and fixed with 150 μl 3% formaldehyde at room temperature for 10 minutes. The cell pellets were resuspended in 150 μl FACS buffer and analyzed by FACS. Representative FACS titrations of antibodies 1P5B10, 1P4A3, 1P2E7, 1P3B2 and 1P4A12 on SW480-RON cells are shown in FIG. 1. The EC50 of binding was calculated from the binding curves using a standard 4P fit equation. The apparent affinity for several antibodies is shown in Table 5. These data indicate that the antibodies bind to human RON.

TABLE 5
Apparent affinity calculated from antibody binding to tumor
cells by FACS using a standard 4P fit equation (see FIG. 1)

| subclone | EC50 nM |
|---|---|
| 1P2E7 | 0.09 |
| 1P3B2 | 0.04 |
| 1P4A3 | 0.07 |
| 1P4A12 | 0.07 |
| 1P5B10 | 0.34 |

### Example 5

### Anti-RON Antibodies Block MSP-RON Binding

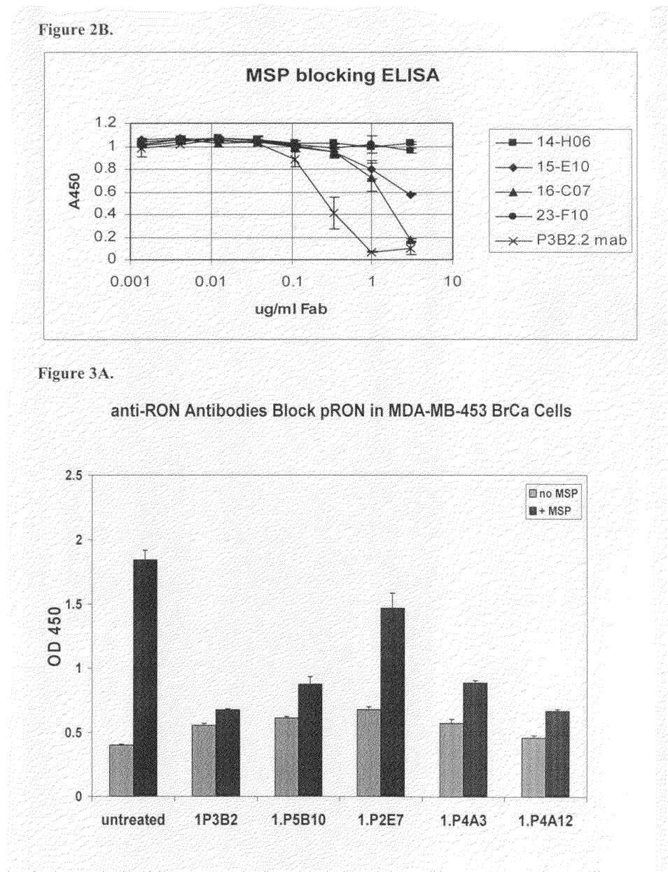

The ability of the antibodies or Fabs to block MSP binding to RON was measured using standard ELISA protocol. Briefly, soluble RON protein (R&D Systems, catalog 1947) was coated on Nunc Maxisorb ELISA plates at a concentration of 1 μg/ml in PBS overnight at 4° C. Plates were washed in PBS, 0.05% Tween 20 4 times with an automatic washer. Plates were then blocked in PBS+1% protease-free, IgG-free BSA (Jacksan Labs 001-000-162) for 1-2 hours at room temperature (RT). After washing, serial dilutions of the antibody or Fab in blocking buffer were added to the plate (200 μl/well). MSP (R&D Systems cat. #4306-MS) at a concentration of 200 ng/ml in block was added to the plate (10 μl/well) and the mixtures were incubated for 1-2 hours at RT. The plate was washed again 4× with automatic washer (PBS, 0.05% tween 20). Next, the plate was incubated in biotinylated goat anti-MSP polyclonal (R&D Systems BAF 352) at a concentration of 0.5 μg/ml in block for 0.5-1 hour at RT. After an additional wash step, streptavidin-HRP (DY998, R&D Systems) diluted 1:200 in block was added and incubated for 0.5-1 hour RT. After the final wash step, the plate was developed with TMB solution (25 ml 100 mM sodium acetate, pH to 6 with citric acid, 4 ul 30% H₂0₂, 250 μl 42 mM TMB in DMSO) for 5 minutes, stopped with 100 ul H₂SO₄. Plates were read at 450 nm wavelength on Molecular Devices Spectramax M5, and Softmax pro V5 software was used to analyze the data. The results of the ELISA assays using murine monoclonal antibodies and human Fabs are shown in FIGS. 2A and 2B, respectively, and demonstrate that murine monoclonal antibodies and the human Fabs can block MSP binding to RON.

### Example 6

### Anti-RON Antibodies Block MSP-Dependent RON Activity

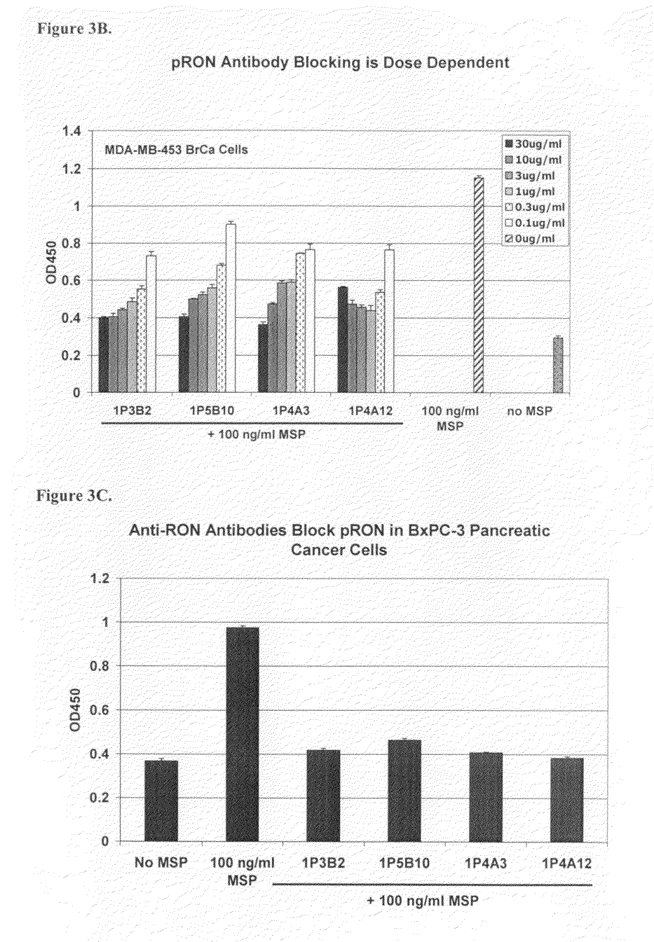

To measure antibody blocking of MSP-induced phosphorylated RON, an ELISA capture method was used in which total RON protein was captured on an ELISA plate, followed by detection with an anti-phospho-tyrosine antibody (pRON DuoSet IC ELISA, R&D Systems, cat# DYC1947). For these experiments, either MDA-MB-453 breast cancer cells (FIGS. 3A-B) or BxPC-3 (FIG. 3C) pancreatic cancer cells were plated in RPMI/10% FCS at approximately 8×10⁵ cells/well in a 6-well tissue culture dish and allowed to adhere overnight. The next day, cells were serum starved for 2.5 hours. Antibodies were then added to the dish at a final concentration of 10 μg/ml (FIGS. 3A and 3C) or at varying concentrations (FIG. 3B) and incubated for 15 minutes, and then MSP was added at a final concentration of 100 ng/ml for an additional 15 minutes. Plates were then harvested according to manufacturer's protocol. The results of the anti-phospho-tyrosine antibody detection are shown in FIGS. 3A-C and demonstrate that murine monoclonal antibodies block MSP-induced RON phosphorylation in MDA-MB-453 breast cancer cells, there is a dose-dependent blockade of RON phosphorylation in MDA-MB-453 cells, and the antibodies block MSP-induced RON phosphorylation in BxPC-3 pancreatic cancer cells.

### Example 7

### Anti-RON Antibodies Block MSP-Independent RON Activity

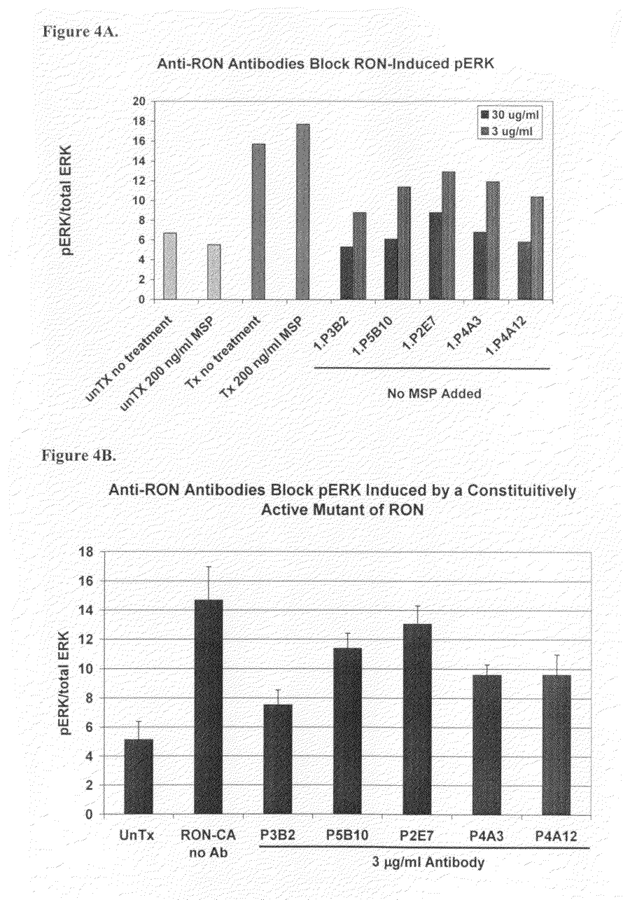

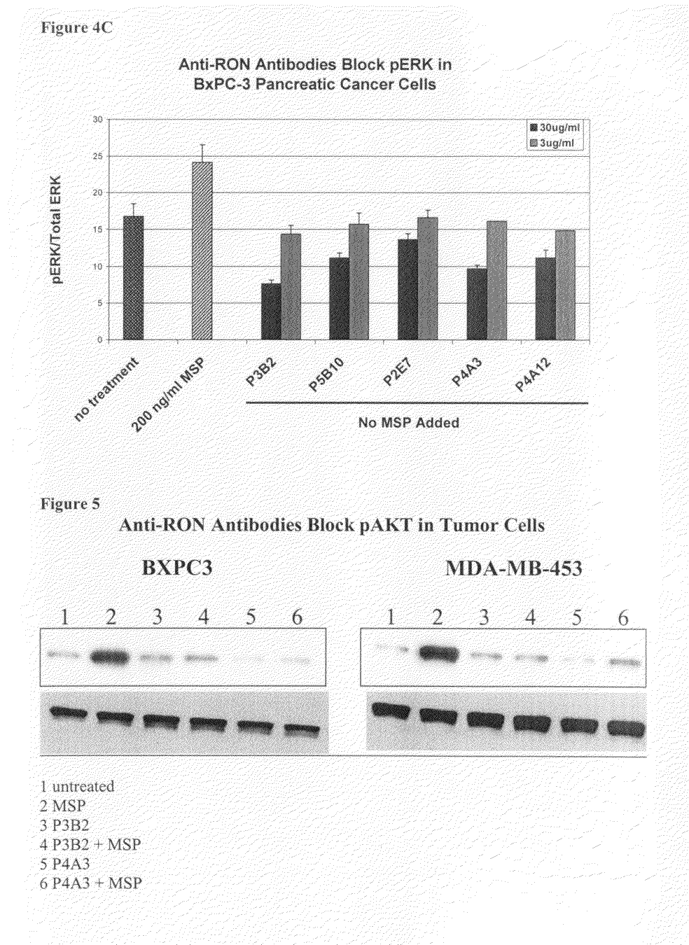

To measure the blocking potential of anti-RON antibodies on MSP-independent signaling, a pERK ELISA assay was used (PERK 1/2 Immunoassay, R&D Systems cat #KCB1018). Phospho-ERK is a known downstream signal mediator of the RON pathway. 293E cells were plated for transfection (3×10⁶ cells in a 10 cm dish) and allowed to adhere overnight in DMEM/10% FBS. On day 2, cells were transfected with empty vector, plasmid encoding wildtype RON (FIG. 4A), or a plasmid encoding RONCA (constitutively active M1254T kinase domain mutant; FIG. 4B). On day 3, transfected cells were plated in black/clear bottom 96-well tissue culture plate and treated with antibodies at 30 or 3 μg/ml (or MSP at 200 ng/ml) in serum-free media. On days 4 and 5, the plates were processed according to manufacturer's instructions. Results for wildtype RON and constitutively active RON are shown in FIGS. 4A and 4B, respectively, and demonstrate that the murine monoclonal antibodies prevent phosphorylation of pERK. These data indicate that the antibodies block MSP-independent RON signaling.

Similar experiments were performed using BxPC-3 pancreatic cancer cells. In these experiments, the cells were plated directly into the black/clear-bottom 96-well plates, and no transfection steps were preformed. The results are shown in FIG. 4C. Similar blocking activity of the antibodies was observed with MSP added to the wells with the antibodies (data not shown).

### Example 8

### Anti-RON Antibodies Block pAKT in Tumor Cells

The blocking potential of anti-RON antibodies on pAKT signaling in tumor cells was evaluated using two tumor cell lines: BXPC3 and MDA-MB-453. In these experiments, one million cells/well were plated in a 6 well dish in the morning then serum starved overnight for 18 hours in DMEM+1% BSA. Media was removed and anti-RON antibodies were added at 10 ug/ml in DMEM. Cells were incubated at 37° C. for 30 minutes. MSP was added to a final concentration of 200 ng/ml and incubated for 30 minutes at 37° C. Cell lysates were prepared and 15 ug was run out on a 10% tris-glycine gel and transferred to nitrocellulose. The western blot was probed with Phospho-AKT (Ser473) antibody (Cell Signalling #9271) (FIG. 5, top panel) and then stripped and reprobed with AKT antibody (Cell Signalling #9272) (FIG. 5, bottom panel). Results obtained using antibodies 1P3B2 and 1P4A3 are shown in FIG. 5 and demonstrate that murine monoclonal antibodies prevent phosphorylation of pAKTin tumor cells.

### Example 9

### Anti-RON Antibodies Bind to RON Splice Variant

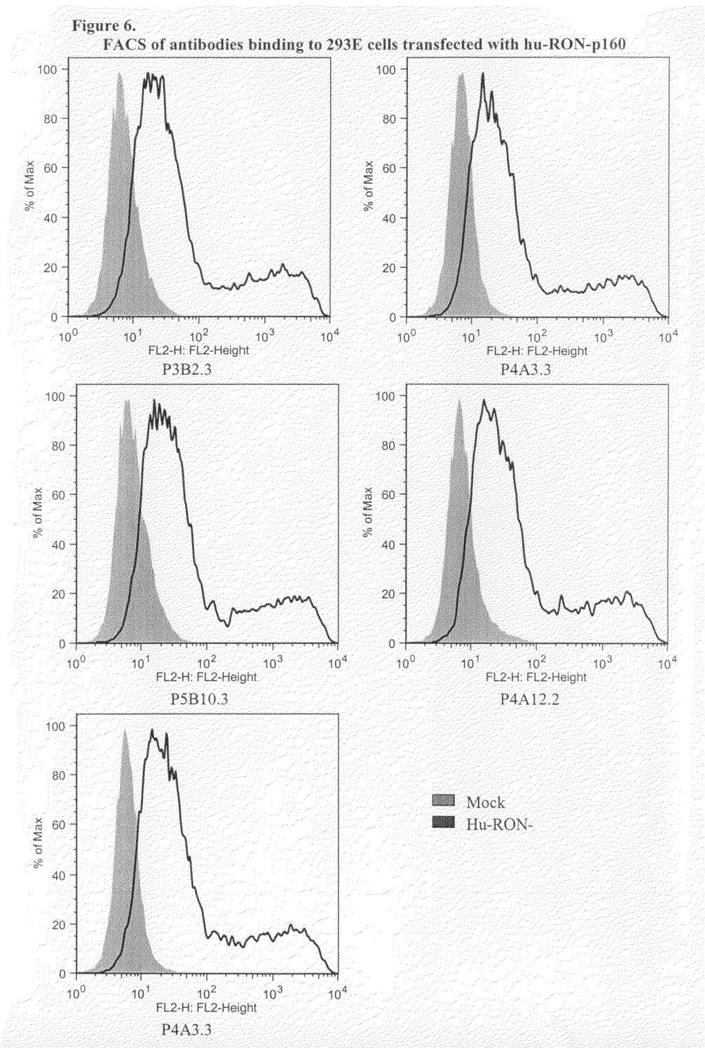

In order to determine if anti-RON antibodies bind to the human RON splice variant RONdelta160, 293E cells were transfected with Hu-RON-P160 (MZ236) construct or vector alone (mock). Twenty-four hours post-transfection, cells were removed from the plate with PBS-5 mM EDTA and stained with antibodies at concentration of 5 ug/ml, followed by anti-mouse secondary antibody conjugated to PE and analyzed by FACS. The results of the FACS analysis are shown in FIG. 6 and demonstrate that murine monoclonal antibodies bind to RONdelta160.

### Example 10

### Anti-RON Antibodies Bind to Soluble Antigen with High Affinity

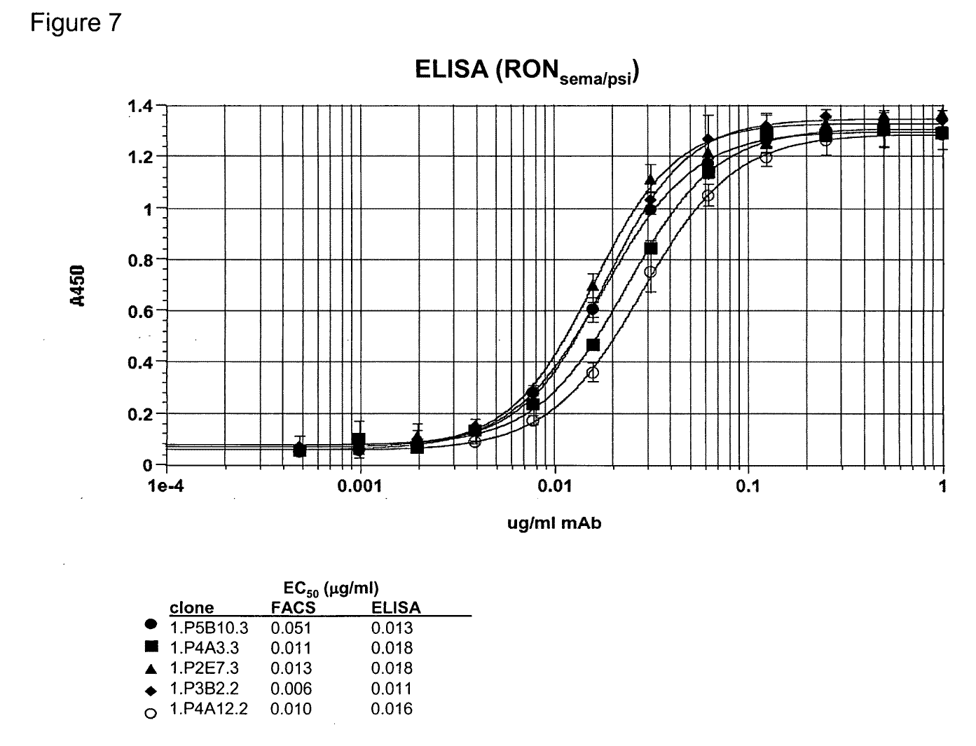

The ability of the antibodies to bind to soluble RON was measured using a standard ELISA protocol. Briefly, soluble RON protein (R&D Systems, catalog 1947) was coated on Nunc Maxisorb ELISA plates at a concentration of 1 mg/ml in PBS overnight at 4° C. Plates were washed 4 times with an automatic washer in PBS, 0.05% Tween 20. Plates were then blocked in PBS+1% BSA for 1-2 hours at room temperature (RT). After washing, serial dilutions of the antibodies in blocking buffer were added to the plate (100 ul/well) and incubated for 1-2 hours at RT. The plate was washed again 4× with automatic washer (PBS, 0.05% tween 20). Next, the plate was incubated with HRP-labelled donkey anti-mouse polyclonal antibody (Jackson Labs 715-035-130) at a dilution of 1:4000 in blocking buffer (100 ul/well) for 0.5-1 hour RT. The plate was washed again 4× with automatic washer (PBS, 0.05% Tween 20). After the final wash step, the plate was developed with TMB solution (25 ml 100 mM sodium acetate, pH to 6 with citric acid, 4 ul 30% H202, 250 ul 42 mM TMB in DMSO) for 5 minutes. The reaction was stopped with 100 ul H₂SO₄. Plates were read at 450 nm wavelength on Molecular Devices Spectramax M5 and Softmax pro V5 software was used to analyze the data. The results are shown in FIG. 7 and demonstrate that the antibodies bind soluble RON with high affinity.

### Example 11

### Anti-RON Antibodies Bind RON-Expressing Cells

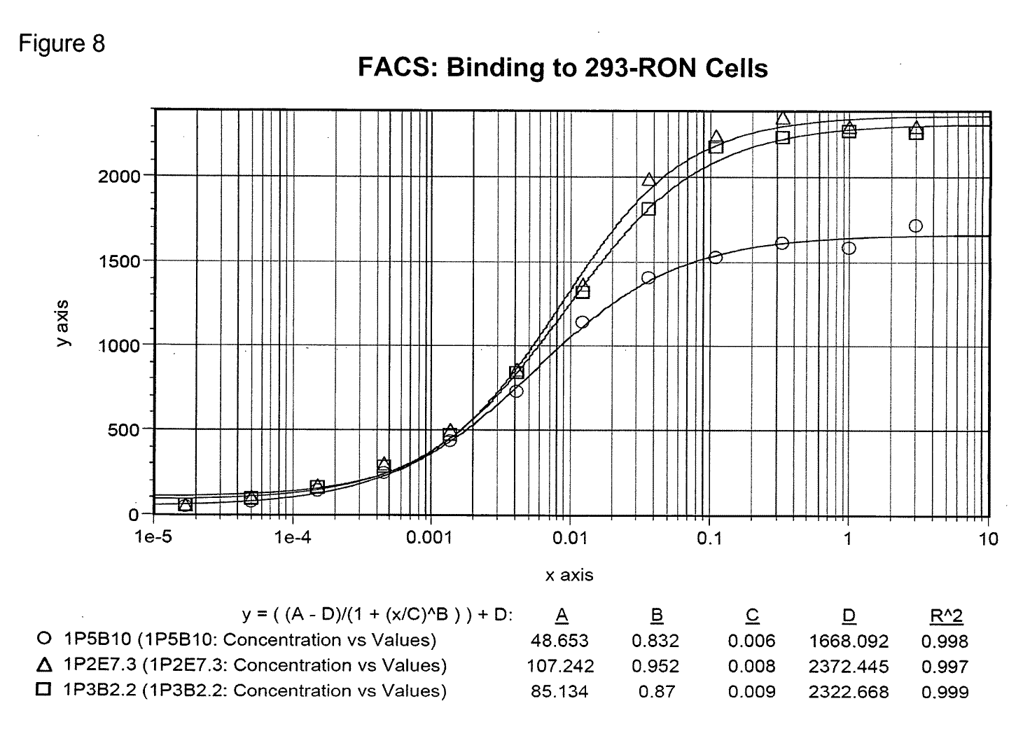

The binding of RON antibodies to RON expressing 293E cells was analyzed by flow cytometry, and an apparent affinity was calculated. Confluent 293E/human RON cells were detached with trypsin and resuspended at 0.6×10⁷/ml with FACS buffer (1% FCS, 0.05% sodium azide, 1×PBS). The cells were added to a 96-well round bottom polypropylene plate at 50 μl/well. Antibody dilutions were made in FACS buffer at 2× final concentration and added to the plate at 50 μl/well. The plate was incubated for 1 hour at 4° C., followed by washing 3× with FACS buffer. The cell pellets were then resuspended in 100 μl of phycoerythrin (PE) Fab′2 fragment goat anti-mouse IgG Fcg secondary antibody (diluted 1/200) and incubated for 1 hour at 4° C. in the dark. The cells were then washed 3× with FACS buffer and fixed with 200 ul 1% paraformaldehyde in PBS and analyzed by FACS. The EC50 of binding was calculated from the binding curves. The results are shown in FIG. 8 and demonstrate that the antibodies bind RON-expressing 293 cells with high affinity.

### Example 12

### Anti-RON Antibodies Block MSP Binding to RON-Expressing Cells

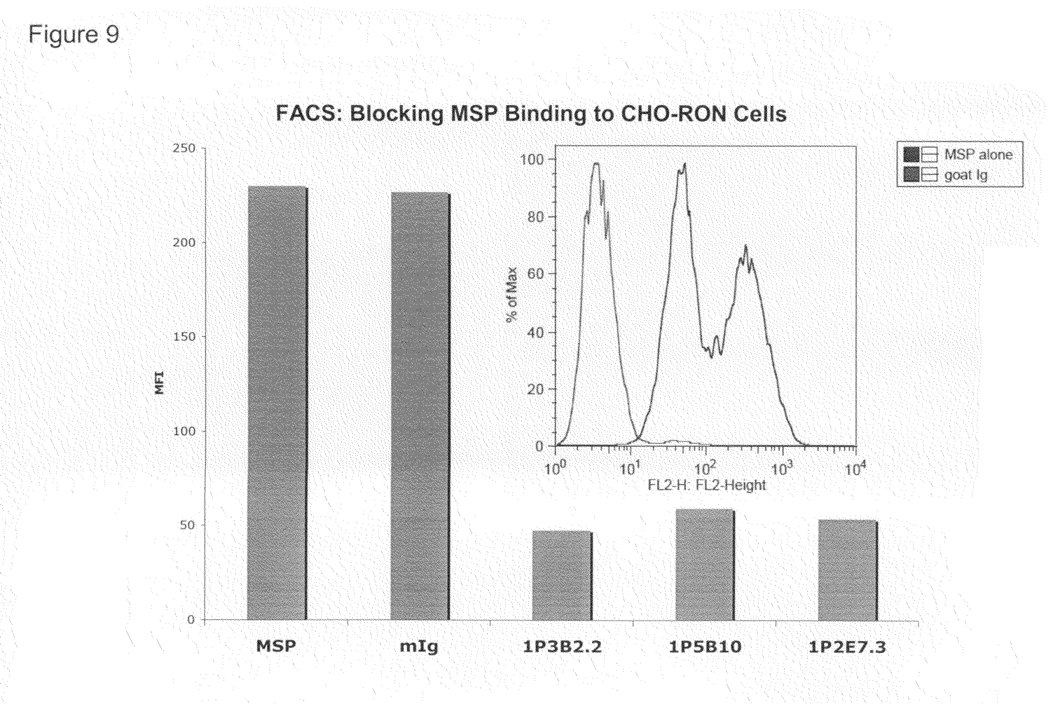

The ability of RON antibodies to block MSP binding to RON-expressing CHO cells was analyzed by flow cytometry. Confluent CHO/human RON cells were detached with trypsin and resuspended at 0.6×10⁷/ml with FACS buffer (1% FCS, 0.05% sodium azide, 1×PBS). The cells were added to a 96-well round bottom polypropylene plate at 50 ul/well. Antibody dilutions were made up in FACS buffer at 2× final concentration, and added to the plate at 50 ul/well. The plate was incubated for 1 hour at 4° C., followed by washing 1× with FACS buffer. MSP (1 ug/ml) was added to the cell pellets in a volume of 50 ul/well and incubated for 30 minutes at room temperature, followed by washing 1× with FACS buffer. Goat anti human MSP (10 ug/ml) was added to the cell pellets for 30 minutes at room temperature, followed by washing 2× in FACs buffer. The cell pellets were then resuspended in 100 ul of PE Fab′2 fragment donkey anti goat IgG (H+L) secondary antibody (diluted 1/200) and incubated for 30 minutes at room temperature in the dark, followed by washing 2× in FACs buffer. The cells were then fixed with 200 ul 1% paraformaldehyde in PBS and analyzed by FACS. The results are shown in FIG. 9 and demonstrate that anti-RON antibodies decreased MSP binding to CHO cells expressing RON protein.

### Example 13

### Anti-RON Antibodies Block MSP-Induced pERK and pAKT Signaling Tumor Cells

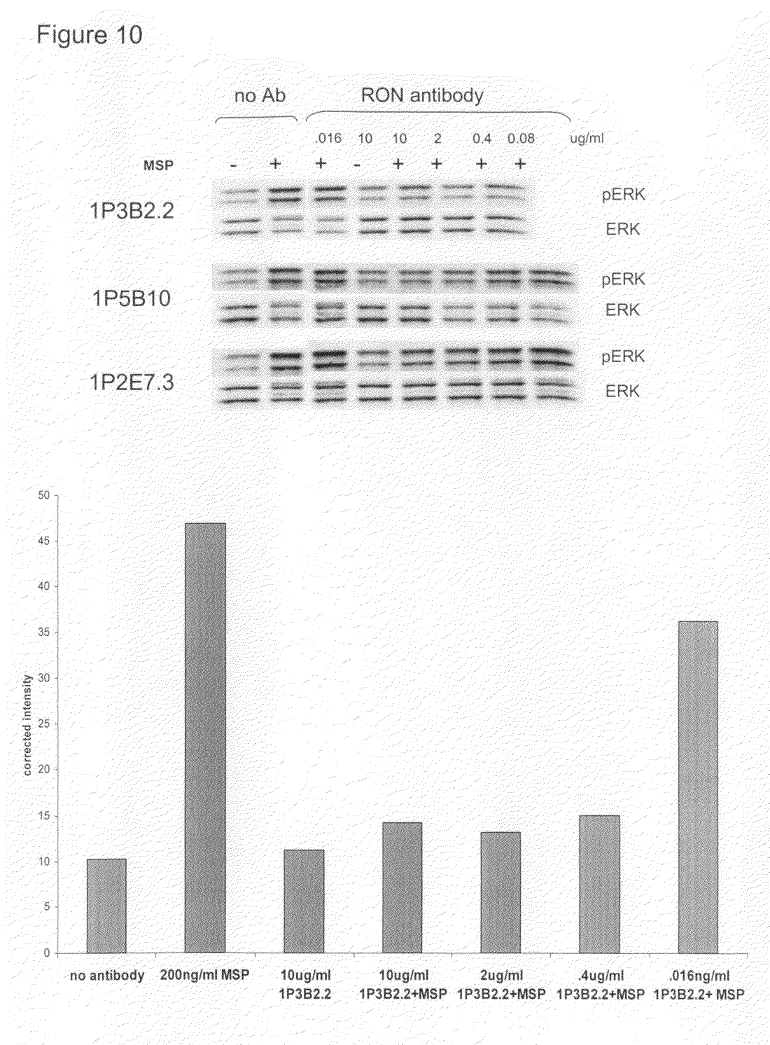

To measure antibody blocking of MSP-induced pERK and pAKT signaling, a two-color infrared fluorescent Western blot method was used. For these experiments cells were plated in complete media/10% FBS at approximately 5×10⁵ cells/well in a 12-well tissue culture dish and allowed to adhere overnight. The next day, cells were serum starved for 3 hours in DMEM. Antibodies were then added to the dish at the concentrations indicated in FIG. 10 and incubated for 15 minutes. MSP was then added at a final concentration of 200 ng/ml and cells were incubated for an additional 15 minutes. Total cell lysates were prepared and a 20 ug sample was analyzed on a 10% tris-glycine gel and transferred to nitrocellulose.

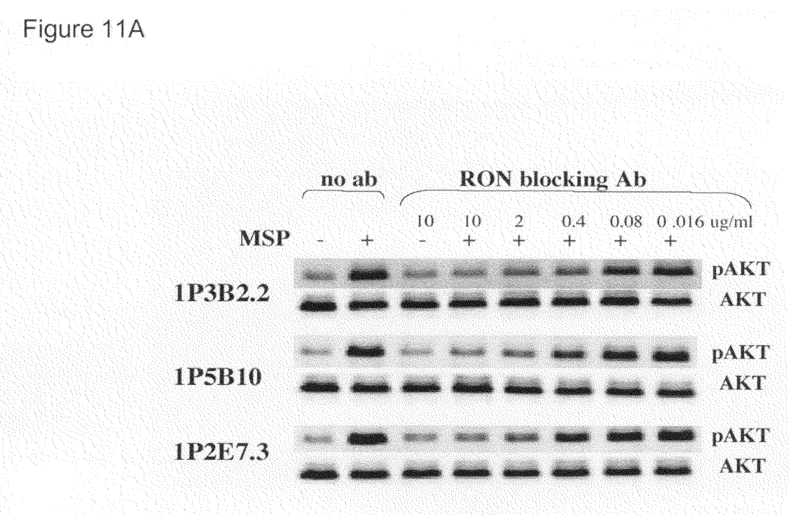

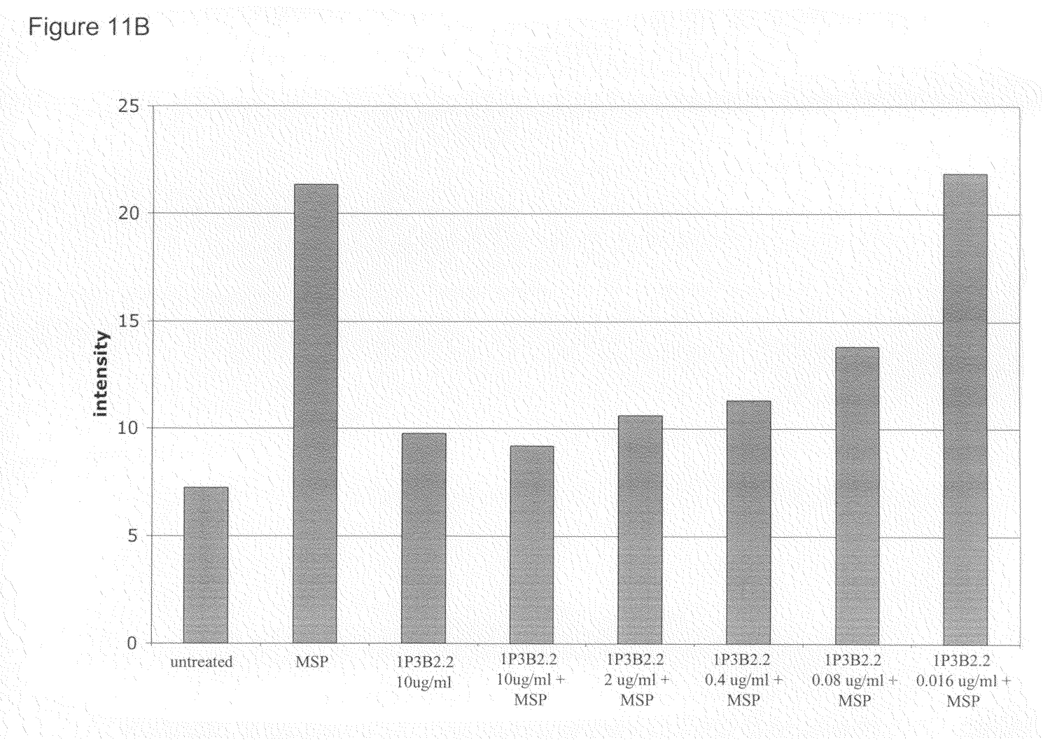

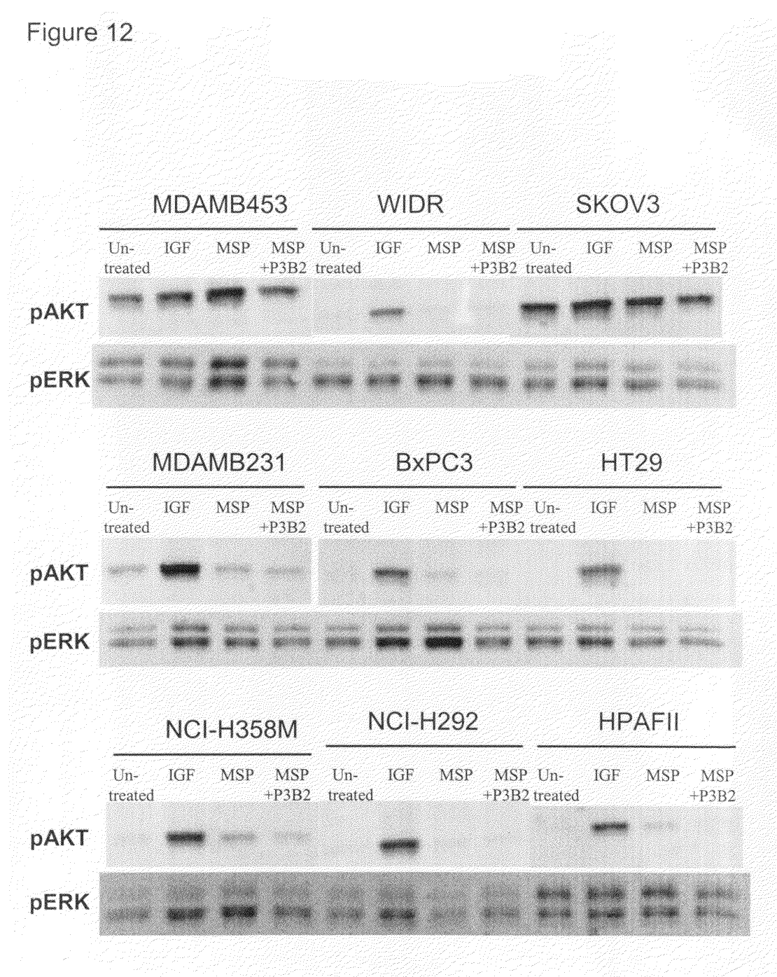

In order to assess phospho-ERK levels, blots were probed simultaneously with phospho-p44/42 Map Kinase (Thr 202/Tyr 204) rabbit antibody (Cell Signalling #9101) and p44/42 Map Kinase (L34F12) mouse mAb (Cell Signalling #4696). In order to assess phospho-AKT levels, blots were probed simultaneously with phospho-AKT (Ser473) (587F11) mouse mAb (Cell Signalling #9271) and AKT rabbit antibody (Cell Signalling #9272). IR Dye labeled secondary antibodies were used for detection in both pERK and pAKT blots, and the blots were scanned using the Odyssey imager (Li-cor Biosciences). The results, as shown in FIGS. 10 and 11, demonstrate that anti-RON antibodies decrease phoso-ERK and phospho-AKT levels. This indicates that anti-RON antibodies can block ERK and AKT signaling in MDA-MB-453 cells. These experiments were also performed in other tumor cell types, and as shown in FIG. 12, anti-RON antibodies also decreased ERK and AKT signaling in other tumor cells.

### Example 14

### Human MSP Binds Soluble Human and Cyno RON

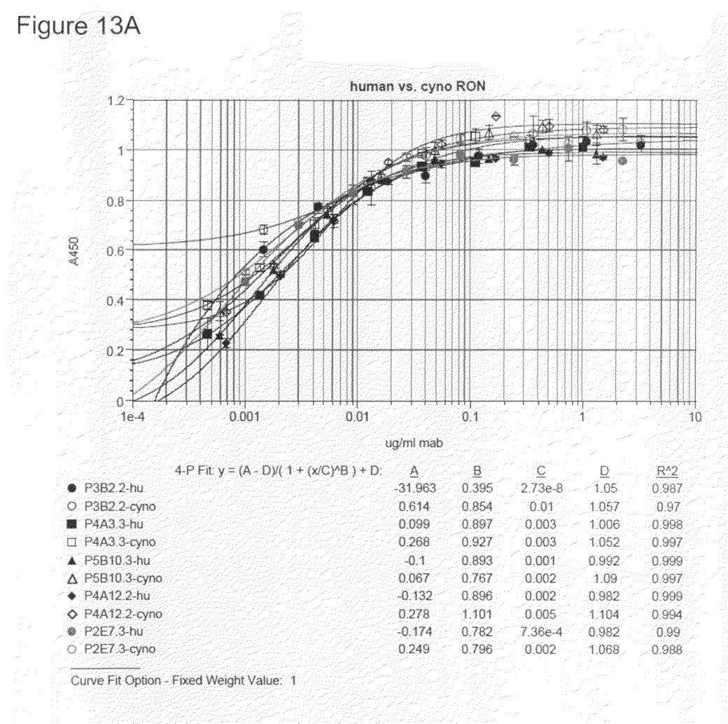

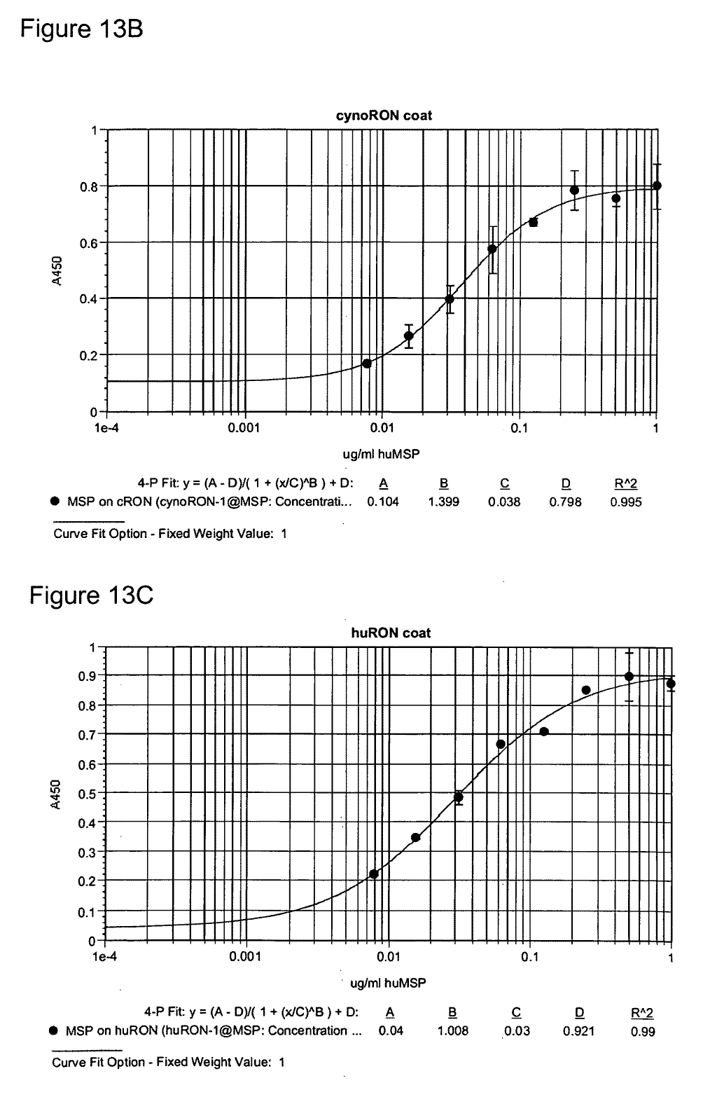

The ability of a human MSP (R&D systems cat. #4306) to bind soluble human and cynomolgus (Macaca fascicularis) RON (“cyno RON”) was measured using standard ELISA protocol. Briefly, soluble human RON protein (R&D Systems, catalog 1947) or soluble cyno RON protein was coated on Nunc Maxisorb ELISA plates at a concentration of 1 mg/ml in PBS overnight at 4° C. For cyno RON ELISAs, the corresponding protein sequence from cyno RON was produced and purified from CHO cells. Plates were washed 4 times with an automatic washer in PBS, 0.05% Tween 20. Plates were then blocked in PBS+1% protease-free, IgG-free BSA (Jacksan Labs 001-000-162) for 1-2 hours at room temperature (RT). After washing, serial dilutions of MSP (R&D Systems cat. #4306-MS) in blocking buffer were added to the plate (100 ml/well) and incubated for 1-2 hours at RT. The plate was washed again 4× with automatic washer (PBS, 0.05% Tween 20). Next, the plate was incubated in biotinylated goat anti-MSP polyclonal (R&D Systems BAF 352) antibody at a concentration of 0.5 mg/ml in blocking buffer for 0.5-1 hour RT. After an additional wash step, streptavidin-HRP (DY998, R&D Systems) diluted 1:200 in blocking buffer was added and incubated for 0.5-1 hour at RT. After the final wash step, the plate was developed with TMB solution (25 ml 100 mM sodium acetate, pH to 6 with citric acid, 4 ul 30% H₂0₂, 250 ul 42 mM TMB in DMSO) for 5 minutes and stopped with 100 ul H₂SO₄. Plates were read at 450 nm wavelength on a Molecular Devices Spectramax M5, and Softmax pro V5 software was used to analyze the data. As shown in FIGS. 13B and C, MSP binds to both cynoRON and human RON. FIG. 13A demonstrates that anti-RON antibodies bind to cynoRON as well as to human RON. The antibody binding experiments were performed as described in Example 10.

### Example 15

### Anti-RON Antibodies Block Invasion of Cells

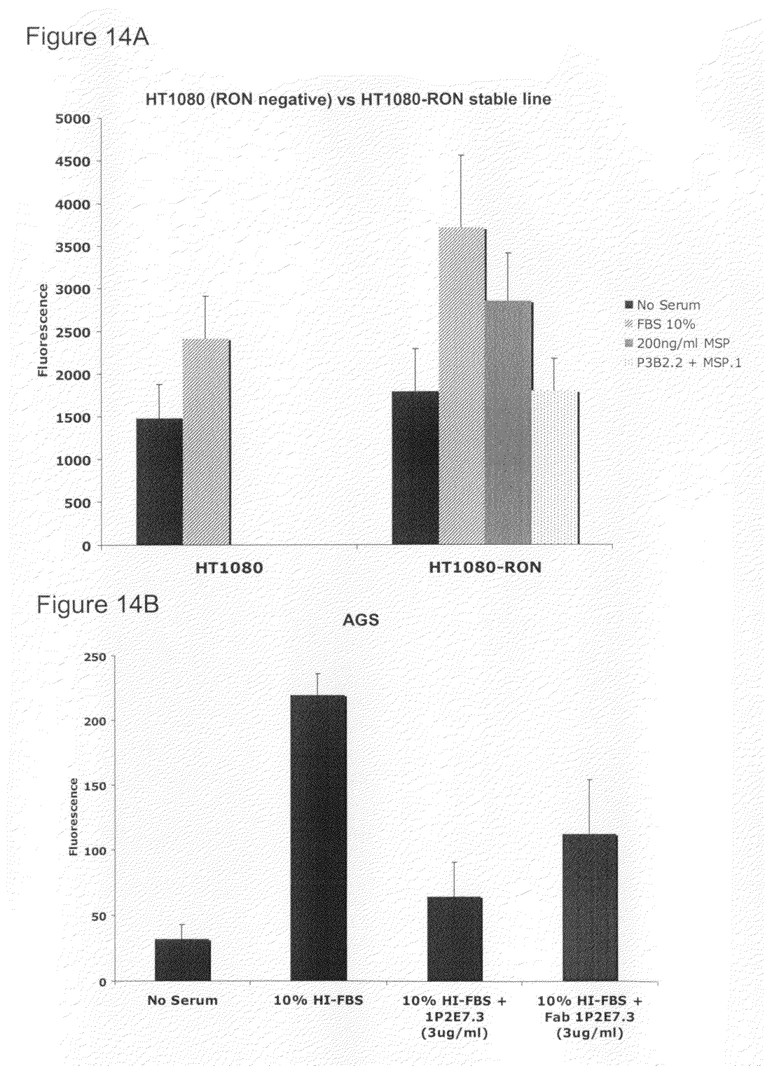

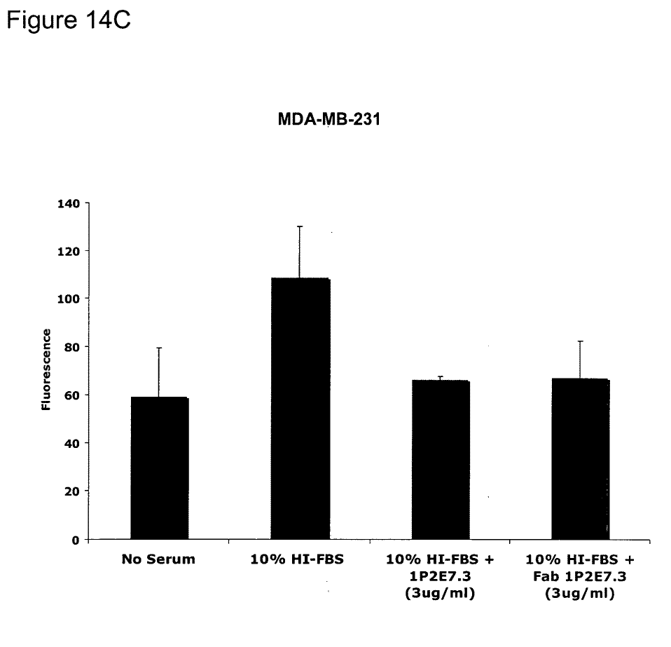

To measure antibody blocking of fetal bovine serum (FBS)-induced or MSP-induced invasion of tumor cells, an 8.0-micron matrigel coated Fluoroblok™ transwell insert was used (BD catalog #354166). Briefly, matrigel coated inserts were rehydrated with 500 ul PBS for 2 hours at 37° C. in a non-CO₂ environment. After rehydration, PBS was removed. Tumor cells that had been serum starved were detached with trypsin and resuspended in serum free media at 5×10⁴ cells/ml. Tumor cells were then pre-incubated with antibodies for 30 minutes prior to addition to the assay. Next, 750 ul of chemoattractant (10% Heat Inactivated-FBS or 200 ng/ml MSP) was added to the bottom chamber and 500 ul of cells +/−antibodies were added to the top chamber. The cells and chemoattractant were incubated for 48 hours at 37° C. in 5% CO₂. Following incubation, medium was removed from the top chambers. The insert was then transferred to a second 24 well plate containing 500 ul/well of 4 ug/ml calcein acetoxymethyl ester (AM) in Hanks Balanced Salt Solution (HBSS) and incubated for 1 hour at 37° C. 5% CO₂. Fluorescence of invaded cells was read at wavelengths of 494/517 nm (Ex/Em) using a TECAN GENios Pro fluorescence plate reader. The results are shown in FIG. 14 and demonstrate that anti-RON antibodies can block invasion of tumor cells.

### Example 16

### Mapping Anti-RON Antibodies

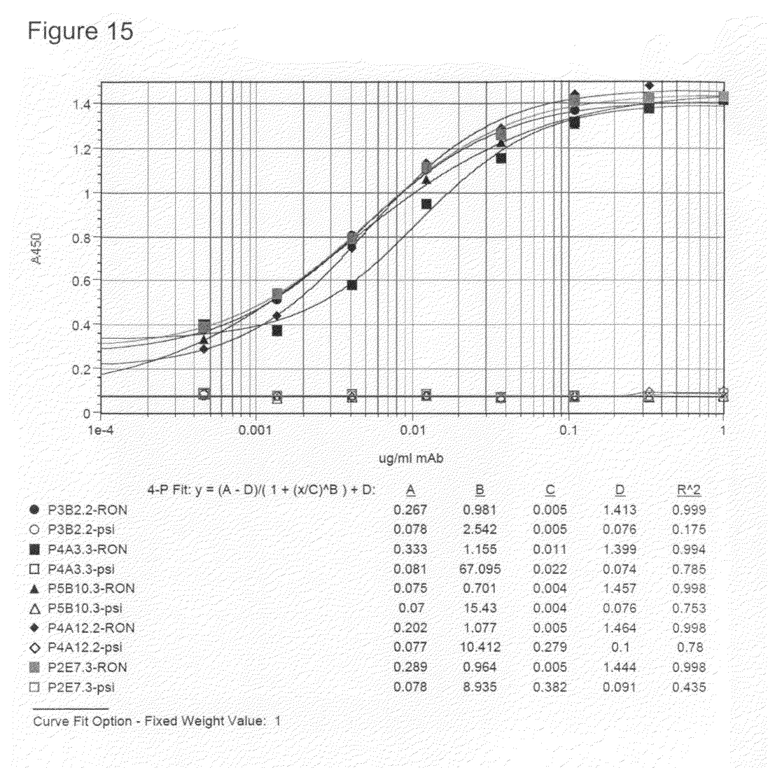

The PSI domain of RON (G502-P544) was expressed in E. coli as a thiroedexin-fusion protein. The protein was purified on NiNTA agarose (qiagen) followed by preparative size exclusion chromatography (SEC). This protein was used to coat ELISA plates and analyze antibodies for binding using the same protocol described above for binding of antibodies to soluble RON in Example 10. The results shown in FIG. 15 demonstrate that while anti-RON antibodies bind to a soluble RON protein containing the SEMA and PSI domains, the antibodies did not bind to the PSI domain alone.

### Example 17

### Anti-Murine RON Antibodies Bind to Mouse RON on 293 Cells

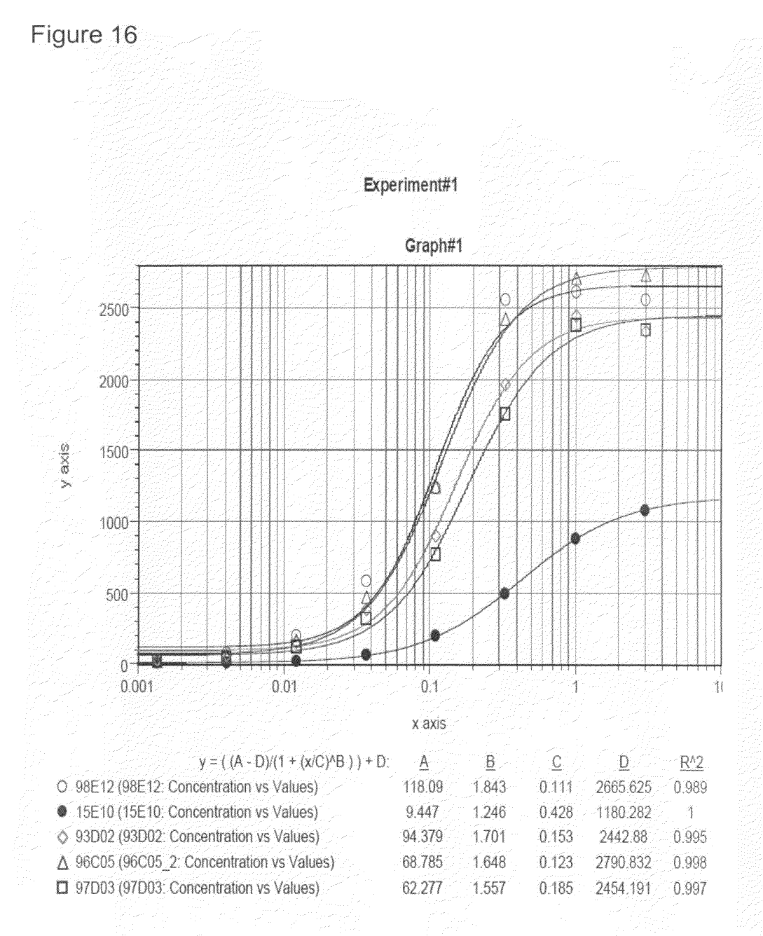

The binding of RON antibodies to murine RON-expressing 293E cells was analyzed by flow cytometry, and an apparent affinity was calculated. Confluent 293E/murine RON cells were detached with trypsin and resuspended at 0.6×10⁷/ml with FACS buffer (1% FCS, 0.05% sodium azide, 1×PBS). The cells were added to a 96-well round bottom polypropylene plate at 50 ul/well. Antibody dilutions were made up in FACS buffer at 2× final concentration, and added to the plate at 50 ul/well. The plate was incubated for 1 hour at 4° C., followed by washing 3× with FACS buffer. The cell pellets were then resuspended in 100 ul of PE Fab′2 fragment goat anti-mouse IgG Fcg secondary antibody (diluted 1/200 ), and incubated for 1 hour at 4° C. in the dark. The cells were then washed 3× with FACS buffer and fixed with 200 ul 1% paraformaldehyde in PBS and analyzed by FACS. The EC50 of binding was calculated from the binding curves. The data is shown in FIG. 16 and demonstrates that the antibodies bind to cells expressing murine RON with high affinity.

### Example 18

### Anti-Murine RON Antibodies Block pAKT Signaling

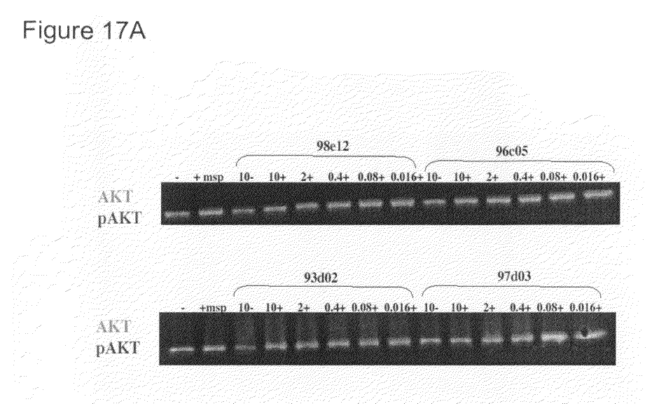

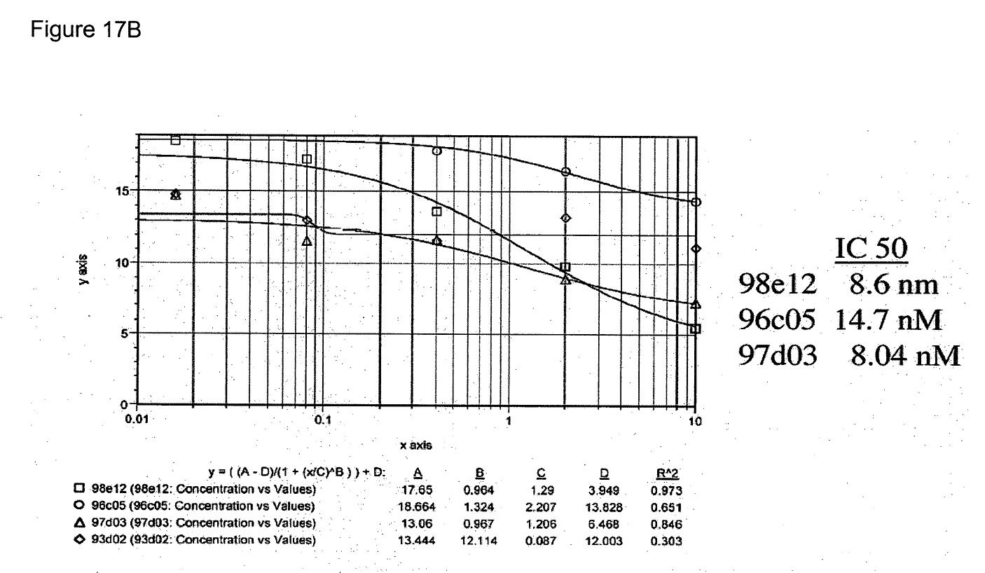

In order to determine if anti-murine RON antibodies could also block RON signaling, pAKT signaling was measured in a CHO cell line expressing murine RON. Cells were cultured in the presence or absence of MSP and in varying concentrations of anti-murine RON antibodies. Levels of phosphorylated AKT were assessed using the protocol described in Example 13. The results are shown in FIG. 17. The decreased levels of phospho-AKT in the presence of the RON antibodies indicates that anti-murine RON antibodies block pAKT signaling.

### Example 19

### Anti-Murine RON Antibodies Bind Human RON Weakly

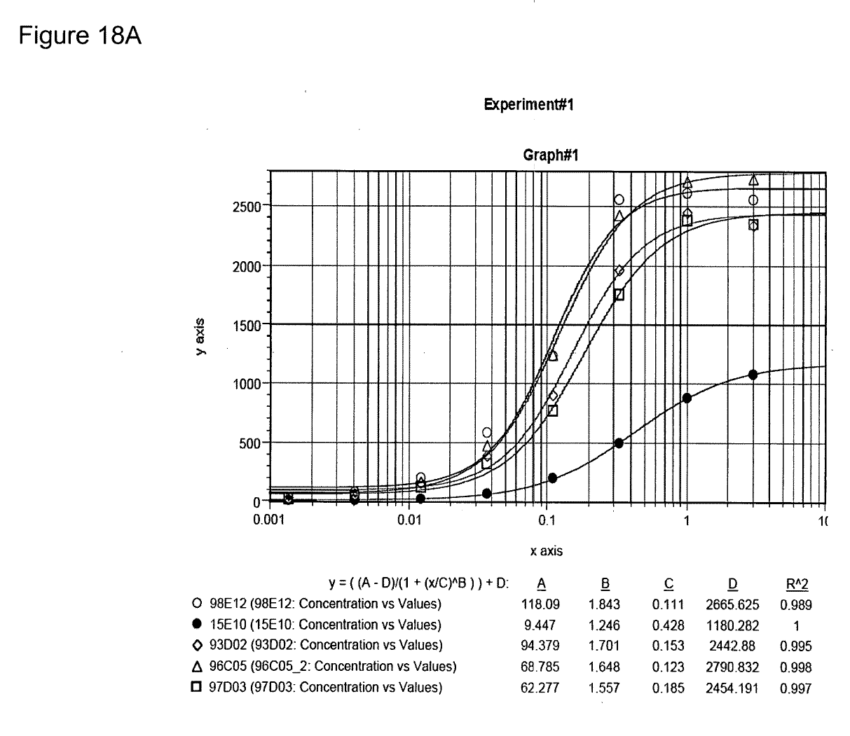

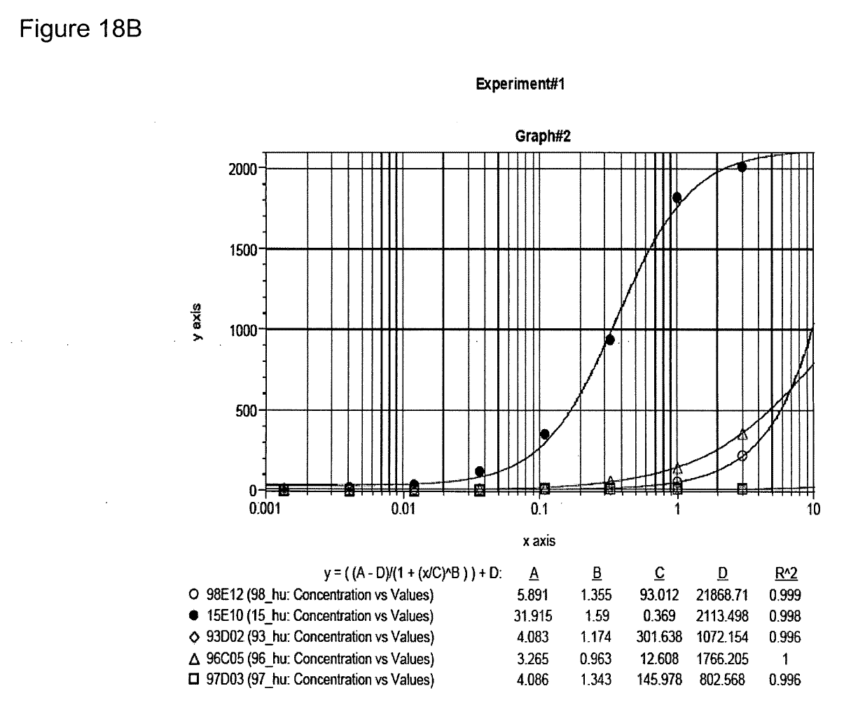

In order to determine if anti-murine RON antibodies also bind to human RON, murine and human RON proteins were expressed in 293 cells. Binding of antibodies to the RON-expressing cells was determined using FACS analysis as described above in Examples 8 and 16. The results shown in FIG. 18 demonstrate that anti-murine RON antibodies bind to both murine and human RON proteins but have a greater affinity for murine RON.

### Example 20

### RON is Expressed and Signals on Multiple Human Tumor Cell Lines

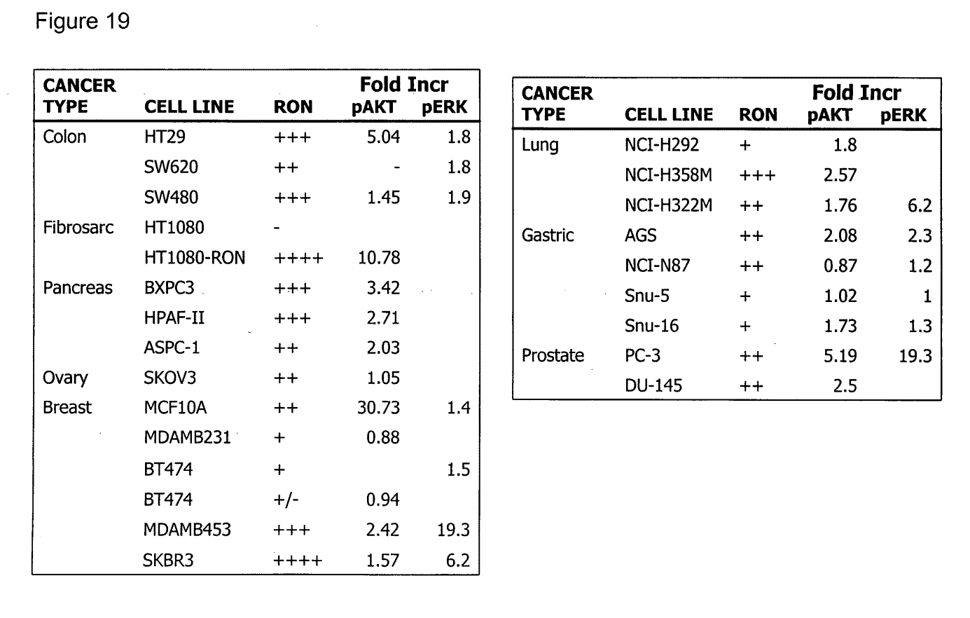

The binding of RON antibodies to RON expressing tumors was analyzed by flow cytometry. In these experiments, confluent tumor cells were detached with trypsin and resuspended at 0.6×10⁷/ml with FACS buffer (1% FCS, 0.05% sodium azide, 1×PBS). The cells were added to a 96-well round bottom polypropylene plate at 50 ul/well. Antibody dilutions were made up in FACS buffer at 2× final concentration, and added to the plate at 50 ul/well. The plate was incubated for 1 hour at 4° C., followed by washing 2× with FACS buffer. The cell pellets were then resuspended in 100 ul of PE Fab′2 fragment goat anti-mouse IgG Fcg secondary antibody (diluted 1/200) and incubated for 30 minutes at room temperature in the dark. The cells were then washed 2× with FACS buffer and fixed with 200 ul 1% paraformaldehyde in PBS and analyzed by FACS. The relative level of RON expression on the cells was determined from the MFI. The results are summarized in the Table shown in FIG. 19. Most of the human tumor cell lines tested expressed RON. In addition, experiments to determine the levels of MSP-induced pAKT and pERK were performed as described in Example 13. FIG. 19 shows the fold increase of MSP-induced pAKT and pERK relative to pAKT and pERK in untreated samples. High levels of MSP-induced pAKT and pERK indicate that RON signals in many of these tumor cell lines.

### Example 21

### Anti-RON Antibodies Slow Tumor Growth

In order to determine the effect of anti-RON antibodies on tumor growth, the effect of an anti-human RON antibody on a breast tumor model was observed. MDA-MB-231 cells (ATCC) breast cancer cells were maintained at 37° C. in a 5% CO₂ environment and cultured without antibiotics in RPMI-1640 media containing 10% FBS. 7-week old female CB17 SCID mice (CB17-Prkdcscid/NCrCrl) were inoculated subcutaneously (SC) into the right flank with 5×10⁶ cells suspended in 200 μl of media (without serum) mixed 1:1 with Matrigel matrix (BD). Body weights and tumor measurements (length (L) and width (W)) were recorded at least twice a week. Tumor volume was calculated using the formula: L×W²/2=mm³. When the majority of tumors reached 150-300 mm³ (Day 29) mice were assigned to treatment and control groups. Tumors were size-matched across groups. Beginning on Day 29, 1P3B2 was administered at 40 mg/kg via intraperitoneal (IP) injection, twice a week for a total of 13 doses. Sterile saline (0.9%) was administered IP (10 ml/kg) to the control group on the same schedule. Additionally, a citrate vehicle (10 mM citrate buffer, pH 5.5, 135 mM sodium chloride) was administered intravenously (IV) (10 ml/kg) to the control group on Day 29 and on Day 50. Dosing of the control group modeled the most stringent of the dosing regimens used in the study and reflects the regimens of other groups contained in the study that are not referenced here.

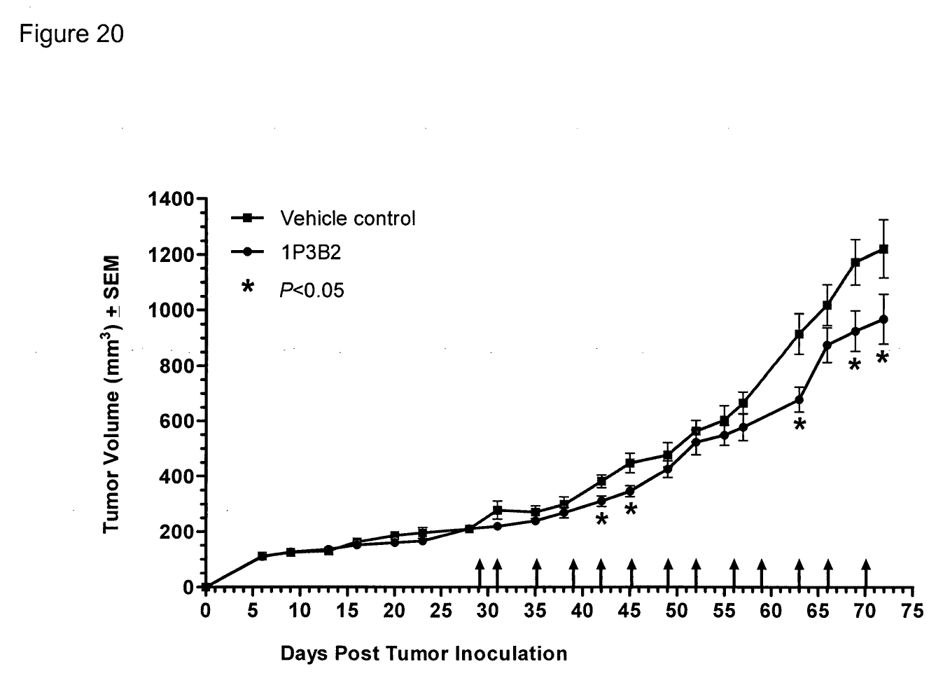

At each data point, the Student's T-test (one-way, two-tail) was used to determine statistical significance of the difference in mean tumor volume of the 1P3B2 test group compared to the mean tumor volume of the control group. The results are graphed in FIG. 20 and show that the tumors in 1P3B2-treated mice were significantly smaller than those in mice treated with saline. This data demonstrates that anti-human RON antibodies decrease tumor growth.

1. An isolated antibody or antigen-binding fragment thereof which specifically binds to the same RON epitope as a reference monoclonal Fab antibody fragment selected from the group consisting of M14-H06, M15-E10, M16-C07, M23-F10, M80-B03, M93-D02, M96-C05, M97-D03, and M98-E12 or a reference monoclonal antibody selected from the group consisting of 1P2E7, 1P3B2, 1P4A3, 1P4A12 and 1P5B10.

2. An isolated antibody or antigen-binding fragment thereof which specifically binds to RON, wherein said antibody or fragment thereof competitively inhibits a reference monoclonal Fab antibody fragment selected from the group consisting of M14-H06, M15-E10, M16-C07, M23-F10, M80-B03, M93-D02, M96-C05, M97-D03, and M98-E12 or a reference monoclonal antibody selected from the group consisting of 1P2E7, 1P3B2, 1P4A3, 1P4A12 and 1P5B10 from binding to RON.

3. The isolated antibody or antigen-binding fragment thereof of claim 1 which specifically binds to RON, wherein said antibody or fragment thereof comprises an antigen binding domain identical to that of a monoclonal Fab antibody fragment selected from the group consisting of M14-H06, M15-E10, M16-C07, M23-F10, M80-B03, M93-D02, M96-C05, M97-D03, and M98-E12 or a reference monoclonal antibody selected from the group consisting of 1P2E7, 1P3B2, 1P4A3, 1P4A12 and 1P5B10.

4-10. (canceled)

11. The isolated antibody or fragment thereof of claim 1 which specifically binds to RON, wherein the VH and VL of said antibody or fragment thereof comprise, respectively, amino acid sequences identical, except for 20 or fewer conservative amino acid substitutions each, to reference amino acid sequences selected from the group consisting of: SEQ ID NO:4 and SEQ ID NO:9; SEQ ID NO:14 and SEQ ID NO:19; SEQ ID NO:24 and SEQ ID NO:29; SEQ ID NO:34 and SEQ ID NO:39; SEQ ID NO:44 and SEQ ID NO:49; SEQ ID NO:54 and SEQ ID NO:59; SEQ ID NO:64 and SEQ ID NO:69; SEQ ID NO:74 and SEQ ID NO:79; SEQ ID NO:84 and SEQ ID NO:89; SEQ ID NO:94 and SEQ ID NO:99, SEQ ID NO:115 and SEQ ID NO:120, SEQ ID NO:125 and SEQ ID NO:130, SEQ ID NO:135 and SEQ ID NO:140, and SEQ ID NO:145 and SEQ ID NO:150.

12-24. (canceled)

25. The isolated antibody or fragment thereof of claim 1 which specifically binds to RON, wherein the VH of said antibody or fragment thereof comprises VH-CDR1, VH-CDR2, and VH-CDR3 amino acid sequences selected from the group consisting of: SEQ ID NOs: 5, 6, and 7; SEQ ID NOs: 15, 16, and 17; SEQ ID NOs: 25, 26, and 27; SEQ ID NOs: 35, 36, and 37; SEQ ID NOs: 45, 46, and 47; SEQ ID NOs: 55, 56, and 57; SEQ ID NOs: 65, 66, and 67; SEQ ID NOs: 75, 76, and 77; SEQ ID NOs: 85, 86, and 87; SEQ ID NOs: 95, 96, and 97; SEQ ID NOs: 116, 117, and 118; SEQ ID NOs: 126, 127, and 128; SEQ ID NOs: 136, 137, and 138; and SEQ ID NOs: 146, 147, and 148 except for one, two, three, or four amino acid substitutions in at least one of said VH-CDRs.

26. (canceled)

27. The isolated antibody or fragment thereof of claim 1 which specifically binds to RON, wherein the VL of said antibody or fragment thereof comprises VL-CDR1, VL-CDR2, and VL-CDR3 amino acid sequences selected from the group consisting of: SEQ ID NOs: 10, 11, and 12; SEQ ID NOs: 20, 21, and 22; SEQ ID NOs: 30, 31, and 32; SEQ ID NOs: 40, 41, and 42; SEQ ID NOs: 50, 51, and 52; SEQ ID NOs: 60, 61, and 62; SEQ ID NOs: 70, 71, and 72; SEQ ID NOs: 80, 81, and 82; SEQ ID NOs: 90, 91, and 92; SEQ ID NOs: 100, 101, and 102; SEQ ID NOs: 121, 122, and 123; SEQ ID NOs: 131, 132, and 133; SEQ ID NOs: 141, 142, and 143; and SEQ ID NOs: 151, 152, and 153 except for one, two, three, or four amino acid substitutions in at least one of said VL-CDRs.

28-57. (canceled)

58. The antibody or fragment thereof of claim 1, which blocks MSP from binding to RON.

59-62. (canceled)

63. The antibody or fragment thereof of claim 1, which inhibits RON-mediated cell proliferation.

64. The antibody or fragment thereof of claim 1, which inhibits tumor cell growth, tumor cell invasion or tumor cell metastasis.

65. The antibody or fragment thereof of claim 1, which induces apoptosis.

66. (canceled)

67. The antibody or fragment thereof of claim 1, wherein said antibody is conjugated to an agent selected from the group consisting of cytotoxic agent, a therapeutic agent, cytostatic agent, a biological toxin, a prodrug, a peptide, a protein, an enzyme, a virus, a lipid, a biological response modifier, pharmaceutical agent, a lymphokine, a heterologous antibody or fragment thereof, a detectable label, polyethylene glycol (PEG), and a combination of two or more of any said agents.

68-69. (canceled)

70. A composition comprising the antibody or fragment thereof of claim 1, and a carrier.

71-96. (canceled)

97. An isolated polynucleotide comprising a nucleic acid which encodes an antibody VH polypeptide, wherein said VH polypeptide comprises VH-CDR1, VH-CDR2, and VH-CDR3 amino acid sequences selected from the group consisting of: SEQ ID NOs: 5, 6, and 7; SEQ ID NOs: 15, 16, and 17; SEQ ID NOs: 25, 26, and 27; SEQ ID NOs: 35, 36, and 37; SEQ ID NOs: 45, 46, and 47; SEQ ID NOs: 55, 56, and 57; SEQ ID NOs: 65, 66, and 67; SEQ ID NOs: 75, 76, and 77; SEQ ID NOs: 85, 86, and 87; SEQ ID NOs: 95, 96, and 97; SEQ ID NOs: 116, 117, and 118; SEQ ID NOs: 126, 127, and 128; SEQ ID NOs: 136, 137, and 138; and SEQ ID NOs: 146, 147, and 148; and wherein an antibody or antigen binding fragment thereof comprising said VH specifically binds to RON.

98. An isolated polynucleotide comprising a nucleic acid which encodes an antibody VL polypeptide, wherein said VL polypeptide comprises VL-CDR1, VL-CDR2, and VL-CDR3 amino acid sequences selected from the group consisting of: 10, 11, and 12; SEQ ID NOs: 20, 21, and 22; SEQ ID NOs: 30, 31, and 32; SEQ ID NOs: 40, 41, and 42; SEQ ID NOs: 50, 51, and 52; SEQ ID NOs: 60, 61, and 62; SEQ ID NOs: 70, 71, and 72; SEQ ID NOs: 80, 81, and 82; SEQ ID NOs: 90, 91, and 92; SEQ ID NOs: 100, 101, and 102; SEQ ID NOs: 121, 122, and 123; SEQ ID NOs: 131, 132 and 133; SEQ ID NOs: 141, 142, and 143; and SEQ ID NOs: 151, 152, and 153; and wherein an antibody or antigen binding fragment thereof comprising said VL specifically binds to RON.

99-151. (canceled)

152. An isolated polypeptide encoded by the polynucleotide of claim 97.

153-156. (canceled)

157. A composition comprising an isolated VH encoding polynucleotide and an isolated VL encoding polynucleotide, wherein said VH encoding polynucleotide and said VL encoding polynucleotide, respectively, comprise nucleic acids encoding amino acid sequences identical, except for less than 20 conservative amino acid substitutions, to reference amino acid sequences selected from the group consisting of SEQ ID NO:4 and SEQ ID NO:9; SEQ ID NO:14 and SEQ ID NO:19; SEQ ID NO:24 and SEQ ID NO:29; SEQ ID NO:34 and SEQ ID NO:39; SEQ ID NO:44 and SEQ ID NO:49; SEQ ID NO:54 and SEQ ID NO:59; SEQ ID NO:64 and SEQ ID NO:69; SEQ ID NO:74 and SEQ ID NO:79; SEQ ID NO:84 and SEQ ID NO:89; SEQ ID NO:94 and SEQ ID NO:99; SEQ ID NO: 115 and SEQ ID NO:120; SEQ ID NO: 125 and SEQ ID NO:130; SEQ ID NO: 135 and SEQ ID NO:140; and SEQ ID NO: 145 and SEQ ID NO:150; and wherein an antibody or fragment thereof encoded by said VH and VL encoding polynucleotides specifically binds RON.

158-223. (canceled)

224. A host cell comprising the composition of claim 157.

225. A method of producing an antibody or fragment thereof which specifically binds RON, comprising culturing the host cell of claim 224, and recovering said antibody, or fragment thereof.

226. An antibody or fragment thereof which specifically binds RON, produced by the method of claim 225.

227. A method for treating a hyperproliferative disorder in an animal, comprising administering to an animal in need of treatment a composition comprising:
a) the isolated antibody or fragment thereof of claim 1; and
b) a pharmaceutically acceptable carrier.

228. The method of claim 227, wherein said hyperproliferative disease or disorder is selected from the group consisting of cancer, a neoplasm, a tumor, a malignancy, or a metastasis thereof.

229-239. (canceled)

240. An isolated polypeptide encoded by the polynucleotide of claim 98.
